(19) United States
(12) Patent Application Publication
(10) Pub. No.: US 2007/0203183 A1
(43) Pub. Date: Aug. 30, 2007

(54) Diaryl piperidines as CB1 modulators

(75) Inventors: Jack D. Scott, Scotch Plains, NJ (US); Jay Weinstein, Upper Montclair, NJ (US); Michael W. Miller, Scotch Plains, NJ (US); Andrew W. Stamford, Chatham, NJ (US); Eric J. Gilbert, Scotch Plains, NJ (US); Yan Xia, Edison, NJ (US); William J. Greenlee, Teaneck, NJ (US); Sarah Wei Li, Belle Mead, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/652,227

(22) Filed: Jan. 11, 2007

Related U.S. Application Data
(60) Provisional application No. 60/759,091, filed on Jan. 13, 2006.
(60) Provisional application No. 60/802,990, filed on May 24, 2006.

(51) Int. Cl. A61K 31/454; C07D 211/54; A61K 31/445; C07D 401/02

(52) U.S. Cl. 514/317; 514/326; 546/216; 546/207

(57) ABSTRACT

A compound having the general structure of Formula (I): 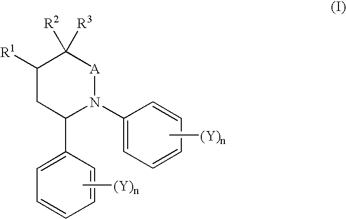 or a pharmaceutically acceptable salt, solvate, or ester thereof, are useful in treating diseases, disorders, or conditions such as metabolic syndrome (e.g., obesity, waist circumference, lipid profile, and insulin sensitivity), neuroinflammatory disorders, cognitive disorders, psychosis, addictive behavior, gastrointestinal disorders, and cardiovascular conditions.

This application claims the benefit of U.S. Provisional Application No. 60/759,091, filed Jan. 13, 2006 and Provisional Application No. 60/802,990, filed May 24, 2006.

### FIELD OF THE INVENTION

The present invention relates to diaryl piperidine compounds useful as CB₁ modulators (e.g., CB₁ antagonists, agonists or inverse agonists), pharmaceutical compositions comprising such compounds, and methods of treatment using the compounds and compositions to treat conditions such as metabolic syndrome, neuroinflammatory disorders, cognitive or psychiatric disorders, psychosis, addictive behaviors such as eating disorders, alcoholism and drug dependence, gastrointestinal disorders, cardiovascular conditions, weight reduction, lowering of waist circumference, treatment of dyslipidemia, insulin sensitivity, diabetes mellitus, hypertriglyceridemia, inflammation, migraine, nicotine dependence, Parkinson's disease, schizophrenia, sleep disorder, attention deficit hyperactivity disorder, male sexual dysfunction, premature ejaculation, premenstrual syndrome, seizure, epilepsy & convulsion, non-insulin dependent diabetes, dementia, major depressive disorder, bulimia nervosa, drug dependence, septic shock, cognitive disorder, endocrine disorders, eczema, emesis, allergy, glaucoma, hemorrhagic shock, hypertension, angina, thrombosis, atherosclerosis, restenosis, acute coronary syndrome, angina pectoris, arrhythmia, heart failure, cerebral ischemia, stroke, myocardial infarction, glomerulonephritis, thrombotic and thromboembolytic stroke, peripheral vascular diseases, neurodegenerative disease, osteoporosis, pulmonary disease, autoimmune disease, hypotension, arthropathy, cancer, demyelinating diseases, Alzheimer's disease, hypoactive sexual desire disorder, bipolar disorder, hyperlipidemia, narcotic dependence, Huntington's chorea, pain, multiple sclerosis, anxiety disorder, bone disorders such as osteoporosis, Paget's disease, rheumatoid arthritis, ulcerative colitis, irritable bowel syndrome and inflammatory bowel diseases.

### BACKGROUND OF THE INVENTION

The CB₁ receptor is one of the most abundant neuromodulatory receptors in the brain, and is expressed at high levels in the hippocampus, cortex, cerebellum, and basal ganglia (e.g., Wilson et al., Science, 2002, vol. 296, 678-682). Selective CB₁ receptor antagonists, for example pyrazole derivatives such as rimonabant (e.g., U.S. Pat. No. 6,432,984), can be used to treat various conditions, such as obesity and metabolic syndrome (e.g., Bensaid et al., Molecular Pharmacology, 2003 vol. 63, no. 4, pp. 908-914; Trillou et al., Am. J. Physiol. Regul. Integr. Comp. Physiol. 2002 vol. 284, R345-R353; Kirkham, Am. J. Physiol. Regul. Integr. Comp. Physiol. 2002 vol. 284, R343-R344), neuroinflammatory disorders (e.g., Adam, et al., Expert Opin. Ther. Patents, 2002, vol. 12, no. 10, 1475-1489; U.S. Pat. No. 6,642,258), cognitive disorders and psychosis (e.g., Adam et al., Expert Opin. Ther. Pat., 2002, vol. 12, pp. 1475-1489), addiction (e.g., smoking cessation; U.S. Patent Publ. 2003/0087933), gastrointestinal disorders (e.g., Lange et al., J. Med. Chem. 2004, vol. 47, 627-643) and cardiovascular conditions (e.g., Porter et al., Pharmacology and Therapeutics, 2001 vol. 90, 45-60; Sanofi-Aventis Publication, Bear Steams Conference, New York, Sep. 14, 2004, pages 19-24).

However, there is still a need for improved cannabinoid agents, particularly selective CB₁ receptor antagonists, with fewer side-effects and improved efficacy. It is therefore an object of the present invention to provide substituted piperazines useful in the treatment of diseases or conditions mediated by CB₁ receptors.

U.S. Patent Application Publication U.S. 2004/0167185 describes Edg-3 receptor inhibitors including substituted piperidines. U.S. Patent Application Publication U.S. 2002/0128476 and U.S. Patent Application Publication U.S. 2004/0180927 describe 3-piperidinone and 3-piperidinol cysteine protease inhibitors. U.S. Patent Application Publication U.S. 2001/0006972 describes aryl piperidine NK-1 receptor antagonists. U.S. Patent Application Publication U.S. 2003/0171588 describes piperidine-3-carboxamide derivatives. U.S. Pat. No. 5,234,895 describes 2-arylpyridone herbicides. U.S. Pat. No. 5,185,349 describes lactam ACAT inhibitors. U.S. Pat. No. 4,839,360 describes 1,6-diaryl-2-piperidones. U.S. Pat. No. 6,369,077 describes protease inhibitors. U.S. Pat. No. 5,332,817 describes 3-aminopiperidine derivatives. WO 03/062392 describes Edg-2, Edg-3, Edg-4, and Edg-7 antagonists. U.S. Pat. No. 5,580,883 describes piperidine derivatives which present nerve degeneration. U.S. Pat. No. 6,441,001 describes piperidine derivatives as CCR3 modulators. Weis et al., Tetrahedron, 59 (2003) 1403-1411, describe the synthesis of diphenylpyralines. Josephsohn et al., J. Am. Chem. Soc., 125 (2003) 4018-4019 describe methods of preparing diarylpiperidines. However, the compounds disclosed in each of the above references differ substantially from the compounds of the present invention.

### SUMMARY OF THE INVENTION

In its many embodiments, the present invention provides a novel class of substituted piperazine compounds as selective CB₁ receptor antagonists for treating various conditions including, but not limited to metabolic syndrome, neuroinflammatory disorders, cognitive or psychiatric disorders, psychosis, addictive behaviors such as eating disorders, alcoholism and drug dependence, gastrointestinal disorders, cardiovascular conditions, weight reduction, lowering of waist circumference, treatment of dyslipidemia, insulin sensitivity, diabetes mellitus, hypertriglyceridemia, inflammation, migraine, nicotine dependence, Parkinson's disease, schizophrenia, sleep disorder, attention deficit hyperactivity disorder, male sexual dysfunction, premature ejaculation, premenstrual syndrome, seizure, epilepsy & convulsion, non-insulin dependent diabetes, dementia, major depressive disorder, bulimia nervosa, drug dependence, septic shock, cognitive disorder, endocrine disorders, eczema, emesis, allergy, glaucoma, hemorrhagic shock, hypertension, angina, thrombosis, atherosclerosis, restenosis, acute coronary syndrome, angina pectoris, arrhythmia, heart failure, cerebral ischemia, stroke, myocardial infarction, glomerulonephritis, thrombotic and thromboembolytic stroke, peripheral vascular diseases, neurodegenerative disease, osteoporosis, pulmonary disease, autoimmune disease, hypotension, arthropathy, cancer, demyelinating diseases, Alzheimer's disease, hypoactive sexual desire disorder, bipolar disorder, hyperlipidemia, narcotic dependence, Huntington's chorea, pain, multiple sclerosis, anxiety disorder, bone disorders such as osteoporosis, Paget's disease, rheumatoid arthritis, ulcerative colitis, irritable bowel syndrome and inflammatory bowel diseases.

The selective CB₁ receptor antagonists of the present invention are piperazine derivatives having the structure of Formula (I): 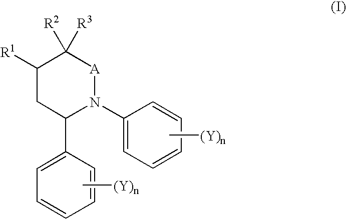
or a pharmaceutically acceptable salt, solvate, or ester thereof, wherein:
A is —CH₂— or —C(O)—;
R¹ is selected from the group consisting of H, —N(R⁴)(R⁵), unsubstituted heterocyclyl, heterocyclyl substituted with one or more X groups, —N₃, and —O—R⁷;
R² is selected from the group consisting of H, —(C(R⁶)₂)ₚ-aryl, cycloalkylalkyl, cycloalkylalkyl substituted with Z, —(C(R⁶)₂)_{q}-heterocyclyl, —(C(R⁶)₂)ₚ—S(O)₂-heterocyclyl, and —C(R⁶)₂—O—R⁷,
   wherein the aryl portion of said —C(R⁶)₂-aryl of R² is unsubstituted or substituted with one or more Y groups,
   wherein the heterocyclyl portion of said —(C(R⁶)₂)ₚ—S(O)₂-heterocyclyl of R² is unsubstituted or substituted with one or more X groups,
   wherein the heterocyclyl portion of said —(C(R⁶)₂)_{q}-heterocyclyl of R² is unsubstituted or substituted with one or more X groups;
R³ is selected from the group consisting of H, —C(R⁶)₂-aryl, —C(R⁶)₂—O—R⁷, —(C(R⁶)₂)_{q}—C(O)—N(R²)₂, —(C(R⁶)₂)ₚ—N(R⁹)—C(O)—(C(R⁶)₂)_{q}—R⁶, —(C(R⁶)₂)_{q}—S(O)₂—N(R⁹)—(C(R⁶)₂)_{q}—R¹⁵, —(C(R⁶)₂)_{q}—N(R⁹)—S(O)₂—(C(R⁶)₂)_{q}—R¹⁵ and —(C(R⁶)₂)_{q}—N(R⁸)₂,
   wherein the aryl portion of said —C(R⁶)₂-aryl of R³ is unsubstituted or substituted with one or more Y groups;
   with the following independent provisos:
   (i) at least one of R¹, R², and R³ is not H;
   (ii) when R¹ is —OH, at least one of R² and R³ is not H; and
   (iii) when A is —C(O)—, at least one of R² and R³ is not H;
or, R² and R³ together with the ring carbon atom to which they are shown attached form an unsubstituted heterocyclyl ring or a heterocyclyl ring substituted with one or more X groups;
R⁴ is selected from the group consisting of H, —C(O)-alkyl, and alkyl;
R⁵ is selected from the group consisting of —C(R⁶)₂)ₘ-G, —S(O)₂-alkyl, —S(O)₂-cycloalkyl, alkyl, —S(O)-cycloalkyl, —C(O)-cycloalkyl, —S(O)₂-aryl, —S(O)₂—(C(R⁶)₂)ₘ-aryl, —S(O)₂-heteroaryl, —C(O)alkyl, —C(O)-aryl, —C(O)—O-alkyl, —C(O)O-aryl, —C(O)—(C(R⁶)₂)ₘ-aryl, —C(O)-cycloalkylene-aryl, —C(O)-heteroaryl, —C(O)-heteroarylalkyl, —C(O)—(C(R⁶)₂)ₘ—O-aryl, —C(O)-(benzo-fused cycloalkyl), —S(O)₂-(benzo-fused heterocyclyl), —C(O)—N(R⁹)—(C(R⁶)₂)ₘ-aryl, —C(O)—N(R⁹)-aryl, cycloalkyl, benzo-fused cycloalkyl, unsubstituted aryl, aryl substituted with one or more Y groups, unsubstituted heterocyclyl, and heterocyclyl substituted with one or more X groups,
   wherein the aryl or heteroaryl portion of said —S(O)₂-aryl, —S(O)₂—(C(R⁶)₂)ₘ-aryl, —S(O)₂-heteroaryl, —C(O)-aryl, —C(O)—(C(R⁶)₂)ₘ-aryl, —C(O)-cycloalkylene-aryl, —C(O)-heteroaryl, —C(O)—(C(R⁶)₂)ₘ—O-aryl, —C(O)—N(R⁹)—(C(R⁶)₂)ₘ-aryl, or —C(O)—N(R⁹)-aryl of R⁵ is unsubstituted or substituted with one or more Y groups;
   wherein the heterocyclyl portion of —S(O)₂-(benzo-fused heterocyclyl)aryl of R⁵ is unsubstituted or substituted with one or more X groups;
each R⁶ is independently selected from the group consisting of H and alkyl;
R⁷ is selected from the group consisting of H, alkyl, unsubstituted heteroaryl and heteroaryl substituted with one or more Y groups, unsubstituted aryl, and aryl substituted with one or more Y groups;
each R⁸ is independently selected from the group consisting of H, alkyl, arylalkyl, heteroarylalkyl, unsubstituted aryl, unsubstituted heteroaryl, —C(O)-alkyl, —C(O)-aryl, —C(O)-cycloalkyl, —C(O)N(R⁹)₂, —S(O)₂-aryl, —S(O)₂-heteroaryl, —SO₂N(R⁹)₂, —S(O)₂-cycloalkyl, aryl and heteroaryl substituted with one or more Y groups, and —S(O)₂-alkyl,
   wherein the aryl portion of said arylalkyl, —C(O)-aryl or —S(O)₂-aryl and the heteroaryl portion of said heteroarylalkyl, —S(O)₂-heteroaryl of R⁸ is unsubstituted or substituted with one or more Y groups,
   wherein the alkyl portion of said arylalkyl and heteroarylalkyl is unsubstituted or substituted with one or more X groups with the proviso that X substituted on said alkyl portion is NOT Cbz or Boc;
each R⁹ is independently selected from the group consisting of H, alkyl, haloalkyl, hydroxyalkyl, cycloalkyl, unsubstituted aryl and unsubstituted heteroaryl;
each R¹² is independently selected from the group consisting of H, alkyl, cycloalkylalkyl, —(C(R⁶)₂)_{q}—C(O)R¹³, benzoheterocyclyl, benzocycloalkyl, —(C(R⁶)₂)_{q}—N(R⁹)—C(O)R¹³, —(C(R⁶)₂)_{q}—N(R¹⁴)₂, arylalkyl, heteroarylalkyl, HO-alkyl-, alkyl-O—, aryl-O—, Y-alkylenyl-O—, W—O-alkylenyl, heterocyclylalkyl, unsubstituted cycloalkyl, cycloalkyl substituted with one or more X groups, unsubstituted heterocyclyl, heterocyclyl substituted with one or more X groups, unsubstituted heteroaryl, heteroaryl substituted with one or more Y groups, unsubstituted aryl and aryl substituted with one or more Y groups, and
   wherein the aryl and heteroaryl portion of said arylalkyl and heteroarylalkyl is unsubstituted or substituted with one or more Y groups,
   wherein the alkyl portion of said cycloalkylalkyl, arylalkyl and heteroarylalkyl is unsubstituted or substituted with one or more X groups with the proviso that X substituted on said alkyl portion is NOT Cbz or Boc,
   wherein the cycloalkyl of said cycloalkylalkyl is unsubstituted or substituted with one or more X groups,
   wherein the benzo portion of said benzoheterocyclyl can be optionally substituted with one or more Y groups and the heterocyclyl portion of benzoheterocyclyl can be optionally substituted with one or more X groups,
   wherein the benzo portion of said benzocycloalkyl can be optionally substituted with one or more Y groups and the cycloalkyl portion of benzocycloalkyl can be optionally substituted with one or more X groups;
   with the following provisos that
   for —N(R¹⁴)₂ of R¹², the two R¹⁴ groups, with the ring nitrogen atom to which they are shown attached, form an unsubstituted heterocyclyl ring or a heterocyclyl ring substituted with one or more X groups;
each R¹³ is independently selected from the group consisting of H, alkyl, cycloalkylalkyl, arylalkyl, heteroarylalkyl, HO-alkyl-, alkyl-O—, aryl-O—, unsubstituted cycloalkyl, cycloalkyl substituted with one or more X groups, unsubstituted heterocyclyl, heterocyclyl substituted with one or more X groups, unsubstituted heteroaryl, heteroaryl substituted with one or more Y groups, unsubstituted aryl and aryl substituted with one or more Y groups
   wherein the aryl and heteroaryl portion of said arylalkyl and heteroarylalkyl is unsubstituted or substituted with one or more Y groups,
   wherein the alkyl portion of said cycloalkylalkyl, arylalkyl and heteroarylalkyl is unsubstituted or substituted with one or more X groups with the proviso that X substituted on said alkyl portion is NOT Cbz or Boc,
   wherein the cycloalkyl of said cycloalkylalkyl is unsubstituted or substituted with one or more X groups;
each R¹⁴ is independently selected from the group consisting of H, Boc, unsubstituted alkyl, alkyl substituted with one or more X groups, unsubstituted cycloalkyl, cycloalkyl substituted with one or more Y groups, unsubstituted aryl, aryl substituted with one or more Y groups, heterocyclyl, unsubstituted heteroaryl and heteroaryl substituted with one or more Y groups;
   each R¹⁵ is independently selected from the group consisting of H, alkyl, —N(R⁴)(R⁵), (C(R⁶)₂)_{q}—N(R¹⁴)₂, alkylenyl-CF₃, —CF₃, cycloalkylalkyl, unsubstituted cycloalkyl, cycloalkyl substituted with one or more X groups, unsubstituted heterocyclyl, heterocyclyl substituted with one or more X groups, benzoheterocyclyl, benzocycloalkyl, unsubstituted heteroaryl, heteroaryl substituted with one or more Y groups, unsubstituted aryl and aryl substituted with one or more Y groups,
   wherein the alkyl portion of said cycloalkylalkyl is unsubstituted or substituted with one or more X groups with the proviso that X substituted on said alkyl portion is NOT Cbz or Boc,
   wherein the cycloalkyl of said cycloalkylalkyl is unsubstituted or substituted with one or more X groups,
   wherein the benzo portion of said benzoheterocyclyl can be optionally substituted with one or more Y groups and the heterocyclyl portion of benzoheterocyclyl can be optionally substituted with one or more X groups,
   wherein the benzo portion of said benzocycloalkyl can be optionally substituted with one or more Y groups and the cycloalkyl portion of benzocycloalkyl can be optionally substituted with one or more X groups;
each R¹⁶ is independently selected from the group consisting of H, alkyl, cycloalkylalkyl, —(C(R⁶)₂)ₚ—C(O)R¹³, —(C(R⁶)₂)ₚ—N(R⁹)—C(O)R¹³, —(C(R⁶)₂)ₚ—N(R¹⁴)₂, arylalkyl, heteroarylalkyl, HO-alkyl-, alkyl-O—, aryl-O—, unsubstituted cycloalkyl, cycloalkyl substituted with one or more X groups, unsubstituted heterocyclyl, heterocyclyl substituted with one or more X groups, unsubstituted heteroaryl, heteroaryl substituted with one or more Y groups, unsubstituted aryl and aryl substituted with one or more Y groups, and
   wherein the aryl and heteroaryl portion of said arylalkyl and heteroarylalkyl is unsubstituted or substituted with one or more Y groups,
   wherein the alkyl portion of said cycloalkylalkyl, arylalkyl and heteroarylalkyl is unsubstituted or substituted with one or more X groups with the proviso that X substituted on said alkyl portion is NOT Cbz or Boc,
   wherein the cycloalkyl of said cycloalkylalkyl is unsubstituted or substituted with one or more X groups,
   for —N(R¹⁴)₂, the two R¹⁴ groups, with the ring nitrogen atom to which they are shown attached, form an unsubstituted heterocyclyl ring or a heterocyclyl ring substituted with one or more X groups;
G is selected from the group consisting of H, alkyl, unsubstituted aryl, aryl substituted with one or more Y groups, —CN, cycloalkyl, —O—R⁷, —S—R⁷, unsubstituted heteroaryl, heteroaryl substituted with one or more Y groups, —N(R⁸)₂, unsubstituted heterocyclyl, and heterocyclyl substituted with one or more X groups;
each W is independently selected from the group consisting of hydrogen, alkyl, aryl, —C(O)-alkyl, —C(O)—O-alkyl, —C(R⁶)₂—N(R⁶)₂, and —C(R⁶)₂—N(R⁶)—S(O)₂—R⁶;
each X is independently selected from the group consisting of hydrogen, —OH, alkyl, arylalkyl, heteroarylalkyl, Cbz, Boc, alkylsulfonyl, acetyl, —C(O)—R², —C(O)—N(R⁹)₂, —C(O)-heteroaryl, heteroaryl, —S(O)₂-cycloalkyl, —C(O)-alkyl, —C(O)—O-alkyl, —C(R⁶)₂)ₘ-aryl and aryl
   wherein the aryl and heteroaryl portion of said arylalkyl and heteroarylalkyl is unsubstituted or substituted with one or more Y groups,
   wherein the alkyl portion of said arylalkyl and heteroarylalkyl is unsubstituted or substituted with one or more X groups with the proviso that X substituted on said alkyl portion is NOT Cbz or Boc,
      wherein said heteroaryl or the heteroaryl portion of said —C(O)-heteroaryl of X is unsubstituted or substituted with one or more substituents selected from the group consisting of halogen, —OH, —O-alkyl, haloalkyl, and —CN, and
      wherein said aryl or the aryl portion of said —C(R⁶)₂)ₘ-aryl of X is unsubstituted or substituted with one or more substituents selected from the group consisting of halogen, —OH, —O-alkyl, haloalkyl, and —CN
      wherein in a single X moiety, ═O, can replace two available hydrogens on the same carbon on a ring system;
each Y is independently selected from the group consisting of hydrogen, halogen, alkyl, aryl, —C(O)-alkyl, —O-alkyl, —O-heteroaryl, —O-aryl, —O—R⁹, haloalkyl, —O-haloalkyl, —CN, —C(O)—O-alkyl, —N(R⁶)₂, —C(R⁶)₂—N(R⁶)₂, —S(O)₂-heterocyclyl, —S(O)₂-heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each Z is independently selected from the group consisting of hydrogen, alkyl, arylalkyl, heteroarylalkyl, —C(O)—N(R⁹)₂, —C(O)-heteroaryl, heteroaryl, —S(O)₂-cycloalkyl, —C(O)-alkyl, —C(R⁶)₂)ₘ-aryl, —N(R⁶)—S(O)₂—R⁹ and aryl
   wherein the aryl and heteroaryl portion of said arylalkyl and heteroarylalkyl is unsubstituted or substituted with one or more Y groups,
   wherein the alkyl portion of said arylalkyl and heteroarylalkyl is unsubstituted or substituted with one or more X groups with the proviso that X substituted on said alkyl portion is NOT Cbz or Boc,
      wherein said heteroaryl or the heteroaryl portion of said —C(O)-heteroaryl of Z is unsubstituted or substituted with one or more substituents selected from the group consisting of halogen, —OH, —O-alkyl, haloalkyl, and —CN, and
      wherein said aryl or the aryl portion of said —C(R⁶)₂)ₘ-aryl of Z is unsubstituted or substituted with one or more substituents selected from the group consisting of halogen, —OH, —O-alkyl, haloalkyl, and —CN;
      wherein in a single Z moiety, ═O, can replace two available hydrogens on the same carbon on a ring system;
each n, p and q is independently an integer from 0-5; and
m is an integer from 1-5.

In another embodiment, the present invention is directed to a pharmaceutical composition comprising a therapeutically effective amount of at least one compound of Formula (I), or a pharmaceutically acceptable salt, solvate, or ester thereof, and at least one pharmaceutically acceptable carrier.

In another embodiment, the present invention is directed to a method of treating a disease or disorder in a patient, such as metabolic syndrome, neuroinflammatory disorders, cognitive or psychiatric disorders, psychosis, addictive behaviors such as eating disorders, alcoholism and drug dependence, gastrointestinal disorders, cardiovascular conditions, weight reduction, lowering of waist circumference, treatment of dyslipidemia, insulin sensitivity, diabetes mellitus, hypertriglyceridemia, inflammation, migraine, nicotine dependence, Parkinson's disease, schizophrenia, sleep disorder, attention deficit hyperactivity disorder, male sexual dysfunction, premature ejaculation, premenstrual syndrome, seizure, epilepsy & convulsion, non-insulin dependent diabetes, dementia, major depressive disorder, bulimia nervosa, drug dependence, septic shock, cognitive disorder, endocrine disorders, eczema, emesis, allergy, glaucoma, hemorrhagic shock, hypertension, angina, thrombosis, atherosclerosis, restenosis, acute coronary syndrome, angina pectoris, arrhythmia, heart failure, cerebral ischemia, stroke, myocardial infarction, glomerulonephritis, thrombotic and thromboembolytic stroke, peripheral vascular diseases, neurodegenerative disease, osteoporosis, pulmonary disease, autoimmune disease, hypotension, arthropathy, cancer, demyelinating diseases, Alzheimer's disease, hypoactive sexual desire disorder, bipolar disorder, hyperlipidemia, narcotic dependence, Huntington's chorea, pain, multiple sclerosis, anxiety disorder, bone disorders such as osteoporosis, Paget's disease, rheumatoid arthritis, ulcerative colitis, irritable bowel syndrome and inflammatory bowel diseases. The method comprises administering to the patient an effective amount of at least one compound of Formula (I), or a pharmaceutically acceptable salt, solvate, or ester thereof.

In another embodiment, the present invention is directed to a method of treating a disease or disorder in a patient, such as metabolic syndrome, neuroinflammatory disorders, cognitive or psychiatric disorders, psychosis, addictive behaviors such as eating disorders, alcoholism and drug dependence, gastrointestinal disorders, cardiovascular conditions, weight reduction, lowering of waist circumference, treatment of dyslipidemia, insulin sensitivity, diabetes mellitus, hypertriglyceridemia, inflammation, migraine, nicotine dependence, Parkinson's disease, schizophrenia, sleep disorder, attention deficit hyperactivity disorder, male sexual dysfunction, premature ejaculation, premenstrual syndrome, seizure, epilepsy & convulsion, non-insulin dependent diabetes, dementia, major depressive disorder, bulimia nervosa, drug dependence, septic shock, cognitive disorder, endocrine disorders, eczema, emesis, allergy, glaucoma, hemorrhagic shock, hypertension, angina, thrombosis, atherosclerosis, restenosis, acute coronary syndrome, angina pectoris, arrhythmia, heart failure, cerebral ischemia, stroke, myocardial infarction, glomerulonephritis, thrombotic and thromboembolytic stroke, peripheral vascular diseases, neurodegenerative disease, osteoporosis, pulmonary disease, autoimmune disease, hypotension, arthropathy, cancer, demyelinating diseases, Alzheimer's disease, hypoactive sexual desire disorder, bipolar disorder, hyperlipidemia, narcotic dependence, Huntington's chorea, pain, multiple sclerosis, anxiety disorder, bone disorders such as osteoporosis, Paget's disease, rheumatoid arthritis, ulcerative colitis, irritable bowel syndrome and inflammatory bowel diseases. The method comprises administering to the patient an effective amount of at least one compound of Formula (I), or a pharmaceutically acceptable salt, solvate, or ester thereof, in combination with at least one other pharmaceutical compound, such as a cholesterol lowering compound.

### DETAILED DESCRIPTION OF THE INVENTION

In a first embodiment, the present invention is directed to a compound of Formula (I), or a pharmaceutically acceptable salt, solvate, or ester thereof, as described herein.

In another embodiment of a compound of Formula (I), or a pharmaceutically acceptable salt, solvate, or ester thereof,
A is —CH₂— or —C(O)—;
R¹ is selected from the group consisting of H, —N(R⁴)(R⁵), unsubstituted (C₂-C₁₀)heterocyclyl, (C₂-C₁₀)heterocyclyl substituted with one or more X groups, —N₃, and —O—R⁷;
R² is selected from the group consisting of H, —(C(R⁶)₂)ₚ—(C₆-C₁₀)aryl, (C₃-C₆)cycloalkyl(C₁-C₆)alkyl, (C₃-C₆)cycloalkyl(C₁-C₆)alkyl substituted with Z, —(C(R⁶)₂)_{q}—(C₂-C₁₀)heterocyclyl, —(C(R⁶)₂)ₚ—S(O)₂—(C₂-C₁₀)heterocyclyl, and —C(R⁶)₂—O—R⁷,
   wherein the (C₆-C₁₀)aryl portion of said —C(R⁶)₂—(C₆-C₁₀)aryl of R² is unsubstituted or substituted with one or more Y groups,
   wherein the (C₂-C₁₀)heterocyclyl portion of said —(C(R⁶)₂)ₚ—S(O)₂—(C₂-C₁₀)heterocyclyl of R² is unsubstituted or substituted with one or more X groups,
   wherein the (C₂-C₁₀)heterocyclyl portion of said —(C(R⁶)₂)_{q}—(C₂-C₁₀)heterocyclyl of R² is unsubstituted or substituted with one or more X groups;
R³ is selected from the group consisting of H, —C(R⁶)₂—(C₆-C₁₀)aryl, —C(R⁶)₂—O—R⁷, —(C(R⁶)₂)_{q}—C(O)—N(R¹²)₂, —(C(R⁶)₂)ₚ—N(R⁹)—C(O)—(C(R⁶)₂)_{q}—R¹⁶, —(C(R⁶)₂)_{q}—S(O)₂—N(R⁹)—(C(R⁶)₂)_{q}—R¹⁵, —(C(R⁶)₂)_{q}—N(R⁹)—S(O)₂—(C(R⁶)₂)_{q}—R¹⁵ and —(C(R⁶)₂)_{q}—N(R⁸)₂,
   wherein the (C₆-C₁₀)aryl portion of said —C(R⁶)₂—(C₆-C₁₀)aryl of R³ is unsubstituted or substituted with one or more Y groups;
   with the following independent provisos:
   (i) at least one of R¹, R², and R³ is not H;
   (ii) when R¹ is —OH, at least one of R² and R³ is not H; and
   (iii) when A is —C(O)—, at least one of R² and R³ is not H;
or, R² and R³ together with the ring carbon atom to which they are shown attached form an unsubstituted (C₂-C₁₀)heterocyclyl ring or a (C₂-C₁₀)heterocyclyl ring substituted with one or more X groups;
R⁴ is selected from the group consisting of H, —C(O)—(C₁-C₆)alkyl, and (C₁-C₆)alkyl;
R⁵ is selected from the group consisting of —C(R⁶)₂)ₘ-G, —S(O)₂—(C₁-C₆)alkyl, —S(O)₂—(C₃-C₆)cycloalkyl, (C₁-C₆)alkyl, —S(O)—(C₃-C₆)cycloalkyl, —C(O)—(C₃-C₆)cycloalkyl, —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₁-C₆)alkyl, —C(O)—(C₆-C₁₀)aryl, —C(O)—O—(C₁-C₆)alkyl, —C(O)—O—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C₂-C₁₀)heteroaryl(C₁-C₆)alkyl, —C(O)—(C(R⁶)₂)ₘ—O—(C₆-C₁₀)aryl, —C(O)-(benzo-fused (C₃-C₆)cycloalkyl), —S(O)₂-(benzo-fused (C₂-C₁₀)heterocyclyl), —C(O)—N(R⁹)—(C(R⁶)₂)ₘ—(C₆-C₁₀)aryl, —C(O)—N(R⁹)—(C₆-C₁₀)aryl, (C₃-C₆)cycloalkyl, benzo-fused (C₃-C₆)cycloalkyl, unsubstituted (C₆-C₁₀)aryl, (C₆-C₁₀)aryl substituted with one or more Y groups, unsubstituted (C₂-C₁₀)heterocyclyl, and (C₂-C₁₀)heterocyclyl substituted with one or more X groups,
   wherein the (C₆-C₁₀)aryl or (C₂-C₁₀)heteroaryl portion of said —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C(R⁶)₂)ₘ—O—(C₆-C₁₀)aryl, —C(O)—N(R⁹)—(C(R⁶)₂)ₘ—(C₆-C₁₀)aryl, or —C(O)—N(R⁹)—(C₆-C₁₀)aryl of R⁵ is unsubstituted or substituted with one or more Y groups;
   wherein the heterocyclyl portion of —S(O)₂-(benzo-fused (C₂-C₁₀)heterocyclyl)aryl of R⁵ is unsubstituted or substituted with one or more X groups;
each R⁶ is independently selected from the group consisting of H and (C₁-C₆)alkyl;
R⁷ is selected from the group consisting of H, (C₁-C₆)alkyl, unsubstituted (C₂-C₁₀)heteroaryl and (C₂-C₁₀)heteroaryl substituted with one or more Y groups, unsubstituted (C₆-C₁₀)aryl, and (C₆-C₁₀)aryl substituted with one or more Y groups;
each R⁸ is independently selected from the group consisting of H, (C₁-C₆)alkyl, (C₆-C₁₀)aryl(C₁-C₆)alkyl, (C₂-C₁₀)heteroaryl(C₁-C₆)alkyl, unsubstituted (C₆-C₁₀)aryl, unsubstituted (C₂-C₁₀)heteroaryl, —C(O)—(C₁-C₆)alkyl, —C(O)—(C₆-C₁₀)aryl, —C(O)—(C₃-C₆)cycloalkyl, —C(O)N(R⁹)₂, —S(O)₂—(C₆-C₁₀)aryl, —S(O)₂—(C₂-C₁₀)heteroaryl, —SO₂N(R⁹)₂, —S(O)₂—(C₃-C₆)cycloalkyl, (C₆-C₁₀)aryl and (C₂-C₁₀)heteroaryl substituted with one or more Y groups, and —S(O)₂—(C₁-C₆)alkyl,
   wherein the (C₆-C₁₀)aryl portion of said (C₆-C₁₀)aryl(C₁-C₆)alkyl, —C(O)—(C₆-C₁₀)aryl or —S(O)₂—(C₆-C₁₀)aryl and the (C₂-C₁₀)heteroaryl portion of said (C₂-C₁₀)heteroaryl(C₁-C₆)alkyl, —S(O)₂—(C₂-C₁₀)heteroaryl of R⁸ is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each R¹² is independently selected from the group consisting of H, (C₁-C₆)alkyl, (C₃-C₆)cycloalkyl(C₁-C₆)alkyl, —(C(R⁶)₂)_{q}—C(O)R¹³, benzo(C₂-C₁₀)heterocyclyl, benzocyclo(C₁-C₆)alkyl, —(C(R⁶)₂)_{q}—N(R⁹)—C(O)R¹³, —(C(R⁶)₂)_{q}—N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, Y—(C₁-C₆)alkylenyl-O—, W—O—(C₁-C₆)alkylenyl, (C₂-C₁₀)heterocyclyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups,
   wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
   wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
   with the following provisos that
   for —N(R¹⁴)₂ of R¹², the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each R¹³ is independently selected from the group consisting of H, (C₁-C₆)alkyl, (C₃-C₆)cycloalkyl(C₁-C₆)alkyl, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups;
each R¹⁴ is independently selected from the group consisting of H, Boc, unsubstituted (C₁-C₆)alkyl, (C₁-C₆)alkyl substituted with one or more X groups, unsubstituted (C₃-C₆)cycloalkyl, (C₃-C₆)cycloalkyl substituted with one or more Y groups, unsubstituted (C₆-C₁₀)aryl, (C₆-C₁₀)aryl substituted with one or more Y groups, (C₂-C₁₀)heterocyclyl, unsubstituted (C₂-C₁₀)heteroaryl and (C₂-C₁₀)heteroaryl substituted with one or more Y groups;
each R¹⁵ is independently selected from the group consisting of H, (C₁-C₆)alkyl, —N(R⁴)(R⁵), (C(R⁶)₂)_{q}—N(R¹⁴)₂, (C₁-C₆)alkylenyl-CF₃, —CF₃, (C₃-C₆)cycloalkyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, benzo(C₂-C₁₀)heterocyclyl, benzocyclo(C₁-C₆)alkyl, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups,
   wherein the (C₁-C₆)alkyl portion of said (C₃-C₆)cycloalkyl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups,
   wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
   wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
each R¹⁶ is independently selected from the group consisting of H, (C₁-C₆)alkyl, (C₃-C₆)cycloalkyl(C₁-C₆)alkyl, —(C(R⁶)₂)ₚ—C(O)R¹³, —(C(R⁶)₂)ₚ—N(R⁹)—C(O)R¹³, —(C(R⁶)₂)ₚ—N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups,
   for —N(R¹⁴)₂, the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
G is selected from the group consisting of H, (C₁-C₆)alkyl, unsubstituted (C₆-C₁₀)aryl, (C₆-C₁₀)aryl substituted with one or more Y groups, —CN, (C₃-C₆)cycloalkyl, —O—R⁷, —S—R⁷, unsubstituted (C₂-C₁₀)heteroaryl, (C₂-C₁₀)heteroaryl substituted with one or more Y groups, —N(R⁸)₂, unsubstituted (C₂-C₁₀)heterocyclyl, and (C₂-C₁₀)heterocyclyl substituted with one or more X groups;
each W is independently selected from the group consisting of hydrogen, (C₁-C₆)alkyl, (C₆-C₁₀)aryl, —C(O)—(C₁-C₆)alkyl, —C(O)O—(C₁-C₆)alkyl, —C(R⁶)₂—N(R⁶)₂, and —C(R⁶)₂—N(R⁶)—S(O)₂—R⁶;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)—O—(C₁-C₆)alkyl, —(C(R⁶)₂)ₘ—(C₆-C₁₀)aryl and (C₆-C₁₀)aryl
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
      wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
      wherein said (C₆-C₁₀)aryl or the (C₆-C₁₀)aryl portion of said —C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
      wherein in a single X moiety, ═O, can replace two available hydrogens on the same carbon on a ring system;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each Z is independently selected from the group consisting of hydrogen, (C₁-C₆)alkyl, (C₆-C₁₀)aryl(C₁-C₆)alkyl, (C₂-C₁₀)heteroaryl(C₁-C₆)alkyl, —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(R⁶)₂)ₘ—(C₆-C₁₀)aryl, —N(R⁶)—S(O)₂—R⁹ and (C₆-C₁₀)aryl
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
      wherein said (C₂-C₁₀)heteroaryl or the (C₂-C₁₀)heteroaryl portion of said —C(O)—(C₂-C₁₀)heteroaryl of Z is unsubstituted or substituted with one or more substituents selected from the group consisting of halogen, —OH, —O—(C₁-C₆)alkyl, halo(C₁-C₆)alkyl, and —CN, and
      wherein said (C₆-C₁₀)aryl or the (C₆-C₁₀)aryl portion of said —C(R⁶)₂)ₘ—(C₆-C₁₀)aryl of Z is unsubstituted or substituted with one or more substituents selected from the group consisting of halogen, —OH, —O—(C₁-C₆)alkyl, halo(C₁-C₆)alkyl, and —CN;
      wherein in a single Z moiety, ═O, can replace two available hydrogens on the same carbon on a ring system;
each n, p and q is independently an integer from 0-5; and
m is an integer from 1-5.

In another embodiment, the compound of Formula (I) is a compound having the structural Formula (IA): 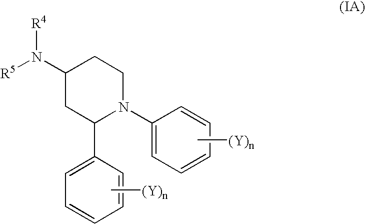
or a pharmaceutically acceptable salt, solvate or ester thereof, wherein:
R⁴ is selected from the group consisting of H, —C(O)—(C₁-C₆)alkyl, and (C₁-C₆)alkyl;
R⁵ is selected from the group consisting of —C(R⁶)₂)ₘ-G, —S(O)₂—(C₁-C₆)alkyl, —S(O)₂—(C₃-C₆)cycloalkyl, (C₁-C₆)alkyl, —S(O)—(C₃-C₆)cycloalkyl, —C(O)—(C₃-C₆)cycloalkyl, —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₁-C₆)alkyl, —C(O)—(C₆-C₁₀)aryl, —C(O)—O—(C₁-C₆)alkyl, —C(O)—O—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C₂-C₁₀)heteroaryl(C₁-C₆)alkyl, —C(O)—(C(R⁶)₂)ₘ—O—(C₆-C₁₀)aryl, —C(O)-(benzo-fused (C₃-C₆)cycloalkyl), —S(O)₂-(benzo-fused (C₂-C₁₀)heterocyclyl), —C(O)—N(R⁹)—(C(R⁶)₂)ₘ—(C₆-C₁₀)aryl, —C(O)—N(R⁹)—(C₆-C₁₀)aryl, (C₃-C₆)cycloalkyl, benzo-fused (C₃-C₆)cycloalkyl, unsubstituted (C₆-C₁₀)aryl, (C₆-C₁₀)aryl substituted with one or more Y groups, unsubstituted (C₂-C₁₀)heterocyclyl, and (C₂-C₁₀)heterocyclyl substituted with one or more X groups,
   wherein the (C₆-C₁₀)aryl or heteroaryl portion of said —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C(R⁶)₂)ₘ—O—(C₆-C₁₀)aryl, —C(O)—N(R⁹)—(C(R⁶)₂)ₘ—(C₆-C₁₀)aryl, or —C(O)—N(R⁹)—(C₆-C₁₀)aryl of R⁵ is unsubstituted or substituted with one or more Y groups;
   wherein the heterocyclyl portion of —S(O)₂-(benzo-fused (C₂-C₁₀)heterocyclyl)aryl of R⁵ is unsubstituted or substituted with one or more X groups;
each R⁶ is independently selected from the group consisting of H and (C₁-C₆)alkyl;
R⁷ is selected from the group consisting of H, (C₁-C₆)alkyl, unsubstituted (C₆-C₁₀)aryl, and (C₆-C₁₀)aryl substituted with one or more Y groups;
each R⁸ is independently selected from the group consisting of H, (C₁-C₆)alkyl, —C(O)—(C₆-C₁₀)aryl, —S(O)₂—(C₆-C₁₀)aryl, and —S(O)₂—(C₁-C₆)alkyl;
each R⁹ is independently selected from the group consisting of H, (C₁-C₆)alkyl, (C₃-C₆)cycloalkyl, unsubstituted (C₆-C₁₀)aryl, and (C₆-C₁₀)aryl substituted with one or more Y groups;
G is selected from the group consisting of H, (C₁-C₆)alkyl, unsubstituted (C₆-C₁₀)aryl, (C₆-C₁₀)aryl substituted with one or more Y groups, —CN, (C₃-C₆)cycloalkyl, —O—R⁷, —S—R⁷, unsubstituted (C₂-C₁₀)heteroaryl, (C₂-C₁₀)heteroaryl substituted with one or more Y groups, —N(R⁸)₂, unsubstituted (C₂-C₁₀)heterocyclyl, and (C₂-C₁₀)heterocyclyl substituted with one or more X groups;
each X is independently selected from the group consisting of (C₁-C₆)alkyl, —C(O)—N(R⁹)₂, —C(O)—(C₂-C₁₀)heteroaryl, (C₂-C₁₀)heteroaryl, —(C(R⁶)₂)ₘ—(C₆-C₁₀)aryl, and (C₆-C₁₀)aryl,
   wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O-alkyl, haloalkyl, and —CN, and
   wherein said (C₆-C₁₀)aryl or the (C₆-C₁₀)aryl portion of said —(C(R⁶)₂)ₘ—(C₆-C₁₀)aryl of X is unsubstituted or substituted with one or more substituents selected from the group consisting of halogen, —OH, —O-alkyl, haloalkyl, and —CN;
each Y is independently selected from the group consisting of halogen, (C₁-C₆)alkyl, (C₆-C₁₀)aryl, —C(O)—(C₁-C₆)alkyl, —O—R⁹, (C₁-C₆)haloalkyl, —O—(C₁-C₆)haloalkyl, —CN, —C(O)—O—(C₁-C₆)alkyl, —N(R⁶)₂, and —C(R⁶)₂—N(R⁸)₂; or
two of said Y groups attached to adjacent carbon atoms form a —O—CH₂—O— or —O—CH₂CH₂—O— group;
each n is independently an integer from 0-5; and
m is an integer from 1-5.

In another embodiment, the compound of Formula (I) is a compound having the structural Formula (IA): 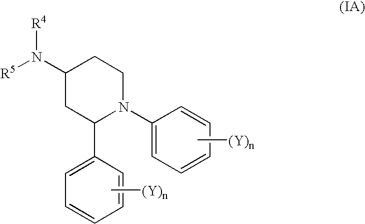
or a pharmaceutically acceptable salt, solvate, or ester thereof, wherein:
R⁴ is H;
R⁵ is selected from the group consisting of —C(R⁶)₂)ₘ-G, —S(O)₂—(C₁-C₆)alkyl, —S(O)₂—(C₃-C₆)cycloalkyl, (C₁-C₆)alkyl, —S(O)—(C₃-C₆)cycloalkyl, —C(O)—(C₃-C₆)cycloalkyl, —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₁-C₆)alkyl, —C(O)—(C₆-C₁₀)aryl, —C(O)O—(C₁-C₆)alkyl, —C(O)—O—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C₂-C₁₀)heteroaryl(C₁-C₆)alkyl, —C(O)—(C(R⁶)₂)ₘ—O—(C₆-C₁₀)aryl, —C(O)-(benzo-fused (C₃-C₆)cycloalkyl), —S(O)₂-(benzo-fused (C₂-C₁₀)heterocyclyl), —C(O)—N(R⁹)—(C(R⁶)₂)ₘ—(C₆-C₁₀)aryl, —C(O)—N(R⁹)—(C₆-C₁₀)aryl, (C₃-C₆)cycloalkyl, benzo-fused (C₃-C₆)cycloalkyl, unsubstituted (C₆-C₁₀)aryl, (C₆-C₁₀)aryl substituted with one or more Y groups, unsubstituted (C₂-C₁₀)heterocyclyl, and (C₂-C₁₀)heterocyclyl substituted with one or more X groups,
   wherein the (C₆-C₁₀)aryl or heteroaryl portion of said —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C(R⁶)₂)ₘ—O—(C₆-C₁₀)aryl, —C(O)—N(R⁹)—(C(R⁶)₂)ₘ—(C₆-C₁₀)aryl, or —C(O)—N(R⁹)—(C₆-C₁₀)aryl of R⁵ is unsubstituted or substituted with one or more Y groups;
   wherein the heterocyclyl portion of —S(O)₂-(benzo-fused (C₂-C₁₀)heterocyclyl)aryl of R⁵ is unsubstituted or substituted with one or more X groups;
each R⁶ is independently selected from the group consisting of H and (C₁-C₆)alkyl;
R⁷ is selected from the group consisting of H, (C₁-C₆)alkyl, unsubstituted (C₆-C₁₀)aryl, and (C₆-C₁₀)aryl substituted with one or more Y groups;
each R⁸ is independently selected from the group consisting of H, (C₁-C₆)alkyl, —C(O)—(C₆-C₁₀)aryl, —S(O)₂—(C₆-C₁₀)aryl, and —S(O)₂—(C₁-C₆)alkyl;
each R⁹ is independently selected from the group consisting of H, (C₁-C₆)alkyl, (C₃-C₆)cycloalkyl, unsubstituted (C₆-C₁₀)aryl, and (C₆-C₁₀)aryl substituted with one or more Y groups;
G is selected from the group consisting of H, (C₁-C₆)alkyl, unsubstituted (C₆-C₁₀)aryl, (C₆-C₁₀)aryl substituted with one or more Y groups, —CN, (C₃-C₆)cycloalkyl, —O—R⁷, —S—R⁷, unsubstituted (C₂-C₁₀)heteroaryl, (C₂-C₁₀)heteroaryl substituted with one or more Y groups, —N(R⁸)₂, unsubstituted (C₂-C₁₀)heterocyclyl, and (C₂-C₁₀)heterocyclyl substituted with one or more X groups;
each X is independently selected from the group consisting of (C₁-C₆)alkyl, —C(O)—N(R⁹)₂, —C(O)—(C₂-C₁₀)heteroaryl, (C₂-C₁₀)heteroaryl, —(C(R⁶)₂)ₘ—(C₆-C₁₀)aryl, and (C₆-C₁₀)aryl,
   wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O-alkyl, haloalkyl, and —CN, and
   wherein said (C₆-C₁₀)aryl or the (C₆-C₁₀)aryl portion of said —(C(R⁶)₂)ₘ—(C₆-C₁₀)aryl of X is unsubstituted or substituted with one or more substituents selected from the group consisting of halogen, —OH, —O-alkyl, haloalkyl, and —CN;
each Y is independently selected from the group consisting of halogen, (C₁-C₆)alkyl, (C₆-C₁₀)aryl, —C(O)—(C₁-C₆)alkyl, —O—R⁹, (C₁-C₆)haloalkyl, —O—(C₁-C₆)haloalkyl, —CN, —C(O)—O—(C₁-C₆)alkyl, —N(R⁶)₂, and —C(R⁶)₂—N(R⁸)₂; or
two of said Y groups attached to adjacent carbon atoms form a —O—CH₂—O— or —O—CH₂CH₂—O— group;
each n is independently an integer from 0-5; and
m is an integer from 1-5.

In another embodiment, the compound of Formula (I) is a compound having the structural Formula (IA): 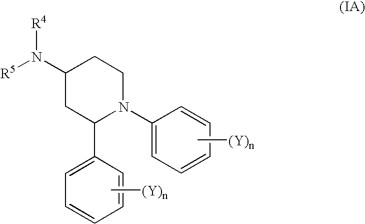
or a pharmaceutically acceptable salt, solvate, or ester thereof, wherein:
R⁴ is H;
R⁵ is selected from the group consisting of —C(R⁶)₂)ₘ-G, —S(O)₂—(C₁-C₆)alkyl, —S(O)—(C₃-C₆)cycloalkyl, —C(O)—(C₃-C₆)cycloalkyl, —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₆-C₁₀)aryl, —C(O)—O—(C₁-C₆)alkyl, —C(O)—O—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C₂-C₁₀)heteroaryl(C₁-C₆)alkyl, —C(O)—(C(R⁶)₂)ₘ—O—(C₆-C₁₀)aryl, —C(O)-(benzo-fused (C₃-C₆)cycloalkyl), —S(O)₂-(benzo-fused (C₂-C₁₀)heterocyclyl), —C(O)—N(R⁹)—(C(R⁶)₂)ₘ—(C₆-C₁₀)aryl, —C(O)—N(R⁹)—(C₆-C₁₀)aryl, (C₃-C₆)cycloalkyl, and benzo-fused (C₃-C₆)cycloalkyl;
each R⁶ is independently selected from the group consisting of H and (C₁-C₆)alkyl;
R⁷ is selected from the group consisting of H, (C₁-C₆)alkyl and unsubstituted (C₆-C₁₀)aryl;
each R⁹ is independently selected from the group consisting of H, (C₁-C₆)alkyl, (C₃-C₆)cycloalkyl, unsubstituted (C₆-C₁₀)aryl, and (C₆-C₁₀)aryl substituted with one or more Y groups;
G is selected from the group consisting of H, (C₁-C₆)alkyl, unsubstituted (C₆-C₁₀)aryl, (C₆-C₁₀)aryl substituted with one or more Y groups, —CN, (C₃-C₆)cycloalkyl, —O—R⁷, —S—R⁷, unsubstituted (C₂-C₁₀)heteroaryl, unsubstituted (C₂-C₁₀)heterocyclyl, and (C₂-C₁₀)heterocyclyl substituted with one or more X groups;
each X is independently selected from the group consisting of (C₁-C₆)alkyl and (C₆-C₁₀)aryl substituted with one or more substituents selected from the group consisting of halogen, —OH, —O-alkyl, haloalkyl, and —CN; and
each Y is independently selected from the group consisting halogen, (C₁-C₆)alkyl, (C₆-C₁₀)aryl, —O—R⁹, (C₁-C₆)haloalkyl, —O—(C₁-C₆)haloalkyl, —CN, —C(O)—O—(C₁-C₆)alkyl, —C(O)—(C₁-C₆)alkyl, —N(R⁶)₂, and —C(R⁶)₂—N(R⁸)₂; or
two of said Y groups attached to adjacent carbon atoms form a —O—CH₂—O— or —O—CH₂CH₂—O— group.
each n is independently an integer from 0-5; and
m is an integer from 1-5.

In another embodiment, the compound of Formula (I) is a compound having the structural Formula (IA): 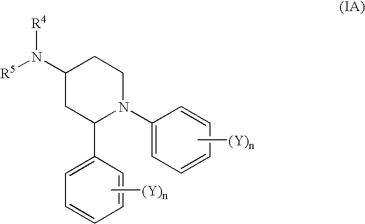
or a pharmaceutically acceptable salt, solvate, or ester thereof, wherein:
R⁴ is H;
R⁵ is selected from the group consisting of —C(R⁶)₂)ₘ-G, —S(O)₂—CH₃, —S(O)₂-phenyl, —S(O—C(R⁶)₂-phenyl, —S(O)₂-thiophenyl, —C(O)phenyl, —C(O)—C(R⁶)₂-phenyl, —C(O)cyclopropylene-phenyl, —C(O)-(benzo-fused cyclohexyl), —C(O)-furanyl, —C(O)C(R⁶)₂—O-phenyl, —C(O)—(C(R⁶)₂)₂-phenyl, —C(O)—N(R⁹)-phenyl, —C(O)N(R⁹)—C(R⁶)₂-phenyl, cyclobutyl, cyclopentyl, cyclohexyl, and indanyl,
   wherein the phenyl, thiophenyl, and furanyl portions of said —S(O)₂-phenyl, —S(O)₂—C(R⁶)₂-phenyl, —S(O)₂-thiophenyl, —C(O)-phenyl, —C(O)—C(R⁶)₂-phenyl, —C(O)-cyclopropylene-phenyl, —C(O)-furanyl, —C(O)—C(R⁶)₂—O-phenyl, —C(O)—(C(R⁶)₂)₂-phenyl, —C(O)—N(R⁹)phenyl, or —C(O)—N(R⁹)—C(R⁶)₂-phenyl of R⁵ are unsubstituted or substituted with one or more Y groups;
each R⁶ is independently selected from the group consisting of H, —CH₃, —CH₂CH₃, and —CH₂(CH₃)₂;
R⁷ is selected from the group consisting of H, —CH₃, —CH(CH₃)₂, —CH₂CH₂CH₂CH₃, and unsubstituted phenyl;
Each R⁹ is independently selected from the group consisting of H, —CH₃, —CH(CH₃)₂, —CH₂CH₂CH₂CH₃, unsubstituted phenyl, and phenyl substituted with one or more Y groups;
G is selected from the group consisting of H, —CH₃, —CH₂CH₃, —C(CH₃)₃, unsubstituted phenyl, phenyl substituted with one or more Y groups, —CN, cyclohexyl, —O—R⁷, —S—R⁷, furanyl, thiophenyl, pyridinyl, benzothiophenyl, and pyrrolidinyl substituted with one or more X groups;
each X is independently selected from the group consisting of —CH₃ and phenyl substituted with one or more Cl; and
each Y is independently selected from the group consisting of F, Cl, —OCF₃, —OCH₃, phenyl, —C(O)—CH₃, —CH₃, —CN, —NH₂, and —CF₃; or
two of said Y groups attached to adjacent carbon atoms form a —O—CH₂—O— group;
each n is independently an integer from 0-5; and
m is an integer from 1-5.

In another embodiment, the compound of Formula (I) is a compound having the structural Formula (IB): 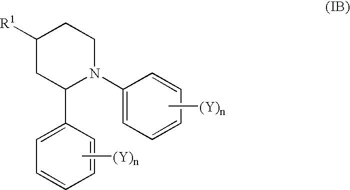
or a pharmaceutically acceptable salt, solvate, or ester thereof, wherein:
R¹ is selected from the group consisting of unsubstituted (C₂-C₁₀)heterocyclyl, (C₂-C₁₀)heterocyclyl substituted with one or more X groups, —N₃, and —OR⁷;
   with the following proviso:
   (i) when R¹ is —OH, n is independently an integer of from 1-5;
each R⁶ is independently selected from the group consisting of H and (C₁-C₆)alkyl;
R⁷ is selected from the group consisting of H, (C₁-C₆)alkyl, unsubstituted (C₆-C₁₀)aryl, and (C₆-C₁₀)aryl substituted with one or more Y groups;
   with the proviso that when R⁷ is H, each n is independently an integer of from 1-5;
each R⁸ is independently selected from the group consisting of H, (C₁-C₆)alkyl, —C(O)—(C₆-C₁₀)aryl, —S(O)₂—(C₆-C₁₀)aryl, and —S(O)₂—(C₁-C₆)alkyl;
each X is independently selected from the group consisting of (C₁-C₆)alkyl, —C(O)—N(R⁸)₂, —C(O)—(C₂-C₁₀)heteroaryl, (C₂-C₁₀)heteroaryl, —(C(R⁶)₂)—(C₆-C₁₀)aryl, and (C₆-C₁₀)aryl
   wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O-alkyl, haloalkyl, and —CN, and
   wherein said (C₆-C₁₀)aryl or the (C₆-C₁₀)aryl portion of said —(C(R⁶)₂)—(C₆-C₁₀)aryl of X is unsubstituted or substituted with one or more substituents selected from the group consisting of halogen, —OH, —O-alkyl, haloalkyl, and —CN; and
n is an integer from 0-5.

In another embodiment, the compound of Formula (I) is a compound having the structural Formula (IC): 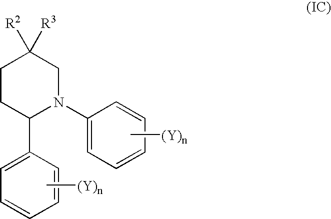
or a pharmaceutically acceptable salt, solvate or ester thereof, wherein:
R² is selected from the group consisting of H, —C(R⁶)₂—(C₆-C₁₀)aryl, and —C(R⁶)₂—O—R⁷,
   wherein the (C₆-C₁₀)aryl portion of said —C(R⁶)₂—(C₆-C₁₀)aryl of R² is unsubstituted or substituted with one or more Y groups;
R³ is selected from the group consisting of H, —C(R⁶)₂—(C₆-C₁₀)aryl, —C(R⁶)₂—O—R⁷, —O—R⁷, and —C(R⁶)₂—N(R⁸)₂,
   wherein the (C₆-C₁₀)aryl portion of said —C(R⁶)₂—(C₆-C₁₀)aryl of R³ is unsubstituted or substituted with one or more Y groups; or
R² and R³ together with the ring carbon atom to which they are shown attached form an unsubstituted (C₂-C₁₀)heterocyclyl ring or a (C₂-C₁₀)heterocyclyl ring substituted with one or more X groups;
   with the following proviso:
   (ii) at least one of R² and R³ is not H;
each R⁶ is independently selected from the group consisting of H and (C₁-C₆)alkyl;
R⁷ is selected from the group consisting of H, (C₁-C₆)alkyl, unsubstituted (C₆-C₁₀)aryl, and (C₆-C₁₀)aryl substituted with one or more Y groups;
each R⁸ is independently selected from the group consisting of H, (C₁-C₆)alkyl, —C(O)—(C₆-C₁₀)aryl, —S(O)₂—(C₆-C₁₀)aryl, —S(O)₂—(C₂-C₁₀)heteroaryl, and —S(O)₂—(C₁-C₆)alkyl,
   wherein the (C₆-C₁₀)aryl portion of said —C(O)—(C₆-C₁₀)aryl or —S(O)₂—(C₆-C₁₀)aryl and the (C₂-C₁₀)heteroaryl portion of said —S(O)₂—(C₂-C₁₀)heteroaryl of R⁸ is unsubstituted or substituted with one or more Y groups;
each Y is independently selected from the group consisting of halogen, (C₁-C₆)alkyl, —C(O)—(C₁-C₆)alkyl, —O—R⁹, —O—C(R⁶)₂—O—, (C₁-C₆)haloalkyl, —O—(C₁-C₆)haloalkyl, —CN, —C(O)—O—(C₁-C₆)alkyl, —C(R⁶)₂—N(R⁸)₂, and —C(R⁶)₂—N(R⁶)—S(O)₂—R⁶; and
each n is independently an integer from 0-5.

In another embodiment, the compound of Formula (I) is a compound having the structural Formula (IC): 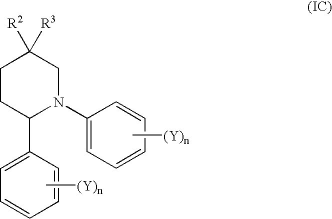
or a pharmaceutically acceptable salt, solvate or ester thereof, wherein:
R² is H;
R³ is selected from the group consisting of —C(R⁶)₂—(C₆-C₁₀)aryl, —C(R⁶)₂—O—R⁷, and —C(R⁶)₂—N(R⁸)₂,
   wherein the (C₆-C₁₀)aryl portion of said —C(R⁶)₂—(C₆-C₁₀)aryl of R³ is unsubstituted or substituted with one or more Y groups;
each R⁶ is H;
R⁷ is (C₆-C₁₀)aryl substituted with one or more Y groups;
each R⁸ is independently selected from the group consisting of H, —S(O)₂—(C₆-C₁₀)aryl, and —S(O)₂—(C₁-C₆)alkyl;
each R⁹ is independently selected from the group consisting of H, (C₁-C₆)alkyl, (C₃-C₆)cycloalkyl, unsubstituted (C₆-C₁₀)aryl, and (C₆-C₁₀)aryl substituted with one or more Y groups;
each Y is independently selected from the group consisting of halogen, —C(R⁶)₂—N(R⁸)₂, and —C(R⁶)₂—N(R⁶)—S(O)₂—R⁶; and
each n is independently an integer from 0-5.

In another embodiment, the compound of Formula (I) is a compound having the structural Formula (ID): 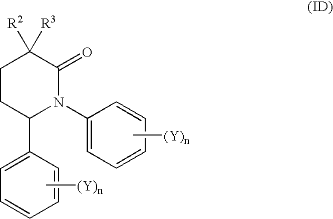
or a pharmaceutically acceptable salt, solvate, or ester thereof, wherein:
R² is selected from the group consisting of H, —C(R⁶)₂—(C₆-C₁₀)aryl, and —C(R⁶)₂—O—R⁷,
   wherein the (C₆-C₁₀)aryl portion of said —C(R⁶)₂—(C₆-C₁₀)aryl of R² is unsubstituted or substituted with one or more Y groups;
R³ is selected from the group consisting of —C(R⁶)₂—(C₆-C₁₀)aryl, —C(R⁶)₂—O—R⁷, —O—R⁷, and —C(R⁶)₂—N(R⁸)₂,
   wherein the (C₆-C₁₀)aryl portion of said —C(R⁶)₂—(C₆-C₁₀)aryl of R³ is unsubstituted or substituted with one or more Y groups;
each R⁶ is independently selected from the group consisting of H and (C₁-C₆)alkyl;
R⁷ is selected from the group consisting of H, (C₁-C₆)alkyl, unsubstituted (C₆-C₁₀)aryl, and (C₆-C₁₀)aryl substituted with one or more Y groups;
each R⁸ is independently selected from the group consisting of H, (C₁-C₆)alkyl, —C(O)—(C₆-C₁₀)aryl, —S(O)₂—(C₆-C₁₀)aryl, and —S(O)₂—(C₁-C₆)alkyl;
each Y is independently selected from the group consisting of halogen, (C₁-C₆)alkyl, —O—R⁹, —O—C(R⁶)₂—O—, (C₁-C₆)haloalkyl, —CN, —C(R⁶)₂—N(R⁸)₂, and —C(R⁶)₂—N(R⁶)—S(O)₂—R⁶; and
each n is independently an integer from 0-5.

In another embodiment, the compound of Formula (I) is selected from the group consisting of: 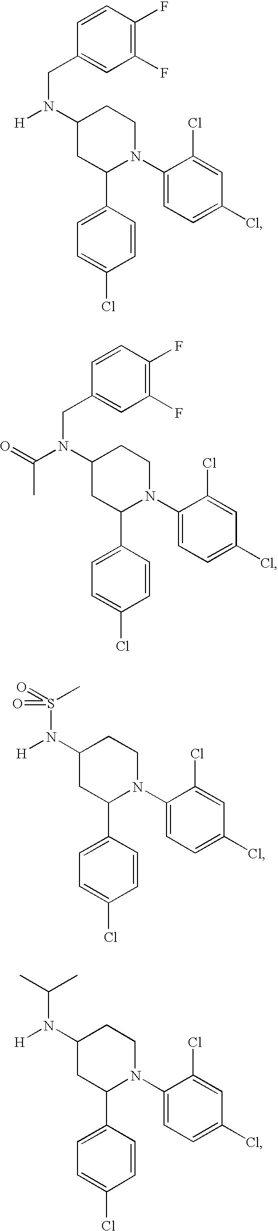 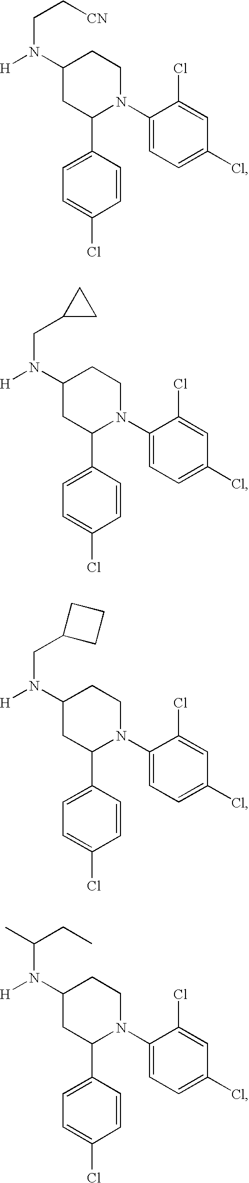 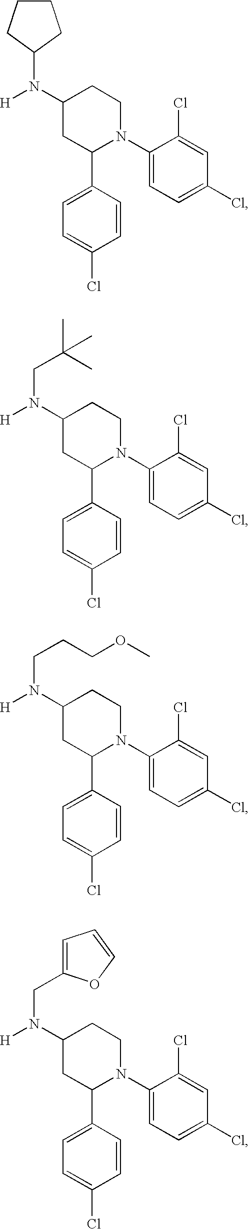 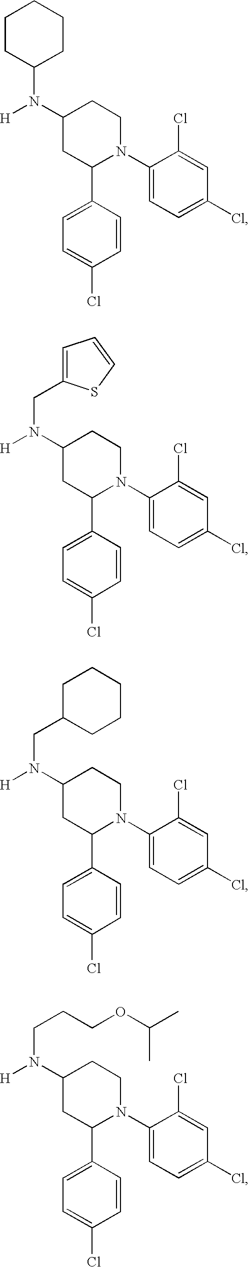 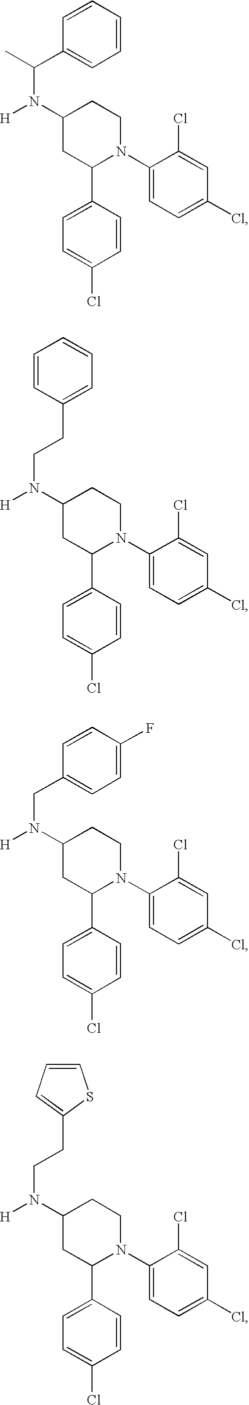 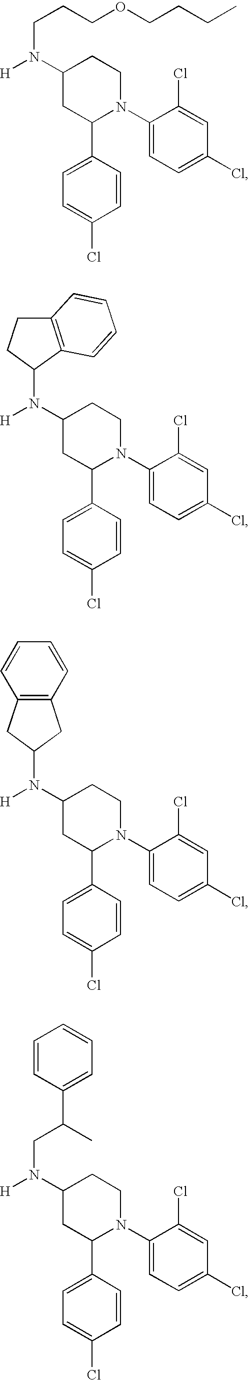 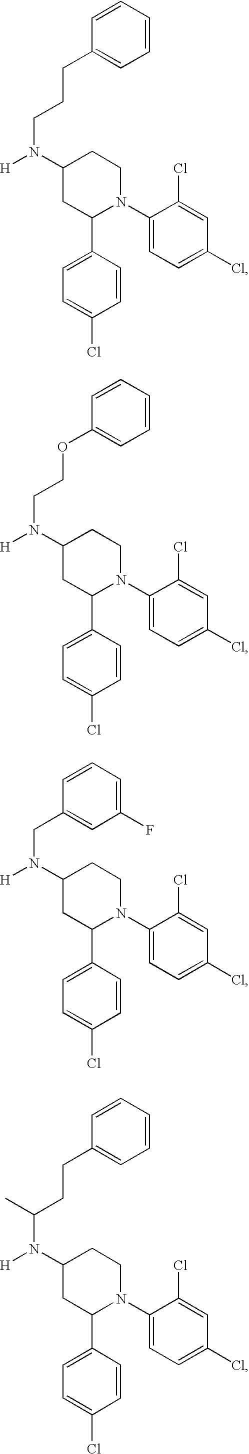 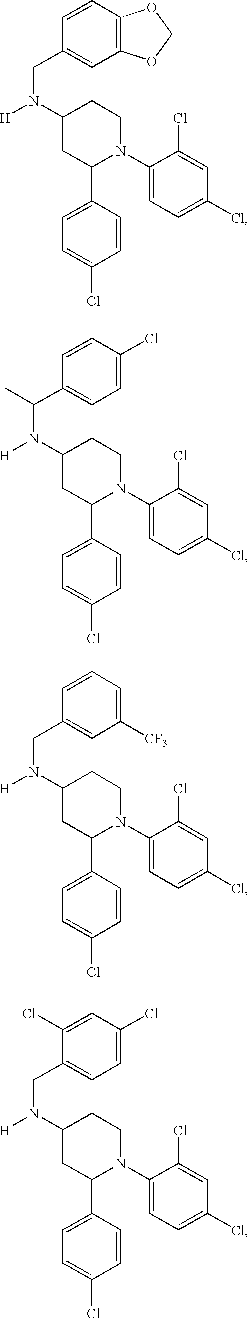 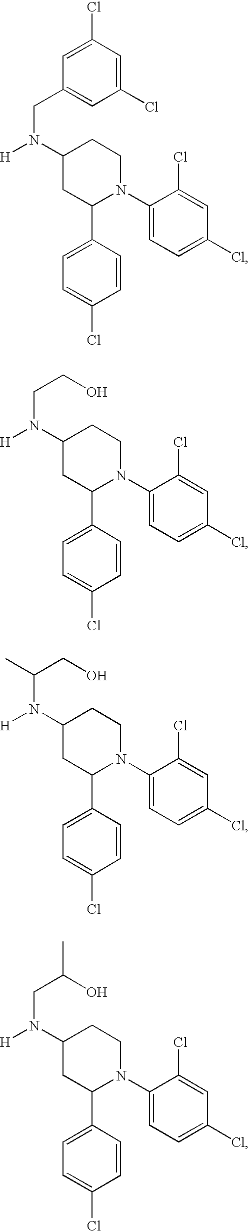 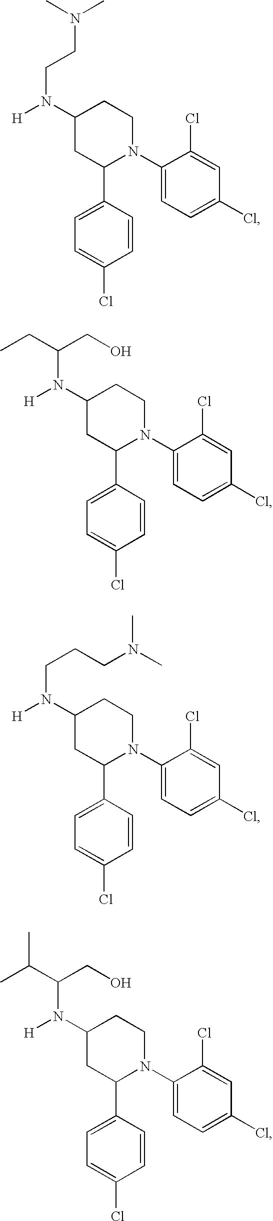 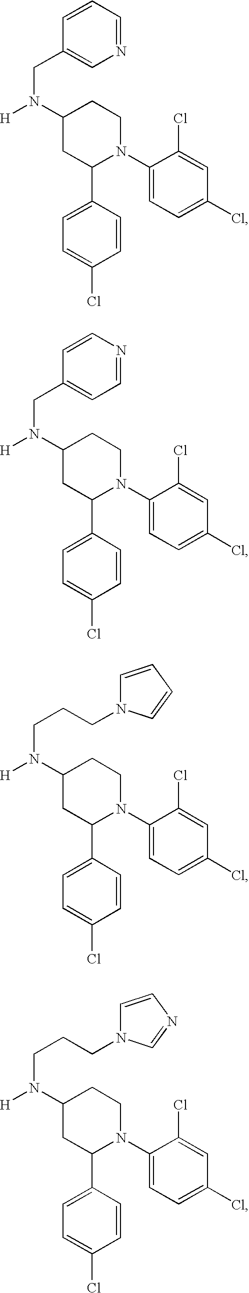 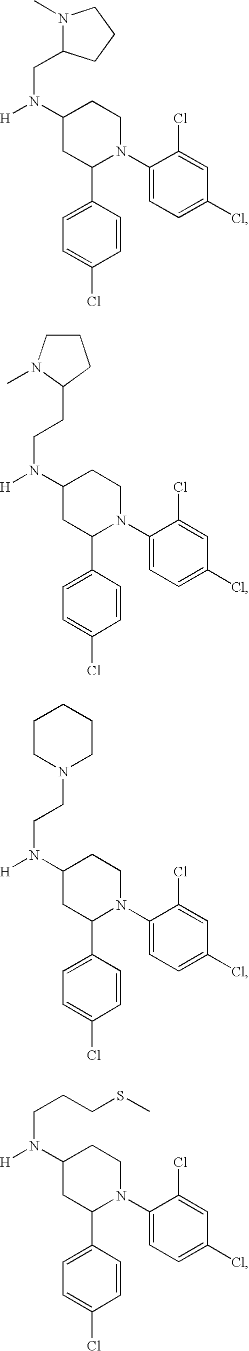 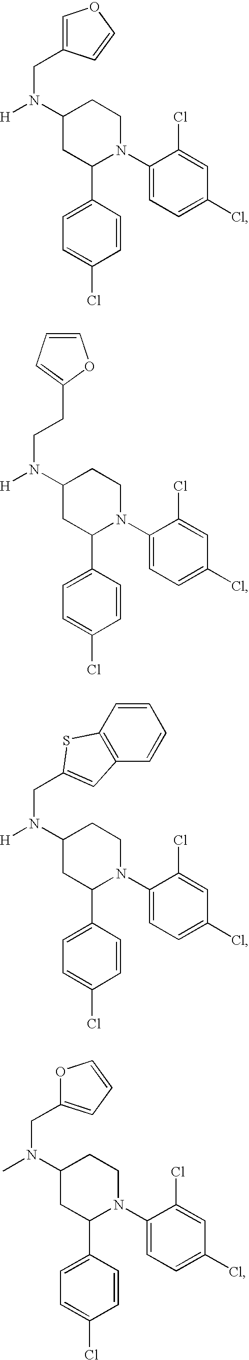 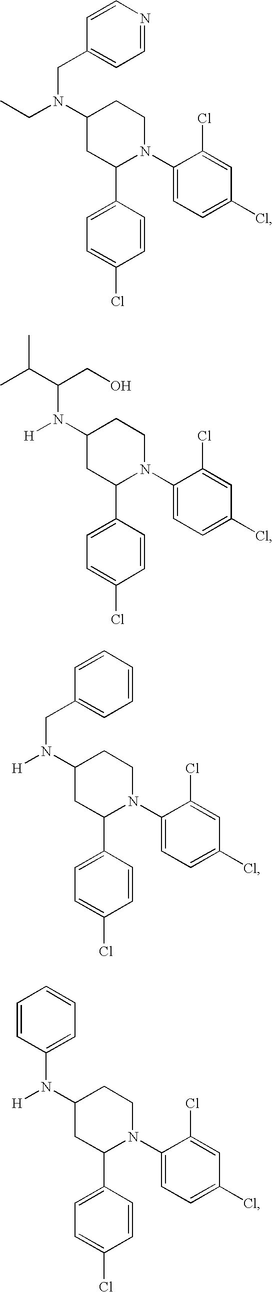 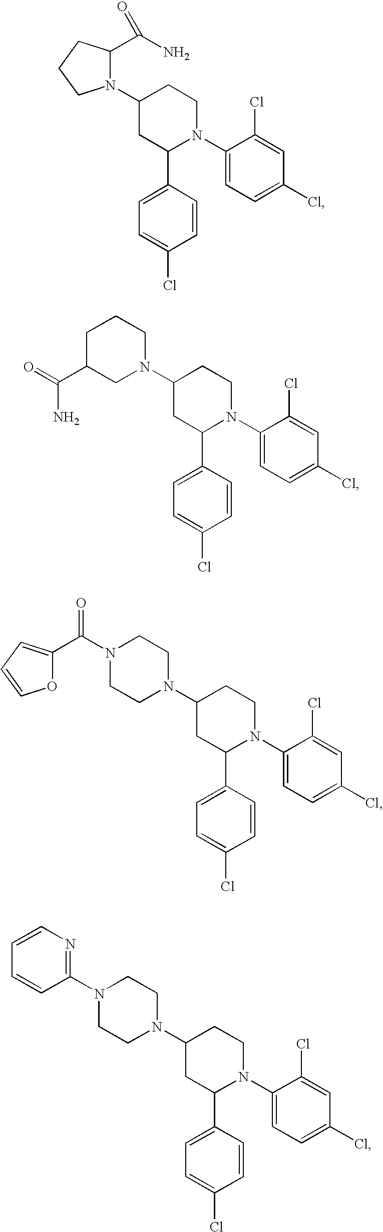 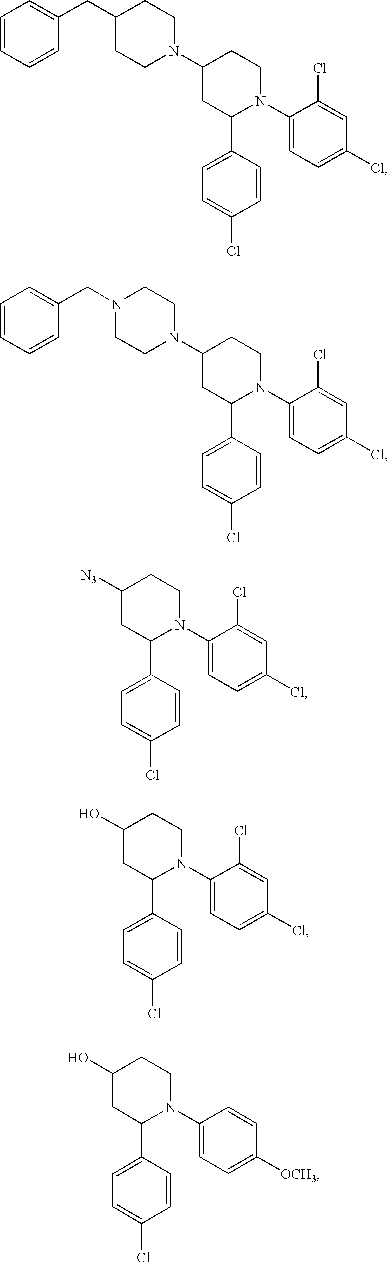 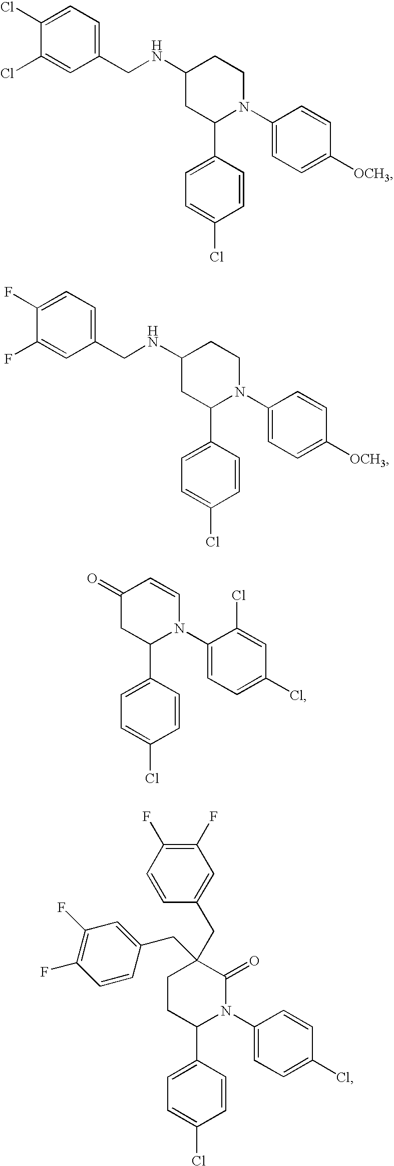 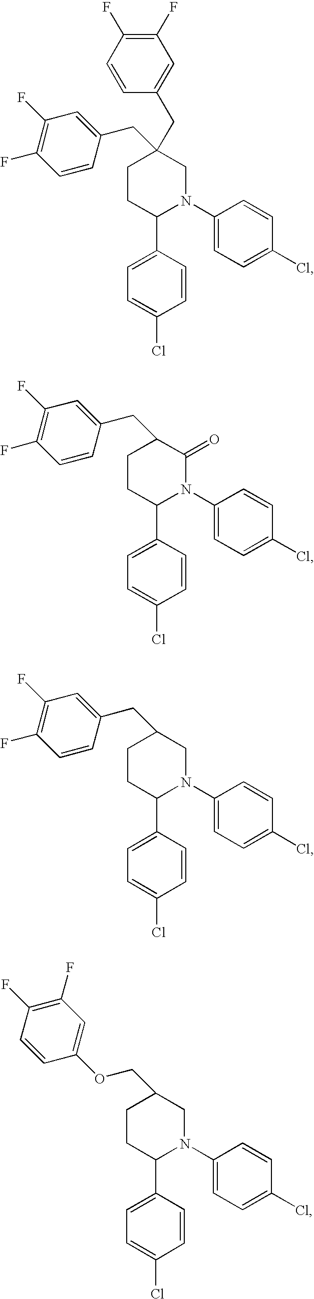 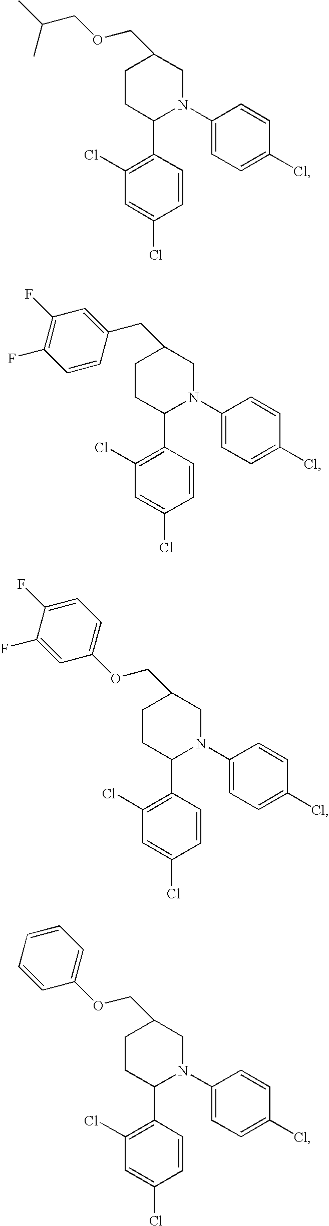 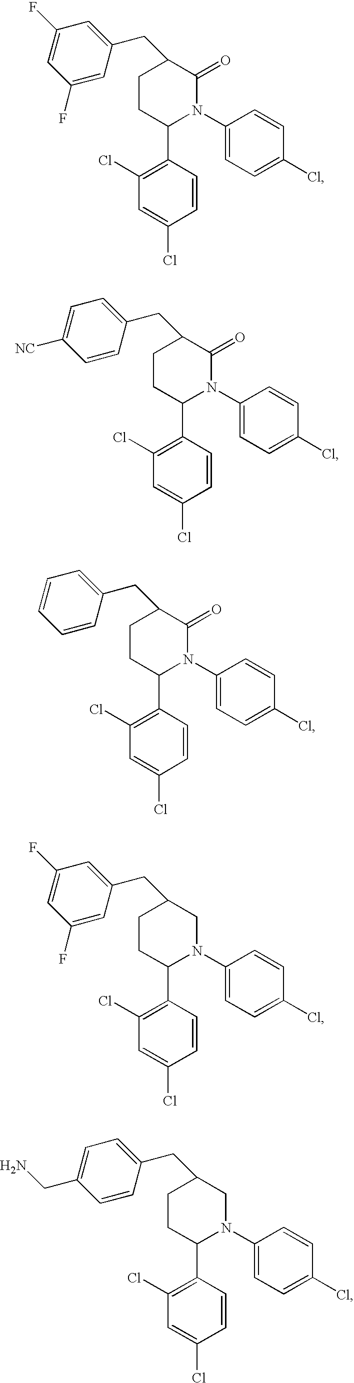 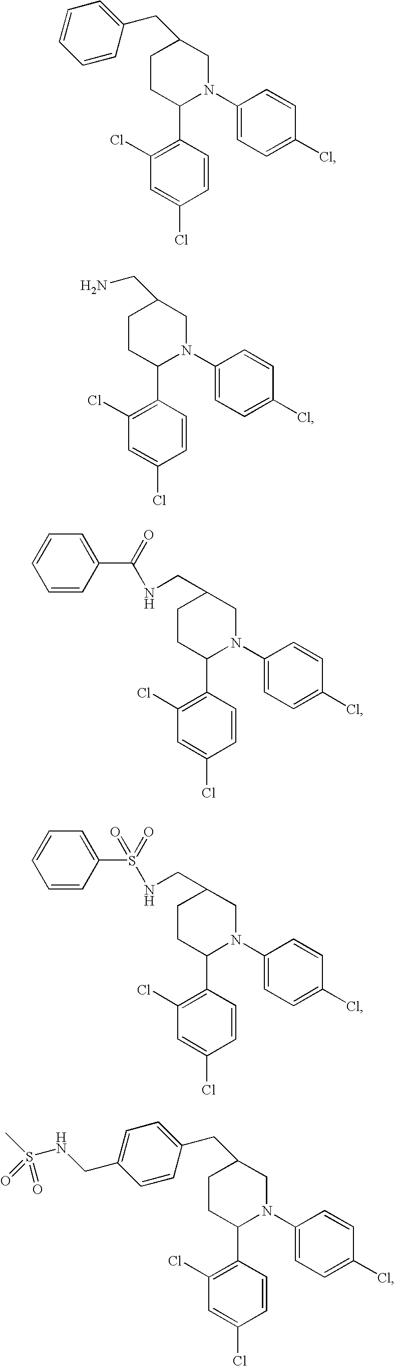 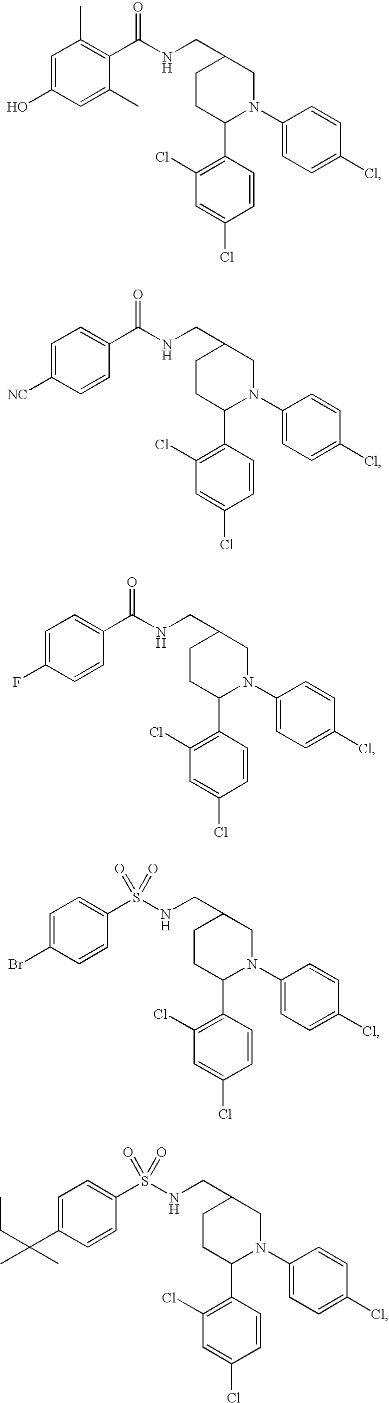 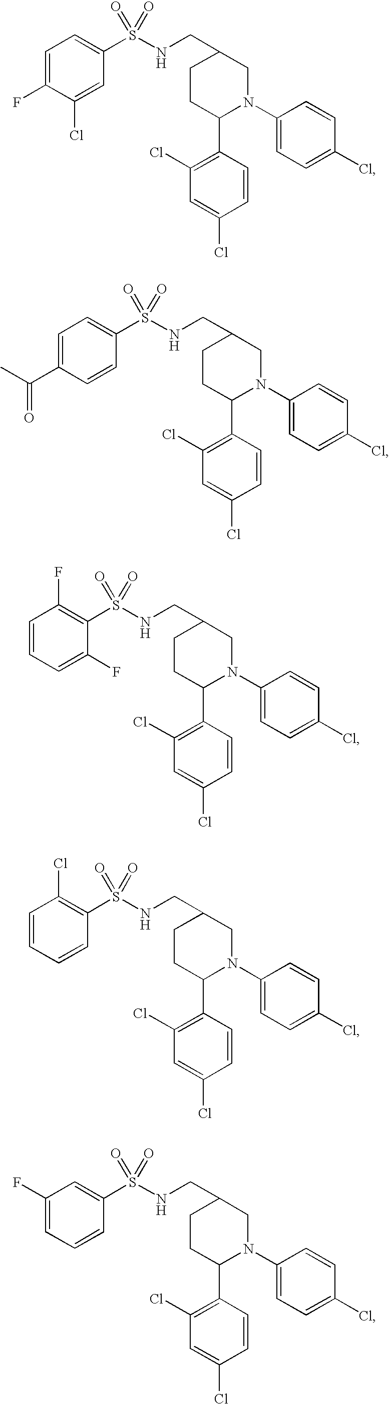 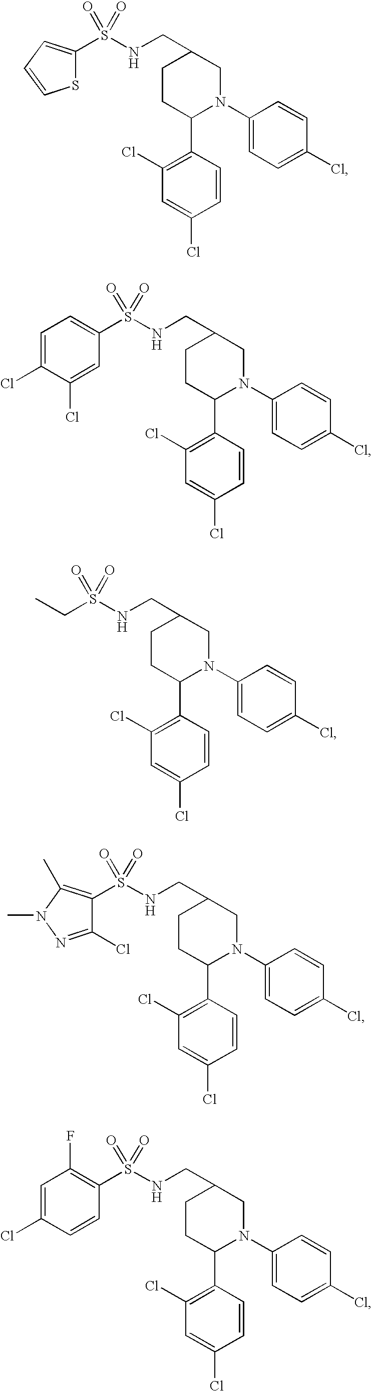 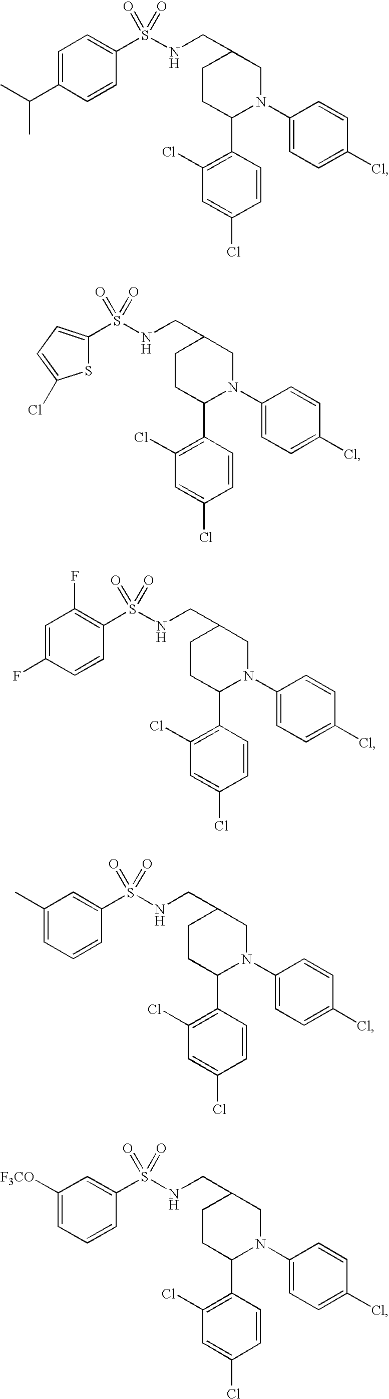 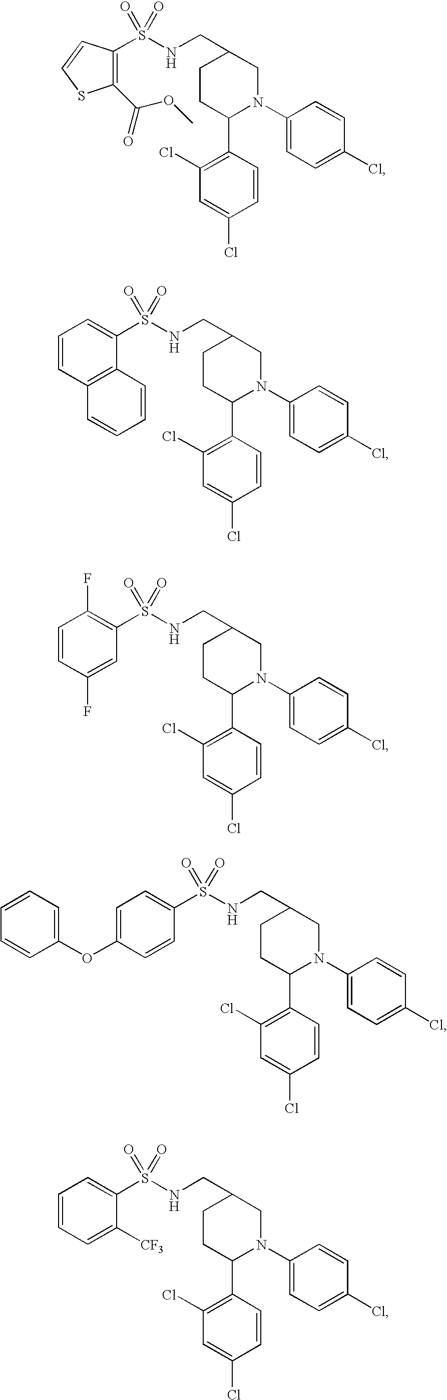 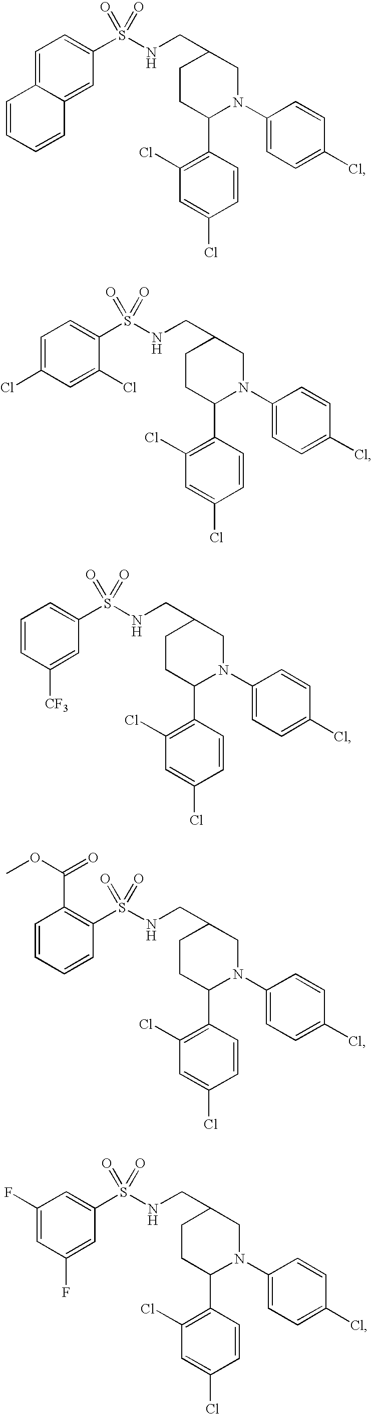 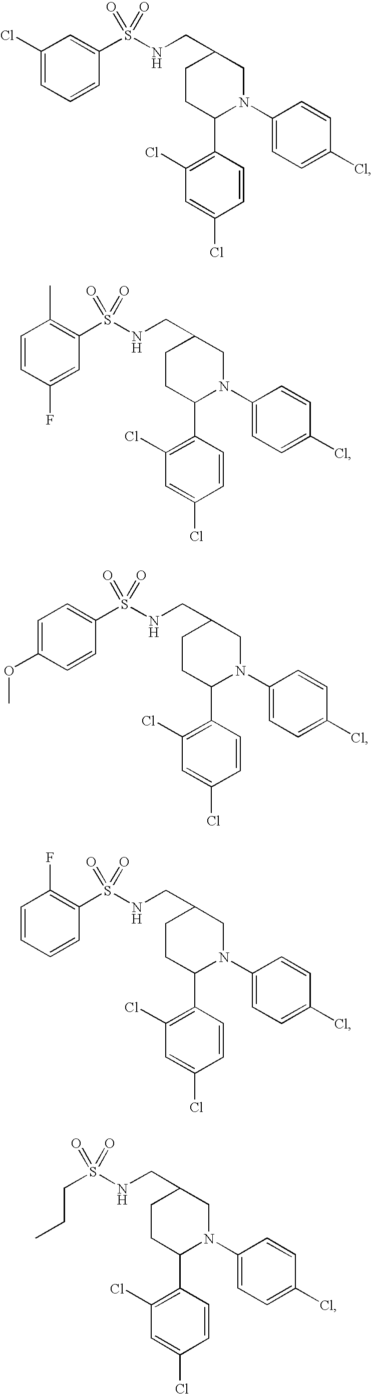 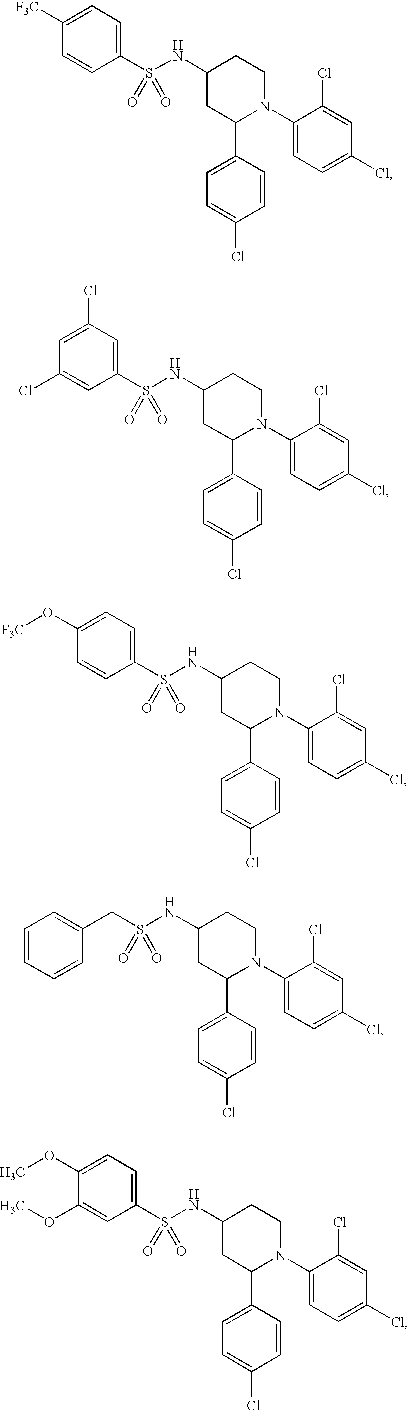 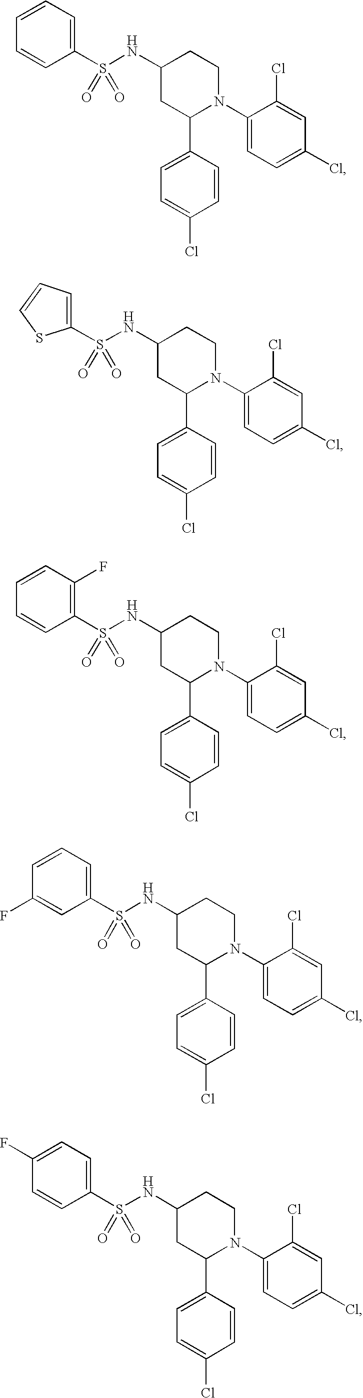 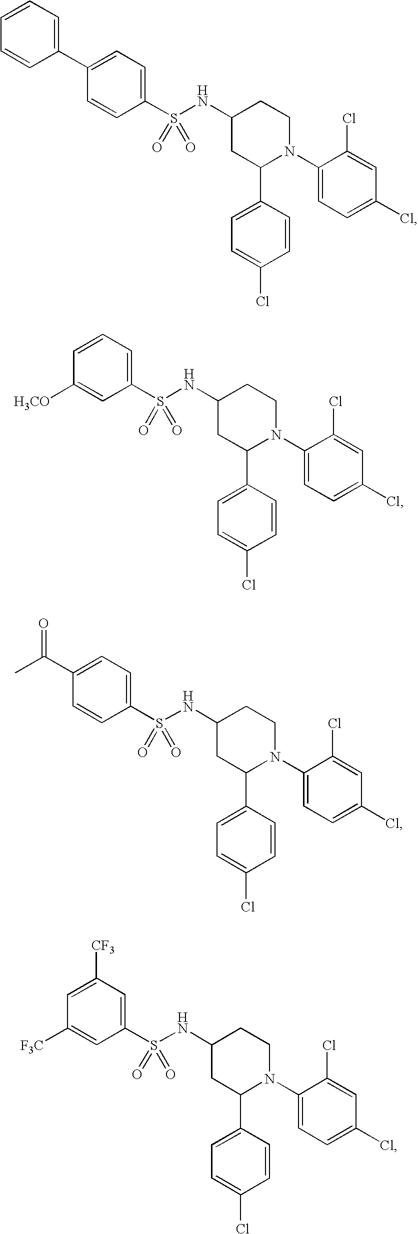 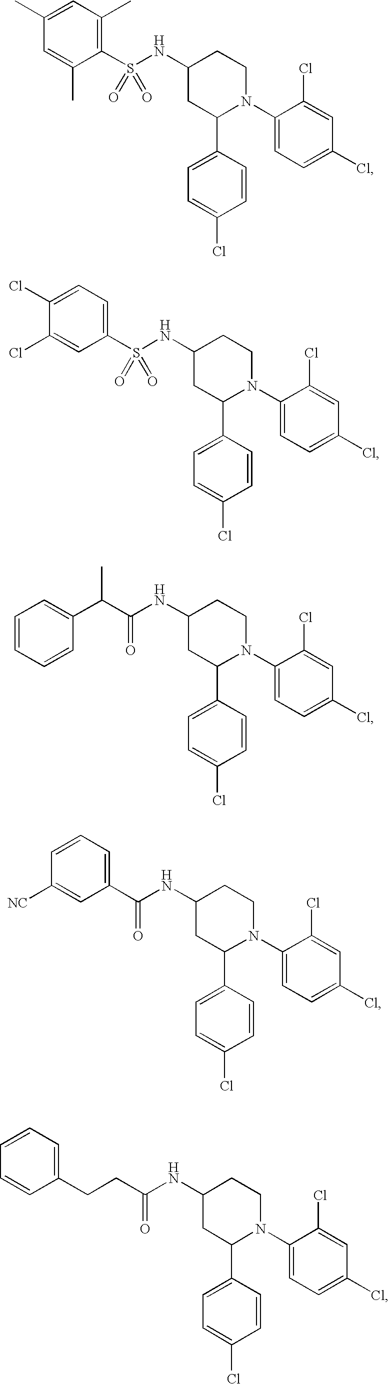 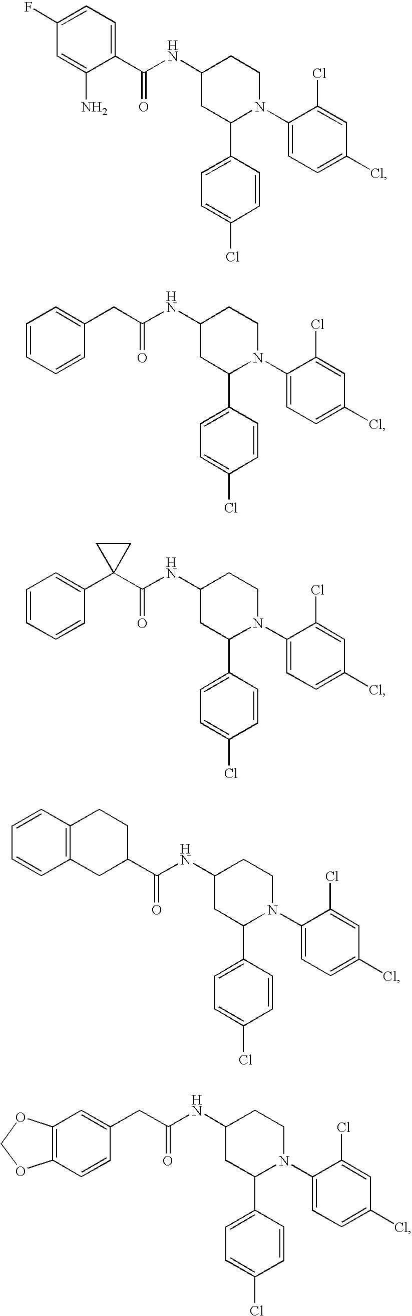 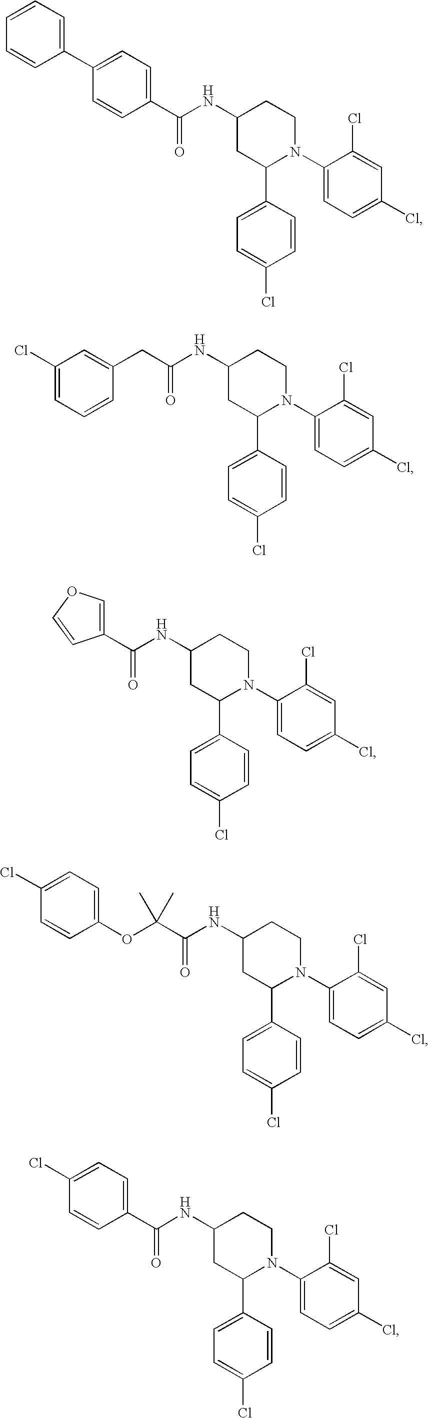 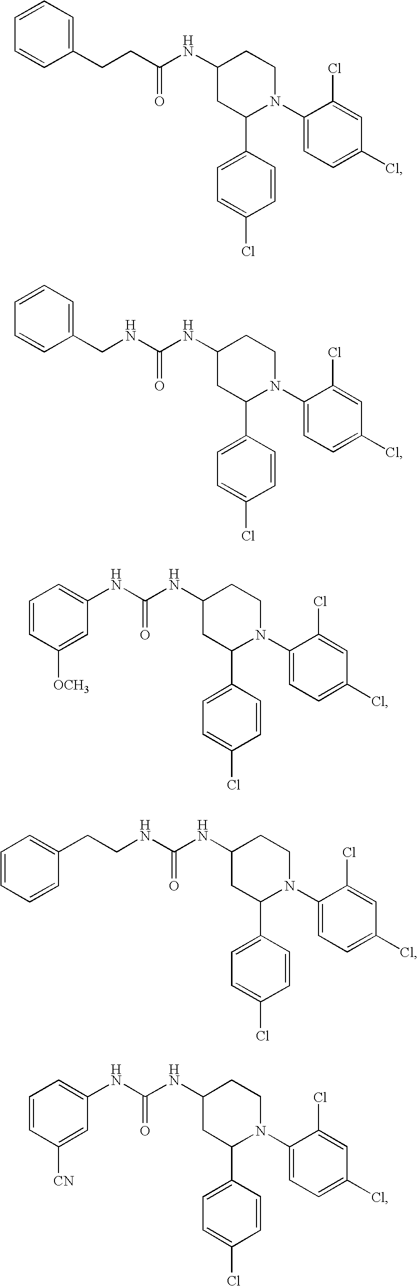 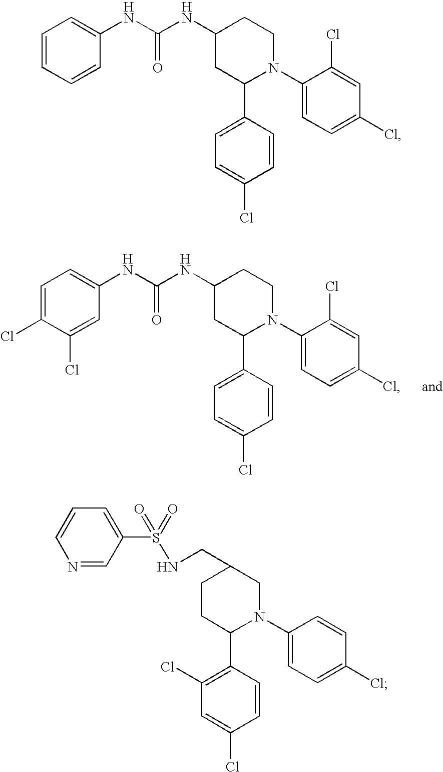
or pharmaceutically acceptable salts, solvates, or esters thereof.

When A is —CH₂—, the compounds of Formula (I) have the structure of Formula (II):

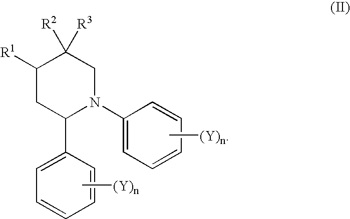

It will be recognized by one of skill in the art that compounds of Formula (II) include all stereoisomers of such compounds. A non-limiting list of stereoisomers of Formula (II) can include:

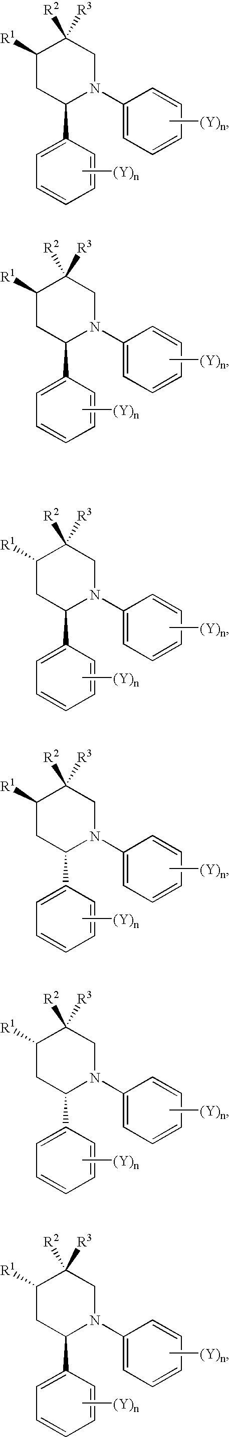

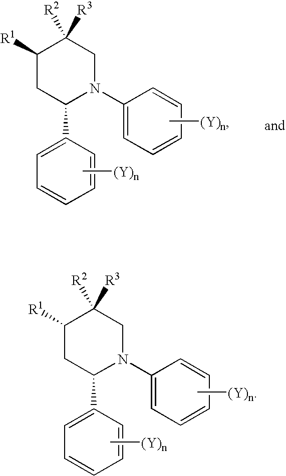

When A is —C(O)—, the compounds of Formula (I) have the structure of Formula (III):

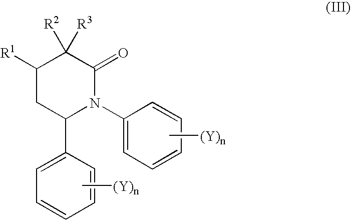

It will be recognized by one of skill in the art that compounds of Formula (III) include all stereoisomers of such compounds. A non-limiting list of stereoisomers of Formula (III) can include:

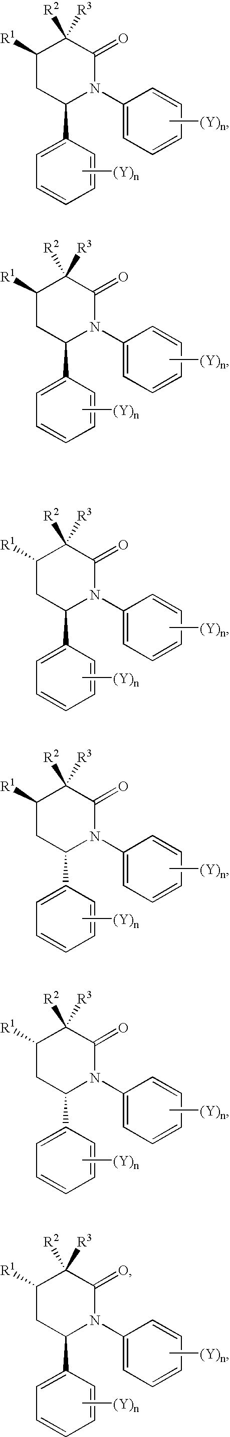

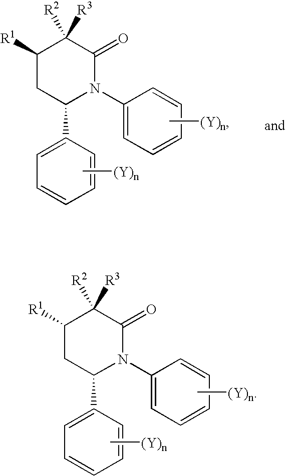

R¹ is selected from the group consisting of H, —N(R⁴)(R⁵), unsubstituted heterocyclyl, heterocyclyl substituted with one or more X groups, —N₃, and —O—R⁷, with the proviso that when R¹ is —OH, n is independently an integer of from 1-5. When R¹ is —N(R⁴)(R⁵), R⁴ and R⁵ are as defined herein. Non-limiting examples of —N(R⁴)(R⁵) of R¹ include: 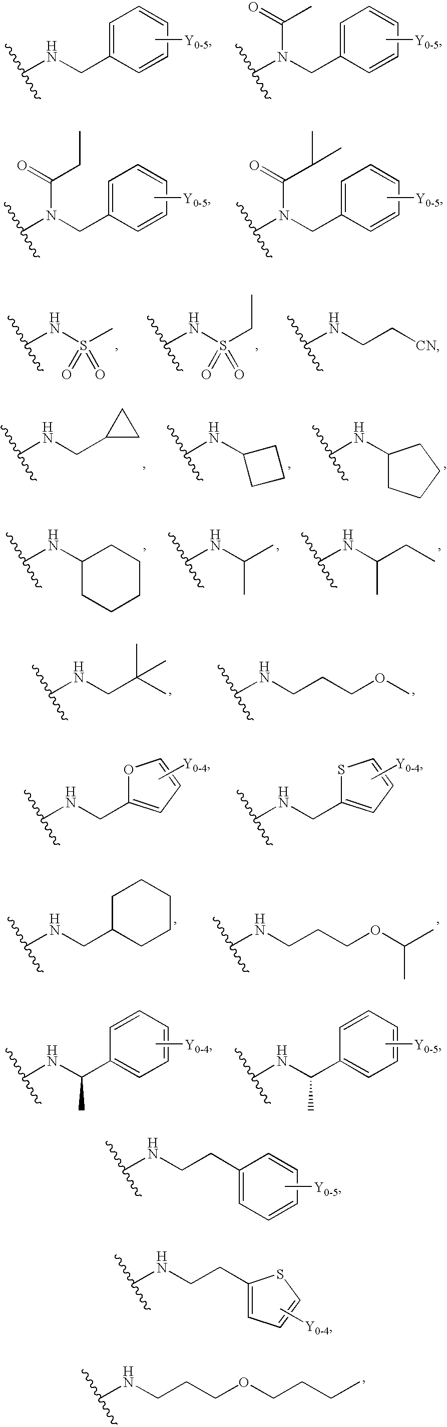 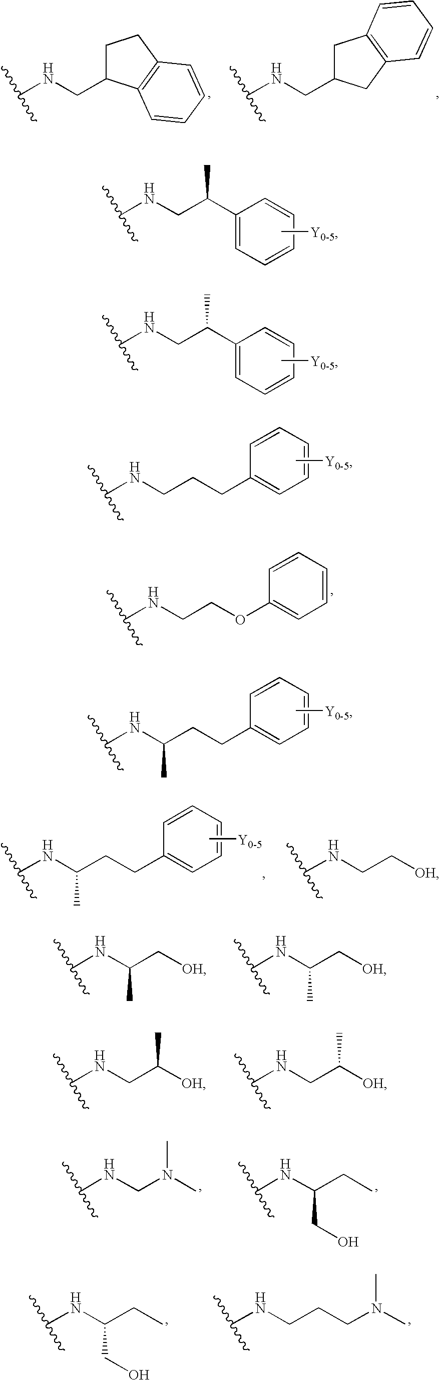 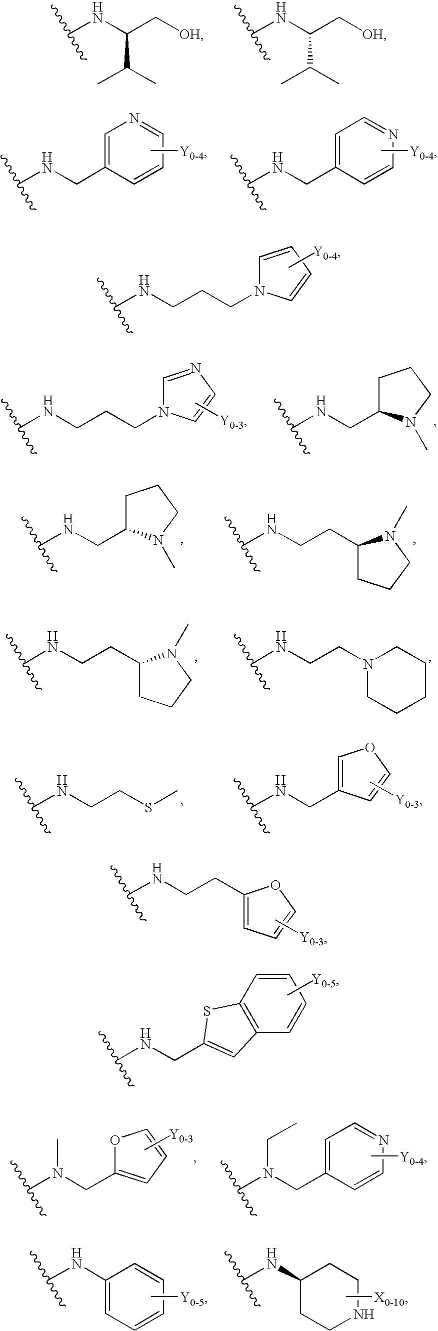 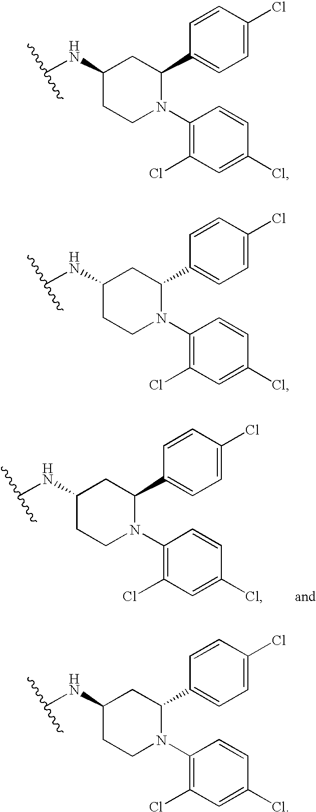
When R¹ is substituted or unsubstituted heterocyclyl, non-limiting examples include: 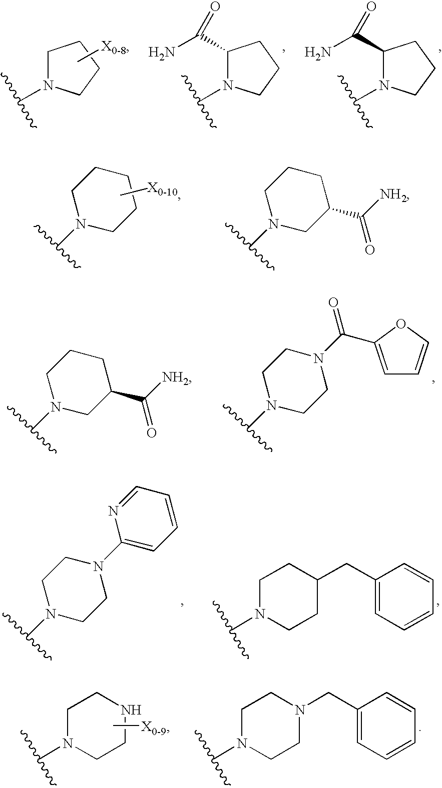
When R¹ is —O—R⁷, R⁷ is defined as described herein. Non-limiting examples of R¹ when R¹ is —O—R⁷ include —OH with the proviso that n is independently an integer of from 1-5, —OCH₃, —O—CH₂CH₃, —O—CH₂(CH₃)₂, —O—C(CH₃)₃, —O—CH₂CH₂CH₃, —O—CH₂CH₂CH₂CH₃, and substituted or unsubstituted —O-phenyl.

R² is selected from the group consisting of H, —C(R⁶)₂-aryl, and —C(R⁶)₂—O—R⁷, wherein the aryl portion of said —C(R⁶)₂-aryl of R² is unsubstituted or substituted with one or more Y groups. When R² is —C(R⁶)₂-aryl or —C(R⁶)₂—O—R⁷, R⁶, R⁷ and aryl are as defined herein. Non-limiting examples of —C(R⁶)₂-aryl or —C(R⁶)₂—O—R⁷ of R² include:

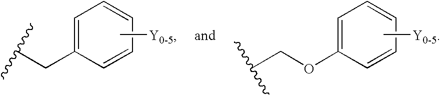

R³ is selected from the group consisting of H, —C(R⁶)₂-aryl, —C(R⁶)₂—O—R¹, —O—R⁷, and —C(R⁶)₂—N(R⁸)₂, wherein the aryl portion of said —C(R⁶)₂-aryl of R³ is unsubstituted or substituted with one or more Y groups. When R³ is —C(R⁶)₂-aryl, —C(R⁶)₂—O—R⁷, —O—R⁷, or —C(R⁶)₂—N(R⁸)₂, R⁶, R⁷, R⁵ and aryl are as defined herein. Non-limiting examples of —C(R⁶)₂-aryl, —C(R⁶)₂—O—R⁷, —O—R⁷, or —C(R⁶)₂—N(R⁸)₂ of R³ include:

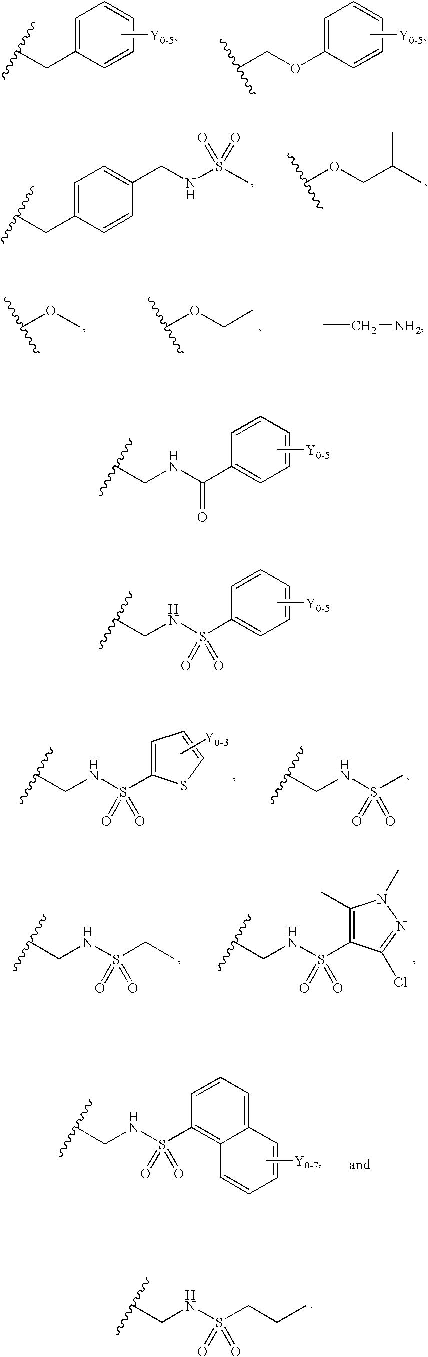

Alternatively, R² and R³ together with the carbon atom to which they are shown attached can form a spiro-fused unsubstituted heterocyclyl ring or a heterocyclyl ring substituted with one or more X groups as defined herein. Non-limiting example of such heterocyclyl rings include piperidyl, piperidinyl, pyrrolidinyl, etc.

R⁴ is selected from the group consisting of H, —C(O)alkyl, and alkyl. Non-limiting examples of —C(O)-alkyl and alkyl of R⁴ include: 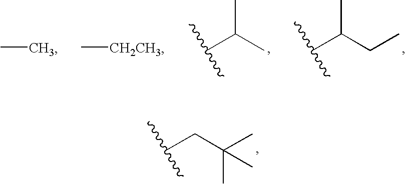
and —C(O)—CH₃.

R⁵ is selected from the group consisting of —C(R⁶)₂)ₘ-G, —S(O)₂-alkyl, —S(O)-cycloalkyl, —C(O)-cycloalkyl, —S(O)₂-aryl, —S(O)₂—(C(R⁶)₂)ₘ-aryl, —S(O)₂-heteroaryl, —C(O)-alkyl, —C(O)-aryl, —C(O)—O—(C₁-C₆)alkyl, —C(O)—O—(C₆-C₁₀)aryl, —C(O)—(C(R⁶)₂)ₘ-aryl, —C(O)-cycloalkylene-aryl, —C(O)-heteroaryl, —C(O)—(C₂-C₁₀)heteroaryl(C₁-C₆)alkyl, —C(O)—(C(R⁶)₂)ₘ—O-aryl, —C(O)-(benzo-fused cycloalkyl), —S(O)₂-(benzo-fused (C₂-C₁₀)heterocyclyl), —C(O)—N(R⁹)—(C(R⁶)₂)ₘ-aryl, —C(O)—N(R⁹)-aryl, cycloalkyl, benzo-fused cycloalkyl, aryl, unsubstituted heterocyclyl, and heterocyclyl substituted with one or more X groups, where m, R⁶, R⁹, G, alkyl, cycloalkyl, benzo-fused cycloalkyl, X, Y aryl, and heterocyclyl are as defined herein. Non-limiting examples of —C(R⁶)₂)ₘ-G of R⁵ include: 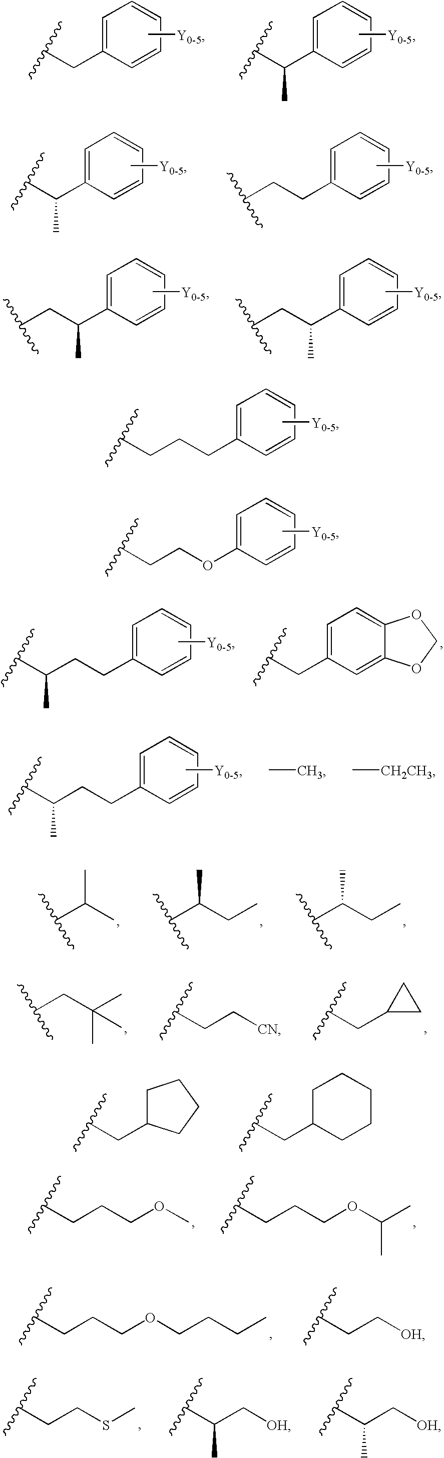 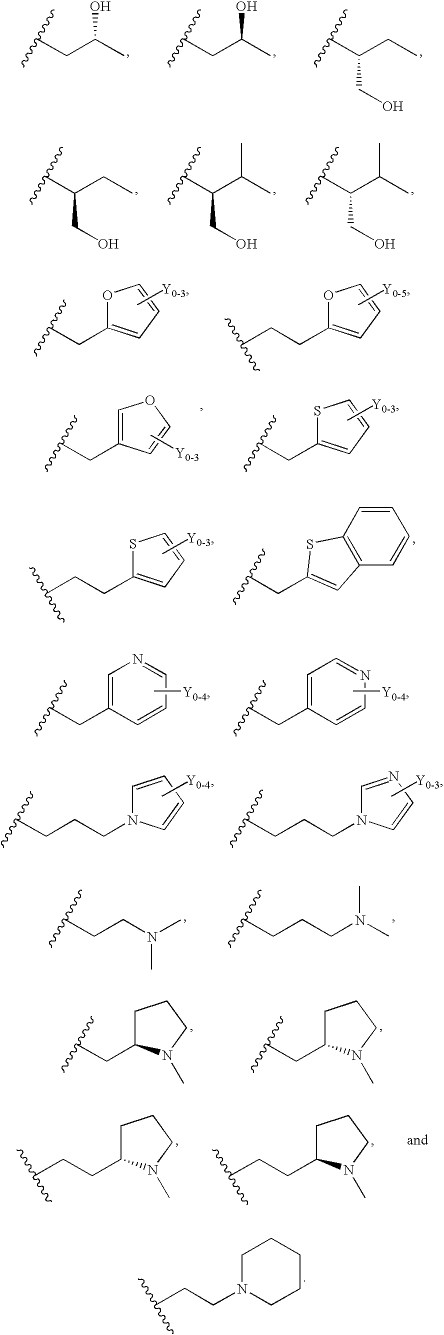
A non-limiting example of —S(O)₂-alkyl of R⁵ includes —S(O)₂—CH₃. Non-limiting examples of —S(O)-cycloalkyl of R⁵ include —S(O)-cyclopropyl, —S(O)-cyclobutyl, —S(O)-cyclopentyl, —S(O)-cyclohexyl, etc. Non-limiting examples of —C(O)-cycloalkyl of R⁵ include —C(O)-cyclopropyl, —C(O)-cyclobutyl, —C(O)-cyclopentyl, —C(O)-cyclohexyl, etc.
Non-limiting examples of —S(O)₂-aryl of R⁵ include: 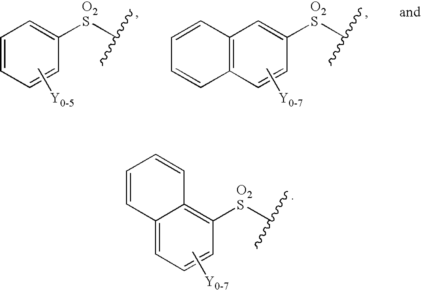
A non-limiting example of —S(O)₂—(C(R⁶)₂)ₘ-aryl of R⁵ includes 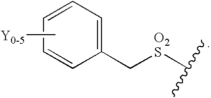
Non-limiting examples of —S(O)₂-heteroaryl of R⁵ includes 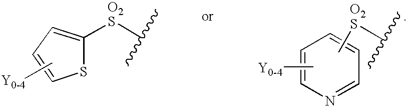
A non-limiting example of —C(O)-alkyl of R⁵ includes —C(O)—CH₃. A non-limiting example of —C(O)aryl of R⁵ includes: 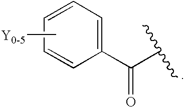
Non-limiting examples of —C(O)—(C(R⁶)₂)ₘ-aryl of R⁵ include: 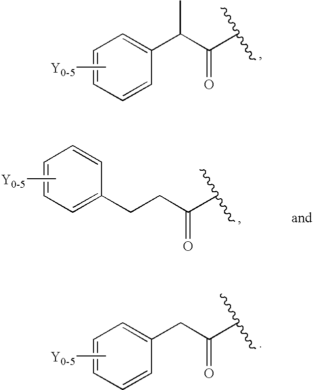
A non-limiting example of —C(O)-cycloalkylene-aryl of R⁵ includes 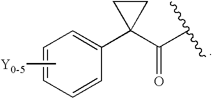
Non-limiting examples of —C(O)-heteroaryl of R⁵ includes 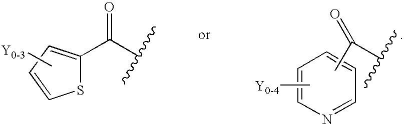
A non-limiting example of —C(O)—(C(R⁶)₂)ₘ—O-aryl of R⁵ includes 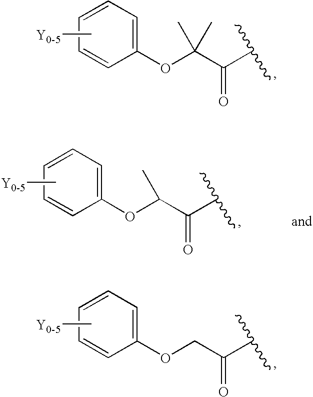
A non-limiting example of —C(O)-(benzo-fused cycloalkyl) of R⁵ includes or 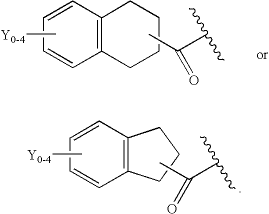
Non-limiting examples of —C(O)—N(R⁹)—(C(R⁶)₂)ₘ-aryl of R⁵ include: 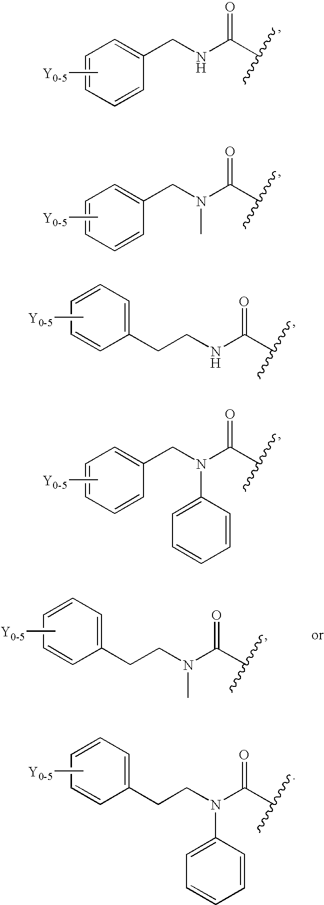
Non-limiting examples of —C(O)—N(R⁹)-aryl or R⁵ include: 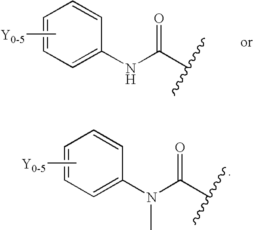
Non-limiting examples of cycloalkyl of R⁵ include: 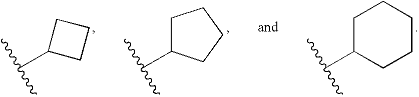
Non-limiting examples of benzo-fused cycloalkyl of R⁵ include: 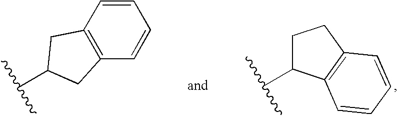
wherein said phenyl portion thereof may be unsubstituted or substituted with one or more Y groups as defined herein. A non-limiting example of an aryl of R⁵ includes unsubstituted phenyl or phenyl substituted with one or more Y groups as defined herein. Non-limiting examples of heterocyclyl of R⁵ include:

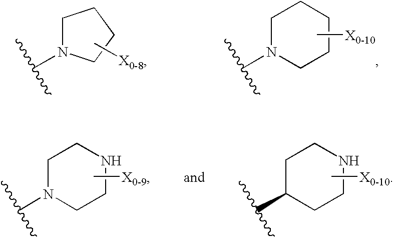

Each R⁶ is independently selected from the group consisting of H and alkyl. Non-limiting examples of R⁶ include H, —CH₃, —CH₂CH₃, —CH₂(CH₃)₂—C(CH₃)₃, and —CH₂C(CH₃)₃.

R⁷ is selected from the group consisting of H, alkyl unsubstituted aryl, and aryl substituted with one or more Y groups. Non-limiting examples of R⁷ include H, —CH₃, —CH₂CH₃, —CH₂(CH₃)₂, —C(CH₃)₃, —CH₂CH₂CH₃, —CH₂CH₂CH₂CH₃, unsubstituted phenyl, and phenyl substituted with one or more Y groups.

Each R⁸ is independently selected from the group consisting of H, alkyl, —C(O)-aryl, —S(O)₂-aryl, and —S(O)₂-heteroaryl, —S(O)₂-alkyl. Non-limiting examples of R⁸ include H, —CH₃, —CH₂CH₃, —CH₂(CH₃)₂, —C(CH₃)₃, —CH₂CH₂CH₃, —CH₂CH₂CH₂CH₃, —C(O)-phenyl, —S(O)₂-phenyl (wherein said phenyl portion may be unsubstituted or substituted with one or more Y groups as defined herein), —S(O)₂-thiophenyl (wherein said thiophenyl portion may be unsubstituted or substituted with one or more Y groups as defined herein), —S(O)₂-imidazolyl (wherein said imidazolyl portion may be unsubstituted or substituted with one or more Y groups as defined herein), —S(O)₂-diazolyl (wherein said diazolyl portion may be unsubstituted or substituted with one or more Y groups as defined herein), —S(O)₂-triazolyl (wherein said triazolyl portion may be unsubstituted or substituted with one or more Y groups as defined herein), —S(O)₂-pyridyl (wherein said pyridyl portion may be unsubstituted or substituted with one or more Y groups as defined herein), —S(O)₂—CH₃, —S(O)₂—CH₂CH₃, and —S(O)₂—CH₂CH₂CH₃.

Each R⁹ is independently selected from the group consisting of H, alkyl, cycloalkyl, and substituted or unsubstituted aryl. Non-limiting examples of R⁹ include H, —CH₃, —CH₂CH₃, —CH₂(CH₃)₂, —C(CH₃)₃, —CH₂C(CH₃)₃, cyclopropyl, cyclobutyl, cyclopentyl, cyclohexyl, phenyl, and naphthyl.

G is selected from the group consisting of H, alkyl, unsubstituted aryl, aryl substituted with one or more Y groups, —CN, cycloalkyl, —O—R⁷, —S—R⁷, unsubstituted heteroaryl, heteroaryl substituted with one or more Y groups, —N(R⁸)₂, unsubstituted heterocyclyl, and heterocyclyl substituted with one or more X groups. When G is alkyl, non-limiting examples of G include —CH₃, —CH₂CH₃, —CH₂(CH₃)₂, —C(CH₃)₃, —CH₂CH₂CH₃, —CH₂CH₂CH₂CH₃. When G is unsubstituted aryl, non-limiting examples include phenyl and naphthyl. When G is substituted aryl, non-limiting examples include: 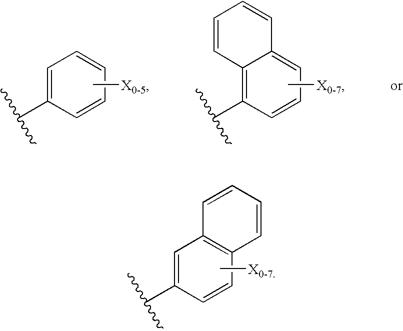
When G is cycloalkyl, non-limiting examples of G include cyclopropyl, cyclobutyl, cyclopentyl, and cyclohexyl. When G is unsubstituted or substituted heteroaryl, non-limiting examples include: 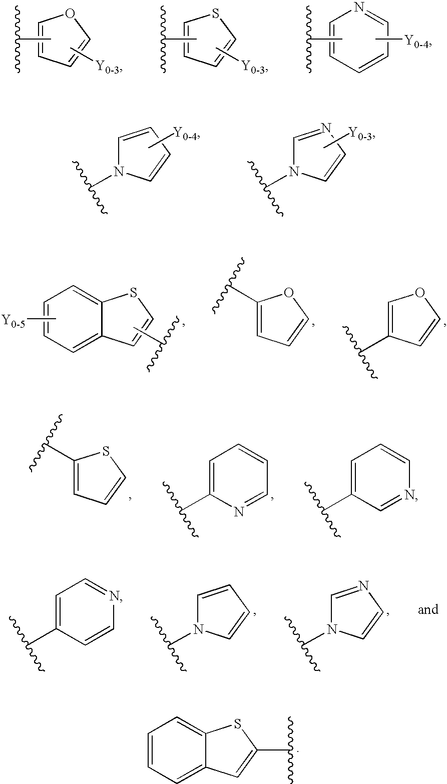
When G is unsubstituted or substituted heterocyclyl, non-limiting examples include any of the unsubstituted or substituted heteroaryls described above, as well as: 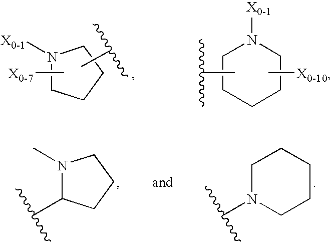
When G is —O—R⁷, —S—R⁷ or —N(R⁸)₂, R⁷ and R⁸ are each defined as described above.

Each X is independently selected from the group consisting of alkyl, —C(O)—N(R⁹)₂, —C(O)-heteroaryl (wherein said heteroaryl portion is optionally substituted with one or more halogen), heteroaryl (wherein said heteroaryl is optionally substituted with one or more halogen), —C(R⁶)₂)ₘ-aryl (wherein said aryl portion is optionally substituted with one or more substituents selected from the group consisting of halogen, —OH, —O-alkyl, haloalkyl, and —CN), and aryl (wherein said aryl portion is optionally substituted with one or more substituents selected from the group consisting of halogen, —OH, —O-alkyl, haloalkyl, and —CN). When X is alkyl, non-limiting examples of X include —CH₃ and —CH₂CH₃. When X is —C(O)—N(R⁹)₂, each R⁹ is independently defined as described above. When X is —C(O)-heteroaryl, non-limiting examples of X include: 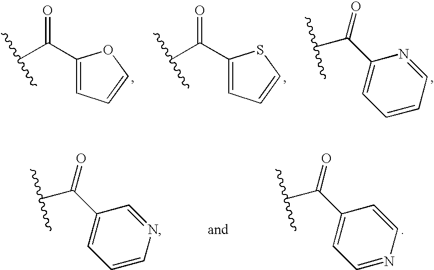
When X is heteroaryl, non-limiting examples of X include: 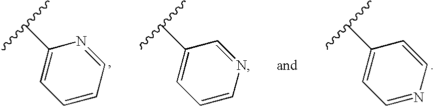
When X is —C(R⁶)₂)ₘ-aryl, R⁶ is defined as described above, non-limiting examples of said aryl portion of —(C(R⁶)₂)-aryl include phenyl, chlorophenyl, dichlorophenyl, and naphthyl; e.g., non-limiting examples of X include benzyl, chlorobenzyl, and dichlorobenzyl. When X is aryl, non-limiting examples of X include phenyl, chlorophenyl, dichlorophenyl, and naphthyl.

Each Y is independently selected from the group consisting of halogen, alkyl, aryl, —C(O)-alkyl, —O—R⁹, haloalkyl, —O-haloalkyl, —CN, and —C(O)O-alkyl, —N(R⁶)₂, —C(R⁶)₂—N(R⁶)₂, and —C(R⁶)₂—N(R⁶)—S(O)₂—R⁶; or two Y groups form a —O—CH₂—O— group. When Y is halogen, non-limiting examples of Y include F, Cl, and Br. When Y is alkyl, non-limiting examples include methyl, ethyl, n-propyl, i-propyl, n-butyl, sec-butyl, i-butyl, t-butyl, etc. When Y is aryl, non-limiting examples include phenyl or naphthyl. When Y is —C(O)-alkyl, non-limiting examples include —C(O)—CH₃, —C(O)—CH₂CH₃, —C(O)—CH₂CH₂CH₃, —C(O)—CH(CH₃)₂, —C(O)—CH₂CH₂CH₂CH₃, —C(O)—CH(CH₃)CH₂CH₃, —C(O)—CH₂CH(CH₃)₂, —C(O)—C(CH₃)₃, etc. When Y is —O—R⁹, R⁹ is defined as described above. When Y is haloalkyl, non-limiting examples of Y include-CF₃, —CHF₂, —CH₂F, —CH₂CF₃, and —CF₂CF₃. When Y is —O-haloalkyl, non-limiting examples include —CF₃, —O—CHF₂, —O—CH₂F, —O—CH₂CF₃, and —O—CF₂CF₃. When Y is —C(O)—O-alkyl non-limiting examples include —C(O)—O—CH₃, —C(O)—O—CH₂CH₃, —C(O)—O—CH₂CH₂CH₃, —C(O)—O—CH(CH₃)₂, —C(O)—O—CH₂CH₂CH₂CH₃, —C(O)—O—CH(CH₃)CH₂CH₃, —C(O)—O—CH₂CH(CH₃)₂, —C(O)—O—C(CH₃)₃, etc. When Y is —N(R⁶)₂ or —C(R⁶)₂—N(R⁶)₂, each R⁶ is defined independently as described above. For example, —C(R⁶)₂—N(R⁶)₂ includes —CH₂NH₂ and —CH₂—N(H)CH₃, and —N(R⁶)₂ includes —NH₂ and —N(CH₃)₂. When Y is —C(R⁶)₂—N(R⁶)—S(O)₂—R⁶, —C(R⁶)₂—N(R⁶)—S(O)₂—R⁶ includes —CH₂—NH—SO₂—CH₃, —CH₂—N(CH₃)—SO₂—CH₃, —CH₂—NH—SO₂—CH₂CH₃, —CH₂—N(CH₃)—SO₂—CH₂CH₃, etc.

The variable “n” can be 0, 1, 2, 3, 4, or 5, and variable “m” can be 1, 2, 3, 4, or 5.

In another embodiment, the compound of Formula (I) is a compound having the following structural Formula: 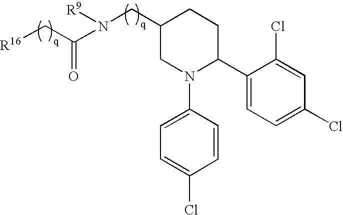
wherein:
each R⁶ is independently selected from the group consisting of H and (C₁-C₆)alkyl;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each R¹² is independently selected from the group consisting of H, (C₁-C₆)alkyl, (C₃-C₆)cycloalkyl(C₁-C₆)alkyl, —(C(R⁶)₂)_{q}—C(O)R¹³, benzo(C₂-C₁₀)heterocyclyl, benzocyclo(C₁-C₆)alkyl, —(C(R⁶)₂)_{q}—N(R⁹)—C(O)R³, —(C(R⁶)₂)_{q}—N(R¹⁴)₂, (C₆-C₁₀)aryl(C₁-C₆)alkyl (C₂-C₁₀)heteroaryl(C₁-C₆)alkyl, HO—(C₁-C₆)alkyl-, (C₁-C₆)alkyl-O—, (C₆-C₁₀)aryl-O—, Y—(C₁-C₆)alkylenyl-O—, W—O—(C₁-C₆)alkylenyl, (C₂-C₁₀)heterocyclyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups,
   wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
   wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
   with the following provisos that
for —N(R¹⁴)₂ of R¹², the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each R¹³ is independently selected from the group consisting of H, (C₁-C₆)alkyl, (C₃-C₆)cycloalkyl(C₁-C₆)alkyl, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups;
each R¹⁴ is independently selected from the group consisting of H, Boc, unsubstituted (C₁-C₆)alkyl, (C₁-C₆)alkyl substituted with one or more X groups, unsubstituted (C₃-C₆)cycloalkyl, (C₃-C₆)cycloalkyl substituted with one or more Y groups, unsubstituted (C₆-C₁₀)aryl, (C₆-C₁₀)aryl substituted with one or more Y groups, (C₂-C₁₀)heterocyclyl, unsubstituted (C₂-C₁₀)heteroaryl and (C₂-C₁₀)heteroaryl substituted with one or more Y groups;
each R¹⁶ is independently selected from the group consisting of H, (C₁-C₆)alkyl, (C₃-C₆)cycloalkyl(C₁-C₆)alkyl, —(C(R⁶)₂)ₚ—C(O)R¹³, —(C(R⁶)₂)ₚ—N(R⁹)—C(O)R¹³, —(C(R⁶)₂)ₚ—N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups,
   for —N(R¹⁴)₂, the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each W is independently selected from the group consisting of hydrogen, (C₁-C₆)alkyl, (C₆-C₁₀)aryl, —C(O)—(C₁-C₆)alkyl, —C(O)—O—(C₁-C₆)alkyl, —C(R⁶)₂—N(R⁶)₂, and —C(R⁶)₂—N(R⁶)—S(O)₂—R⁶;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)—O—(C₁-C₆)alkyl, —C(R⁶)₂)ₘ—(C₆-C₁₀)aryl and (C₆-C₁₀)aryl
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
      wherein said (C₂-C₁₀)heteroaryl or the (C₂-C₁₀)heteroaryl portion of said —C(O)(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
      wherein said (C₆-C₁₀)aryl or the (C₆-C₁₀)aryl portion of said —C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
      wherein in a single X moiety, ═O, can replace two available hydrogens on the same carbon on a ring system;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each n, p and q is independently an integer from 0-5; and
m is an integer from 1-5.

In another embodiment, the compound of Formula (I) is a compound having the following structural Formula: 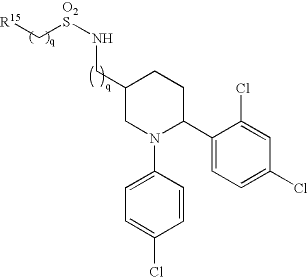
wherein:
R⁴ is selected from the group consisting of H, —C(O)(C₁-C₆)alkyl, and (C₁-C₆)alkyl;
R⁵ is selected from the group consisting of —C(R⁶)₂)ₘ-G, —S(O)₂—(C₁-C₆)alkyl, —S(O)₂—(C₃-C₆)cycloalkyl, (C₁-C₆)alkyl, —S(O)—(C₃-C₆)cycloalkyl, —C(O)—(C₃-C₆)cycloalkyl, —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₁-C₆)alkyl, —C(O)—(C₆-C₁₀)aryl, —C(O)—O—(C₁-C₆)alkyl, —C(O)—O—(C₆-C₁₀)aryl, —C(O)(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C₂-C₁₀)heteroaryl(C₁-C₆)alkyl, —C(O)—(C(R⁶)₂)ₘ—O—(C₆-C₁₀)aryl, —C(O)-(benzo-fused (C₃-C₆)cycloalkyl), —S(O)₂-(benzo-fused (C₂-C₁₀)heterocyclyl), —C(O)—N(R⁹)—(C(R⁶)₂)ₘ—(C₆-C₁₀)aryl, —C(O)—N(R⁹)—(C₆-C₁₀)aryl, (C₃-C₆)cycloalkyl, benzo-fused (C₃-C₆)cycloalkyl, unsubstituted (C₆-C₁₀)aryl, (C₆-C₁₀)aryl substituted with one or more Y groups, unsubstituted (C₂-C₁₀)heterocyclyl, and (C₂-C₁₀)heterocyclyl substituted with one or more X groups,
   wherein the (C₆-C₁₀)aryl or (C₂-C₁₀)heteroaryl portion of said —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C(R⁶)₂)ₘ—O—(C₆-C₁₀)aryl, —C(O)—N(R⁹)—(C(R⁶)₂)ₘ—(C₆-C₁₀)aryl, or —C(O)—N(R⁹)—(C₆-C₁₀)aryl of R⁵ is unsubstituted or substituted with one or more Y groups;
   wherein the heterocyclyl portion of —S(O)₂-(benzo-fused (C₂-C₁₀)heterocyclyl)aryl of R⁵ is unsubstituted or substituted with one or more X groups;
each R⁶ is independently selected from the group consisting of H and (C₁-C₆)alkyl;
R⁷ is selected from the group consisting of H, (C₁-C₆)alkyl, unsubstituted (C₂-C₁₀)heteroaryl and (C₂-C₁₀)heteroaryl substituted with one or more Y groups, unsubstituted (C₆-C₁₀)aryl, and (C₆-C₁₀)aryl substituted with one or more Y groups;
each R⁸ is independently selected from the group consisting of H, (C₁-C₆)alkyl, (C₆-C₁₀)aryl(C₁-C₆)alkyl, (C₂-C₁₀)heteroaryl(C₁-C₆)alkyl, unsubstituted (C₆-C₁₀)aryl, unsubstituted (C₂-C₁₀)heteroaryl, —C(O)—(C₁-C₆)alkyl, —C(O)—(C₆-C₁₀)aryl, —C(O)—(C₃-C₆)cycloalkyl, —C(O)N(R⁹)₂, —S(O)₂—(C₆-C₁₀)aryl, —S(O)₂—(C₂-C₁₀)heteroaryl, —SO₂N(R⁹)₂, —S(O)₂—(C₃-C₆)cycloalkyl, (C₆-C₁₀)aryl and (C₂-C₁₀)heteroaryl substituted with one or more Y groups, and —S(O)₂—(C₁-C₆)alkyl,
   wherein the (C₆-C₁₀)aryl portion of said (C₆-C₁₀)aryl(C₁-C₆)alkyl, —C(O)—(C₆-C₁₀)aryl or —S(O)₂—(C₆-C₁₀)aryl and the (C₂-C₁₀)heteroaryl portion of said (C₂-C₁₀)heteroaryl(C₁-C₆)alkyl, —S(O)₂—(C₂-C₁₀)heteroaryl of R⁸ is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each R¹² is independently selected from the group consisting of H, (C₁-C₆)alkyl, (C₃-C₆)cycloalkyl(C₁-C₆)alkyl, —(C(R⁶)₂)_{q}—C(O)R¹³, benzo(C₂-C₁₀)heterocyclyl, benzocyclo(C₁-C₆)alkyl, —(C(R⁶)₂)_{q}—N(R⁹)—C(O)R¹³—(C(R⁶)₂)_{q}—N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, Y—(C₁-C₆)alkylenyl-O—, W—O—(C₁-C₆)alkylenyl, (C₂-C₁₀)heterocyclyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups,
   wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
   wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
   with the following provisos that
   for —N(R¹⁴)₂ of R¹², the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each R¹³ is independently selected from the group consisting of H, (C₁-C₆)alkyl, (C₃-C₆)cycloalkyl(C₁-C₆)alkyl, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups;
each R¹⁴ is independently selected from the group consisting of H, Boc, unsubstituted (C₁-C₆)alkyl, (C₁-C₆)alkyl substituted with one or more X groups, unsubstituted (C₃-C₆)cycloalkyl, (C₃-C₆)cycloalkyl substituted with one or more Y groups, unsubstituted (C₆-C₁₀)aryl, (C₆-C₁₀)aryl substituted with one or more Y groups, (C₂-C₁₀)heterocyclyl, unsubstituted (C₂-C₁₀)heteroaryl and (C₂-C₁₀)heteroaryl substituted with one or more Y groups;
each R¹⁵ is independently selected from the group consisting of H, (C₁-C₆)alkyl, —N(R⁴)(R⁵), (C(R⁶)₂)_{q}—N(R¹⁴)₂, (C₁-C₆)alkylenyl-CF₃, —CF₃, (C₃-C₆)cycloalkyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, benzo(C₂-C₁₀)heterocyclyl, benzocyclo(C₁-C₆)alkyl, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups,
   wherein the (C₁-C₆)alkyl portion of said (C₃-C₆)cycloalkyl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups,
   wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
G is selected from the group consisting of H, (C₁-C₆)alkyl, unsubstituted (C₆-C₁₀)aryl, (C₆-C₁₀)aryl substituted with one or more Y groups, —CN, (C₃-C₆)cycloalkyl, —O—R⁷, —S—R⁷, unsubstituted (C₂-C₁₀)heteroaryl, (C₂-C₁₀)heteroaryl substituted with one or more Y groups, —N(R⁸)₂, unsubstituted (C₂-C₁₀)heterocyclyl, and (C₂-C₁₀)heterocyclyl substituted with one or more X groups;
each W is independently selected from the group consisting of hydrogen, (C₁-C₆)alkyl, (C₆-C₁₀)aryl, —C(O)—(C₁-C₆)alkyl, —C(O)—O—(C₁-C₆)alkyl, —C(R⁶)₂—N(R⁶)₂, and —C(R⁶)₂—N(R⁶)—S(O)₂—R⁶;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)O—(C₁-C₆)alkyl, —C(R⁶)₂)ₘ—(C₆-C₁₀)aryl and (C₆-C₁₀)aryl
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
      wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
      wherein said (C₆-C₁₀)aryl or the (C₆-C₁₀)aryl portion of said —(C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
      wherein in a single X moiety, ═O, can replace two available hydrogens on the same carbon on a ring system;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each n, p and q is independently an integer from 0-5; and
m is an integer from 1-5.

In another embodiment, the compound of Formula (I) is a compound having the following structural Formula: 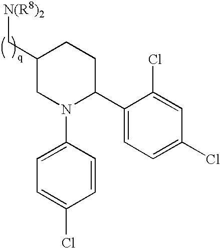
wherein:
each R⁶ is independently selected from the group consisting of H and (C₁-C₆)alkyl;
each R⁸ is independently selected from the group consisting of H, (C₁-C₆)alkyl, (C₆-C₁₀)aryl(C₁-C₆)alkyl, (C₂-C₁₀)heteroaryl(C₁-C₆)alkyl, unsubstituted (C₆-C₁₀)aryl, unsubstituted (C₂-C₁₀)heteroaryl, —C(O)—(C₁-C₆)alkyl, —C(O)—(C₆-C₁₀)aryl, —C(O)—(C₃-C₆)cycloalkyl, —C(O)N(R⁹)₂, —S(O)₂—(C₆-C₁₀)aryl, —S(O)₂—(C₂-C₁₀)heteroaryl, —SO₂N(R⁹)₂, —S(O)₂—(C₃-C₆)cycloalkyl, (C₆-C₁₀)aryl and (C₂-C₁₀)heteroaryl substituted with one or more Y groups, and —S(O)₂—(C₁-C₆)alkyl,
   wherein the (C₆-C₁₀)aryl portion of said (C₆-C₁₀)aryl(C₁-C₆)alkyl, —C(O)—(C₆-C₁₀)aryl or —S(O)₂—(C₆-C₁₀)aryl and the (C₂-C₁₀)heteroaryl portion of said (C₂-C₁₀)heteroaryl(C₁-C₆)alkyl, —S(O)₂—(C₂-C₁₀)heteroaryl of R⁸ is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each q is independently an integer from 0 to 5.

In another embodiment, the compound of Formula (I) is a compound having the following structural Formula: 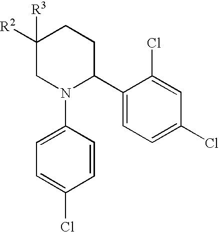
wherein
R² is selected from the group consisting of H, —(C(R⁶)₂)ₚ—(C₆-C₁₀)aryl, (C₃-C₆)cycloalkyl(C₁-C₆)alkyl, (C₃-C₆)cycloalkyl(C₁-C₆)alkyl substituted with Z, —(C(R⁶)₂)_{q}—(C₂-C₁₀)heterocyclyl, —(C(R⁶)₂)ₚ—S(O)₂—(C₂-C₁₀)heterocyclyl, and —C(R⁶)₂—O—R⁷,
   wherein the (C₆-C₁₀)aryl portion of said —C(R⁶)₂—(C₆-C₁₀)aryl of R² is unsubstituted or substituted with one or more Y groups,
   wherein the (C₂-C₁₀)heterocyclyl portion of said —(C(R⁶)₂)ₚ—S(O)₂—(C₂-C₁₀)heterocyclyl of R² is unsubstituted or substituted with one or more X groups,
   wherein the (C₂-C₁₀)heterocyclyl portion of said —(C(R⁶)₂)_{q}—(C₂-C₁₀)heterocyclyl of R² is unsubstituted or substituted with one or more X groups;
R³ is selected from the group consisting of H, —(C(R⁶)₂)_{q}—C(O)—N(R¹²)₂ or —C(R⁶)₂)_{q}—N(R⁸)₂;
each R⁶ is independently selected from the group consisting of H and (C₁-C₆)alkyl;
R⁷ is selected from the group consisting of H, (C₁-C₆)alkyl, unsubstituted (C₂-C₁₀)heteroaryl and (C₂-C₁₀)heteroaryl substituted with one or more Y groups, unsubstituted (C₆-C₁₀)aryl, and (C₆-C₁₀)aryl substituted with one or more Y groups;
each R⁸ is independently selected from the group consisting of H, (C₁-C₆)alkyl, (C₆-C₁₀)aryl(C₁-C₆)alkyl, (C₂-C₁₀)heteroaryl(C₁-C₆)alkyl, unsubstituted (C₆-C₁₀)aryl, unsubstituted (C₂-C₁₀)heteroaryl, —C(O)—(C₁-C₆)alkyl, —C(O)—(C₆-C₁₀)aryl, —C(O)—(C₃-C₆)cycloalkyl, —C(O)N(R⁹)₂, —S(O)₂—(C₆-C₁₀)aryl, —S(O)₂—(C₂-C₁₀)heteroaryl, —SO₂N(R⁹)₂, —S(O)₂—(C₃-C₆)cycloalkyl, (C₆-C₁₀)aryl and (C₂-C₁₀)heteroaryl substituted with one or more Y groups, and —S(O)₂—(C₁-C₆)alkyl,
   wherein the (C₆-C₁₀)aryl portion of said (C₆-C₁₀)aryl(C₁-C₆)alkyl, —C(O)—(C₆-C₁₀)aryl or —S(O)₂—(C₆-C₁₀)aryl and the (C₂-C₁₀)heteroaryl portion of said (C₂-C₁₀)heteroaryl(C₁-C₆)alkyl, —S(O)₂—(C₂-C₁₀)heteroaryl of R⁸ is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each R¹² is independently selected from the group consisting of H, (C₁-C₆)alkyl, (C₃-C₆)cycloalkyl(C₁-C₆)alkyl, —(C(R⁶)₂)_{q}—C(O)R¹³, benzo(C₂-C₁₀)heterocyclyl, benzocyclo(C₁-C₆)alkyl, —(C(R⁶)₂)_{q}—N(R⁹)—C(O)R¹³, (C(R⁸)₂)_{q} N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, Y—(C₁-C₆)alkylenyl-O—, W—O—(C₁-C₆)alkylenyl, (C₂-C₁₀)heterocyclyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups,
   wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
   wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
   with the following provisos that
   for —N(R¹⁴)₂ of R¹², the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each R¹³ is independently selected from the group consisting of H, (C₁-C₆)alkyl, (C₃-C₆)cycloalkyl(C₁-C₆)alkyl, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups;
each R¹⁴ is independently selected from the group consisting of H, Boc, unsubstituted (C₁-C₆)alkyl, (C₁-C₆)alkyl substituted with one or more X groups, unsubstituted (C₃-C₆)cycloalkyl, (C₃-C₆)cycloalkyl substituted with one or more Y groups, unsubstituted (C₆-C₁₀)aryl, (C₆-C₁₀)aryl substituted with one or more Y groups, (C₂-C₁₀)heterocyclyl, unsubstituted (C₂-C₁₀)heteroaryl and (C₂-C₁₀)heteroaryl substituted with one or more Y groups;
each W is independently selected from the group consisting of hydrogen, (C₁-C₆)alkyl, (C₆-C₁₀)aryl, —C(O)—(C₁-C₆)alkyl, —C(O)—O—(C₁-C₆)alkyl, —C(R⁶)₂—N(R⁶)₂, and —C(R⁶)₂—N(R⁶)—S(O)₂—R⁶;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)—O—(C₁-C₆)alkyl, —C(R⁶)₂)ₘ—(C₆-C₁₀)aryl and (C₆-C₁₀)aryl
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
      wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
      wherein said (C₆-C₁₀)aryl or the (C₆-C₁₀)aryl portion of said —(C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
      wherein in a single X moiety, ═O, can replace two available hydrogens on the same carbon on a ring system;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a—CH₂—O— or —O—CH₂CH₂—O— group;
each Z is independently selected from the group consisting of hydrogen, (C₁-C₆)alkyl, (C₆-C₁₀)aryl(C₁-C₆)alkyl, (C₂-C₁₀)heteroaryl(C₁-C₆)alkyl, —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(R⁶)₂)ₘ—(C₆-C₁₀)aryl, —N(R⁶)—S(O)₂—R⁹ and (C₆-C₁₀)aryl
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
      wherein said (C₂-C₁₀)heteroaryl or the (C₂-C₁₀)heteroaryl portion of said —C(O)—(C₂-C₁₀)heteroaryl of Z is unsubstituted or substituted with one or more substituents selected from the group consisting of halogen, —OH, —O—(C₁-C₆)alkyl, halo(C₁-C₆)alkyl, and —CN, and
      wherein said (C₆-C₁₀)aryl or the (C₆-C₁₀)aryl portion of said —C(R⁶)₂)ₘ—(C₆-C₁₀)aryl of Z is unsubstituted or substituted with one or more substituents selected from the group consisting of halogen, —OH, —O—(C₁-C₆)alkyl, halo(C₁-C₆)alkyl, and —CN;
      wherein in a single Z moiety, ═O, can replace two available hydrogens on the same carbon on a ring system;
each n, p and q is independently an integer from 0-5; and
m is an integer from 1-5.

In another embodiment, the compound of Formula (I) is a compound having the following structural Formula: 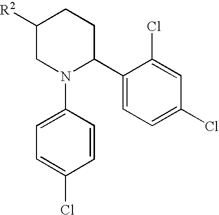
wherein;
R² is —(C(R⁶)₂)_{q}—(C₂-C₁₀)heterocyclyl;
   wherein the (C₂-C₁₀)heterocyclyl portion of said —(C(R⁶)₂)_{q}—(C₂-C₁₀)heterocyclyl of R² is unsubstituted or substituted with one or more X groups;
each R⁶ is independently selected from the group consisting of H and (C₁-C₆)alkyl;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each R¹² is independently selected from the group consisting of H, (C₁-C₆)alkyl, (C₃-C₆)cycloalkyl(C₁-C₆)alkyl, —(C(R⁶)₂)_{q}—C(O)R¹³, benzo(C₂-C₁₀)heterocyclyl, benzocyclo(C₁-C₆)alkyl, —(C(R⁶)₂)_{q}—N(R⁹)—C(O)R¹³, —(C(R⁶)₂)_{q}—N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, Y—(C₁-C₆)alkylenyl-O—, W—O—(C₁-C₆)alkylenyl, (C₂-C₁₀)heterocyclyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups,
   wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
   wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
   with the following provisos that
   for —N(R¹⁴)₂ of R¹², the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each R¹³ is independently selected from the group consisting of H, (C₁-C₆)alkyl, (C₃-C₆)cycloalkyl(C₁-C₆)alkyl, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups;
each R¹⁴ is independently selected from the group consisting of H, Boc, unsubstituted (C₁-C₆)alkyl, (C₁-C₆)alkyl substituted with one or more X groups, unsubstituted (C₃-C₆)cycloalkyl, (C₃-C₆)cycloalkyl substituted with one or more Y groups, unsubstituted (C₆-C₁₀)aryl, (C₆-C₁₀)aryl substituted with one or more Y groups, (C₂-C₁₀)heterocyclyl, unsubstituted (C₂-C₁₀)heteroaryl and (C₂-C₁₀)heteroaryl substituted with one or more Y groups;
each W is independently selected from the group consisting of hydrogen, (C₁-C₆)alkyl, (C₆-C₁₀)aryl, —C(O)—(C₁-C₆)alkyl, —C(O)—O—(C₁-C₆)alkyl, —C(R⁶)₂—N(R⁶)₂, and —C(R⁶)₂—N(R⁶)—S(O)₂—R⁶;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)O—(C₁-C₆)alkyl, —C(R⁶)₂)ₘ—(C₆-C₁₀)aryl and (C₆-C₁₀)aryl
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
      wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
      wherein said (C₆-C₁₀)aryl or the (C₆-C₁₀)aryl portion of said —C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
      wherein in a single X moiety, ═O, can replace two available hydrogens on the same carbon on a ring system;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each n, p and q is independently an integer from 0-5; and
m is an integer from 1-5.

In another embodiment of a compound of Formula (I), or a pharmaceutically acceptable salt, solvate, or ester thereof, R³ is —C(R⁶)₂)_{q}—N(R⁸)₂ or —(C(R⁶)₂)_{q}—(C₂-C₁₀)heterocyclyl.

In another embodiment, the compound of Formula (I) is a compound having the following structural Formula: 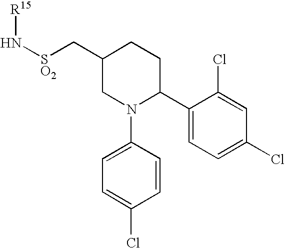
or a pharmaceutically acceptable salt, solvate or ester thereof, wherein:
R¹⁵ is alkyl.

In another embodiment, the compound of Formula (I) is a compound having the following structural Formula: 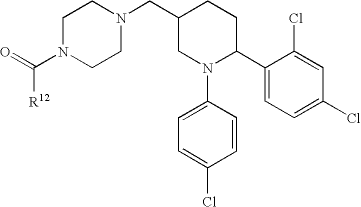
or a pharmaceutically acceptable salt, solvate or ester thereof, wherein:
each R⁶ is independently selected from the group consisting of H and (C₁-C₆)alkyl;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each R¹² is independently selected from the group consisting of H, (C₁-C₆)alkyl, (C₃-C₆)cycloalkyl(C₁-C₆)alkyl, —(C(R⁶)₂)_{q}—C(O)R¹³, benzo(C₂-C₁₀)heterocyclyl, benzocyclo(C₁-C₆)alkyl, —(C(R⁶)₂)_{q}—N(R⁹)—C(O)R¹³, —(C(R⁶)₂)_{q}—N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, Y—(C₁-C₆)alkylenyl-O—, W—O—(C₁-C₆)alkylenyl, (C₂-C₁₀)heterocyclyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups,
   wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
   wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
   with the following provisos that
   for —N(R¹⁴)₂ of R¹², the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each R¹³ is independently selected from the group consisting of H, (C₁-C₆)alkyl, (C₃-C₆)cycloalkyl(C₁-C₆)alkyl, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
   wherein the (C₃-C₆)cycloalkyl of said (C₃-C₆)cycloalkyl(C₁-C₆)alkyl is unsubstituted or substituted with one or more X groups;
each R¹⁴ is independently selected from the group consisting of H, Boc, unsubstituted (C₁-C₆)alkyl, (C₁-C₆)alkyl substituted with one or more X groups, unsubstituted (C₃-C₆)cycloalkyl, (C₃-C₆)cycloalkyl substituted with one or more Y groups, unsubstituted (C₆-C₁₀)aryl, (C₆-C₁₀)aryl substituted with one or more Y groups, (C₂-C₁₀)heterocyclyl, unsubstituted (C₂-C₁₀)heteroaryl and (C₂-C₁₀)heteroaryl substituted with one or more Y groups;
each W is independently selected from the group consisting of hydrogen, (C₁-C₆)alkyl, (C₆-C₁₀)aryl, —C(O)—(C₁-C₆)alkyl, —C(O)—O—(C₁-C₆)alkyl, —C(R⁶)₂—N(R⁶)₂, and —C(R⁶)₂—N(R⁶)—S(O)₂—R⁶;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)—O—(C₁-C₆)alkyl, —C(R⁶)₂)ₘ—(C₆-C₁₀)aryl and (C₆-C₁₀)aryl
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
      wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
      wherein said (C₆-C₁₀)aryl or the (C₆-C₁₀)aryl portion of said —C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
      wherein in a single X moiety, ═O, can replace two available hydrogens on the same carbon on a ring system;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each n, p and q is independently an integer from 0-5; and
m is an integer from 1-5.

In another embodiment, the compound of Formula (I) is a compound having the following structural Formula: 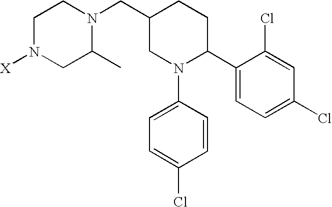
or a pharmaceutically acceptable salt, solvate or ester thereof, wherein:
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
R¹² is (C₁-C₆)alkyl;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)—O—(C₁-C₆)alkyl, —C(R⁶)₂)ₘ—(C₆-C₁₀)aryl and (C₆-C₁₀)aryl
   wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
   wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
      wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
      wherein said (C₆-C₁₀)aryl or the (C₆-C₁₀)aryl portion of said —C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
      wherein in a single X moiety, ═O, can replace two available hydrogens on the same carbon on a ring system.

In another embodiment, the compound of Formula (I) is a compound having the following structural Formula: 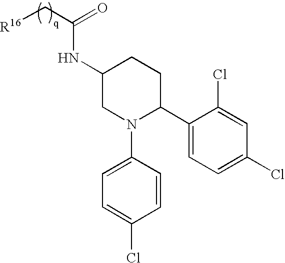
or a pharmaceutically acceptable salt, solvate or ester thereof, wherein:
R¹⁶ is —O—(C₁-C₆)alkyl or (C₂-C₁₀)heteroaryl(C₁-C₆)alkyl
   q is 1 or 2.

In another embodiment, the compound of Formula (I) has the following structure: 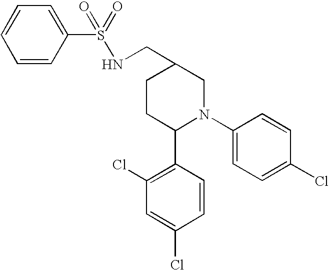
or a pharmaceutically acceptable salt, solvate, or ester thereof.

In another embodiment, the compound of Formula (I) has the following structure: 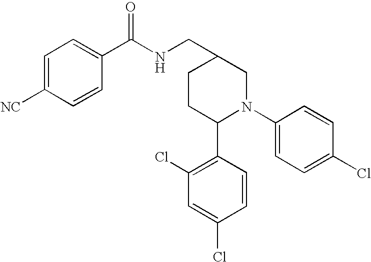
or a pharmaceutically acceptable salt, solvate, or ester thereof.

In another embodiment, the compound of Formula (I) has the following structure: 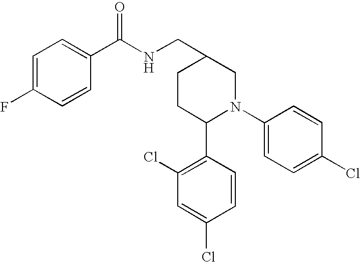
or a pharmaceutically acceptable salt, solvate, or ester thereof.

In another embodiment, the compound of Formula (I) has the following structure: 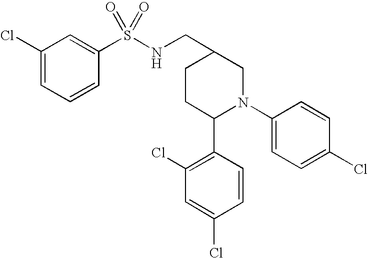
or a pharmaceutically acceptable salt, solvate, or ester thereof.

In another embodiment, the compound of Formula (I) has the following structure: 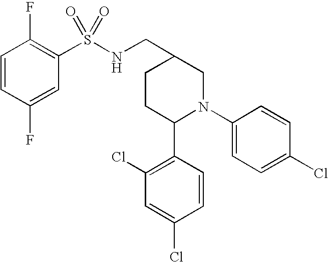
or a pharmaceutically acceptable salt, solvate, or ester thereof.

In another embodiment, the compound of Formula (I) has the following structure: 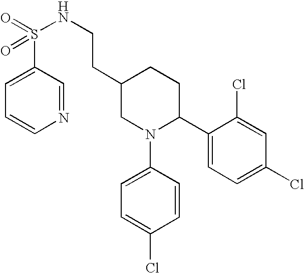
or a pharmaceutically acceptable salt, solvate, or ester thereof.

In another embodiment, the compound of Formula (I) has the following structure: 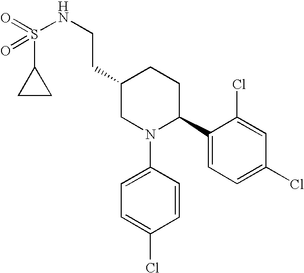
or a pharmaceutically acceptable salt, solvate, or ester thereof.

The compounds of Formula (I), or pharmaceutically acceptable salts, solvates, or esters thereof, are preferably purified to a degree suitable for use as a pharmaceutically active substance. That is, the compounds of Formula (I) can have a purity of 95 wt % or more (excluding adjuvants such as pharmaceutically acceptable carriers, solvents, etc., which are used in formulating the compound of Formula (I) into a conventional form, such as a pill, capsule, IV solution, etc. suitable for administration into a patient). In other embodiments, the purity can be 97 wt % or more, or 99 wt % or more. A purified compound of Formula (I) includes a single isomer having a purity, as discussed above, of 95 wt % or more, 97 wt % or more, or 99 wt % or more, as discussed above. For example, the purified compound of Formula (I) can include a compound of Structure (IA), (IB), (IC), (ID), (II), or (III) (above) having a purity of 95 wt % or more, 97 wt % or more, or 99 wt % or more.

Alternatively, the purified compound of Formula (I) can include a mixture of isomers, each having a structure according to Formula (I), where the amount of impurity (i.e., compounds or other contaminants, exclusive of adjuvants as discussed above) is 5 wt % or less, 3 wt % or less, or 1 wt % or less. For example, the purified compound of Formula (I) can be an isomeric mixture of compounds of Structure (I), where the ratio of the amounts of the two isomers is approximately 1:1, and the combined amount of the two isomers is 95 wt % or more, 97 wt % or more, or 99 wt % or more.

As used above, and throughout this disclosure, the following terms, unless otherwise indicated, shall be understood to have the following meanings:

“DCE” means dichloroethane.

“DIAD” means diisopropylazodicarboxylate.

“DMSO” means dimethylsulfoxide.

“DPPA” means diphenylphosphoryl azide.

“EDCl” means 1-(3-dimethylaminopropyl)-3-ethylcarbodiimide hydrochloride.

“Et” means ethyl.

“EtOH” mean ethanol.

“HOBt” means 1-hydroxybenzotriazole.

“LDA” means lithium diisopropyl amide.

“Me” means methyl.

“MeOH” means methanol.

“MsCl” means mesyl chloride or methanesulfonyl chloride.

“Ms” means mesyl or methanesulfonyl.

“Mammal” means humans and other mammalian animals.

“Patient” includes both human and animals.

“PS-DIEA” means diisopropylethyl amine functionalized polystyrene.

“PS-isocyante” means isocyanate functionalized polystyrene.

“PS-trisamine” means trisamine functionalized polystyrene.

“RT” means room temperature.

“TFAA” means trifluroacetic anhydride.

“THF” means tetrahydrofuran.

“DMF” means N,N-dimethylformamide

“Cbz” means benzyloxycarbonyl

“Boc” means tert-butoxycarbonyl

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkylene” means a difunctional group obtained by removal of a hydrogen atom from an alkyl group that is defined above. Non-limiting examples of alkylene include methylene, ethylene and propylene.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkenylene” means a difunctional group obtained by removal of a hydrogen from an alkenyl group that is defined above. Non-limiting examples of alkenylene include —CH═CH—, —C(CH₃)═CH—, and —CH═CHCH₂—.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Alkynylene” means a difunctional group obtained by removal of a hydrogen from an alkynyl group that is defined above. Non-limiting examples of alkenylene include —C≡C— and —CH₂C≡C—.

“Aryl” means an aromatic monocyclic or multicyclic ring system comprising about 6 to about 14 carbon atoms, or about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, or about 5 to about 10 ring atoms, in which one or more of the ring atoms is an element other than carbon, for example nitrogen, oxygen or sulfur, alone or in combination. In some embodiments, heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl, indazolyl, and the like, in which there is at least one aromatic ring.

“Aralkyl”, “arylalkyl”, or “-alkylene-aryl” means an aryl-alkyl- group in which the aryl and alkyl are as previously described. In some embodiments, aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. In some embodiments, alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like, as well as partially saturated species such as, for example, indanyl, tetrahydronaphthyl and the like.

“Cycloalkyl” can also mean a cycloalkyl wherein a single moiety (e.g., carbonyl) can simultaneously replace two available hydrogens on the same carbon atom on a ring system. A non-limiting example of such moiety is:

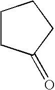

“Cycloalkylene” means a difunctional group obtained by removal of a hydrogen atom from a cycloalkyl group that is defined above. Non-limiting examples of cycloalkylene include

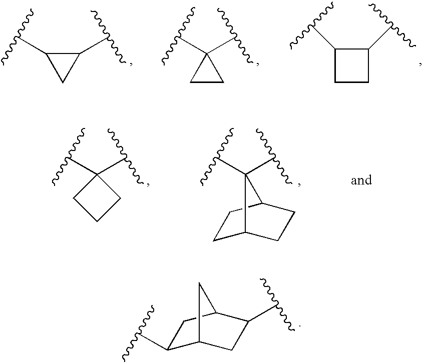

“Halogen” or “halo” means fluorine, chlorine, bromine, or iodine. In some embodiments, halogen is selected from fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

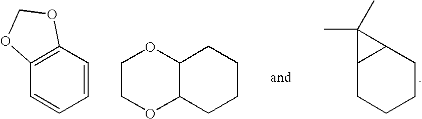

“Heterocyclyl” or “Heterocycloalkyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” can also mean a heterocyclyl wherein a single moiety (e.g., carbonyl) can simultaneously replace two available hydrogens on the same carbon atom on a ring system. Example of such moiety is pyrrolidone:

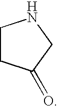

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring: 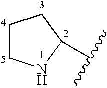
there is no —OH attached directly to carbons marked 2 and 5.

It should also be noted that tautomeric forms of the compounds of Formula (I), including salts, solvates, esters, and prodrugs thereof are also contemplated herein. For example, the moieties: 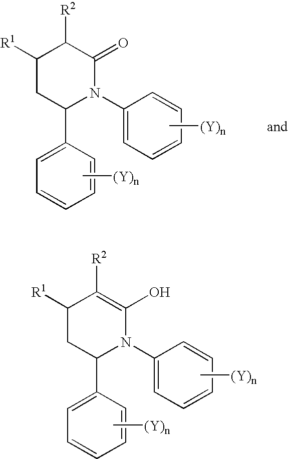
(e.g., when R³ is H) are considered equivalent in certain embodiments of this invention.

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Alkynylalkyl” means an alkynyl-alkyl- group in which the alkynyl and alkyl are as previously described. In some embodiments, alkynylalkyls contain a lower alkynyl and a lower alkyl group. The bond to the parent moiety is through the alkyl. Non-limiting examples of suitable alkynylalkyl groups include propargylmethyl.

“Heteroaralkyl”, “Heteroarylalkyl” or “-alkylene-heteroaryl” means a heteroaryl-alkyl- group in which the heteroaryl and alkyl are as previously described. In some embodiments, heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. In some embodiments, hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. In some embodiments, acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O) group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

“Benzo-fused-cycloalkyl” or “Benzocycloalkyl” means a phenyl ring fused to a cycloalkyl, as defined above, wherein said benzo-fused-cycloalkyl or benzocycloalkyl, can be optionally substituted with 1 to 3 “ring system substituents” as defined above. Non-limiting examples of suitable benzo-fused-cycloalkyl or benzocycloalkyl groups include the following:

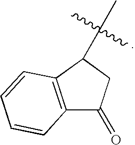

“Benzo-fused-heterocycloalkyl”, “benzo-fused-heterocyclyl” or “benzoheterocyclyl” means a phenyl ring fused to a heterocycloalkyl or heterocyclyl ring, as defined above, wherein said benzo-fused-heterocycloalkyl, benzo-fused-heterocyclyl or benzoheterocyclyl can be optionally substituted with 1 to 3 “ring system substituents” as defined above. Non-limiting examples of suitable benzo-fused-heterocycloalkyl, benzo-fused-heterocyclyl or benzoheterocyclyl groups include the following:

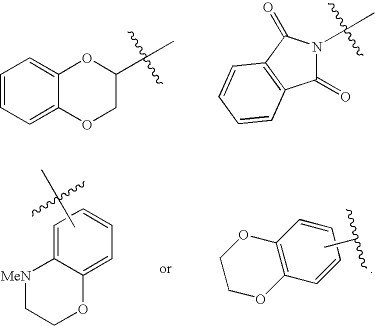

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocyclyl, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference thereto.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as O-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R -carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N- or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I.R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al, Journal of Pharmaceutical Sciences (1977) 66(1)1-19; P. Gould, International J. of Pharmaceutics (1986) 33201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of Formula I, and salts, solvates, esters and prodrugs thereof, may exist in their tautomeric form (for example, as an amide or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula I, and of the salts, solvates, esters and prodrugs of the compounds of Formula I, are intended to be included in the present invention.

The compounds according to the invention have pharmacological properties; in particular, the compounds of Formula I can be CB1 modulators.

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

As used herein, the term “pharmaceutical combination” means a combination of two or more pharmaceutical compounds. Such combination can be in any form. The term “pharmaceutical combination” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units. A pharmaceutical combination can also include two or more pharmaceutical compounds administered separately, e.g., in two or more separate dosage units.

The compounds of Formula (I), or pharmaceutically acceptable salts, solvates, or esters thereof, can be administered in any suitable form, e.g., alone, or in combination with a pharmaceutically acceptable carrier, excipient or diluent in a pharmaceutical composition, according to standard pharmaceutical practice. The compounds of Formula (I), or pharmaceutically acceptable salts, solvates, or esters thereof, can be administered orally or parenterally, including intravenous, intramuscular, interperitoneal, subcutaneous, rectal, or topical routes of administration.

Pharmaceutical compositions comprising at least one compound of Formula (I), or a pharmaceutically acceptable salt, solvate, or ester thereof can be in a form suitable for oral administration, e.g., as tablets, troches, capsules, lozenges, aqueous or oily suspensions, dispersible powders or granules, emulsions, syrups, or elixirs. Oral compositions may be prepared by any conventional pharmaceutical method, and may also contain sweetening agents, flavoring agents, coloring agents, and preserving agents.

The amount of compound of Formula (I), or a pharmaceutically acceptable salt, solvate, or ester thereof, administered to a patient can be determined by a physician based on the age, weight, and response of the patient, as well as by the severity of the condition treated. For example, the amount of compound of Formula (I), or a pharmaceutically acceptable salt, solvate, or ester thereof, administered to the patient can range from about 0.1 mg/kg body weight per day to about 60 mg/kg/d. In some embodiments, the dose is about 0.5 mg/kg/d to about 40 mg/kg/d.

The compounds of Formula (I) may also be used in conjunction with an additional therapeutic agent or agents for the treatment of the diseases, conditions and/or disorders described herein. Thus, in another embodiment, methods of treatment that include administering compounds of the present invention in combination with other therapeutic agents are also provided.

Suitable other therapeutic agents that may be used in combination with compounds of Formula (I) include anti-obesity agents such as apolipoprotein-B secretion/microsomal triglyceride transfer protein (apo-B/MTP) inhibitors, 11β.-hydroxy steroid dehydrogenase-1 (11β-HSD type 1) inhibitors, peptide YY₃₋₃₆ or analogs thereof, MCR-4 agonists, cholecystokinin-A (CCK-A) agonists, monoamine reuptake inhibitors (e.g., sibutramine), sympathomimetic agents, β3 adrenergic receptor agonists, dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), neuropeptide-Y antagonists (e.g., NPY Y5 receptor antagonists, such as the spiro compounds described in U.S. Pat. Nos. 6,566,367; 6,649,624; 6,638,942; 6,605,720; 6,495,559; 6,462,053; 6,388,077; 6,335,345; 6,326,375, and 6,566,367; U.S. Publication Nos. 2002/0151456, 2003/036652, 2004/192705, 2003/036652, 2004/072847, and 2005/033048; and PCT Publication No. WO 03/082190), thyromimetic agents, dehydroepiandrosterone or an analog thereof, glucocorticoid receptor agonists or antagonists, orexin receptor antagonists, glucagon-like peptide-1 receptor agonists, ciliary neurotrophic factors (such as Axokine™ available from Regeneron Pharmaceuticals, Inc., Tarrytown, N.Y. and Procter & Gamble Company, Cincinnati, Ohio), human agouti-related proteins (AGRP), ghrelin receptor antagonists, histamine 3 receptor antagonists or inverse agonists, neuromedin U receptor agonists and the like. Other anti-obesity agents are well known or would be readily apparent to one of ordinary skill in the art.

In one embodiment, compounds of Formula (I) are combined with anti-obesity agents selected from the group consisting of orlistat, sibutramine, bromocriptine, ephedrine, leptin, pseudoephedrine, PYY₃₋₃₆ or an analog thereof, and 2-oxo-N-(5-phenylpyrazinyl)spiro-[isobenzofuran-1(3H), 4′-piperidine]-1′-carboxamide.

Representative anti-obesity agents for use in the combinations, pharmaceutical compositions, and methods of the present invention can be prepared using methods known in the art, for example, sibutramine can be prepared as described in U.S. Pat. No. 4,929,629; bromocriptine can be prepared as described in U.S. Pat. No. 3,752,814 and U.S. Pat. No. 3,752,888; orlistat can be prepared as described in U.S. Pat. No. 5,274,143; U.S. Pat. No. 5,420,305; U.S. Pat. No. 5,540,917; and U.S. Pat. No. 5,643,874; PYY₃₋₃₆ (including analogs) can be prepared as described in U.S. Publication No. 2002/0141985 and WO 03/027637; and the NPY Y5 receptor antagonist 2-oxo-N-(5-phenyl-pyrazinyl)spiro[isobenzofuran-1(3H), 4′-piperidine]-1′-carboxamide can be prepared as described in U.S. Publication No. 2002/0151456. Other useful NPY Y5 receptor antagonists include those described in PCT Publication No. 03/082190, such as 3-oxo-N-(5-phenyl-2-pyrazinyl)-spiro[isobenzofuran-1(3H), 4′-piperidine]-1′-carboxamide; 3-oxo-N-(7-trifluoromethylpyrido[3,2-b]pyridin-2-yl)-spiro-[isobenzofuran-1(3H),4′-piperidine]-1′-carboxamide; N-[5-(3-fluorophenyl)-2-pyrimidinyl]-3-oxospiro-isobenzofuran-1(3H),[4′-piperidine]-1′-carboxamide; trans-3′-oxo-N-(5-phenyl-2-pyrimidinyl)]spiro[cyclohexane-1,1′(3′H)-isobenzofuran]-4-carboxamide; trans-3′-oxo-N-[1-(3-quinolyl)-4-imidazolyl]spiro[cyclohexane-1,1′(3′H)-isobenzofuran]-4-carboxamide; trans-3-oxo-N-(5-phenyl-2-pyrazinyl)spiro[4-azaisobenzofuran-1(3H),1′-cyclohexane]-4′-carboxamide; trans-N-[5-(3-fluorophenyl)-2-pyrimidinyl]-3-oxospiro[5-azaisobenzofuran-1(3H),1′-cyclohexane]-4′-carboxamide; trans-N-[5-(2-fluorophenyl)-2-pyrimidinyl]-3-oxospiro[5-azaisobenzofuran-1(3H),1′-cyclohexane]-4′-carboxamide; trans-N-[1-(3,5-difluorophenyl)-4-imidazolyl]-3-oxospiro[7-azaisobenzofuran-1(3H),1′-cyclohexane]-4′-carboxamide; trans-3-oxo-N-(1-phenyl-4-pyrazolyl)spiro[4-azaisobenzofuran-1(3H),1′-cyclohexane]-4′-carboxamide; trans-N-[1-(2-fluorophenyl)-3-pyrazolyl]-3-oxospiro[6-azaisobenzofuran-1(3H),1′-cyclohexane]-4′-carboxamide; trans-3-oxo-N-(I-phenyl-3-pyrazolyl)spiro[6-azaisobenzofuran-1(3H),1′-cyclohexane]-4′-carboxamide; trans-3-oxo-N-(2-phenyl-1,2,3-triazol-4-yl)spiro[6-azaisobenzofuran-1(3H),1′-cyclohexane]-4′-carboxamide; and pharmaceutically acceptable salts and esters thereof. All of the above recited patents and publications are incorporated herein by reference.

Other suitable therapeutic agents that may be administered in combination with one or more compounds of Formula (I) include therapeutic agents designed to treat tobacco abuse (e.g., nicotine receptor partial agonists, bupropion hypochloride (also known under the tradename Zyban™) and nicotine replacement therapies), agents to treat erectile dysfunction (e.g., dopaminergic agents, such as apomorphine), ADD/ADHD agents (e.g., Ritalin™, Strattera™, Concerta™ and Adderall™), and agents to treat alcoholism, such as opioid antagonists (e.g., naltrexone (also known under the tradename ReVia™) and nalmefene), disulfiram (also known under the tradename Antabuse™), and acamprosate (also known under the tradename Campral™)). In addition, agents for reducing alcohol withdrawal symptoms may also be co-administered, such as benzodiazepines, beta-blockers, clonidine, carbamazepine, pregabalin, and gabapentin (Neurontin™).

Other therapeutic agents that may administered in combination with one or more compounds of Formula (I) include antihypertensive agents, anti-inflammatory agents (e.g., COX-2 inhibitors), antidepressants (e.g., fluoxetine hydrochloride (Prozac™)), cognitive improvement agents (e.g., donepezil hydrochloride (Aircept™) and other acetylcholinesterase inhibitors), neuroprotective agents (e.g., memantine), antipsychotic medications (e.g., ziprasidone (Geodon™), risperidone (Risperdal™), and olanzapine (Zyprexa™)), insulin and insulin analogs (e.g., LysPro insulin), GLP-1 (7-37) (insulinotropin) and GLP-1 (7-36)-NH₂, sulfonylureas and analogs thereof (e.g., chlorpropamide, glibenclamide, tolbutamide, tolazamide, acetohexamide, Glypizide™, glimepiride, repaglinide, meglitinide; biguanides: metformin, phenformin, buformin), α2-antagonists and imidazolines (e.g., midaglizole, isaglidole, deriglidole, idazoxan, efaroxan, fluparoxan), other insulin secretagogues (e.g., linogliride, A-4166), glitazones (e.g., ciglitazone, Actose™, pioglitazone, englitazone, troglitazone, darglitazone, Avandia™, BRL49653), fatty acid oxidation inhibitors (e.g., clomoxir, etomoxir), α-glucosidase inhibitors (e.g., acarbose, miglitol, emiglitate, voglibose, MDL-25,637, camiglibose, MDL-73,945), β-agonists (e.g., BRL 35135, BRL 37344, RO 16-8714, ICI D7114, CL 316,243), phosphodiesterase inhibitors (e.g., L-386,398), lipid-lowering agents (e.g., benfluorex, fenfluramine), vanadate and vanadium complexes (e.g., Naglivan™) and peroxovanadium complexes, amylin antagonists, glucagon antagonists, gluconeogenesis inhibitors, somatostatin analogs, antilipolytic agents (e.g., nicotinic acid, acipimox, WAG 994, pramlintide (Symlin™), AC 2993, nateglinide, aldose reductase inhibitors (e.g., zopolrestat), glycogen phosphorylase inhibitors, sorbitol dehydrogenase inhibitors, sodium-hydrogen exchanger type 1 (NHE-1) inhibitors and/or cholesterol lowering compounds.

Non-limiting examples of cholesterol lowering compounds suitable for administration in combination with one or more compounds of Formula (I) include cholesterol biosynthesis inhibitors, cholesterol absorption inhibitors, HMG-CoA reductase inhibitors, HMG-COA synthase inhibitors, HMG-CoA reductase or synthase gene expression inhibitors, CETP inhibitors, bile acid sequesterants, fibrates, ACAT inhibitors, squalene synthetase inhibitors, squalene epoxidase inhibitors, sterol biosynthesis inhibitors, nicotinic acid derivatives, bile acid sequestrants, inorganic cholesterol sequestrants, AcylCoA:Cholesterol O-acyltransferase inhibitors, cholesteryl ester transfer protein inhibitors, fish oils containing Omega 3 fatty acids, natural water soluble fibers, plant stanols and/or fatty acid esters of plant stanols, low-density lipoprotein receptor activators, anti-oxidants and niacin.

A non-limiting list of cholesterol lowering compounds suitable for administration with one or more compounds of Formula (I) include HMG CoA reductase inhibitor compounds such as lovastatin (for example MEVACOR® which is available from Merck & Co.), simvastatin (for example ZOCOR® which is available from Merck & Co.), pravastatin (for example PRAVACHOL® which is available from Bristol Meyers Squibb), atorvastatin, fluvastatin, cerivastatin, CI-981, rivastatin (sodium 7-(4-fluorophenyl)-2,6-diisopropyl-5-methoxymethylpyridin-3-yl)-3,5-dihydroxy-6-heptanoate), rosuvastatin calcium (CRESTOR® from AstraZeneca Pharmaceuticals), pitavastatin (such as NK-104 of Negma Kowa of Japan); HMG CoA synthetase inhibitors, for example L-659,699 ((E,E)-11-[3′R-(hydroxy-methyl)-4′-oxo-2′R-oxetanyl]-3,5,7R-trimethyl-2,4-undecadienoic acid); squalene synthesis inhibitors, for example squalestatin 1; squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride); sterol biosynthesis inhibitors such as DMP-565; nicotinic acid derivatives (e.g., compounds comprising a pyridine-3-carboxylate structure or a pyrazine-2-carboxylate structure, including acid forms, salts, esters, zwitterions and tautomers) such as niceritrol, nicofuranose and acipimox (5-methylpyrazine-2-carboxylic acid 4-oxide); clofibrate; gemfibrazol; bile acid sequestrants such as cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl)alkylamines and poliglusam, insoluble quaternized polystyrenes, saponins and mixtures thereof; inorganic cholesterol sequestrants such as bismuth salicylate plus montmorillonite clay, aluminum hydroxide and calcium carbonate antacids; ileal bile acid transport (“IBAT”) inhibitors (or apical sodium co-dependent bile acid transport (“ASBT”) inhibitors) such as benzothiepines, for example the therapeutic compounds comprising a 2,3,4,5-tetrahydro-1-benzothiepine 1,1-dioxide structure such as are disclosed in PCT Patent Application WO 00/38727 which is incorporated herein by reference; AcylCoA:Cholesterol O-acyltransferase (“ACAT”) Inhibitors such as avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea), and the compounds described in P. Chang et al., “Current, New and Future Treatments in Dyslipidaemia and Atherosclerosis”, Drugs 2000 July; 60(1); 55-93, which is incorporated by reference herein; Cholesteryl Ester Transfer Protein (“CETP”) Inhibitors such as those disclosed in PCT Patent Application No. WO 00/38721 and U.S. Pat. No. 6,147,090, which are incorporated herein by reference; probucol or derivatives thereof, such as AGI-1067 and other derivatives disclosed in U.S. Pat. Nos. 6,121,319 and 6,147,250, herein incorporated by reference; low-density lipoprotein (LDL) receptor activators such as HOE-402, an imidazolidinyl-pyrimidine derivative that directly stimulates LDL receptor activity, described in M. Huettinger et al., “Hypolipidemic activity of HOE-402 is Mediated by Stimulation of the LDL Receptor Pathway”, Arterioscler. Thromb. 1993; 13:1005-12, herein incorporated by reference; fish oils containing Omega 3 fatty acids (3-PUFA); natural water soluble fibers, such as psyllium, guar, oat and pectin; plant stanols and/or fatty acid esters of plant stanols, such as sitostanol ester used in BENECOL® margarine; and the substituted azetidinone or substituted β-lactam sterol absorption inhibitors.

As used herein, “sterol absorption inhibitor” means a compound capable of inhibiting the absorption of one or more sterols, including but not limited to cholesterol, phytosterols (such as sitosterol, campesterol, stigmasterol and avenosterol), 5α-stanols (such as cholestanol, 5α-campestanol, 5α-sitostanol), and/or mixtures thereof, when administered in a therapeutically effective (sterol and/or 5α-stanol absorption inhibiting) amount to a mammal or human. Particularly useful sterol absorption inhibitors include hydroxy-substituted azetidinone compounds and substituted β-lactam compounds, for example those disclosed in U.S. Pat. Nos. 5,767,115, 5,624,920, 5,668,990, 5,656,624 and 5,688,787, which are herein incorporated by reference in their entirety. These patents, respectively, disclose hydroxy-substituted azetidinone compounds and substituted β-lactam compounds useful for lowering cholesterol and/or in inhibiting the formation of cholesterol-containing lesions in mammalian arterial walls. U.S. Pat. No. 5,756,470, U.S. Patent Application No. 2002/0137690, U.S. Patent Application No. 2002/0137689 and PCT Patent Application No. WO 2002/066464 (each of which is herein incorporated by reference in its entirety) disclose sugar-substituted azetidinones and amino acid substituted azetidinones useful for preventing or treating atherosclerosis and reducing plasma cholesterol levels.

One or more compounds of Formula (I) may also be administered in combination with a naturally occurring compound that acts to lower plasma cholesterol levels. Such naturally occurring compounds are commonly called nutraceuticals and include, for example, garlic extract, Hoodia plant extracts, and niacin.

The dosage of the additional therapeutic agent is generally dependent upon a number of factors including the health of the subject being treated, the extent of treatment desired, the nature and kind of concurrent therapy, if any, and the frequency of treatment and the nature of the effect desired. In one embodiment the dosage range of the additional therapeutic agent is in the range of from about 0.001 mg to about 100 mg per kilogram body weight of the individual per day. In another embodiment, the dosage range of the additional therapeutic agent is from about 0.1 mg to about 10 mg per kilogram body weight of the individual per day. However, some variability in the general dosage range may also be required depending upon the age and weight of the subject being treated, the intended route of administration, the particular additional therapeutic agent being administered and the like. The determination of dosage ranges and optimal dosages for a particular patient is also well within the ability of one of ordinary skill in the art.

According to the methods of the invention, one or more compounds Formula (I), or one or more compounds of Formula (I) in combination with one or more additional therapeutic agents is administered to a subject in need of such treatment, for example in the form of a pharmaceutical composition. When one or more compounds of Formula (I) is administered with one or more additional therapeutic agents, the compound of the present invention and at least one other therapeutic agent (e.g., anti-obesity agent, nicotine receptor partial agonist, dopaminergic agent, or opioid antagonist) may be administered either separately or in the pharmaceutical composition comprising both. In one embodiment, such administration is oral. In other embodiments, such administration is parenteral or transdermal.

When a combination of one or more compounds of Formula (I) and at least one other therapeutic agent are administered together, such administration can be sequential in time or simultaneous. For sequential administration, one or more compounds of Formula (I) and the additional therapeutic agent can be administered in any order. In one embodiment, such administration is oral. In another embodiment, such administration is oral and simultaneous. When one or more compounds of Formula (I) and one or more additional therapeutic agents are administered sequentially, the administration of each can be by the same or by different methods.

In one embodiment, one or more compounds of Formula (I) or a combination of one or more compounds of Formula (I) and at least one additional therapeutic agent (referred to herein as a “combination”) is administered in the form of a pharmaceutical composition. Accordingly, one or more compounds of Formula (I) or a combination can be administered to a patient separately or together in any conventional oral, rectal, transdermal, parenteral, (for example, intravenous, intramuscular, or subcutaneous) intracisternal, intravaginal, intraperitoneal, intravesical, local (for example, powder, ointment or drop), or buccal, or nasal, dosage form.

### EXAMPLES

The synthesis of 2-aryl-4-amino-N-aryl-piperidines according to structural Formula (IA) is shown in Scheme 1. Diene A and imine B are reacted in the presence of a promoter (e.g., ZnCl₂ or Nafion H) to furnish the enone C. The enone C can be reduced (e.g., with NaBH₄) to the alcohol D. The alcohol D can be oxidized by methods known in the art to the ketone E. Reductive amination of the ketone E with various amines furnishes the desired 4-amino-2-aryl-N-aryl-piperidines F.

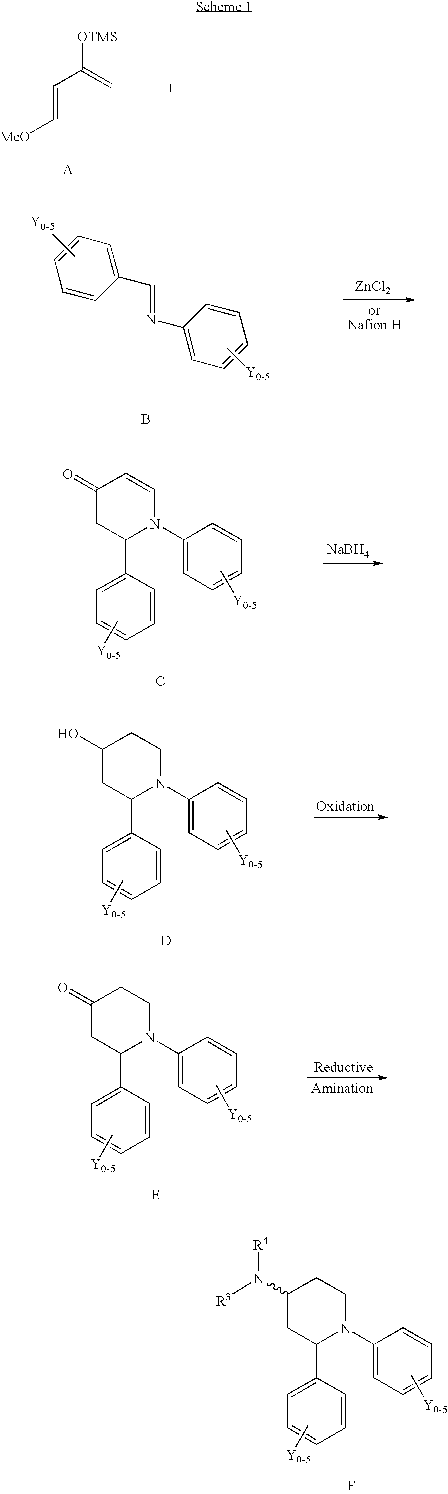

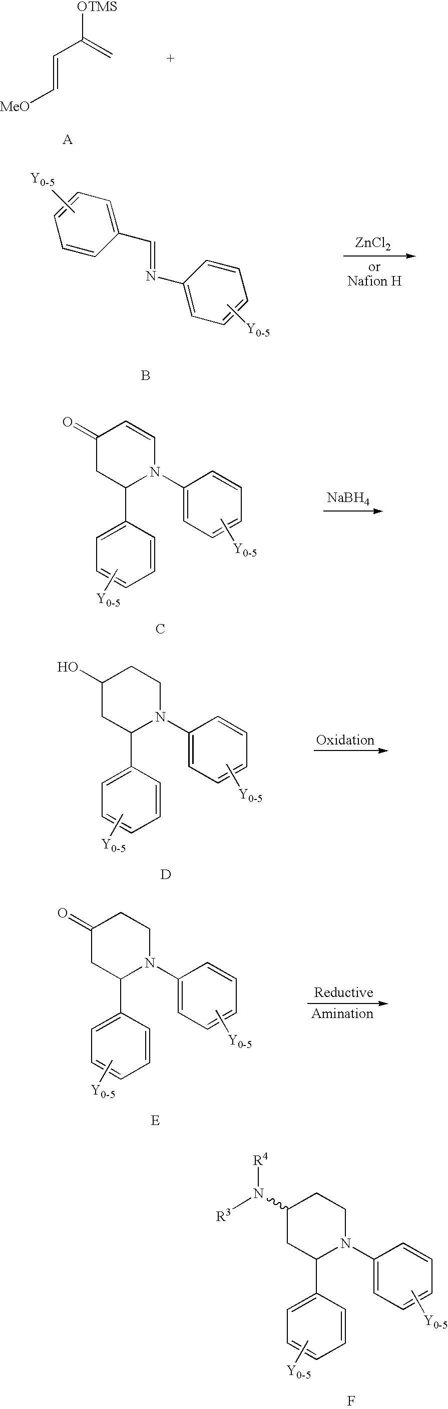

Alcohol D can be converted into the azide G using conditions known in the art (e.g., MsCl and NaN₃). The azide G can be reduced to the primary amine H (e.g., step-wise with PPh₃ and H₂O, or with H₂/PtO₂). Alternatively, amine H can be prepared via reductive amination of ketone E with, e.g., NH₄OAc/NaCNBH₃. The primary amine in H can be functionalized under conditions known in the art.

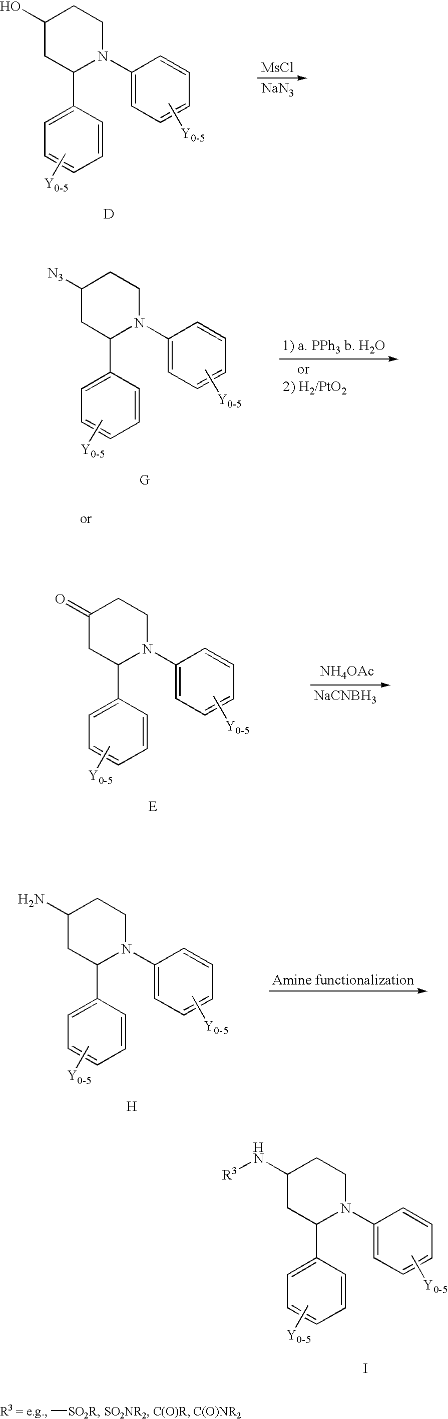

### Preparation of Examples 1 and 2

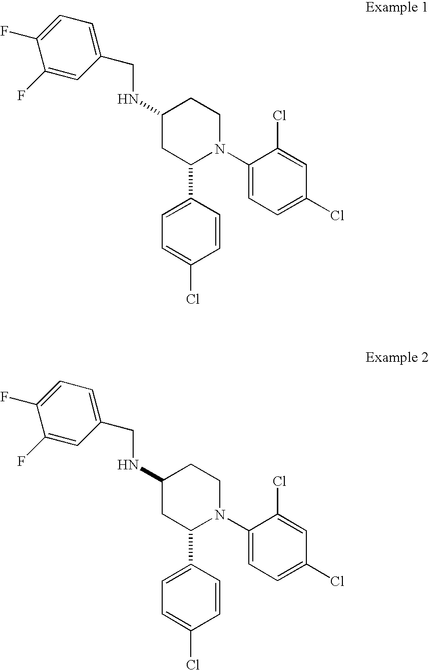 Step 1:

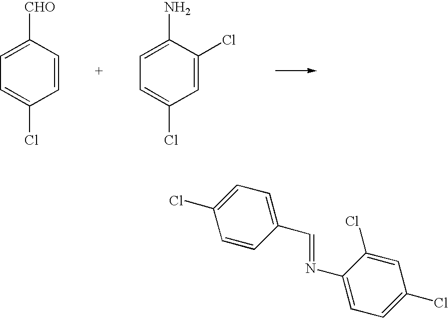

A solution of 2,4-dichloroaniline (10.0 g, 61.7 mmol) and 4-chlorobenzaldehyde (9.6 g, 67.9 mmol) in toluene (150 mL) with a Dean-Stark trap attached was heated to reflux for 24 hr. The solution was cooled to RT and treated with activated carbon, filtered and concentrated to afford (4-chloro-benzylidene)(2,4-dichlorophenyl)amine.
Step 2:

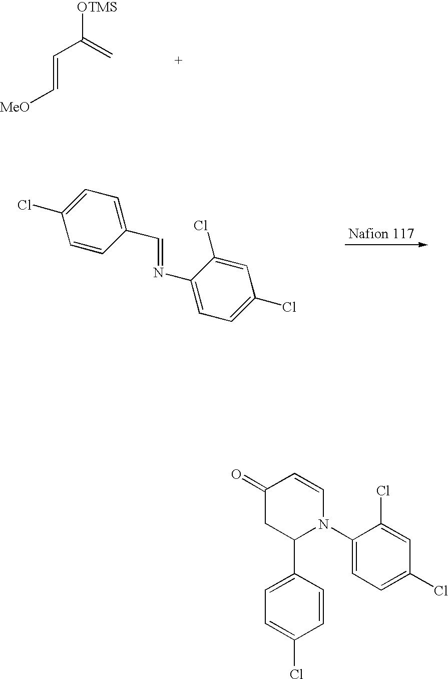

Nafion® 117 (33 mg), trans-methoxy-3-(trimethylsilyloxy)-1,3-butadiene (0.15 mL), and (4-chloro-benzylidene)-(2,4-dichlorophenyl)amine (71 mg) were taken up in CH₂Cl₂ and stirred at 25° C. for 16 hours. The mixture was filtered, the Nafion® 117 was washed with CH₂Cl₂, and the resulting solution was concentrated. The residue was purified via thin-layer preparative chromatography (5/2 hexanes/EtOAc, SiO₂) gave 50 mg (56%) of the enone as a yellow oil.
Step 3:

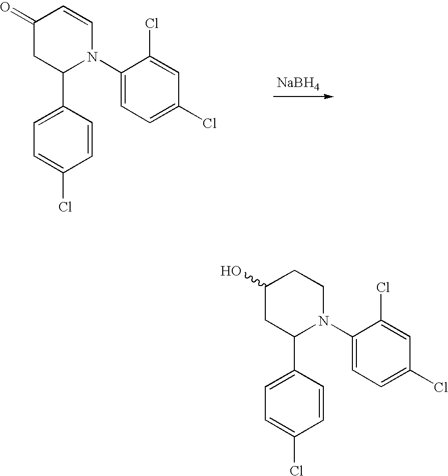

The enone prepared in Step 2 (148 mg) was taken up in EtOH/THF (1/1, 2 mL), and NaBH₄ (40 mg) was added to the solution. The solution was stirred at 25° C. for 16 hours. The reaction mixture was quenched with 1 M HCl_{(aq.)} and CH₂Cl₂. After 0.5 h stirring at 25° C., the mixture was neutralized with NaHCO₃. The layers were separated and the aqueous layer was extracted with CH₂Cl₂. The combined organic layers were dried (MgSO₄). Filtration and concentration gave a yellow oil. Purification via flash chromatography (95/5 CH₂Cl₂/EtOAc, SiO₂) gave 100 mg of the alcohol (67%) as a mixture of diastereomers.
Step 4:

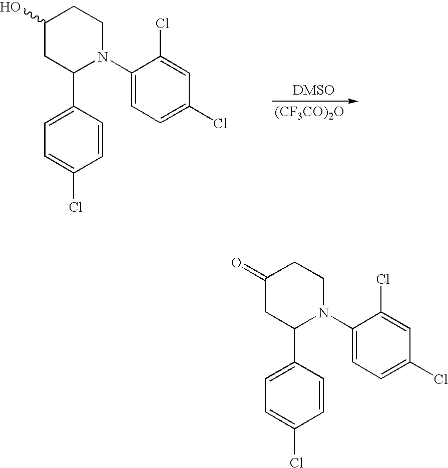

DMSO (0.89 mL) in CH₂Cl₂ (3 mL) was cooled to −60° C. After 15 minutes, TFAA (0.6 mL) was added at −60° C. After 10 minutes, a solution of the alcohol prepared in Step 3 in CH₂Cl₂ was added. After an additional 10 minutes, Et₃N (0.9 mL) was added, and the solution was stirred at 25° C. (0.5 h). The solution was diluted with H₂O and extracted with CH₂Cl₂. The combined organic layers were dried (MgSO₄), filtered, and concentrated. Purification via thin-layer preparative chromatography (95/5 CH₂Cl₂/EtOAc, SiO₂) furnished the ketone (quant. Yield).
Step 5:

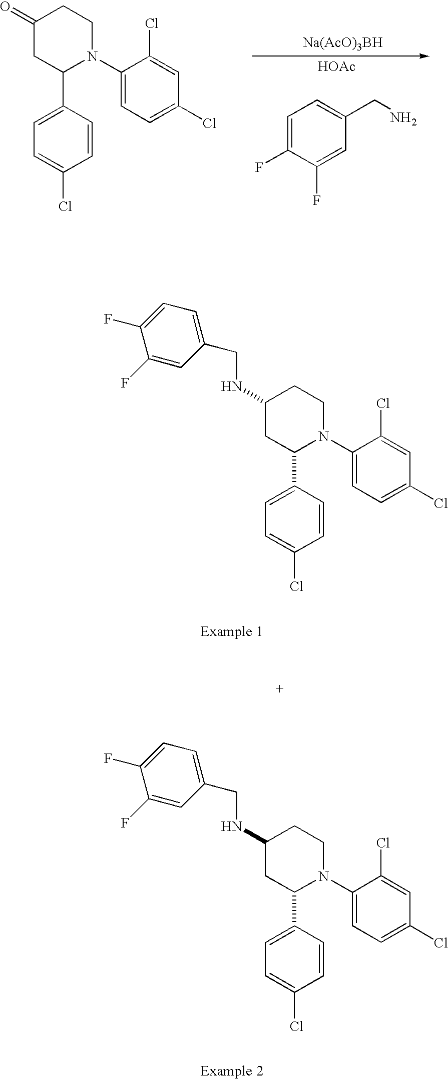

3,4-Difluorobenzylamine (23 mg), the ketone prepared in Step 4 (46 mg), Na(AcO)₃BH (28 mg), and HOAc (60 μL) were taken up in CH₂Cl₂ and stirred at 25° C. (16 h). The solution was diluted with CH₂Cl₂ and washed with sat. NaHCO₃ (aq.). The combined organic layers were dried (MgSO₄), filtered, and concentrated. Purification via thin-layer preparative chromatography (9/1 CH₂Cl₂/EtOAc, SiO₂) gave 27 mg of Example 2 (2,4-trans). Further purification of mixed fractions (hexanes/Et₂O, SiO₂) gave 9 mg of Example 1 (2,4-cis).

### Preparation of Examples 3 and 4

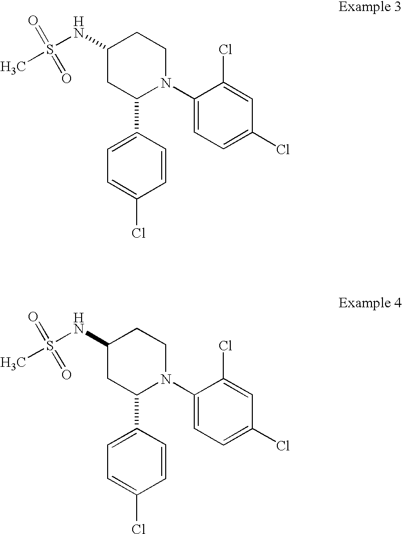 Step 1:

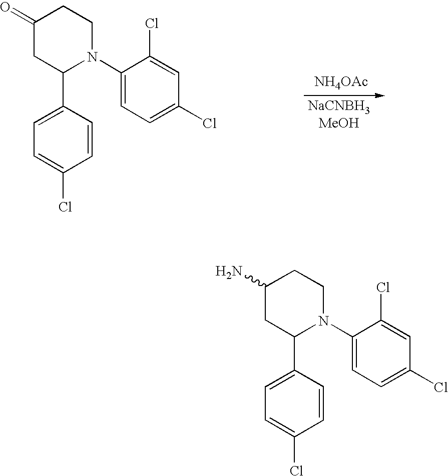

The ketone (60 mg) from Step 4 of Examples 1 and 2 (see above), NH₄OAc (13 mg), and NaCNBH₃ (25 mg) was taken up in MeOH (1.5 mL), and the solution was stirred at 25° C. (24 h). The reaction was quenched with 0.01 N HCl (aq.). The reaction was concentrated and basified with sat. Na₂CO₃ (aq.). The solution was extracted with Et₂O. The combined organic layers were dried (Na₂SO₄), filtered, and concentrated to give a yellow oil. Purification via thin-layer chromatography (8/2 Et₂O/hexane, SiO₂) gave the primary amine (14 mg) as a mixture of diastereomers.
Step 2:

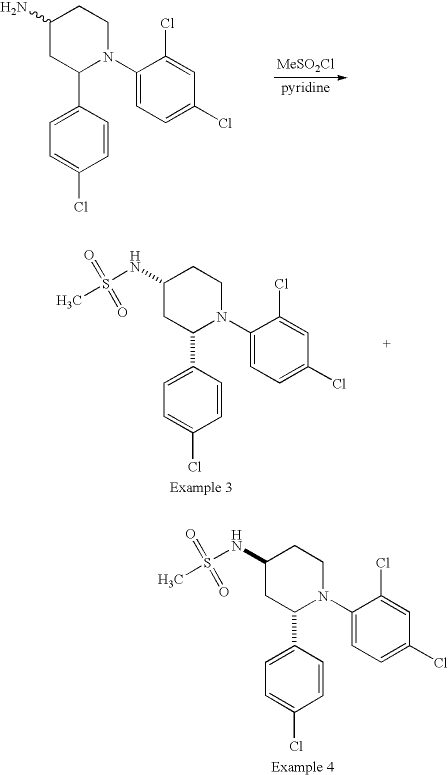

The amine (14 mg) prepared in Step 1, MeSO₂Cl (6 mg), and pyridine (0.2 mL) were taken up in CH₂CL₂ and stirred at 25° C. (18 h). The solution was concentrated, and the residue was taken up in CH₂Cl₂ and washed with sat Na₂CO₃ (aq.). The combined organic layers were dried (Na₂SO₄), filtered, and concentrated. Purification via thin-layer preparative chromatography (Et₂O, SiO₂) gave the cis isomer Example 3 (4 mg) and trans isomer Example 4 (1 mg).

### Reaction of Ketone with an Amine Library to Furnish 2-aryl-4-amino Substituted Analogs

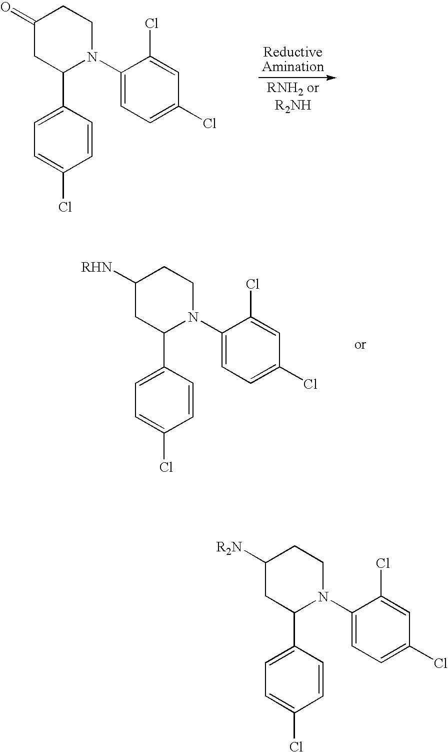

MP-Triacetoxyborohydride resin (Argonaut Technologies) (49 mg, 0.1 mmol) was added to 96-wells of a deep well polypropylene microtiter plate followed by a stock solution of the ketone (0.02 mmol) from Step 4 of Examples 1 and 2 in DCE/MeCN (3 mL, 1/1 with 1% AcOH). A stock solution of each of the various amines (100 μL, 0.1 mmol, 1 M in DCE/MeCN, 1/1) were added to the wells; and the microtiter plate was sealed and shaken at 25° C. for 20 h. For primary amines, PS-activated ketone (Aldrich) (3 mmol, 40 mg) was added to the wells and shaken an additional 20 h. For secondary amines, PS-benzyaldehyde (1.5 mmol, 80 mg) was added to the wells and shaken an additional 20 h. The solutions were then filtered thru a polypropylene frit into a 2^{nd} microtiter plate containing MP-TsOH resin (80 mg). After the top plate was washed with MeCN (0.5 mL), the plate was removed; the bottom microtiter plate was sealed and shaken at 25° C. for 2 h. Then the solutions were filtered thru a polypropylene frit, and the resin was washed three times each with DCM and MeOH to remove unreacted reagents. After the plate was allowed to dry for 10 min., the bottom microtiter plate was resealed, and ammonia in methanol (2N, 1 mL) was added to each well. The plate was sealed and shaken at 25° C. for 1 hr. Then, the solutions were filtered thru a polypropylene frit into a 96-well collection plate. The wells of the top plate were then washed with MeOH (0.5 mL), and the plate removed. The resultant solutions in the collection plate were then transferred into 2-dram vials, and the solvents removed in vacuo via a SpeedVac concentrator. The resulting samples were evaluated by LCMS, and those that were >70% pure are listed in the table below (Examples 5-63).

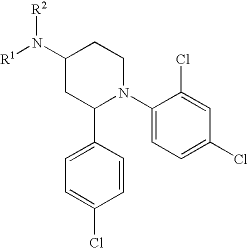

| Ex. # | R¹ | R² | Amine |
|---|---|---|---|
| 5 | 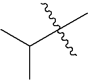 | H | 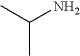 |
| 6 | 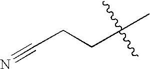 | H | 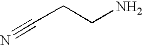 |
| 7 | 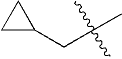 | H | 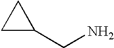 |
| 8 | 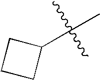 | H | 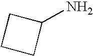 |
| 9 | 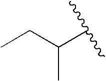 | H | 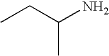 |
| 10 | 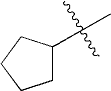 | H | 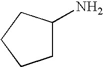 |
| 11 | 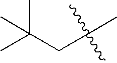 | H | 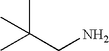 |
| 12 | 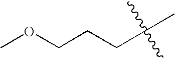 | H | 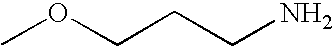 |
| 13 | 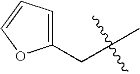 | H | 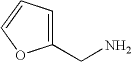 |
| 14 | 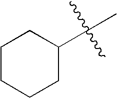 | H | 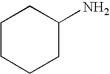 |
| 15 | 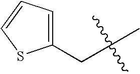 | H | 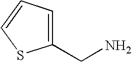 |
| 16 | 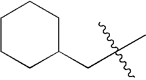 | H | 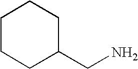 |
| 17 | 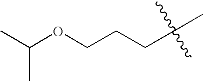 | H | 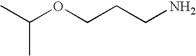 |
| 18 | 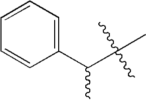 | H | 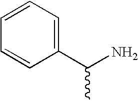 |
| 19 | 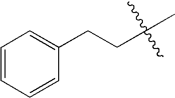 | H | 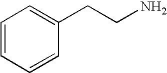 |
| 20 | 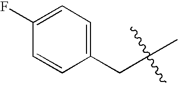 | H | 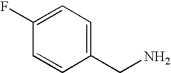 |
| 21 | 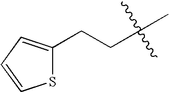 | H | 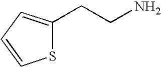 |
| 22 | 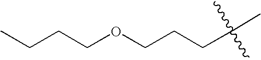 | H | 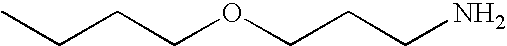 |
| 23 | 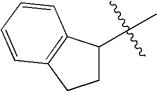 | H | 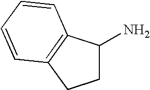 |
| 24 | 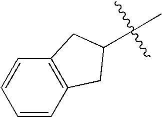 | H | 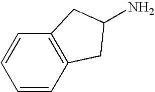 |
| 25 | 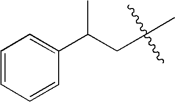 | H | 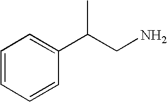 |
| 26 | 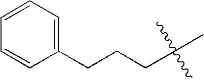 | H | 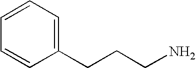 |
| 27 | 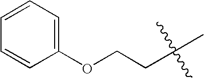 | H | 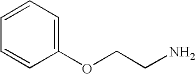 |
| 28 | 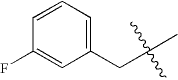 | H | 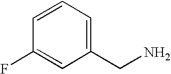 |
| 29 | 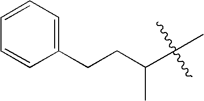 | H | 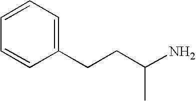 |
| 30 | 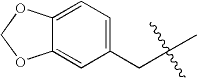 | H | 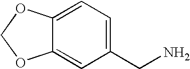 |
| 31 | 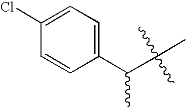 | H | 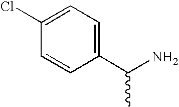 |
| 32 | 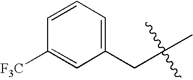 | H | 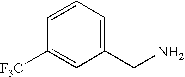 |
| 33 | 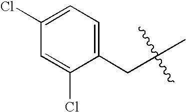 | H | 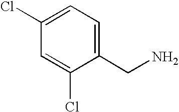 |
| 34 | 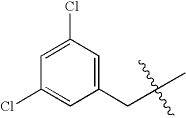 | H | 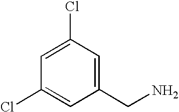 |
| 35 | 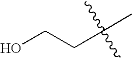 | H | 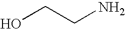 |
| 36 | 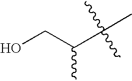 | H | 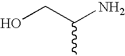 |
| 37 | 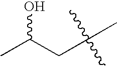 | H | 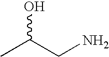 |
| 38 | 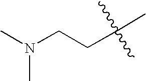 | H | 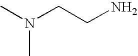 |
| 39 | 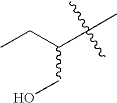 | H | 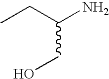 |
| 40 | 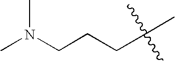 | H | 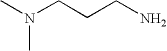 |
| 41 | 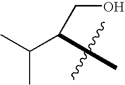 | H | 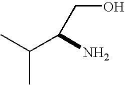 |
| 42 | 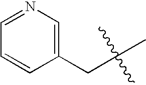 | H | 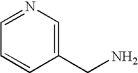 |
| 43 | 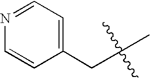 | H | 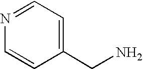 |
| 44 | 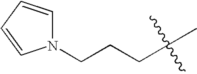 | H | 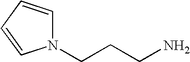 |
| 45 | 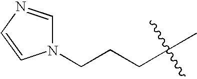 | H | 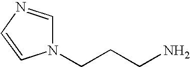 |
| 46 | 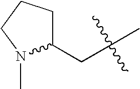 | H | 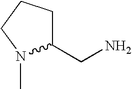 |
| 47 | 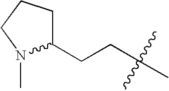 | H | 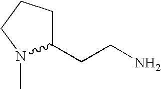 |
| 48 | 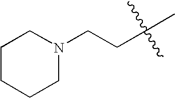 | H | 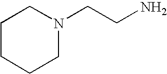 |
| 49 | 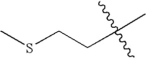 | H | 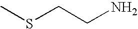 |
| 50 | 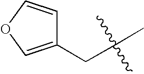 | H | 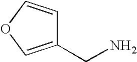 |
| 51 | 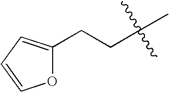 | H | 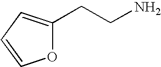 |
| 52 | 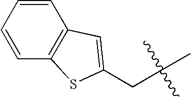 | H | 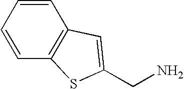 |
| 53 | 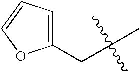 | CH₃ | 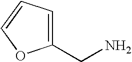 |
| 54 | 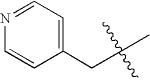 | CH₃CH₂ | 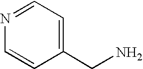 |
| 55 | 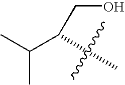 | H | 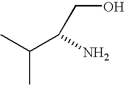 |
| 56 | 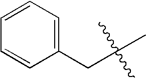 | H | 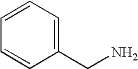 |
| 57 | 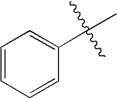 | H | 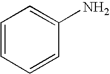 |

Compounds were tested as a 3/2 mixture of cis/trans.

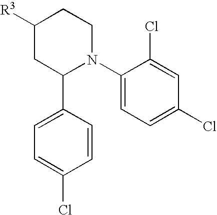

| Ex. # | R³ | Amine |
|---|---|---|
| 58 | 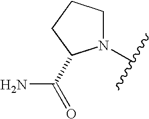 | 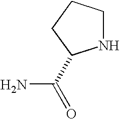 |
| 59 | 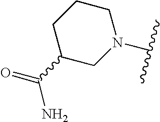 | 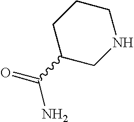 |
| 60 | 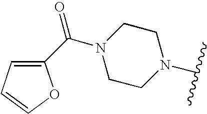 | 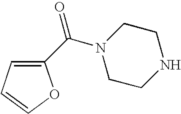 |
| 61 | 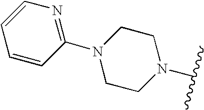 | 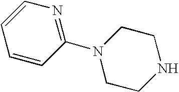 |
| 62 | 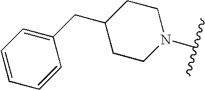 | 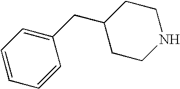 |
| 63 | 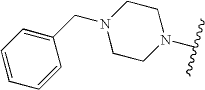 | 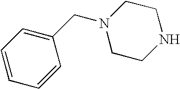 |

Compounds were tested as a 3/2 mixture of cis/trans.

### Preparation of Examples 64-67

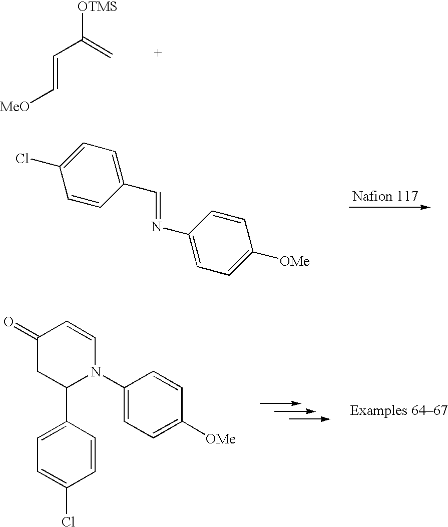

Example 64-67 were prepared in a manner similar to that described above for Examples 1 and 2, except that (4-chloro-benzylidene)-(4-methoxyphenyl)amine was used instead of (4-chloro-benzylidene)-(2,4-dichlorophenyl)amine. The resulting enone was reduced to the corresponding alcohol (i.e., Example 64), or the alcohol was subsequently oxidized and then reacted with the appropriate amine.

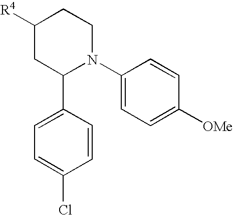

| Ex. # | R⁴ |
|---|---|
| 64 | OH |
| 65 | 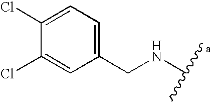 |
| 66 | 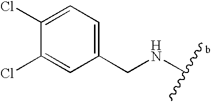 |
| 67 | 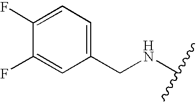 |

ₐ2,4-cis isomer
_{b}2,4-trans isomer
If not specified, compounds were tested as a 3/2 mixture of cis/trans

### Preparation of Examples 68-71

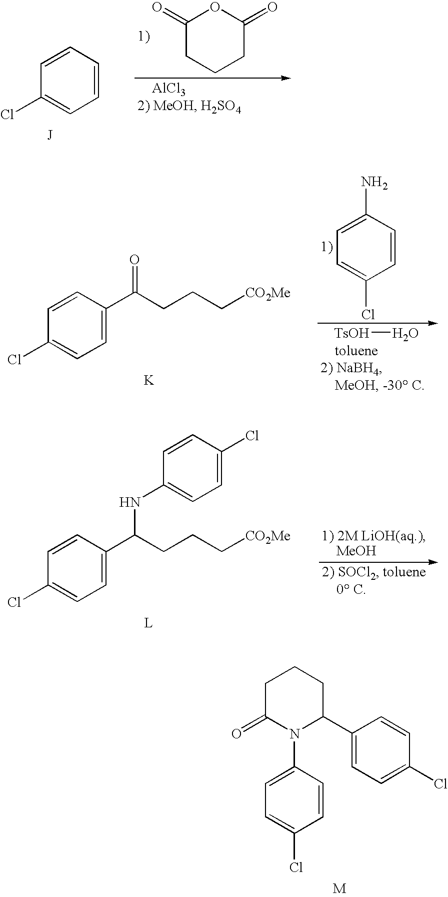 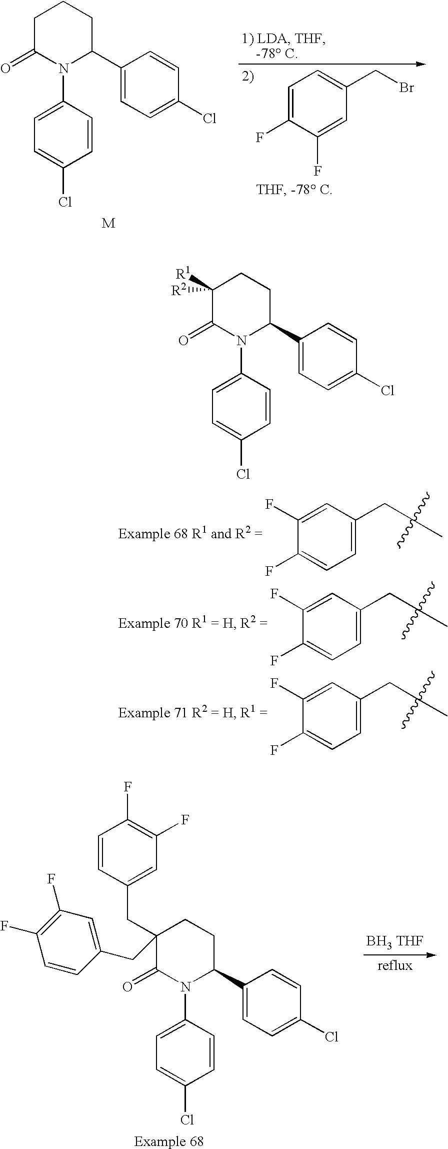 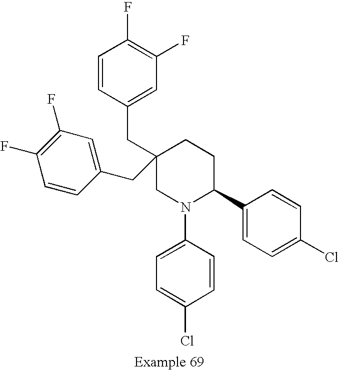 Step 1:

To a solution of glutaric anhydride (21.3 g, 114 mmol) in chlorobenzene (158 g, 1.40 mol) was added AlCl₃ (50.0 g, 375 mmol). The mixture was stirred at RT using a mechanical stirrer for 1.5 days. The reaction mixture was slowly poured into ice cold concentrated HCl. The mixture was stirred at 0° C. for 1 h. The solid was removed by filtration, and the solid was then washed with water and dried on a filter for 2 h. The solid was then dried under vacuum overnight to afford a keto acid (25 g) as a tan solid.

To a solution of the keto acid (13.8 g, 61 mmol) in MeOH (200 mL) was added conc. H₂SO₄ (0.5 mL). The solution was heated to 75° C. for 2.5 h. The solution was concentrated, and partitioned between EtOAc and NaHCO₃ (aq.). The aqueous layer was extracted with EtOAc (3×). The combined organic layers were washed with brine, dried over Na₂SO₄, filtered and concentrated to afford K (8.1 g) as a yellow solid.

Step 2:

To a solution of K (8.0 g, 33.3 mmol) in toluene (100 mL) was added 4-chloroaniline (5.93 g, 46.6 mmol) and p-toluenesulfonic acid monohydrate (253 mg, 1.33 mmol). The solution was heated to reflux for 1.5 d with a Dean-Stark trap attached. The solution was cooled and concentrated. To the resultant oil was added MeOH (100 mL) followed by NaHCO₃ (1.0 g). The solution was cooled to −30° C. and NaBH₄ (2.4 g) was added over 1 hour in portions; the solution was then stirred at −30° C. for an additional 1 h. The solution was warmed to room temperature and water was added. The aqueous layer was extracted with EtOAc (3×). The combined organic layers were washed with brine, dried over Na₂SO₄, filtered and concentrated to afford L. The material was used without purification.

Step 3:

To a solution of L (8.6 g, 24.5 mmol) in MeOH (150 mL) was added 2M LiOH (aq.) (37 mL, 73.4 mmol). The solution was stirred at RT for 4 h. The solution was adjusted to pH 6 with the addition of 4M HCl (aq.). The solution was extracted with EtOAc (3×). The combined organic layers were washed with brine, dried over Na₂SO₄, filtered and concentrated to afford an orange oil. This oil was taken up in dry toluene (200 mL) and cooled to 0° C. Pyridine (5.05, 63.9 mmol) was added followed by the addition (over 1 h) of a solution of thionyl chloride (3.03 g, 26 mmol) in dry toluene (10 mL). The resultant solution was stirred for an additional 1 h at 0° C. The solution was poured into H₂O and extracted with EtOAc. The organic layer was washed with 1 M HCl, followed by saturated aqueous NaHCO₃ and brine. The organic layer was dried over Na₂SO₄, filtered and concentrated. The crude product was purified by flash chromatography gradient elution (SiO₂: 100:0 to 40:60 hexanes:ethyl acetate) to afford M (5.3 g) as an orange crystalline solid.

### Examples 68, 70 and 71

To a solution of LDA (3.2 mmol) in dry THF (20 mL) at −78° C. was added M (510 mg, 1.6 mmol) in dry THF (5 mL). This solution was allowed to stir at −78° C. for 1 h. To this solution was added 3,4-difluorobenzyl bromide (364 mg, 1.76 mmol) and the solution was stirred at −78° C. for 4 h. Water was added and the solution was allowed to warm to RT. The aqueous layer was extracted with EtOAc (3×). The combined organic layers were then washed with brine, dried over Na₂SO₄, filtered and concentrated. The crude product was purified by flash chromatography using gradient elution (SiO₂: 100:0 to 1:1 hexanes/EtOAc) to afford 68 (105 mg), 70 (30 mg) and 71 (41 mg).

### Preparation of Example 69

To a solution of 68 (63 mg, 0.11 mmol) in THF (4 mL) was added borane THF complex (1 M solution in THF, 0.33 mmol). The solution was heated to reflux for 4 h. The solution was cooled to RT and water was added. The aqueous layer was extracted with EtOAc (3×). The combined organic layers were washed with brine, dried over Na₂SO₄, filtered and concentrated. The crude product was purified by prep TLC (SiO₂: 4:1 hexanes/EtOAc) to afford 69 (6 mg).

### Preparation of Example 72

Example 72 was prepared in a manner similar to that of Example 69, except that Example 70 was the starting material instead of Example 68.

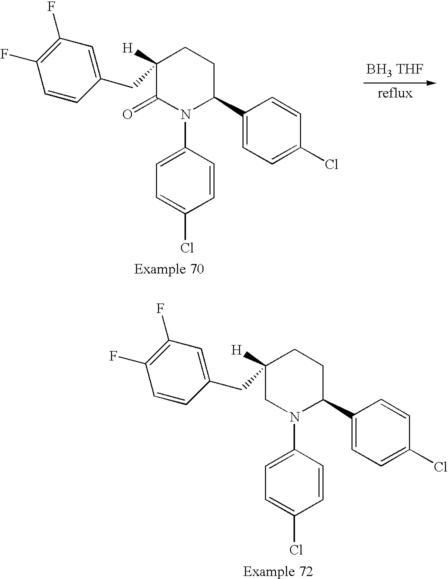

### Preparation of Examples 73 and 74

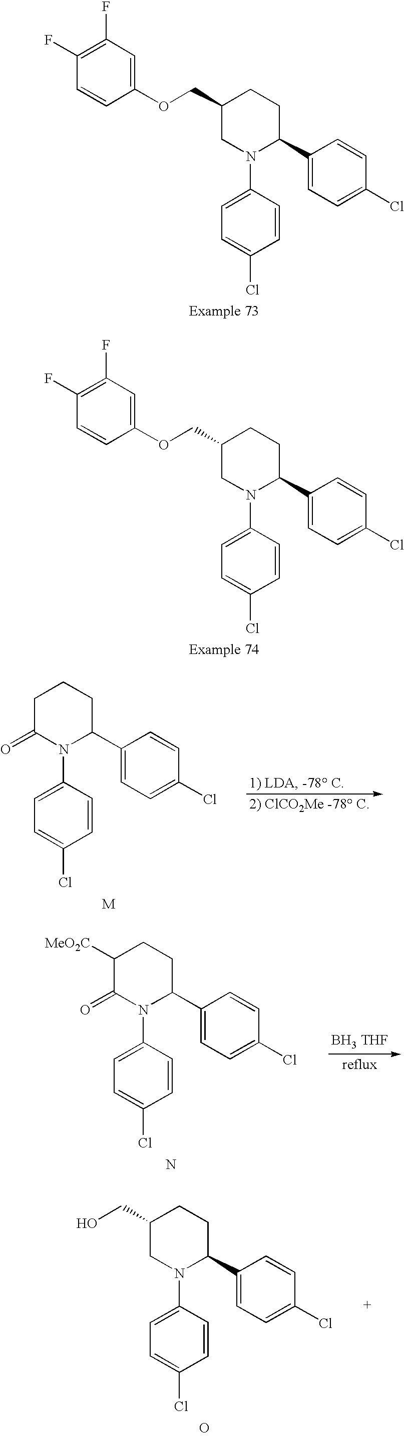 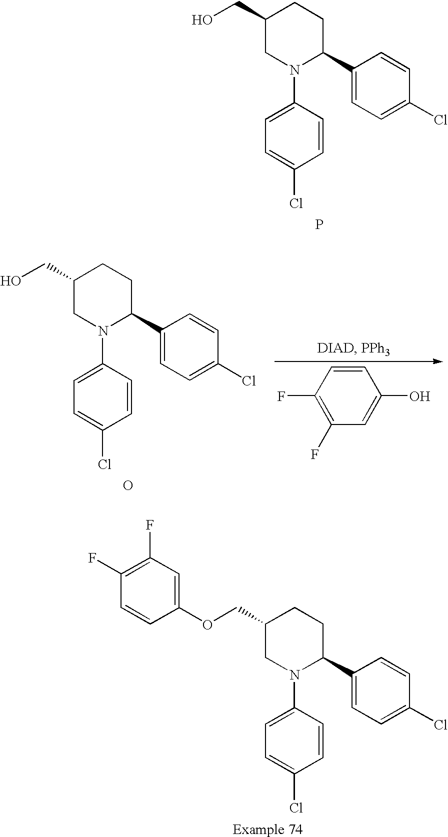 Step 1:

To a solution of LDA (6.1 mmol) in anhydrous THF (10 mL) at −78° C. was added Compound M (1.3 g, 4.1 mmol) in anhydrous THF (5 mL). The solution was allowed to stir at −78° C. for 1 h. To this solution was added methyl chloroformate (9.4 mmol). The solution was stirred at −78° C. for 1.5 h and warmed to RT and allowed to stir for an additional 1 h. The reaction was quenched with saturated NH₄Cl (aq.) and allowed to stir at RT overnight. The mixture was concentrated and partitioned between EtOAc and water. The aqueous layer was extracted with EtOAc (3×). The combined organic layers were dried over Na₂SO₄, filtered and concentrated. The crude product was purified by flash chromatography using gradient elution (SiO₂: 100:0 to 30:70 hexanes/EtOAc) to afford Compound N (480 mg) as a mixture of diastereomers.

Step 2:

To a solution of N (500 mg, 1.3 mmol) in THF was added borane THF complex (1 M solution in THF, 3.9 mmol). The solution was heated to reflux for 2 h. The solution was cooled to RT and excess MeOH was added. The solution was concentrated. The product was partitioned between CH₂Cl₂ and NaHSO₄ (aq.). The aqueous layer was extracted with CH₂Cl₂ (3×). The combined organic layers were dried over Na₂SO₄, filtered and concentrated. The crude product was purified by flash chromatography using gradient elution (SiO₂: 100:0 to 70:30 hexanes/EtOAc) to afford the trans diastereomer 0 (110 mg) and the cis diastereomer P (170 mg).

Step 3:

To a solution of the trans diastereomer 0 (60 mg, 0.2 mmol) in THF (3 mL) at 0° C. was added DIAD (diisopropylazodicarboxylate) (43 mg, 0.21 mmol) and this solution was stirred at 0° C. for 15 min. To this solution was added PPh₃ (61 mg, 0.23 mmol) and 3,4-difluorophenol (30 mg, 0.23 mmol). The solution was allowed to warm up to RT overnight. The solution was concentrated and partitioned between EtOAc and 1 N NaOH. The aqueous layer was extracted with EtOAc (3×). The combined organic layers were dried over Na₂SO₄, filtered and concentrated. The crude material was purified by repeated preparative TLC (SiO₂: 20% EtOAc/hexanes) to afford Example 74 (10 mg).

Example 73 was prepared according to Step 3, above, except that cis diastereomer P was used instead of trans diastereomer O.

### Preparation of Example 75

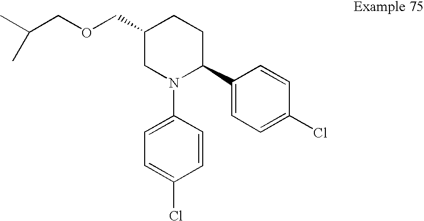

To a suspension of KOH (26 mg, 0.47 mmol) in DMSO (1 mL) was added a solution of 0 (30 mg, 0.1 mmol) followed by 1-bromo-2-methylpropane (16 mg, 0.12 mmol). The solution was allowed to stir at RT overnight. Water and brine was added and the mixture was extracted with EtOAc (3×). Dried combined organic layers over Na₂SO₄, filtered and concentrated. The crude material was purified by preparative TLC (SiO₂: 23% EtOAc/hexanes) to afford Example 75 (4 mg).

### Preparation of Examples 76 and 77

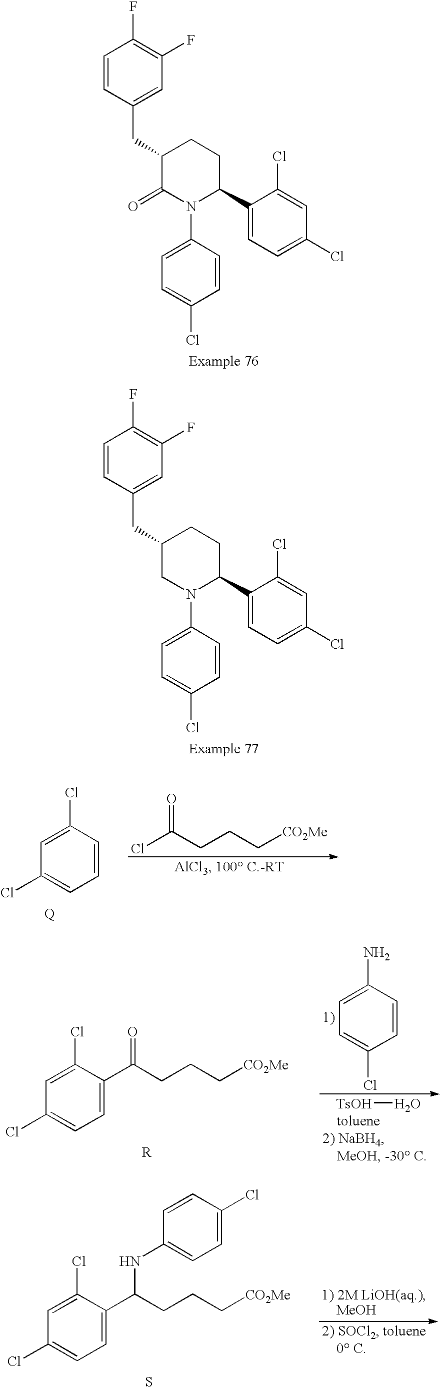 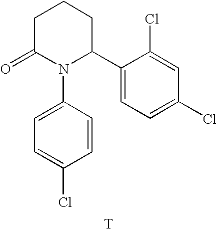 Step 1:

To a stirred suspension of AlCl₃ (19.2 g, 144 mmol) in 1,3-dichlorobenzene (Q) (43.2 g, 294 mmol) was added glutaric acid monomethyl ester chloride (12 g, 72 mmol). The resultant mixture was heated to 100° C. for 4 hours. The solution was allowed to slowly cool to room temperature and was stirred overnight at this temperature. To the solution was slowly added ice water followed by 1 N HCl (aq.). The aqueous layer was extracted with CH₂Cl₂. The organic layer was dried over Na₂SO₄, filtered, and concentrated. The crude material was purified by flash chromatography using gradient elution (SiO₂: 100:0 to 85:15 hexanes:ethyl acetate) to afford ketone R (9.9 g, 50% yield) as a light yellow oil.

Step 2:

To a solution of the ketone R (10.5 g, 38.2 mmol) in toluene (150 mL) was added 4-chloroaniline (5.6 g, 43.9 mmol) and p-toluenesulfonic acid monohydrate (290 mg, 1.5 mmol). The solution was heated to reflux overnight with a Dean-Stark trap attached. The solution was cooled to room temperature and concentrated. To the resultant oil was added MeOH (150 mL) followed by NaHCO₃ (1.3 g). The solution was cooled to −30° C. and NaBH₄ (3.5 g) was added over 1 hour in portions. The solution was then stirred at −30° C. for an additional 30 min. The solution was warmed to room temperature and water was added. The aqueous layer was extracted with EtOAc (3×). The combined organic layers were washed with brine, dried over Na₂SO₄, filtered and concentrated to afford amino ester S. The material was used without purification.

Step 3:

To a solution of the crude amino ester S (35 mmol) in methanol (150 mL) was added 2M LiOH (aq.) (57 mL, 115 mmol). The solution was stirred at room temperature for 4 h. The pH was adjusted to approx 6 using 4N HCl (aq.) The solution was extracted with EtOAc (3×). The combined organic layers were washed with brine, dried over Na₂SO₄, filtered and concentrated. To the crude product was added anhydrous toluene (100 mL) and pyridine (8.3 g, 105 mmol). The resultant solution was cooled to 0° C. To this solution was added dropwise a solution of SOCl₂ (3.1 mL, 42 mmol) in toluene (15 mL). After the addition was complete the solution was stirred for an additional 1 h. To the solution was added 1 M HCl (aq.). The aqueous layer was extracted with EtOAc (3×). The combined organic layers were washed with brine, dried over Na₂SO₄, filtered and concentrated. The crude material was purified by flash chromatography using gradient elution (SiO₂: 100:0 to 1:1 hexanes:ethyl acetate) to afford lactam T (3.4 g, 25% yield over 4 steps) as a white solid.

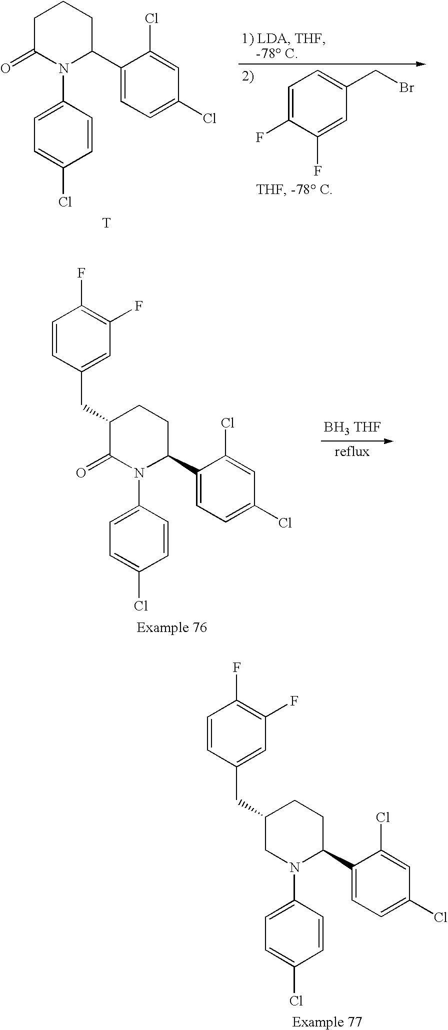

### Example 76

To a solution of LDA (2.52 mmol) in anhydrous THF (10 mL) at −78° C. was added a solution of the lactam T (600 mg, 1.68 mmol) in anhydrous THF (2 mL). The solution was stirred at −78° C. for 1 h. 3,4-difluorobenzyl bromide (2.52 mmol) was added slowly. After TLC confirmed the absence of lactam T (approx 30 min) the reaction was quenched with sat. NH₄Cl (aq.). The mixture was then extracted with EtOAc (3×). The combined organic layers were dried over Na₂SO₄, filtered and concentrated. The crude material was purified by flash chromatography using gradient elution (SiO₂: 100:0 to 70:30 hexanes:ethyl acetate) to afford 210 mg Example 76 (26% yield).

### Example 77

To a solution of Example 76 in THF was added a solution of BH₃.THF complex (1 M solution in THF, 1.3 mL). The solution was heated to reflux for 2 h. To this solution was added MeOH and the solution was concentrated. The crude product was partitioned between CH₂Cl₂ and H₂O. The aqueous layer was extracted with CH₂Cl₂ (3×). The combined organic layers were dried over Na₂SO₄, filtered and concentrated. The crude material was purified by flash chromatography using gradient elution (SiO₂: 100:0 to 96:4 hexanes:ethyl acetate) to afford 170 mg of Example 77 (83% yield).

### Preparation of Examples 78 and 79

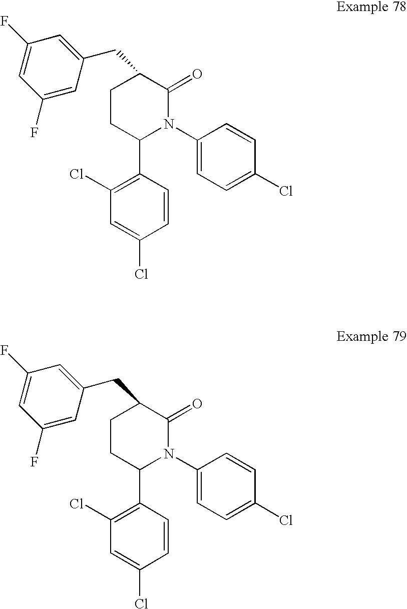

Examples 78 and 79 were prepared using procedures similar to those described above for Example 76, except that 3,5-difluorobenzyl bromide was used instead of 3,4-difluorobenzyl bromide.

### Preparation of Example 80

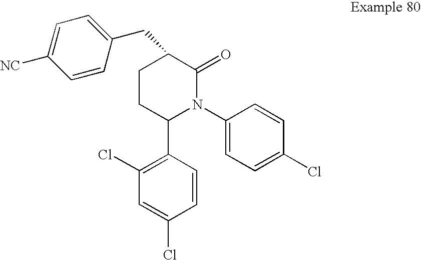

Example 80 was prepared using a procedure similar to that described above for Example 76, except that 4-cyanobenzyl bromide was used instead of 1-bromo-2-methylpropane.

### Preparation of Example 81

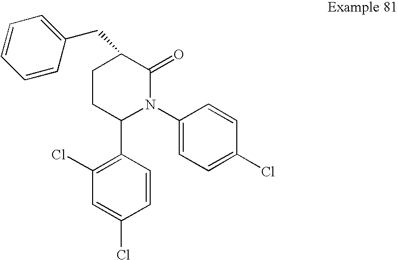

Example 81 was prepared using a procedure similar to that described above for Example 76, except that benzyl bromide was used instead of 1-bromo-2-methylpropane.

### Preparation of Example 82

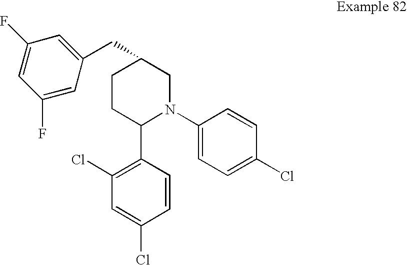

Example 82 was prepared using a procedure similar to that described above for Example 77, except that Example 78 was used as the starting material instead of Example 76.

### Preparation of Example 83

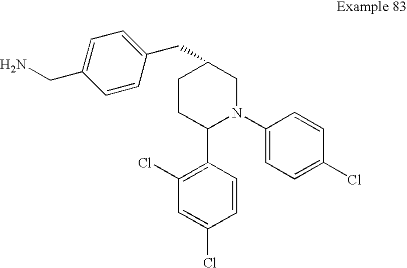

To a solution of Example 80 (160 mg, 0.33 mmol) in THF (2 mL) was added BH₃.THF complex (1 M solution in THF, 1.0 mL). The solution was heated to reflux for 2 h. To this solution was added MeOH and the solution was concentrated. The crude product was partitioned between CH₂Cl₂ and H₂O. The aqueous layer was extracted with CH₂Cl₂ (3×). The combined organic layers were dried over Na₂SO₄, filtered and concentrated. The crude product was purified by prep TLC (SiO₂: 4:1 hexanes:EtOAc) to afford Example 83 (18 mg).

### Preparation of Example 84

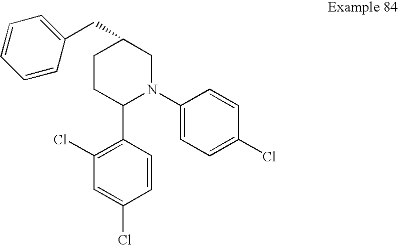

Example 84 was prepared using a procedure similar to that described above for Example 77, except that Example 81 was used as the starting material instead of Example 76.

### Preparation of Examples 85 and 86

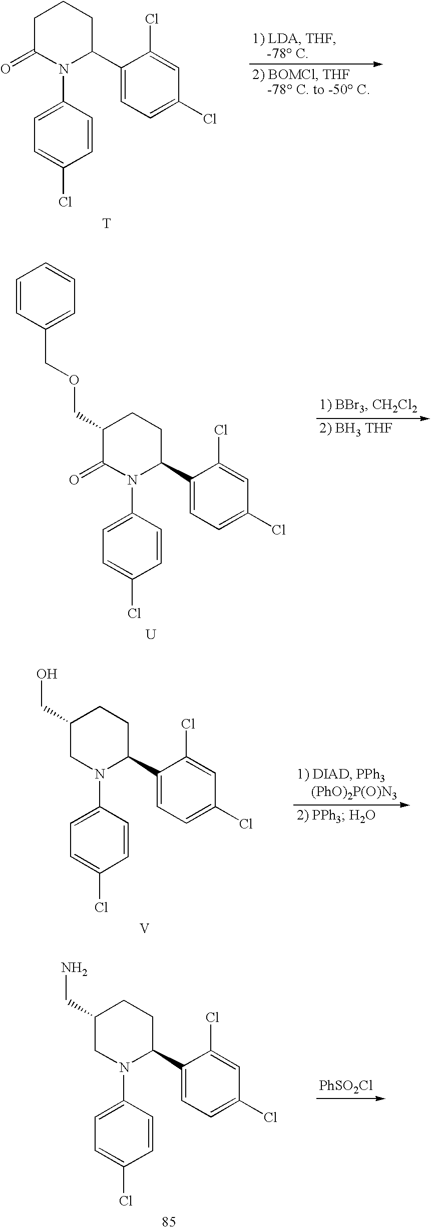 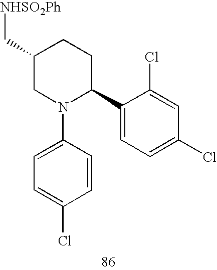 Step 1:

To a solution of LDA (4.23 mmol) in anhydrous THF at −78° C. was added a solution of the lactam T (1.0 g, 2.82 mmol) in anhydrous THF (2 mL). The solution was stirred at −78° C. for 1 h. To this solution was added benzyl chloromethyl ether (530 mg, 3.4 mmol). The solution was warmed to −50° C. and allowed to stir at this temperature for 1 h. Saturated NH₄Cl was added and the mixture was extracted with EtOAc (3×). The combined organic layers were washed with brine, dried over Na₂SO₄, filtered and concentrated. The crude material was purified by flash chromatography using gradient elution (SiO₂: 100:0 to 75:25 hexanes:ethyl acetate) to afford 567 mg ether U (42% yield).

Step 2:

To a solution of ether U (567 mg, 1.25 mmol) in anhydrous CH₂Cl₂ at 0° C. was added BBr₃ (1 M solution in hexanes, 1.87 mmol). The solution was warmed to RT and allowed to stir at this temperature for 2 h. To this solution was added NaHCO₃ (aq.). The mixture was extracted with CH₂Cl₂. The organic layer was dried over Na₂SO₄, filtered and concentrated. The crude material was purified by flash chromatography using gradient elution (100:0 to 1:9 hexanes:ethyl acetate) to afford 400 mg product (83% yield). The product was dissolved in anhydrous THF (5 mL). To this solution was added borane THF complex (1 M in THF, 3.1 mL). The solution was heated to reflux for 2 h. The solution was cooled to RT and 1 M HCl was slowly added. The resultant mixture was stirred at RT for 30 min. The solution was basified by the addition of NaHCO₃ (aq.) and extracted with CH₂Cl₂ (3×). The combined organic layers were dried over Na₂SO₄, filtered and concentrated. The crude material was purified by flash chromatography using gradient elution (100:0 to 65:35 hexanes:ethyl acetate) to afford 310 mg of alcohol V (67% yield for the 2 steps).

### Example 85

To a solution of the alcohol V (320 mg, 0.88 mmol) in THF (3 mL) at 0° C. was added PPh₃ (460 mg, 1.76 mmol) followed by the addition of DIAD (356 mg, 1.76 mmol). After the formation of a white precipitate (ca. 2 min) DPPA (484 mg, 1.76 mmol) was added. The mixture was warmed to RT and allowed to stir an additional 1.5 h. Water (3 drops) was added to the reaction mixture and the solution was concentrated. The crude material was purified by flash chromatography using gradient elution (SiO₂: 100:0 to 95:5 hexanes:EtOAc) to afford the azide (270 mg).

To a solution of the azide (70 mg, 0.18 mmol) stirred at RT for 1 h followed by an additional 1.5 h at 60° C., water (0.094 mL) was added and the mixture was stirred at 45° C. for 2.5 days. To this mixture was added Na₂SO₄ (ca. 50 mg) and the mixture was stirred at RT for several minutes. The mixture was filtered and concentrated. The crude material was purified by preparative TLC (SiO₂: 90:9.3:0.7 CH₂Cl₂:MeOH:conc. NH₄OH(aq.)) to afford Example 85 (53 mg).

### Example 86

To a solution of Example 85 (53 mg, 0.14 mmol) in CH₂Cl₂ (2 mL) was added Et₃N (10 drops) followed by benzene sulfonyl chloride (76 mg, 0.43 mmol). The solution was stirred at RT overnight and concentrated. The crude product was purified by prep TLC (SiO₂: 3:1 hexanes:EtOAc) to afford Example 86 (53 mg, 74% yield).

### Preparation of Example 87

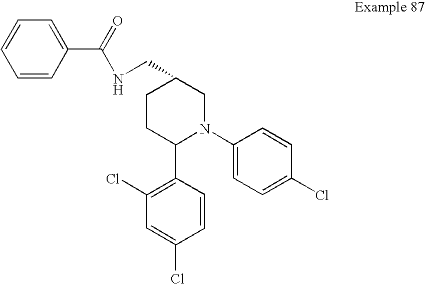

Example 87 was prepared using procedures similar to those for preparing Example 86, except that benzoyl chloride was the reagent used instead of benzene sulfonyl chloride.

### Preparation of Example 88

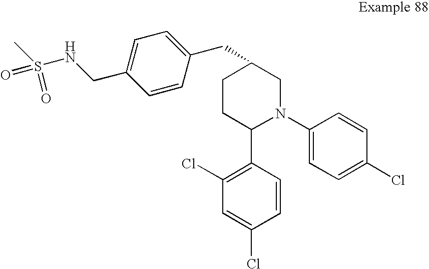

To a solution of Example 83 (15 mg, 0.033 mmol) in CH₂Cl₂ (1 mL) was added pyridine (3 drops) and methane sulfonyl chloride (7 mg, 0.66 mmol). The solution was heated to reflux and stirred overnight. The solution was then concentrated and partitioned between EtOAc and NaHCO₃ (aq.). The aqueous layer was extracted with EtOAc. The organic layer was dried over Na₂SO₄, filtered and concentrated. The crude product was purified by prep TLC (SiO₂: 3:1 hexanes:EtOAc) to afford 88 (10 mg).

### Preparation of Example 89

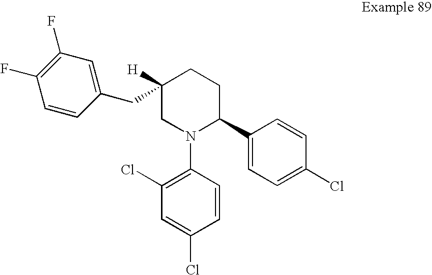

To a solution of Example 72 (50 mg, 0.116 mmol) in CH₂Cl₂ (3 mL) was added in portions over 2 h sulfuryl chloride (42 mg, 0.318 mmol). The solution was allowed to stir at RT for an additional 1 h. Water was added and the aqueous layer was extracted with CH₂Cl₂ (3×). The combined organic layers were dried over Na₂SO₄, filtered and concentrated. The crude product was purified by repeated prep TLC (SiO₂; 4:1 and 8:1 hexanes:EtOAc) to afford Example 89 (1 mg).

### Preparation of Example 90

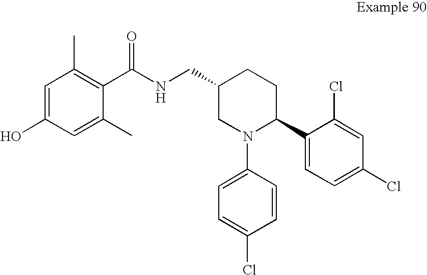

To a solution of Example 85 (35 mg, 0.095 mmol) in MeCN (1.5 mL) was added EDCl (27 mg, 0.14 mmol), HOBt (20 mg, 0.14 mmol), iPr₂NEt (61 mg, 0.48 mmol) and 4-hydroxy-2,6-dimethyl benzoic acid (31 mg, 0.19 mmol). 4-hydroxy-2,6-dimethyl benzoic acid was prepared by the method described in U.S. Pat. No. 6,391,865B1, which is herein incorporated by reference. The solution was allowed to stir at RT overnight. The solution was concentrated and partitioned between water and CH₂Cl₂. The aqueous layer was extracted with CH₂Cl₂ (3×). The combined organic layers were dried over Na₂SO₄, filtered and concentrated. The crude product was purified by preparative TLC (1:1 EtOAc:hexanes) to afford 37 mg of Example 90.

### Preparation of Example 91

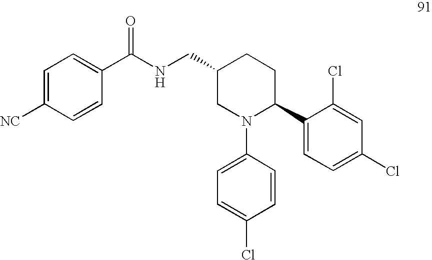

Example 91 was prepared using procedures similar to those used to prepare Example 90, except 4-cyanobenzoic acid was used instead of 4-hydroxy-2,6-dimethyl benzoic acid.

### Preparation of Example 92

Example 92 was prepared using procedures similar to those used to prepare Example 90, except 4-fluorobenzoic acid was used instead of 4-hydroxy-2,6-dimethyl benzoic acid.

### Examples 93 through 124

Sulfonamide analogs were prepared by the reaction of Example 85 with a sulfonyl chloride library as indicated below.

PS-DIEA (33 mg, 0.11 mmol) (Argonaut Technologies) was added to a 96-well microtiter plate followed by a stock solution of Example 85 (0.022 mmol) in dioxane/THF (1 mL 7:3 dioxane/THF). A stock solution of one of the various sulfonyl chlorides listed in the table below (0.088 mmol, 0.5M in THF) was added to each well of the microtiter plate and the plate was sealed and shaken overnight. PS-isocyante (44 mg, 0.066 mmol) (Argonaut Technologies), PS-trisamine (32 mg, 0.13 mmol) (Argonaut Technologies) and MeCN (0.5 mL) were added to each well. The plate was resealed and shaken overnight. The solutions were filtered through a polypropylene frit into a 96 well collection plate and the resin was washed with MeCN (3×0.5 mL). The resultant solutions were transferred into 2-dram vials and the solvents were removed in vacuo via a SpeedVac concentrator. The resulting samples were evaluated by LCMS and those that were >70% pure are listed in the table below:

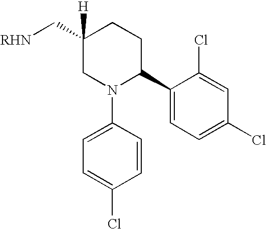

| Ex. | R | Sulfonyl chloride |
|---|---|---|
| 93 | 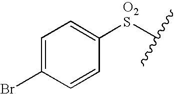 | 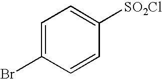 |
| 94 | 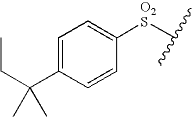 | 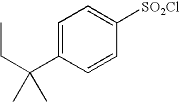 |
| 95 | 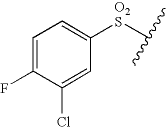 | 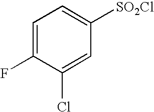 |
| 96 | 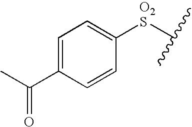 | 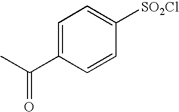 |
| 97 | 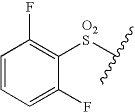 | 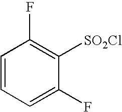 |
| 98 | 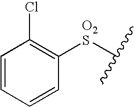 | 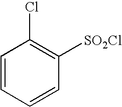 |
| 99 | 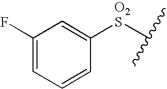 | 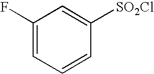 |
| 100 | 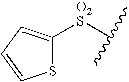 | 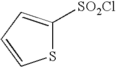 |
| 101 | 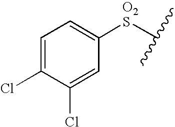 | 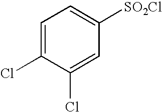 |
| 102 | 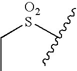 | 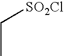 |
| 103 | 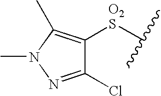 | 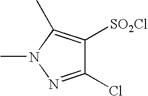 |
| 104 | 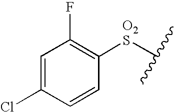 | 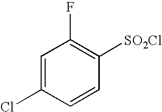 |
| 105 | 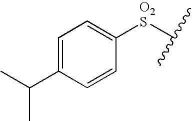 | 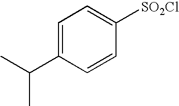 |
| 106 | 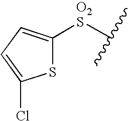 | 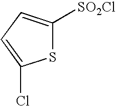 |
| 107 | 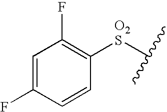 | 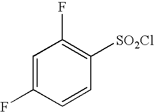 |
| 108 | 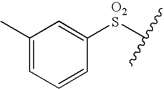 | 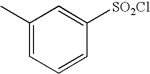 |
| 109 | 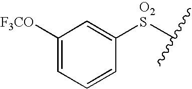 | 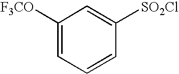 |
| 110 | 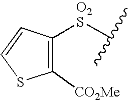 | 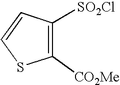 |
| 111 | 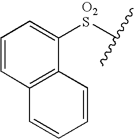 | 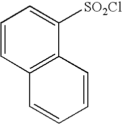 |
| 112 | 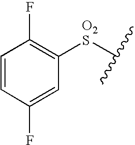 | 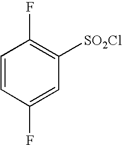 |
| 113 | 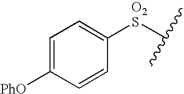 | 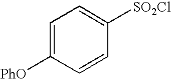 |
| 114 | 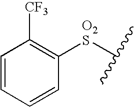 | 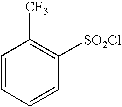 |
| 115 | 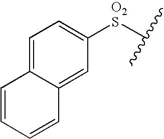 | 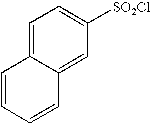 |
| 116 | 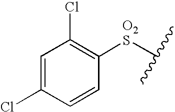 | 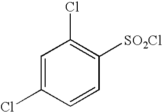 |
| 117 | 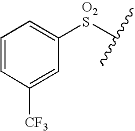 | 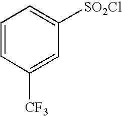 |
| 118 | 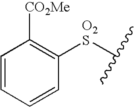 | 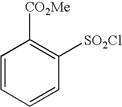 |
| 119 | 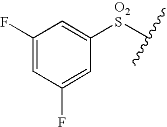 | 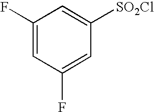 |
| 120 | 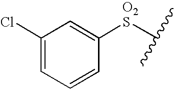 | 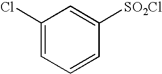 |
| 121 | 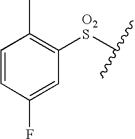 | 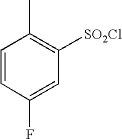 |
| 122 | 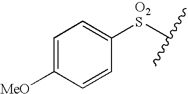 | 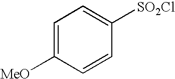 |
| 123 | 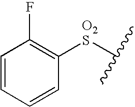 | 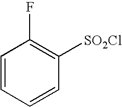 |
| 124 | 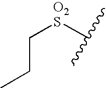 | 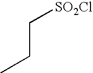 |

### Preparation of Example 125

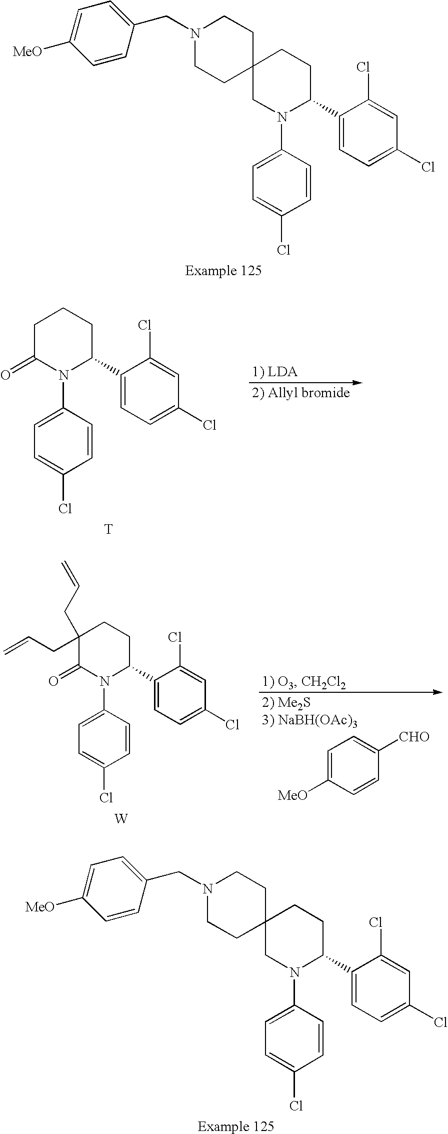 Step 1:

A solution of lactam T (940 mg, 2.65 mmol) in THF (20 mL) at −78° C. was added to a solution of LDA (7.95 mmol) in THF (20 mL). The resultant solution was stirred at −78° C. for 30 min. Allyl bromide (737 mg, 6.09 mmol) was added and the solution was stirred at −78° C. for 30 min. The reaction was quenched with pH 6.0 buffer and the mixture was allowed to warm to RT. The aqueous layer was extracted with EtOAc (3×). The combined organic layers were washed with brine, dried over Na₂SO₄, filtered and concentrated. The crude product was purified by flash chromatography (SiO₂: gradient elution 100:0 to 80:20 hexanes:EtOAc) to afford 540 mg W.

Step 2:

To a solution of W (640 mg, 1.47 mmol) in CH₂Cl₂ at −78° C. was bubbled 03 until the solution turned blue. The solution was then degassed with N₂ and excess Me₂S was added. The solution was warmed to RT and stirred overnight. The solution was concentrated and partitioned between CH₂Cl₂ and water. The aqueous layer was extracted with CH₂Cl₂ (3×). The combined organic layers were dried over Na₂SO₄, filtered and concentrated. The crude product was redissolved in 1,2-dichloroethane (20 mL). To this solution was added 4-methoxybenzylamine (303 mg, 2.2 mmol) and NaBH(OAc)₃ (934 mg, 4.4 mmol). The resultant mixture was stirred at RT for 96 h. The solution was diluted with CH₂Cl₂ and the organic layer was washed with 1 M NaOH. The aqueous layer was extracted with CH₂Cl₂ (2×). The combined organic layers were dried over Na₂SO₄, filtered and concentrated. The crude product was purified by preparative TLC (1:1 Acetone:hexanes) to afford 140 mg Example 125.

### Preparation of Example 126

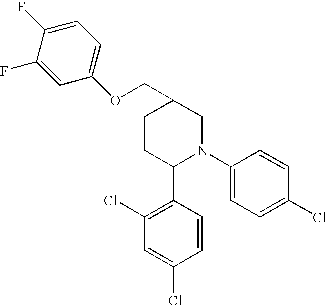

### Example 126

Example 126 was prepared using procedures similar to those used to prepare Example 74, except alcohol V was used instead of alcohol 0 in Step 3.

### Preparation of Example 127

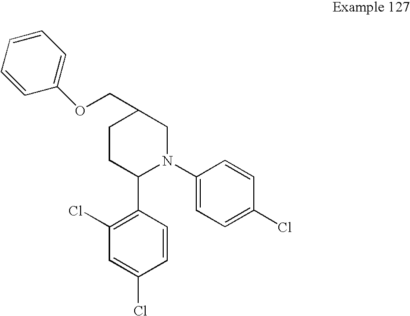

Example 127 was prepared using procedures similar to those used to prepare Example 126, except phenol was used instead of 3,4-difluorophenol.

### Preparation of Examples 128-131

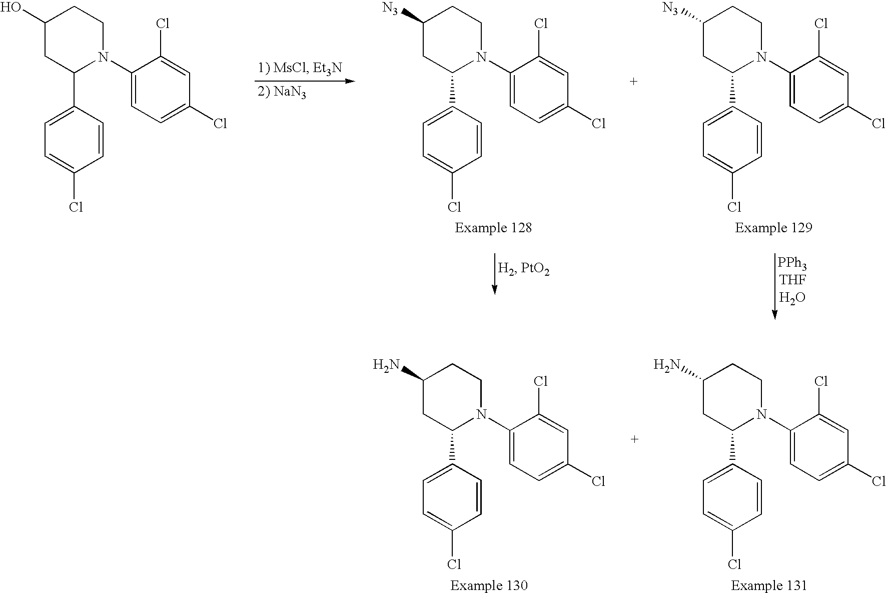 Step 1:

To a solution of the alcohol from Step 2 of Examples 1 and 2 (43 mg, 0.12 mmol) in CH₂Cl₂ (0.7 mL) at 0° C. was added Et₃N (31 mg, 0.30 mmol) and methane sulfonyl chloride (18 mg, 0.16 mmol). The mixture was stirred at 0° C. for 1 hour followed by an additional 1 hr at RT. Water was added and the aqueous layer was extracted with CH₂Cl₂. The organic layer was dried over Na₂SO₄, filtered and concentrated.

Step 2:

To a solution of the mesylate from step 1 (38 mg, 0.088 mmol) in DMF (0.4 mL) was added sodium azide (12 mg, 0.17 mmol). The solution was heated to 83° C. for 6 hr. The solution was concentrated. The material was partitioned between water and CH₂Cl₂. The organic layer was dried over Na₂SO₄, filtered and concentrated. The crude material was purified by prep TLC (SiO₂, 7:3 hexanes:Et₂O) to afford 6 mg of Example 128 and 6 mg of Example 129.

Step 3:

To a solution of Example 128 (4.8 mg) in MeOH (0.15 mL) was added PtO₂ (1.6 mg) in a round bottom flask and the flask was sealed with a septum. A balloon filled with H₂ was attached to the flask. The mixture was stirred at RT for 2 hr. The catalyst was removed via filtration and the solution was concentrated. The crude product was purified by prep TLC (SiO₂; 95:5:0.1 CH₂Cl₂:MeOH: 7N NH₃/MeOH) to afford 3 mg amine Example 130.

Step 4:

To a solution of Example 129 (0.48 g, 1.26 mmol) in THF (8 mL) was added triphenylphosphine (2 g). The solution was heated to reflux until the stating material was consumed. Water (0.5 mL) was added and the solution was stirred until the intermediate was consumed at which point the mixture was concentrated. The crude product was purified by flash chromatography (100:0 to 0:100 hexanes:Et₂O followed by 95:5:0.1 CH₂Cl₂:MeOH: 7N NH₃/MeOH) to afford Example 131 (448 mg).

### Preparation of Examples 132-148

Sulfonamide analogs were prepared in a manner similar to the procedures described in Examples 93-124, except that the indicated sulfonyl chloride was reacted with either Example 130 or 131 prepared in Steps 3 or 4 above.

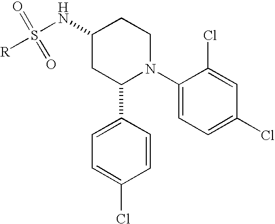

| Ex. | R | Sulfonyl Chloride |
|---|---|---|
| 132 | 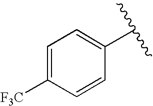 | 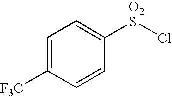 |
| 133 | 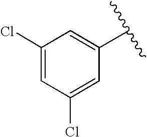 | 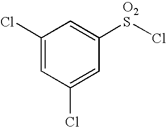 |
| 134 | 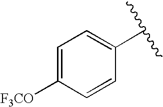 | 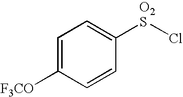 |
| 135 | 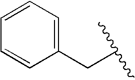 | 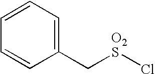 |
| 136 | 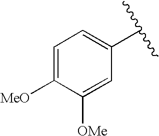 | 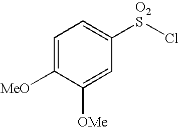 |
| 137 | 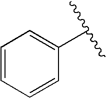 | 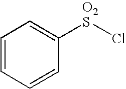 |
| 138 | 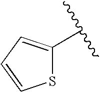 | 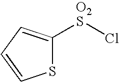 |
| 139 | 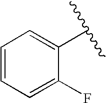 | 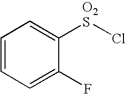 |
| 140 | 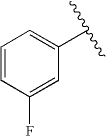 | 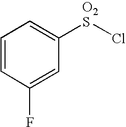 |
| 141 | 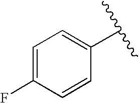 | 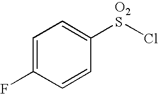 |
| 142 | 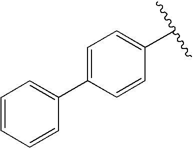 | 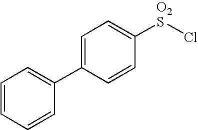 |
| 143 | 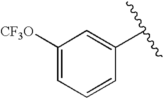 | 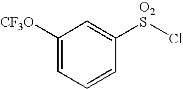 |
| 144 | 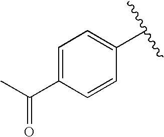 | 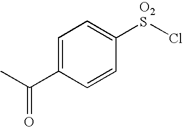 |
| 145 | 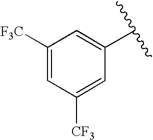 | 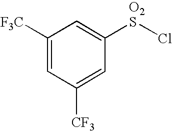 |
| 146 | 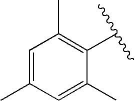 | 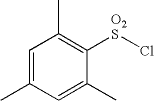 |
| 147 | 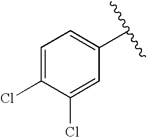 | 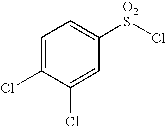 |

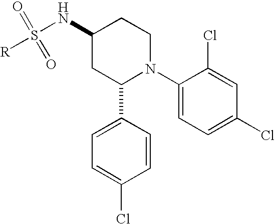

| Ex. | R | Sulfonyl Chloride |
|---|---|---|
| 148 | 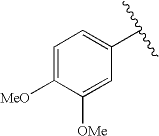 | 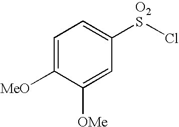 |

### Preparation of Examples 149-162

Amide analogs were prepared by the reaction of either Example 130 or 131 prepared in Steps 3 or 4, above, with a carboxylic acid library as indicated in the table below.

PS-EDC resin (Polymer Laboratories) (48 mg, 0.068 mmol) was added to each well of a 96 deep well polypropylene microtiter plate followed by a stock solution of one of the amines prepared in Step 1 of Examples 3 and 4 (6.0 mg, 0.0169 mmol) in MeCN/THF (3/2, 1 mL) and HOBt (5 mg, 0.025 mmol). To this solution was added a 1 M stock solution of the appropriate carboxylic acid (0.025 mmol). The wells were sealed and the plate was shaken at RT overnight. The solutions were filtered through a polypropylene frit into a second microtiter plate containing PS-Isocyanate resin (Argonaut Technologies) (0.051 mmol) and PS-trisamine (Argonaut Technologies) (0.135 mmol). The top plate was rinsed with MeCN (0.5 mL/well). The bottom plate was sealed and shaken at RT overnight. The solutions were filtered through a polypropylene frit into a 96 well collection plate. The wells of the top plate were washed with MeCN (0.5 mL/well). The resultant solutions in the collection plate were transferred into vials and the solvent was removed in vacuo using a Speedvac. The resulting samples were evaluated by LCMS and those that were >70% pure are shown below:

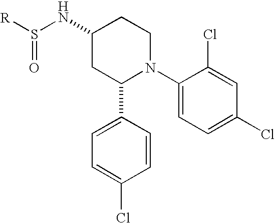

| Ex. | R | Carboxylic Acid |
|---|---|---|
| 149 | 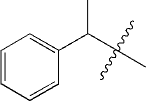 | 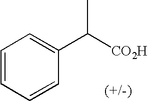 |
| 150 | 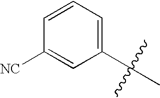 | 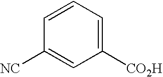 |
| 151 | 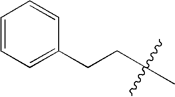 | 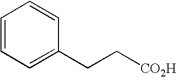 |
| 152 | 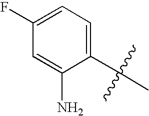 | 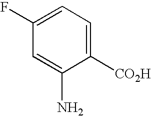 |
| 153 | 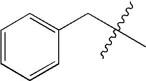 | 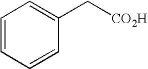 |
| 154 | 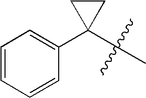 | 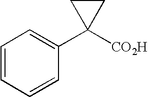 |
| 155 | 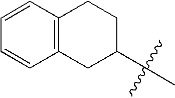 | 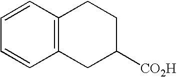 |
| 156 | 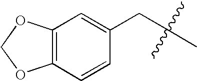 | 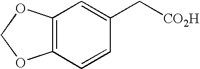 |
| 157 | 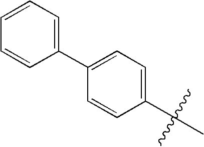 | 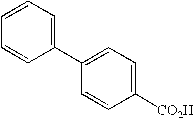 |
| 158 | 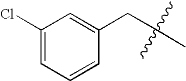 | 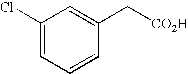 |
| 159 | 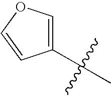 | 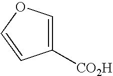 |
| 160 | 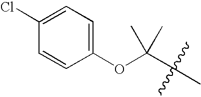 | 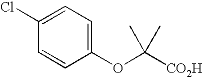 |

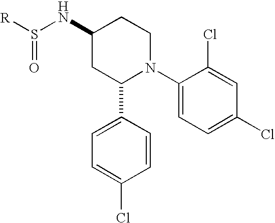

| Ex. | R | Carboxylic Acid |
|---|---|---|
| 161 | 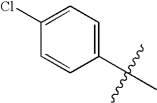 | 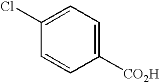 |
| 162 | 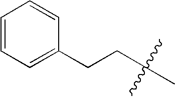 | 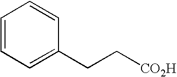 |

### Preparation of Examples 163-167

Urea analogs were prepared by the reaction of Example 131 prepared in Steps 3 above with an isocyanate library as indicated in the table below.

A solution of Example 130 (0.0169 mmol) in dichloroethane:acetonitrile (1:1, 1 mL) was added to 16 wells of a deep well polypropylene microtiter plate. To these wells were added a 0.5 M solution of the appropriate isocyanate (0.051 mmol) in dichloromethane. The plate was sealed and shaken at RT overnight. The solutions were filtered through a polypropylene frit into a second microtiter plate containing PS-Isocyanate resin (Argonaut Technologies) (0.051 mmol) and PS-trisamine (Argonaut Technologies) (0.135 mmol). The top plate was rinsed with MeCN (0.5 mL/well). The bottom plate was sealed and shaken at RT overnight. The solutions were filtered through a polypropylene frit into a 96 well collection plate. The wells of the top plate were washed with MeCN (0.5 mL/well). The resultant solutions in the collection plate were transferred into vials and the solvent was removed in vacuo using a Speedvac. The resulting samples were evaluated by LCMS and those that were >70% pure are shown below.

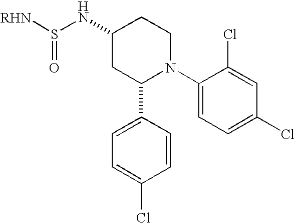

| Ex. | R | Isocyanate |
|---|---|---|
| 163 | 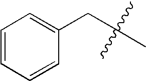 | 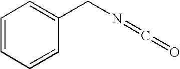 |
| 164 | 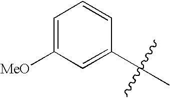 | 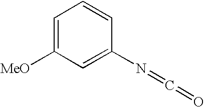 |
| 165 | 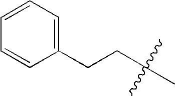 | 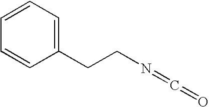 |
| 166 | 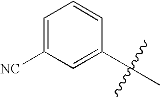 | 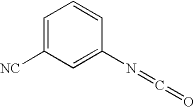 |
| 167 | 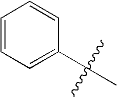 | 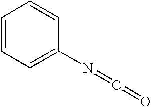 |

### Preparation of Examples 168-169

The urea analogs were prepared in the same method as Examples 163-167 except Example 131 was used as the starting material.

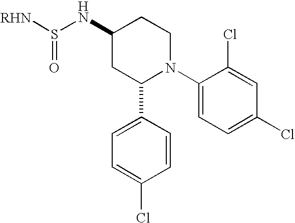

| Ex. | R | Isocyanate |
|---|---|---|
| 168 | 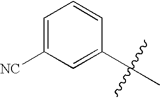 | 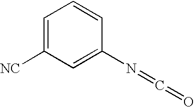 |
| 169 | 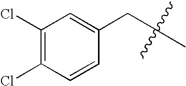 | 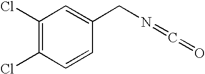 |

### Preparation of Example 170

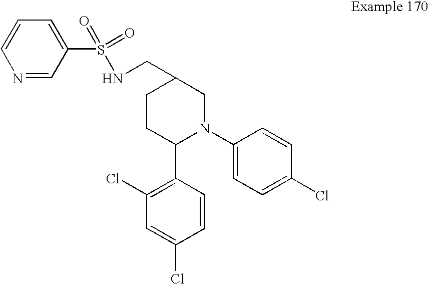

Example 170 was prepared using the procedure for preparing Example 86, except that 3-pyridine sulfonyl chloride hydrochloride salt (Chemical Synthesis Services) was used instead of benzene sulfonyl chloride.

### Preparation of Example 171

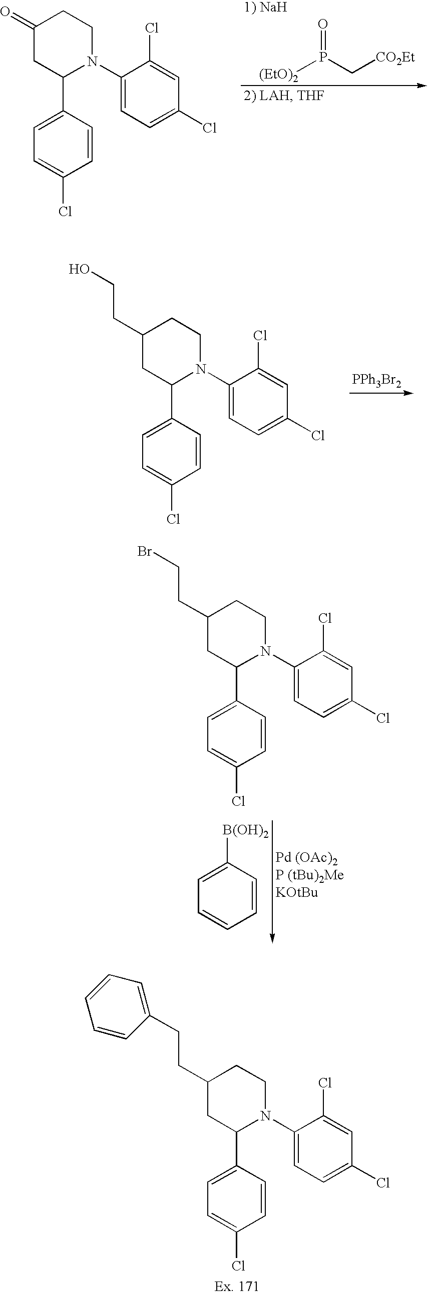

The ketone prepared by the method of Step 4 of Examples 1 & 2 can be converted to 2-[2-(4-chloro-phenyl)-1-(2,4-dichloro-phenyl)-piperidin-4-yl]-ethanol, for example, using the procedure described in J. Med. Chem. (2001), 2707-2718. 2-[2-(4-Chloro-phenyl)-1-(2,4-dichloro-phenyl)-piperidin-4-yl]-ethanol can then be converted to 4-(2-bromo-ethyl)-2-(4-chloro-phenyl)-1-(2,4-dichloro-phenyl)-piperidine with P(Ph)₃Br₂ using conventional methods. 4-(2-Bromo-ethyl)-2-(4-chloro-phenyl)-1-(2,4-dichloro-phenyl)piperidine can then be converted to Example 171, for example using the procedure described in J. Am. Chem. Soc. (2002), 13662-13663.

### Preparation of Example 172

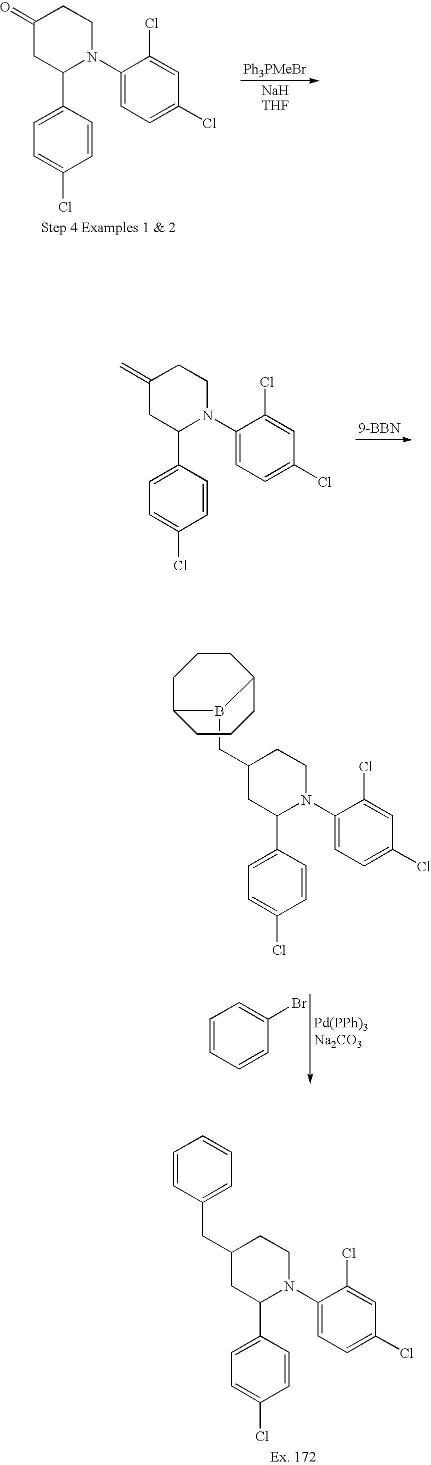

The ketone prepared by the method of Step 4 of Examples 1 & 2 can then be converted to 2-(4-chloro-phenyl)-1-(2,4-dichloro-phenyl)-4-methylene-piperidine using Wittig reaction conditions. 2-(4-Chloro-phenyl)-1-(2,4-dichloro-phenyl)-4-methylene-piperidine can then be reacted with 9-BBN to form 4-(9-Bora-bicyclo[3.3.1]non-9-ylmethyl)-2-(4-chloro-phenyl)-1-(2,4-dichloro-phenyl)-piperidine, which can then be reacted with bromobenzene to provide Example 172.

### Preparation of Examples 173-224

Examples 173-224 were prepared using a procedure similar to that described above for Examples 149-162, except that Example 85 was used as the starting material instead of Examples 130 or 131.

### Preparation of Example 225

Example 225 was prepared using a procedure similar to that described above for Examples 149-162 except the tert-butoxy carbonyl group was removed by the treatment of the intermediate with MP-TsOH in MeOH.

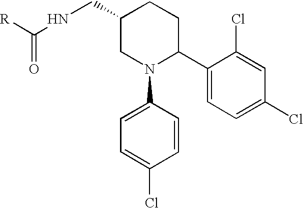

| | | Carboxylic |
|---|---|---|
| Ex. | R | Acid |
| 173 | 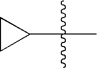 | 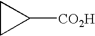 |
| 174 | 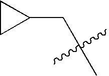 | 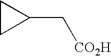 |
| 175 | 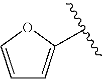 | 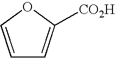 |
| 176 | 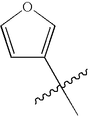 | 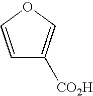 |
| 177 | 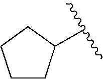 | 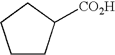 |
| 178 | 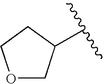 | 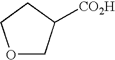 |
| 179 | 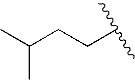 | 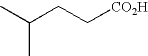 |
| 180 | 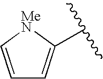 | 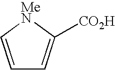 |
| 181 | 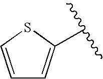 | 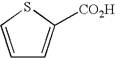 |
| 182 | 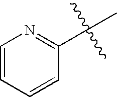 | 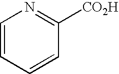 |
| 183 | 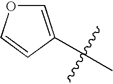 | 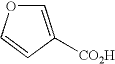 |
| 184 | 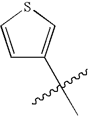 | 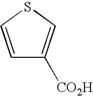 |
| 185 | 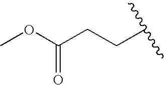 | 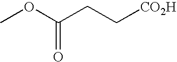 |
| 186 | 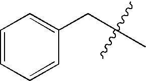 | 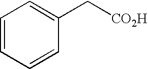 |
| 187 | 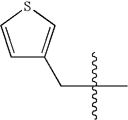 | 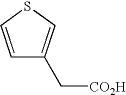 |
| 188 | 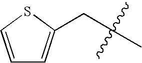 | 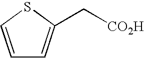 |
| 189 | 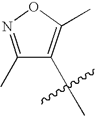 | 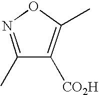 |
| 190 | 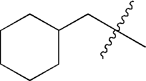 | 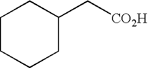 |
| 191 | 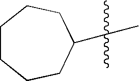 | 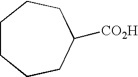 |
| 192 | 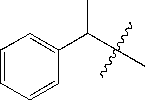 | 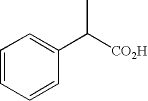 |
| 193 | 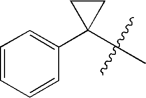 | 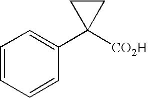 |
| 194 | 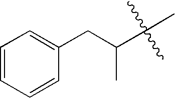 | 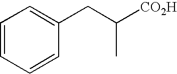 |
| 195 | 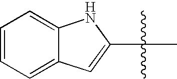 | 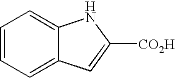 |
| 196 | 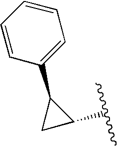 | 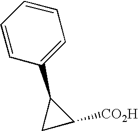 |
| 197 | 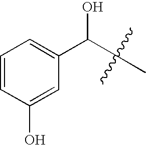 | 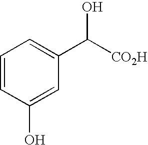 |
| 198 | 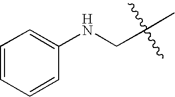 | 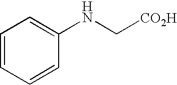 |
| 199 | 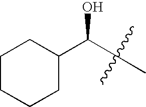 | 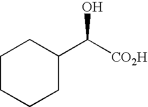 |
| 200 | 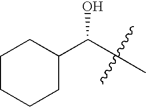 | 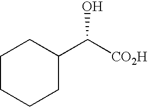 |
| 201 | 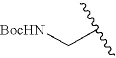 | 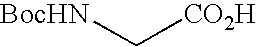 |
| 202 | 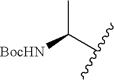 | 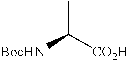 |
| 203 | 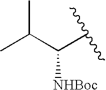 | 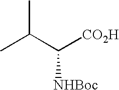 |
| 204 | 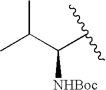 | 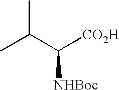 |
| 205 | 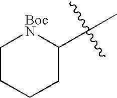 | 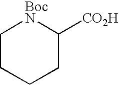 |
| 206 | 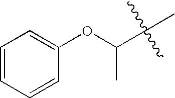 | 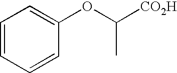 |
| 207 | 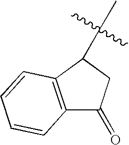 | 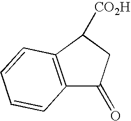 |
| 208 | 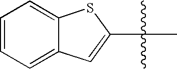 | 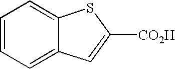 |
| 209 | 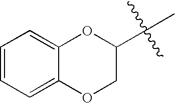 | 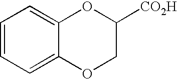 |
| 210 | 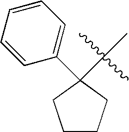 | 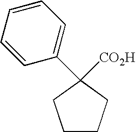 |
| 211 | 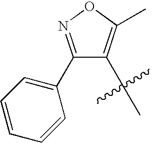 | 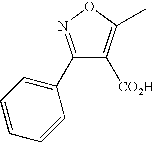 |
| 212 | 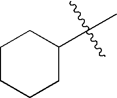 | 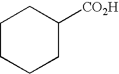 |
| 213 | 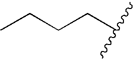 | 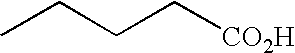 |
| 214 | 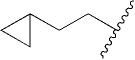 | 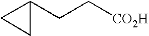 |
| 215 | 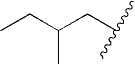 | 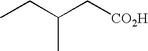 |
| 216 | 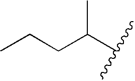 | 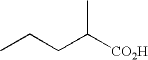 |
| 217 | 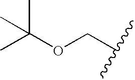 | 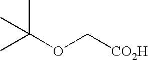 |
| 218 | 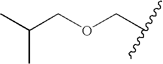 | 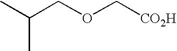 |
| 219 | 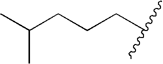 | 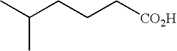 |
| 220 | 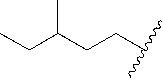 | 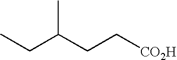 |
| 221 | 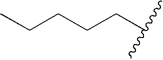 | 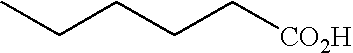 |
| 222 | 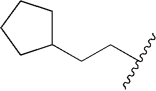 | 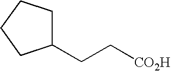 |
| 223 | 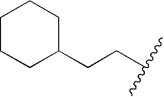 | 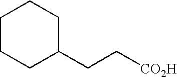 |
| 224 | 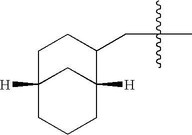 | 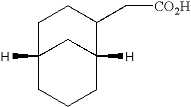 |
| 225 | 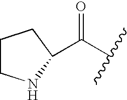 | 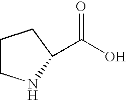 |

### Preparation of Examples 226-241

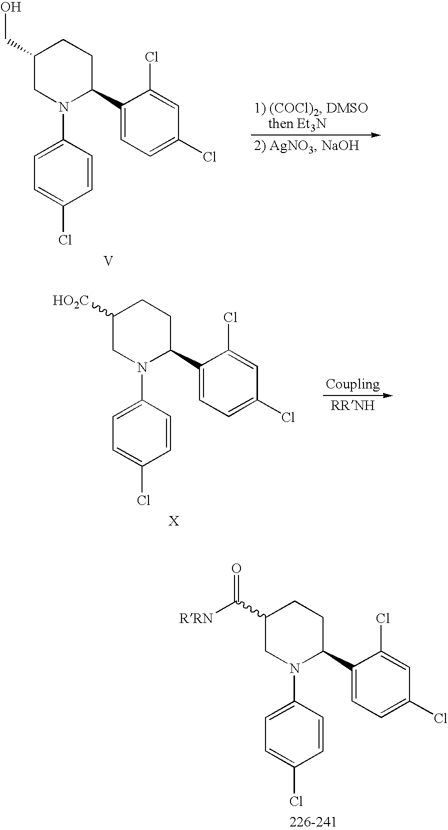 Step 1:

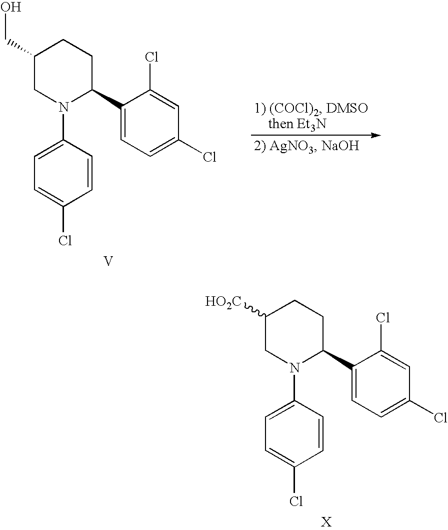

A solution of DMSO (29 μL, 0.34 mmol) in CH₂Cl₂ (0.5 mL) was added to a solution of oxalyl chloride (48 μL, 0.67 mmol) in CH₂Cl₂ (0.5 mL) at −78° C. under nitrogen and stirred for 20 min. A solution of V (from Examples 85 and 86) (50 mg, 0.14 mmol) in CH₂Cl₂ (1.5 mL) was added at −78° C. and stirred for 30 min. A solution of Et₃N (190 μL, 1.4 mmol) in CH₂Cl₂ (2 mL) was added at −78° C. and stirred for 30 min at −78° C. and 15 min at RT. The solution was diluted with CH₂Cl₂, washed with water, dried and concentrated to afford the intermediate aldehyde product (46 mg, 92%).

AgNO₃ (129 mg, 0.76 mmol) was added to a solution of NaOH (61 mg, 1.5 mmol) in H₂O (1 mL) at RT under nitrogen and stirred for 15 min. A solution of the above aldehyde product (140 mg, 0.38 mmol) in ethanol (2.8 mL) was added at 0° C. and stirred for 60 min. The mixture was filtered through celite. The filtrate was concentrated. The residue was dissolved in water, acidified with 3M HCl, and extracted with ether. The organic layer was dried and concentrated to afford X (110 mg, 75%).
Step 2:

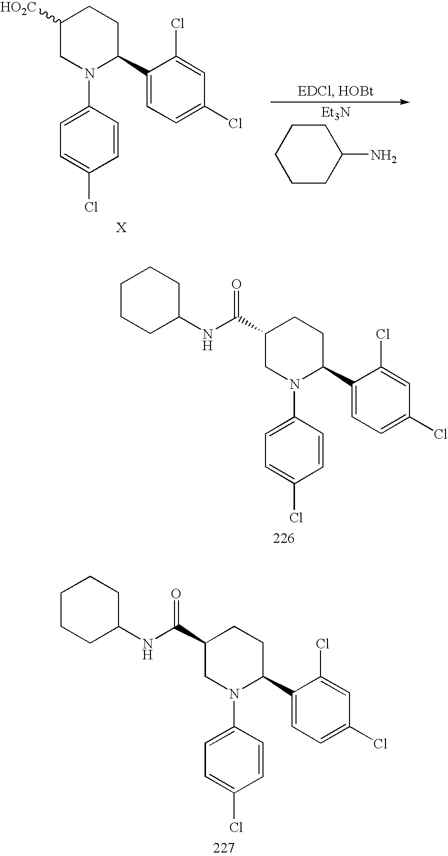

Cyclohexylamine (100 μL, 0.34 mmol) was added to a solution of the acid X (35 mg, 0.09 mmol) in DMF (0.9 mL) at RT followed by Et₃N (190 μL, 1.4 mmol), EDCl (173 mg, 0.90 mmol), and HOBt (62 mg, 0.45 mmol). The mixture was stirred at RT for 2 h. The mixture was concentrated. The residue was dissolved in water and extracted with ether. The organic layer was dried and concentrated. Separation of the residue via flash chromatography (50/50 hexanes/EtOAc, SiO₂) gave 226 (25 mg, 60%) and 227 (6 mg, 14%).

The following amides 228-241 were prepared similarly using the acid X and the appropriate amines.

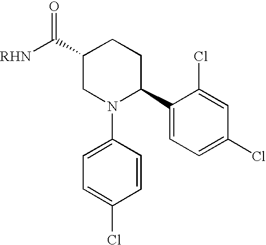

| Ex. # | R | Amine |
|---|---|---|
| 228 | 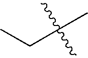 | 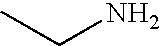 |
| 229 | 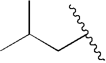 | 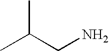 |
| 230 | 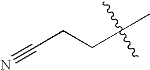 | 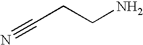 |
| 231 | 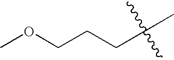 | 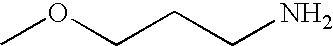 |
| 232 | 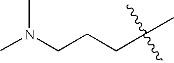 | 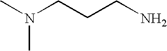 |
| 233 | 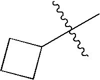 | 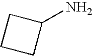 |
| 234 | 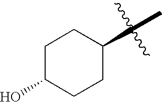 | 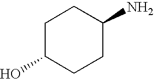 |
| 235 | 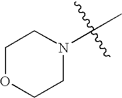 | 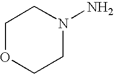 |
| 236 | 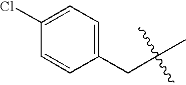 | 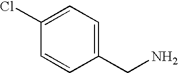 |
| 237 | 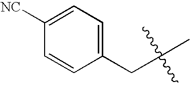 | 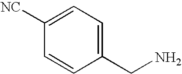 |
| 238 | 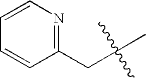 | 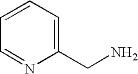 |

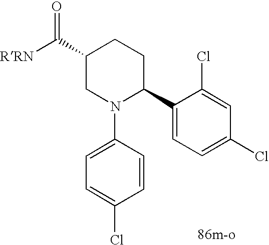

| Ex. # | NRR′ | Amine |
|---|---|---|
| 239 | 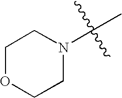 | 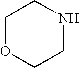 |
| 240 | 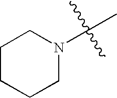 | 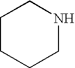 |
| 241 | 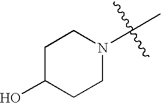 | 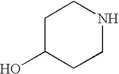 |

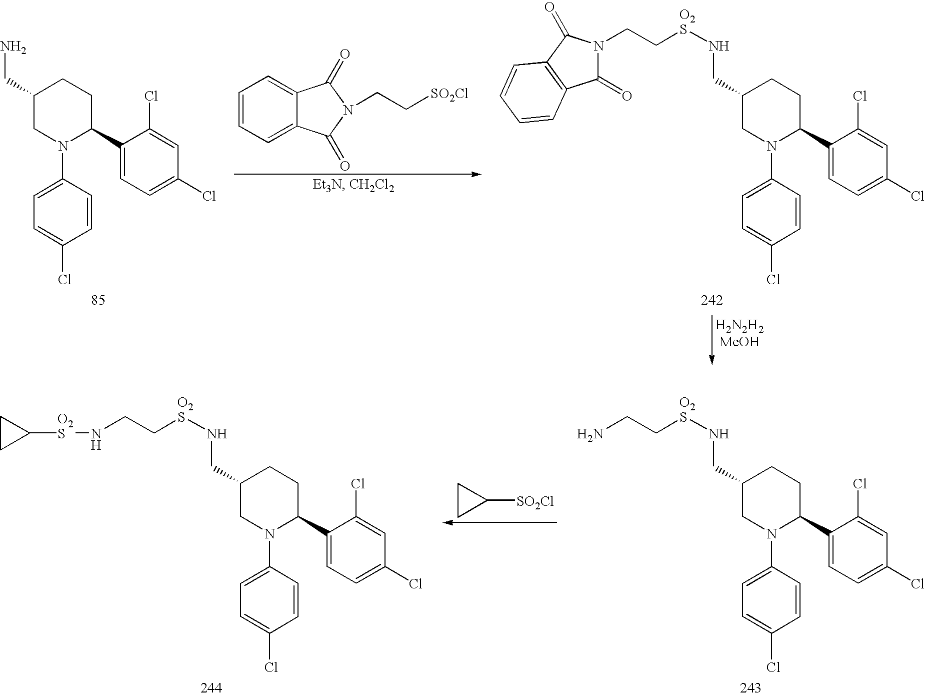

To a solution of Example 85 (200 mg, 0.54 mmol) in CH₂Cl₂ (2 mL) was added ET₃N (10 drops) and 2-phthalimidoethane sulfonyl chloride (Astatech). The solution was stirred at RT overnight. The solution was diluted with CH₂Cl₂. The solution was washed with H₂O. The aqueous layer was extracted with CH₂Cl₂ (3×). The combined organic layers were dried over Na₂SO₄, filtered and concentrated. The crude product was purified by flash chromatography (SiO₂: gradient 1:0 to 1:1 hexanes:EtOAc) to afford 300 mg Example 242.

To a solution of Example 242 (300 mg, 0.50 mmol) in MeOH was added hydrazine (48 mg, 1.5 mmol). The resultant solution was heated to reflux for 3 h at which time additional hydrazine (20 mg) was added and the solution was heated to reflux for an additional 1 h. The solution was then concentrated. To the crude material was added EtOAc and the white precipitate was removed by filtration. The solution was concentrated and the crude product was purified by flash chromatography [SiO₂: gradient 1:0:0 to 95:7:0.7 CH₂Cl₂:MeOH:7N NH₃ (in MeOH)] to afford Example 243 (135 mg).

To a solution of Example 243 (40 mg, 0.084 mmol) in CH₂Cl₂ (2 mL) was added Et₃N (10 drops) and cyclopropyl sulfonyl chloride (Array) (18 mg, 0.13 mmol). The solution was stirred at RT followed by an additional 24 h at reflux. The crude product was purified by preparative TLC [SiO₂: 95:5:0.5 CH₂Cl₂:MeOH:ammonium hydroxide]. to afford Example 244.

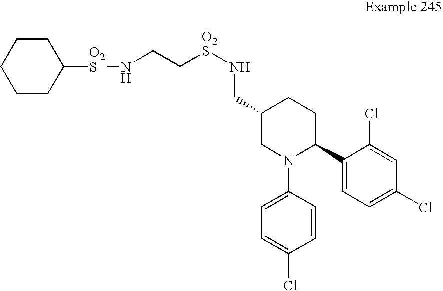

Example 245 was prepared using a procedure similar to that described above for Example 244, except cyclohexyl sulfonyl chloride (Array) was used instead of cyclopropyl sulfonylchloride.

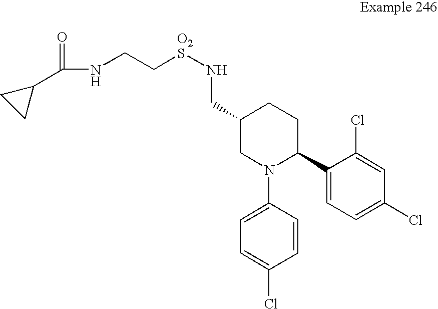

Example 246 was prepared using a procedure similar to that described above for Example 244, except cyclopropanecarbonyl chloride was used instead of cyclopropyl sulfonylchloride.

### Preparation of Example 247

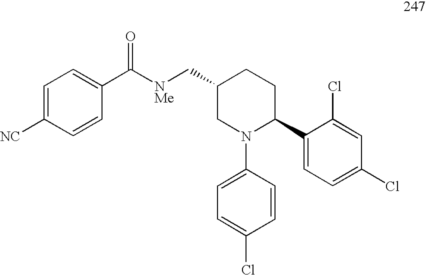

To a solution of Example 91 (81 mg, 0.16 mmol) in DMF was added NaH (4.8 mg, 0.20 mmol) followed by methyl iodide (28 mg, 0.2 mmol). The solution was stirred overnight. The solution was diluted with EtOAc and washed with water. The water layer was extracted with EtOAc (2×). The combined organic layers were dried over Na₂SO₄, filtered and concentrated. The crude product was purified by flash chromatography (SiO₂: gradient 1:0 to 1:1 hexanes:EtOAc) to afford 46 mg of Example 247.

### Preparation of Example 248

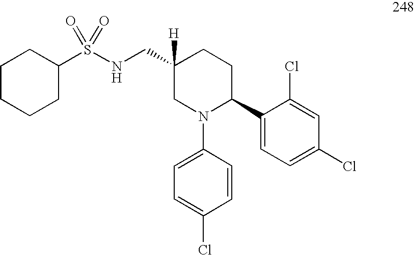

Example 248 was prepared using a procedure similar to that described above for Example 86, except cyclohexanesulfonyl chloride was used instead of benzene sulfonyl chloride.

### Preparation of Example 249

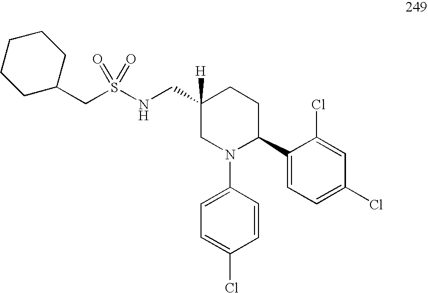

Example 249 was prepared using a procedure similar to that described above for Example 86, except cyclohexylmethanesulfonyl chloride was used instead of benzene sulfonyl chloride.

### Preparation of Example 250

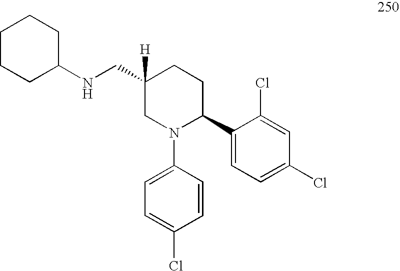

To a solution of Example 85 (50 mg, 0.14 mmol) in CH₂Cl₂ (1 mL) was added cyclohexanone (14 μL, 0.14 mmol) followed by sodium triacetoxyborohydride (34 mg, 0.16 mmol) and acetic acid (2 drops). The solution was stirred at RT overnight. The solution was diluted with NaHCO₃ (aq.) and extracted with EtOAc. The organic layer was dried over Na₂SO₄, filtered and concentrated. The crude product was purified by preparative TLC [SiO₂: 95:5:0.5 CH₂Cl₂:MeOH:ammonium hydroxide] to afford 33 mg Example 250.

### Preparation of Example 251

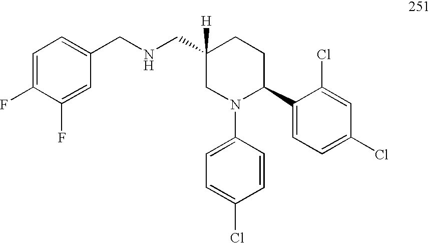

To a solution of Example 85 (50 mg, 0.14 mmol) in CHCl₃ was added MgSO₄ (50 mg) and 3,4 difluorobenzaldehyde (15 μL, 0.14 mmol). The mixture was stirred at RT for 70 h. The mixture was filtered and concentrated. Methanol was added followed by NaBH₄ (6.6 mg, 0.18 mmol). The mixture was stirred at RT for 2 h. The material was partitioned between H₂O and EtOAc. The organic layer was dried over Na₂SO₄, filtered and concentrated. The crude product was purified by flash chromatography (SiO₂: gradient 1:0 to 1:1 hexanes:EtOAc) to afford 50 mg of Example 251.

### Preparation of Example 252

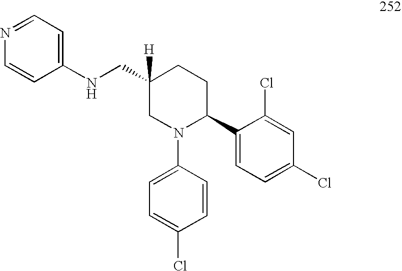

A mixture of Example 85 (50 mg, 0.14 mmol), 4-bromopyridine hydrochloride (31 mg, 0.16 mmol), NaOtBu (26 mg, 0.27 mmol), Pd(OAc)₂ (1.6 mg, 0.006 mmol) and BINAP (2.4 mg, 0.006 mmol) in toluene (1.5 mL) was heated at 70° C. for 2 days. The mixture was filtered and concentrated. The crude product was purified by semi-preparative HPLC (C₁₈: 100:0:1 to 0:100:1H₂O:MeCN:formic acid) to afford Example 252 (7 mg).

### Preparation of Example 253

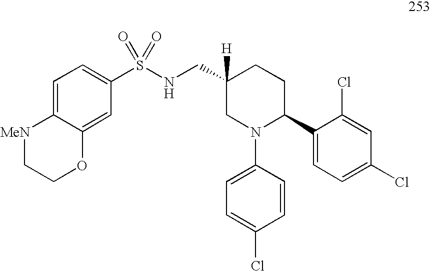

To a solution of Example 85 (50 mg, 0.14 mmol) in CH₂Cl₂ was added 4-methyl-3,4-dihydro-2H-11,4-benzoxazine-7-sulfonyl chloride (Maybridge) (40 mg, 0.16 mmol) and Et₃N (10 drops). The solution was heated to reflux overnight. The solution was concentrated and the crude product was purified by preparative TLC chromatography (SiO₂: 1:1 hexanes:EtOAc) to afford Example 253.

### Preparation of Example 254

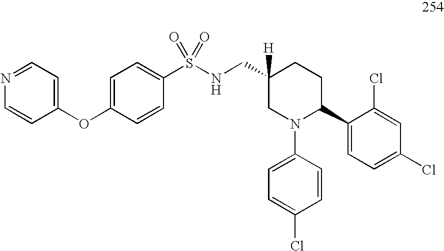

Example 254 was prepared using a procedure similar to that described above for Example 253, except (4-(4-pyridyloxy)phenyl)sulfonyl chloride hydrochloride was used instead of 4-methyl-3,4-dihydro-2H-1,4-benzoxazine-7-sulfonyl chloride.

### Preparation of Example 255

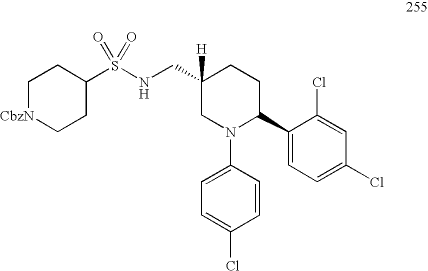

Example 255 was prepared using the procedure for Example 253, except 1-piperidine carboxylic acid, 4-(chlorosulfonyl)-phenylmethyl ester (Magical Scientific; Oklahoma City, Okla.) was used instead of 4-methyl-3,4-dihydro-2H-1,4-benzoxazine-7-sulfonyl chloride.

### Preparation of Examples 256 and 257

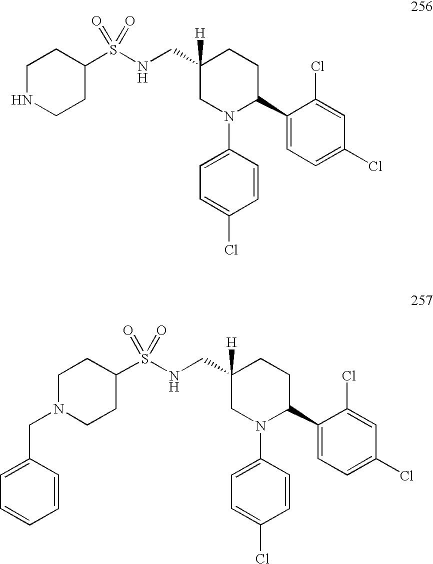

To a solution of Example 255 (135 mg, 0.21 mmol) in CH₂Cl₂ (15 mL) at 0° C. was added boron tribromide (156 mg, 0.6 mmol). The solution was allowed to warm to RT and stirred for 50 min. To this solution was added NaHCO₃ (aq.). The aqueous layer was extracted with CH₂Cl₂ (3×). The combined organic layers were dried over Na₂SO₄, filtered and concentrated. The crude product was purified by flash chromatography (SiO₂: gradient 1:0:0 to 90:11:0.75 CH₂Cl₂:MeOH:ammonium hydroxide) to afford 20 mg of Example 256 and 100 mg Example 257.

### Preparation of Example 258

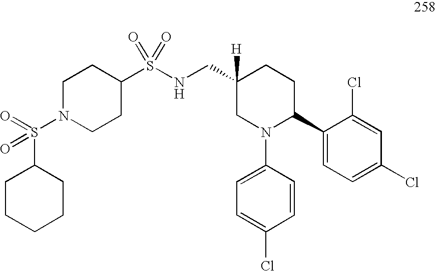

To a solution of Example 256 (30 mg, 0.06 mmol) in CH₂Cl₂ (2 mL) was added Et₃N (10 drops) followed by cyclohexyl sulfonyl chloride (17 mg, 0.09 mmol). The solution was stirred at RT overnight. Additional cyclohexyl sulfonyl chloride (90 mg) was added and the solution was heated to reflux for an additional 24 h. The solution was concentrated. The crude product was purified by preparative TLC chromatography (SiO₂: 6:4 hexanes:EtOAc) to afford 33 mg Example 258.

### Preparation of Example 259

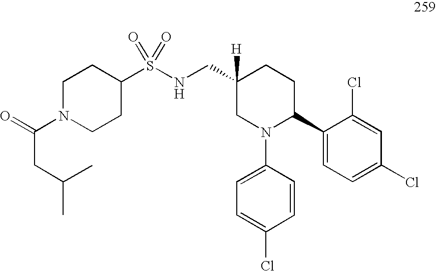

To a solution of Example 256 (30 mg, 0.06 mmol) in CH₂Cl₂ (2 mL) was added Et₃N (10 drops) followed by 3-methyl buturyl chloride (10 mg, 0.09 mmol). The solution was stirred at RT overnight. The solution was concentrated. The crude product was purified by preparative TLC chromatography (SiO₂: 1:1 hexanes:EtOAc) to afford 4 mg Example 259.

### Preparation of Example 260

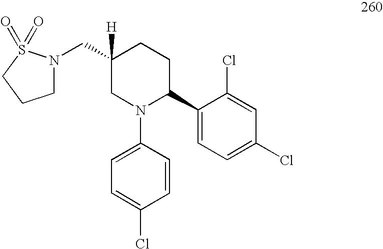

To a solution of Example 85 (30 mg, 0.084 mmol) in CH₂Cl₂ (2 mL) was added Et₃N (10 drops) followed by 3-chloropropyl sulfonyl chloride (22 mg, 0.13 mmol). The solution was stirred at RT overnight. Additional 3-chloropropyl sulfonyl chloride (90 mg) was added and the solution was heated to reflux for another 24 h. The solution was concentrated. The crude product was purified by preparative TLC chromatography (SiO₂: 6:4 hexanes:EtOAc). This product was dissolved in THF (2 mL) and potassium t-butoxide (7 mg, 0.06 mmol) was added. The mixture was heated to reflux for 3 h. The mixture was concentrated. The crude product was purified by preparative TLC chromatography (SiO₂: 65:35 hexanes:EtOAc) to afford 17 mg Example 260.

### Preparation of Example 261

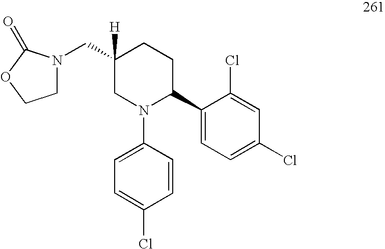

To a solution of Example 85 (26 mg, 0.070 mmol) in CH₂Cl₂ (2 mL) was added Et₃N (8.5 mg, 0.084 mmol) followed by 2-chloroethyl chloroformate (12 mg, 0.084 mmol). The solution was stirred at RT for 48 h. The solution was concentrated. The material was redissolved in CH₂Cl₂ and washed with NaHCO₃ (aq.). The aqueous layer was extracted with CH₂Cl₂ (2×). The combined organic layers were dried over Na₂SO₄, filtered and concentrated. The crude product was dissolved in THF (2 mL) and NaH (6 mg, 0.14 mmol) was added. The solution was heated to reflux for 2 h. Water was added and the mixture was extracted with EtOAc (3×). The combined organic layers were washed with brine, dried over Na₂SO₄, filtered and concentrated. The crude product was purified by preparative TLC chromatography (SiO₂: 1:1 hexanes:EtOAc) to afford 22 mg Example 261.

### Preparation of Example 262

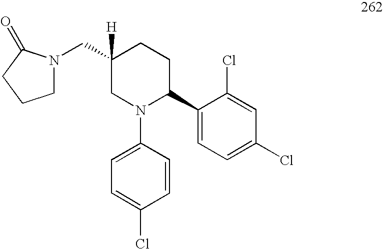

To a solution of Example 85 (30 mg, 0.081 mmol) in CH₂Cl₂ (2 mL) was added Et₃N (8.5 mg, 0.084 mmol) followed by 4-chlorobutryl chloride (14 mg, 0.097 mmol). The solution was stirred at RT for 48 h. The solution was concentrated. The material was redissolved with CH₂Cl₂ and washed with NaHCO₃ (aq.). The aqueous layer was extracted with CH₂Cl₂ (2×). The combined organic layers were dried over Na₂SO₄, filtered and concentrated. The crude product was dissolved in THF (2 mL) and NaH (7 mg, 0.16 mmol) was added. The solution was heated to reflux for 2 h. Water was added and the mixture was extracted with EtOAc (3×). The combined organic layers were washed with brine, dried over Na₂SO₄, filtered and concentrated. The crude product was purified by preparative TLC chromatography (SiO₂: 1:1 hexanes:EtOAc) to afford 20 mg Example 262.

### Preparation of Example 263

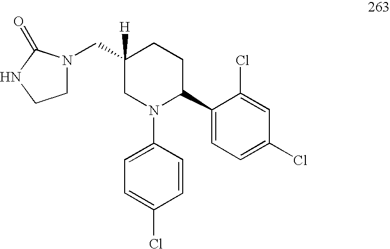

To a solution of Example 85 (54 mg, 0.15 mmol) in CH₂Cl₂ (2 mL) was added Et₃N (17 mg, 0.17 mmol) followed by 2-chloroethyl isocyanate (18 mg, 0.17 mmol). The solution was stirred at RT for 3 h. The solution was concentrated. The solution was diluted with CH₂Cl₂ and washed with NaHCO₃ (aq.). The aqueous layer was extracted with CH₂Cl₂ (2×). The combined organic layers were dried over Na₂SO₄, filtered and concentrated. The crude product was dissolved in THF (2 mL) and NaH (12 mg, 0.30 mmol) was added. The solution was stirred at RT for 48 h. Water was added and the mixture was extracted with EtOAc (3×). The combined organic layers were washed with brine, dried over Na₂SO₄, filtered and concentrated. The crude product was purified by preparative TLC chromatography (SiO₂: 1:2 hexanes:EtOAc) to afford 38 mg Example 263.

### Preparation of Example 264

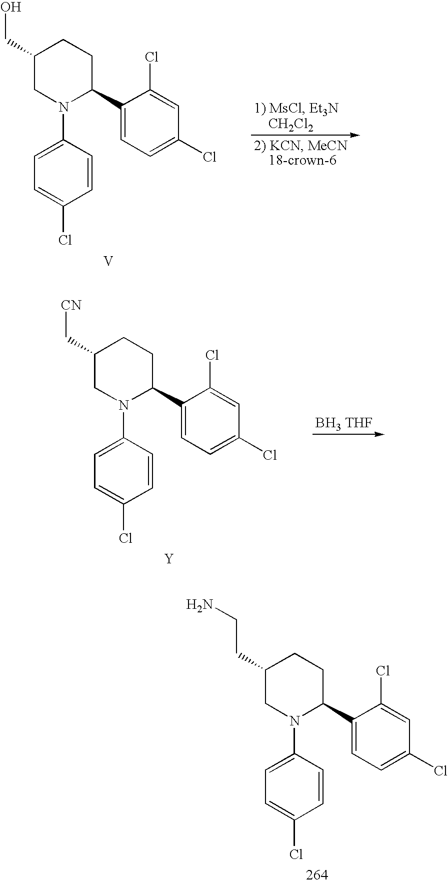 Step 1:

To a solution of V (see Example 85)(2.0 g, 5.4 mmol) in CH₂Cl₂ (20 mL) was added Et₃N (820 mg, 8.1 mmol) followed by methanesulfonyl chloride (680 mg, 5.9 mmol). The solution was stirred at RT overnight. Additional methanesulfonyl chloride (90 mg) was added and the solution was heated to reflux for 24 h. The solution was washed with NaHCO₃ (aq.) dried over Na₂SO₄, filtered and concentrated. The crude product was purified by flash chromatography (SiO₂: gradient elution 1:0 to 3:1 hexanes:EtOAc) to afford 2.36 g mesylate.

Step 2:

To a portion of the mesylate (1.76 g, 3.9 mmol) in MeCN (10 mL) was added potassium cyanide (970 mg, 14.9 mmol) and 18-crown-6 (120 mg). The solution was heated to reflux for 30 h. To the solution was added 1 N NaOH (aq.) and the mixture was extracted with CH₂Cl₂. The organic layer was washed with H₂O (2×). The organic layer was dried over Na₂SO₄, filtered and concentrated. The crude product was purified by flash chromatography (SiO₂: gradient elution 1:0 to 3:1 hexanes:EtOAc) to afford 1.36 g Y.

Step 3:

To a solution of Y (350 mg, 0.92 mmol) in THF (25 mL) was added borane-THF complex (1 M in THF) (2.77 mL, 2.77 mmol). The solution was heated to reflux for 2 h. The solution was cooled to RT and 1 M HCl (aq.) (3 mL) was added slowly. The mixture was stirred at RT for 30 min. The mixture was washed with H₂O. To the organic layer was added NaHCO₃ (aq.) and the mixture was extracted with CH₂Cl₂ (3×). The combined organic layers were dried over Na₂SO₄, filtered and concentrated. The crude product was purified by flash chromatography (SiO₂: gradient elution 1:0:0 to 95:7:0.5 CH₂Cl₂:MeOH:ammonium hydroxide)) to afford 243 mg Example 264.

### Preparation of Example 265

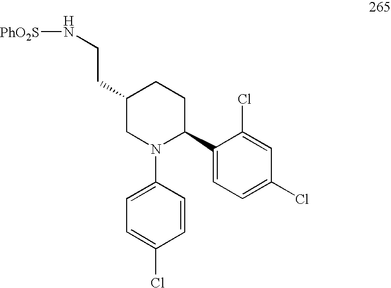

To a solution of Example 264 (40 mg, 0.10 mmol) in CH₂Cl₂ (2 mL) was added Et₃N (10 drops) followed by benzenesulfonyl chloride (28 mg, 0.16 mmol). The solution was stirred at RT for 48 h. The solution was concentrated. The crude product was purified by preparative TLC chromatography (SiO₂:3:1hexanes:EtOAc) to afford 60 mg Example 265.

### Preparation of Example 266

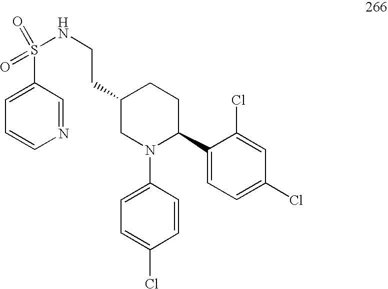

Example 266 was prepared using a procedure similar to that described above for Example 265, except 3-pyridylsufonyl chloride was used instead of benzene sulfonyl chloride.

### Preparation of Example 267

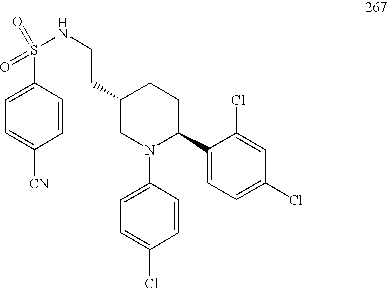

Example 267 was prepared using a procedure similar to that described above for Example 265, except 4-cyanobenzene sulfonyl chloride was used instead of benzene sulfonyl chloride.

### Preparation of Example 268

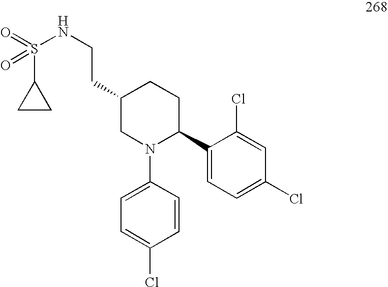

Example 268 was prepared using a procedure similar to that described above for Example 265, except cyclopropane sulfonyl chloride was used instead of benzene sulfonyl chloride.

### Preparation of Example 269

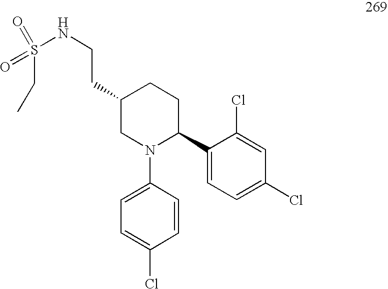

Example 269 was prepared using a procedure similar to that described above for Example 265, except ethane sulfonyl chloride was used instead of benzene sulfonyl chloride.

### Preparation of Example 270

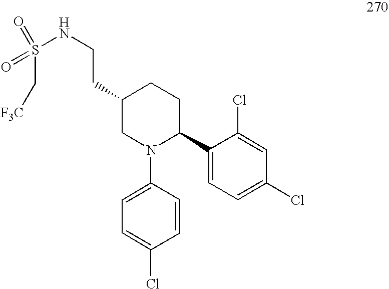

Example 270 was prepared using a procedure similar to that described above for Example 265, except 2,2,2-trifluoroethane sulfonyl chloride was used instead of benzene sulfonyl chloride.

### Preparation of Example 271

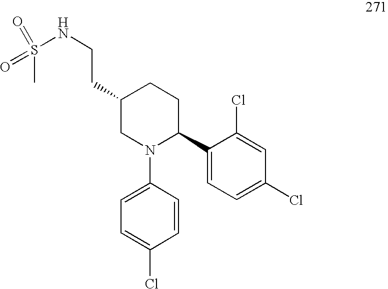

Example 271 was prepared using a procedure similar to that described above for Example 265, except methanesulfonyl chloride was used instead of benzene sulfonyl chloride.

### Preparation of Example 272

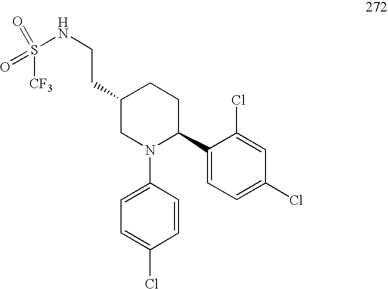

Example 272 was prepared using a procedure similar to that described above for Example 265, except trifluoromethanesulfonyl anhydride was used instead of benzene sulfonyl chloride.

### Preparation of Example 273

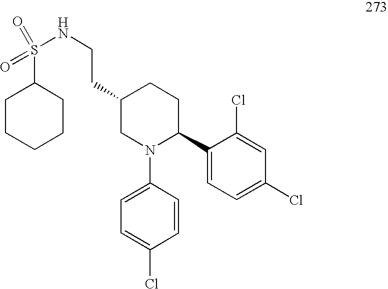

Example 273 was prepared using a procedure similar to that described above for Example 265, except cyclohexanesulfonyl chloride was used instead of benzene sulfonyl chloride.

### Preparation of Example 274

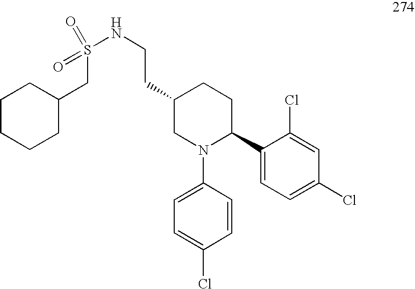

Example 274 was prepared using a procedure similar to that described above for Example 265, except cyclohexylmethanesulfonyl chloride was used instead of benzene sulfonyl chloride.

### Preparation of Example 275

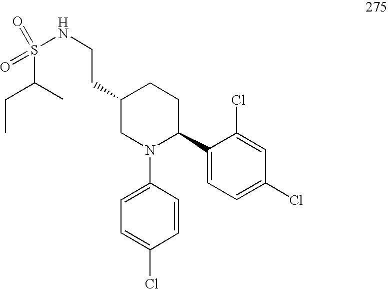

Example 275 was prepared using a procedure similar to that described above for Example 265, except butane-2-sulfonyl chloride was used instead of benzene sulfonyl chloride.

### Preparation of Example 276

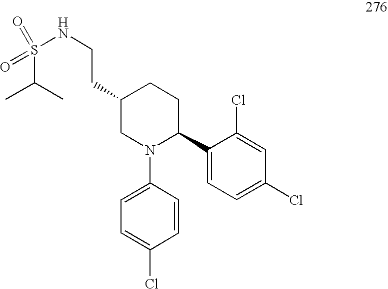

Example 276 was prepared using a procedure similar to that described above for Example 265, except 2-propylsulfonyl chloride was used instead of benzene sulfonyl chloride.

### Preparation of Example 277

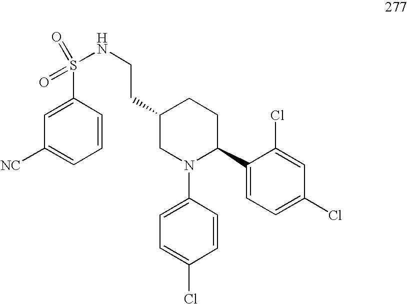

Example 277 was prepared using a procedure similar to that described above for Example 265, except 3-cyanobenzene sulfonyl chloride was used instead of benzene sulfonyl chloride.

### Preparation of Example 278

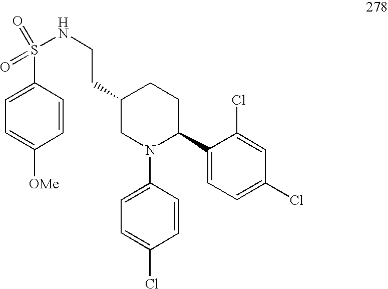

Example 278 was prepared using a procedure similar to that described above for Example 265, except 4-methoxybenzene sulfonyl chloride was used instead of benzene sulfonyl chloride.

### Preparation of Example 279

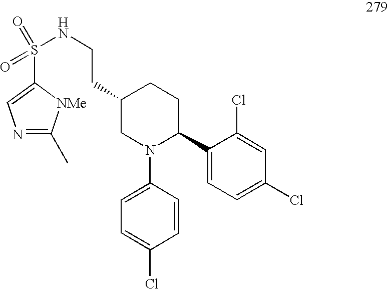

Example 279 was prepared using a procedure similar to that described above for Example 265, except 2,3-dimethyl-3H-imidazole-4-sulfonyl chloride was used instead of benzene sulfonyl chloride.

### Preparation of Examples 280 and 281

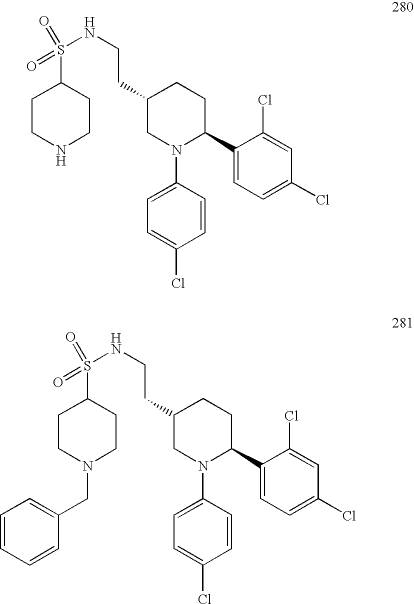

Examples 280 and 281 were prepared using procedures similar to those used above for Examples 255-257, except Example 254 was used instead of Example 85.

### Preparation of Example 282

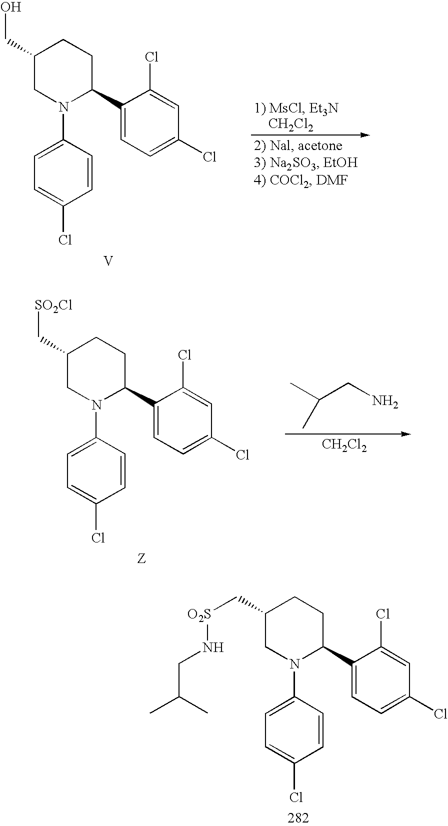 Step 1:

To a solution of V (2.0 g, 5.4 mmol) in CH₂Cl₂ (20 mL) was added Et₃N (820 mg, 8.1 mmol) followed by methanesulfonyl chloride (680 mg, 5.9 mmol). The solution was stirred at RT overnight. Additional methanesulfonyl chloride (90 mg) was added and the solution was heated to reflux for 24 h. The solution was washed with NaHCO₃ (aq.) dried over Na₂SO₄, filtered and concentrated. The crude product was purified by flash chromatography (SiO₂: gradient elution 1:0 to 3:1 hexanes:EtOAc) to afford 2.36 g mesylate.

Step 2:

To a solution of the mesylate from Step 1 (200 mg, 0.45 mmol) in acetone (5 mL) was added sodium iodide (80 mg, 0.53 mmol). The mixture was heated to reflux overnight. To the mixture was added H₂O. The aqueous layer was extracted with CH₂Cl₂. The organic layer was dried over Na₂SO₄, filtered and concentrated. The crude product was purified by preparative TLC (SiO₂: 98:2 hexanes:EtOAc) to afford 175 mg iodide.

Step 3:

To a mixture of the iodide from step 2 (256 mg, 0.54 mmol) in EtOH/H₂O (1:1 4 mL) in a pressure tube was added sodium sulfite (100 mg, 0.79 mmol). The tube was sealed and heated to 100° C. for 4 days. The mixture was concentrated and the crude product was treated with toluene and re-evaporated twice to afford the crude product (ca. 256 mg) that was used without any further purification.

Step 4:

To a mixture of the product from step 3 (256 mg, 0.54 mmol) in CH₂Cl₂ (2 mL) was added a solution of phosgene (1.9 M in toluene) (0.56 mL) followed by DMF (0.05 mL). The mixture was stirred at RT for 1 h. The mixture was concentrated and used without purification.

Step 5:

To the crude product from step 4 (0.27 mmol) in a vial was added excess isobutyl amine. The solution was stirred at RT overnight. Water was added and the mixture was extracted with CH₂Cl₂. The organic layer was dried over Na₂SO₄, filtered and concentrated. The crude product was purified by preparative TLC (77:23 hexanes:EtOAc) to afford Example 282 (46 mg).

### Preparation of Example 283

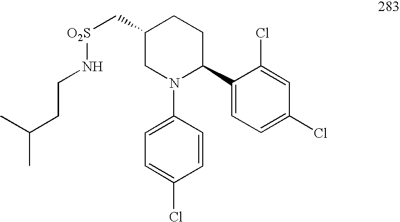

Example 283 was prepared using a procedure similar to that described above for Example 282 step 5, except 3-methyl butyl amine was used instead of isobutylamine.

### Preparation of Example 284

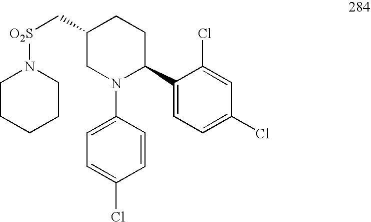

Example 284 was prepared using a procedure similar to that described above for Example 282 step 5, except piperidine was used instead of isobutylamine.

### Preparation of Example 285

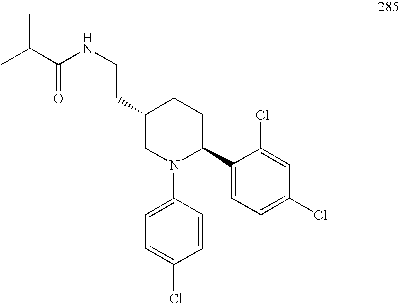

To a solution of Example 264 (40 mg, 0.10 mmol) in MeCN (1.5 mL) was added EDCl (29 mg, 0.15 mmol), HOBt (20 mg, 0.15 mmol), iPr₂NEt (122 mg, 0.96 mmol) and isopropyl carboxylic acid (18 mg, 0.20 mmol). The mixture was stirred at RT overnight. The mixture was concentrated, partitioned between 1 N NaOH (aq.) and EtOAc. The aqueous layer was extracted with EtOAc (3×). The combined organic layers were dried over Na₂SO₄, filtered and concentrated. The crude product was purified by preparative TLC (7:3 hexanes:EtOAc) to afford Example 285 which was converted to the HCl salt (59 mg) via the addition of 2 N HCl in Et₂O to a solution of the free base in CH₂Cl₂ followed by removal of the solvent.

### Preparation of Example 286

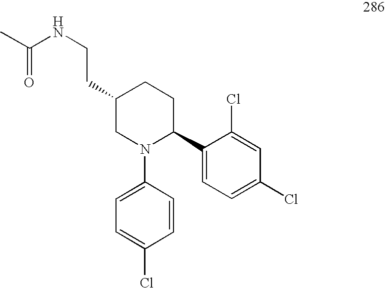

Example 286 was prepared using a procedure similar to that described above for Example 285, except acetic acid was used instead of isopropyl carboxylic acid.

### Preparation of Example 287

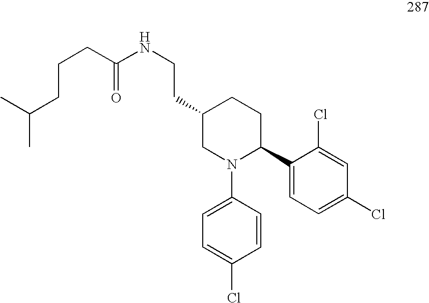

Example 287 was prepared using a procedure similar to that described above for Example 285, except 5-methyl hexanoic acid was used instead of isopropyl carboxylic acid.

### Preparation of Example 288

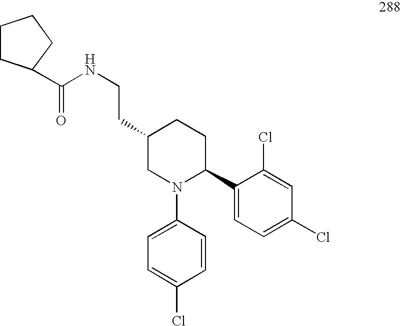

Example 288 was prepared using a procedure similar to that described above for Example 285, except cyclopentyl carboxylic acid was used instead of isopropyl carboxylic acid.

### Preparation of Example 289

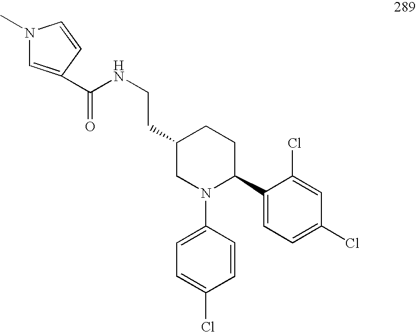

Example 289 was prepared using a procedure similar to that described above for Example 285, except N-Methylpyrrole-3-carboxylic acid was used instead of isopropyl carboxylic acid.

### Preparation of Example 290

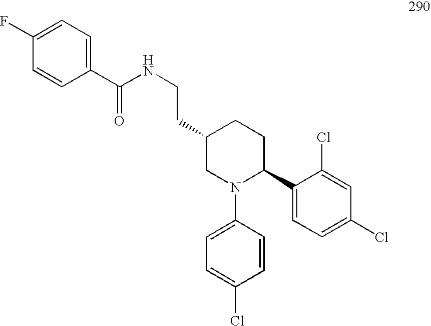

Example 290 was prepared using a procedure similar to that described above for Example 285, except 4-fluorobenzoic acid was used instead of isopropyl carboxylic acid.

### Preparation of Example 291

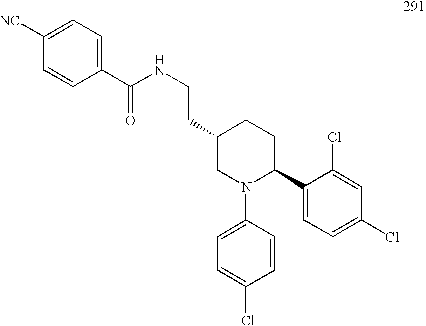

Example 291 was prepared using a procedure similar to that described above for Example 285, except 4-cyanobenzoic acid was used instead of isopropyl carboxylic acid.

### Preparation of Example 292

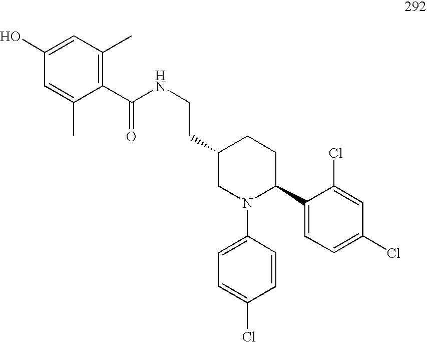

Example 292 was prepared using a procedure similar to that described above for Example 285, except 4-hydroxy-2,6-dimethylbenzoic acid was used instead of isopropyl carboxylic acid.

### Preparation of Example 293

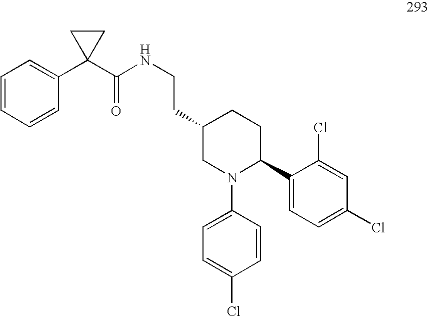

Example 293 was prepared using a procedure similar to that described above for Example 285, except 1-phenyl-cyclopropanecarboxyilc acid was used instead of isopropyl carboxylic acid.

### Preparation of Example 294

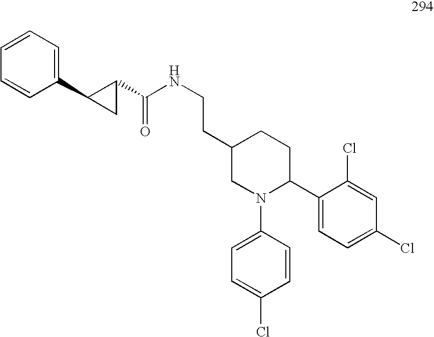

Example 294 was prepared using a procedure similar to that described above for Example 285, except 2-phenyl-cyclopropanecarboxyilc acid was used instead of isopropyl carboxylic acid.

### Preparation of Example 295

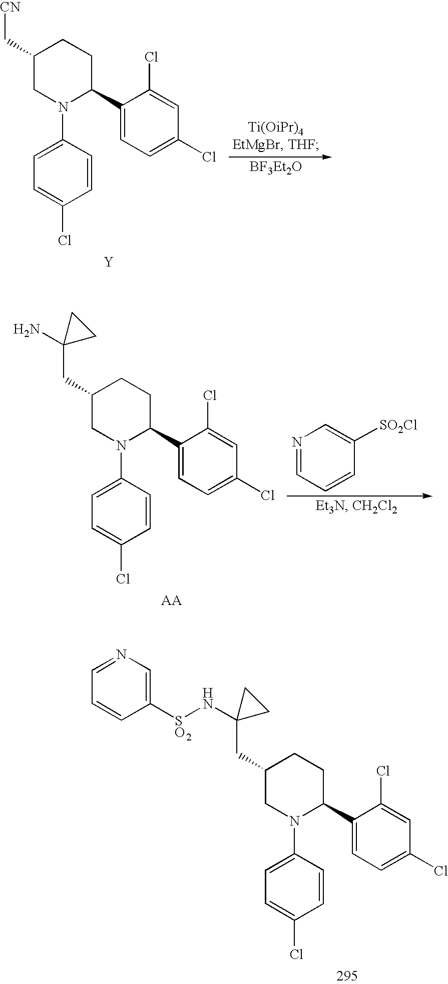 Step 1:

To a solution of Y (200 mg, 0.53 mmol) in anhydrous THF was added titanium(IV)isopropoxide (0.17 mL, 0.58 mmol). To this solution was added a dropwise solution of ethyl magnesium bromide (1 M in Et₂O)(1.1 mL, 1.05 mmol). The solution was stirred at RT for 3 h. To the solution was added borontrifluoide etherate (0.13 mL, 1.05 mmol). The solution was stirred at RT for 1 h. To the solution was added 1 M NaOH (aq.) and the aqueous layer was extracted with EtOAc. The organic layer was dried over Na₂SO₄, filtered and concentrated. The crude product was purified by flash chromatography (SiO₂: gradient elution 100:0 to 75:25 hexanes:EtOAc to elute unreacted Y changing to 95:5:0.5 CH₂Cl₂:MeOH:ammonium hydroxide to elute AA) to afford 100 mg AA.

Step 2:

To a solution of AA (30 mg, 0.07 mmol) in CH₂Cl₂ (2 mL) was added Et₃N (23 mg, 0.32 mmol) followed by 3-pyridine sulfonyl chloride (21 mg, 0.12 mmol). The solution was stirred at RT overnight followed by 4 hours at reflux. The crude material was purified by preparative TLC (SiO₂: 65:35 hexanes:EtOAc) to afford Example 295, which was converted to the HCl salt using a procedure similar to that described above for Example 285 (20 mg).

### Preparation of Example 296

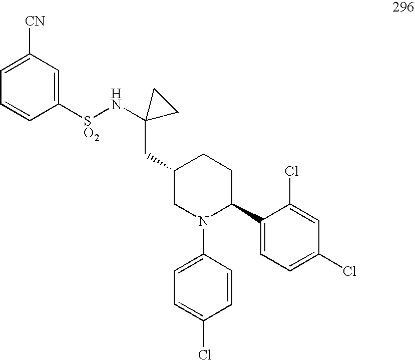

Example 296 was prepared using a procedure similar to that described above for Example 295, step 2, except 3-cyano-benzenesulfonyl chloride was used instead of 3-pyridine sulfonyl chloride.

### Preparation of Example 297

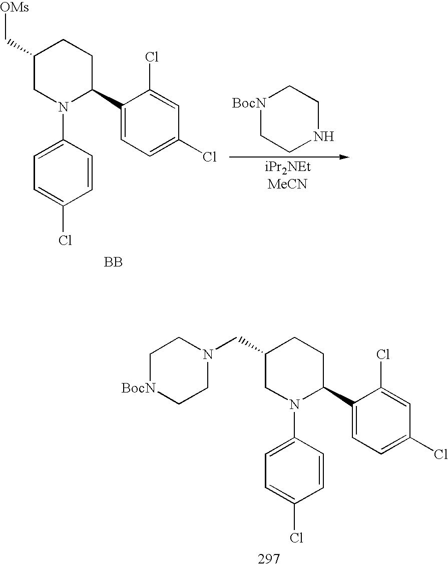

To a solution of BB (See Step 1, Example 282) (1.0 g, 2.2 mmol) in MeCN (15 mL) was added N-Boc piperazine (466 mg, 2.5 mmol) and iPr₂NEt (341 mg, 2.64 mmol). The solution was heated to reflux for 24 h. The solution was concentrated and the crude product was partitioned between CH₂Cl₂ and NaHCO₃ (aq.). The aqueous layer was extracted with CH₂Cl₂ (3×). The combined organic layers were dried over Na₂SO₄, filtered and concentrated. The crude product was purified by flash chromatography (SiO₂: gradient elution 100:0 to 65:35 hexanes:EtOAc) to afford 475 mg Example 297.

### Preparation of Example 298

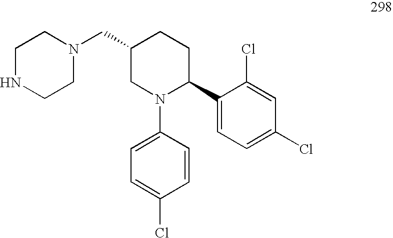

To a solution of Example 297 (475 mg) in MeOH (20 mL) was added 4 N HCl (in dioxane) (5 mL). The solution was stirred at RT for 2 h. The solution was concentrated and the crude material was partitioned between CH₂Cl₂ and NaHCO₃ (aq.). The aqueous layer was extracted with CH₂Cl₂ (3×). The combined organic layers were dried over Na₂SO₄, filtered and concentrated. The crude product was purified by flash chromatography [SiO₂: gradient elution 100:0:0 to 92:8:1 CH₂Cl₂:MeOH: 7N NH₃ (in MeOH)] to afford 320 mg Example 298.

### Preparation of Example 299

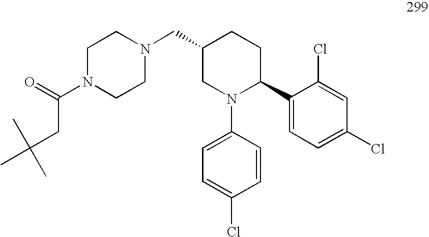

To a solution of Example 298 (41 mg, 0.093 mmol) in MeCN (1 mL) was added EDCl (17 mg, 0.112 mmol), HOBt (15 mg, 0.112 mmol) (13 mg, 0.112 mmol) 3.3 dimethyl butyric Acid and iPr₂NEt (14 mg, 0.112 mmol). The solution was stirred at RT overnight. The solution was concentrated and the crude product was partitioned between 1 M NaOH (aq.) and EtOAc. The aqueous layer was extracted with EtOAc (3×). The combined organic layers were washed with brine, dried over Na₂SO₄, filtered and concentrated. The crude product was purified by preparative TLC (SiO₂: 3:1 hexanes:EtOAc) to afford 42 mg Example 299.

### Preparation of Example 300

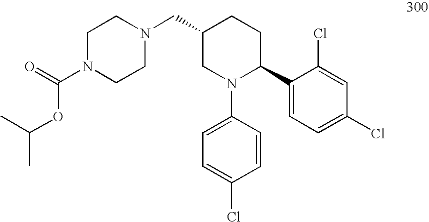

To a solution of Example 298 (29 mg, 0.066 mmol) in CH₂Cl₂ (2 mL) was added isopropyl chloroformate (1 M solution in toluene; 80 uL, 0.080 mmol) and Et₃N (8.7 mg, 0.080 mmol). The solution was stirred at RT overnight. The solution was diluted with CH₂Cl₂. The organic layer was washed with NaHCO₃ (aq.). The aqueous layer was back extracted with CH₂Cl₂ (2×). The combined organic layers were dried over Na₂SO₄, filtered and concentrated. The crude product was purified by preparative TLC (SiO₂: 3:1 hexanes:EtOAc) to afford 30 mg Example 300.

### Preparation of Example 301

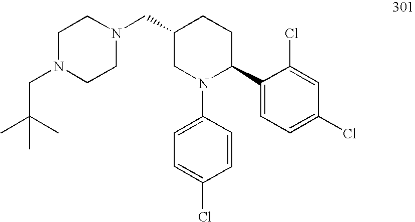

To a solution of Example 298 (25 mg, 0.057 mmol) in 1,2-dichloroethane (1 mL) was added 3,3-dimethyl butrylaldehyde (7 mg, 0.068 mmol) followed by NaBH(OAc)₃ (14 mg, 0.068 mmol). The solution was stirred at RT overnight. The solution was diluted with CH₂Cl₂. The organic layer was washed with 1 M NaOH (aq.). The aqueous layer was back extracted with CH₂Cl₂ (2×). The combined organic layers were dried over Na₂SO₄, filtered and concentrated. The crude product was purified by preparative TLC (SiO₂: 1:2 hexanes:EtOAc) to afford Example 301.

### Preparation of Example 302

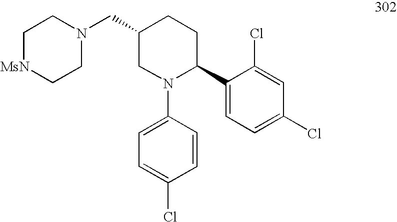

To a solution of Example 298 (29 mg, 0.066 mmol) in CH₂Cl₂ (2 mL) was added methanesulfonyl chloride (9 mg, 0.079 mmol) followed by Et₃N (10 mg, 0.099 mmol). The solution was stirred at RT for 2.5 days. The solution was diluted with CH₂Cl₂. The organic layer was washed with 1 NaHCO₃ (aq.). The aqueous layer was back extracted with CH₂Cl₂ (2×). The combined organic layers were dried over Na₂SO₄, filtered and concentrated. The crude product was purified by preparative TLC (SiO₂: 2:1 hexanes:EtOAc) to afford 20 mg Example 302.

### Preparation of Example 303

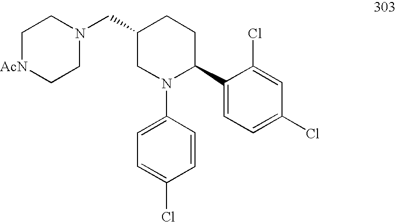

To a solution of Example 298 (21 mg, 0.048 mmol) in CH₂Cl₂ (2 mL) was added acetic anhydride (6 mg, 0.058 mmol) followed by Et₃N (7 mg, 0.072 mmol). The solution was stirred at RT for 2.5 days. The solution was diluted with CH₂Cl₂. The organic layer was washed with 1 NaHCO₃ (aq.). The aqueous layer was back extracted with CH₂Cl₂ (2×). The combined organic layers were dried over Na₂SO₄, filtered and concentrated. The crude product was purified by preparative TLC (SiO₂: 1:1 hexanes:EtOAc) to afford 18 mg Example 303.

### Preparation of Example 304

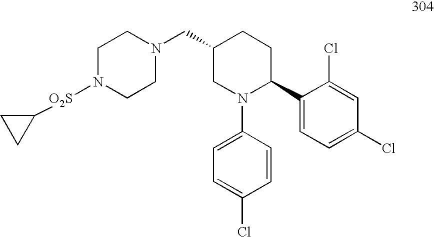

Example 304 was prepared using a procedure similar to that described above for Example 302, except cyclopropanesulfonyl chloride was used instead of methanesulfonyl chloride.

### Preparation of Examples 305-352

Examples 305-352 were prepared using a procedure similar to that described above for preparing Examples 149-162, except that Example 298 was used as the starting material instead of Examples 130 or 131.

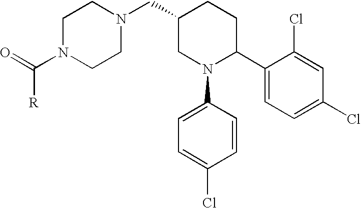

| | | Carboxylic |
|---|---|---|
| Ex. | R | Acid |
| 305 | 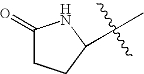 | 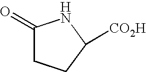 |
| 306 | 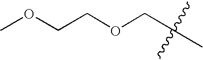 | 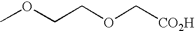 |
| 307 | 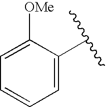 | 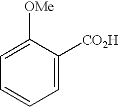 |
| 308 | 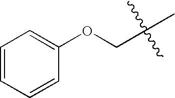 | 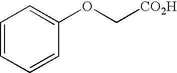 |
| 309 | 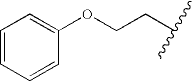 | 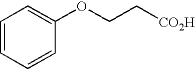 |
| 310 | 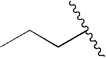 | 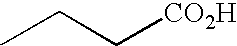 |
| 311 | 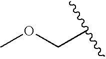 | 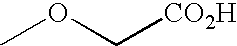 |
| 312 | 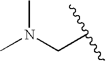 | 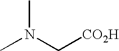 |
| 313 | 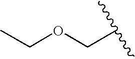 | 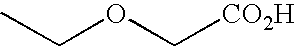 |
| 314 | 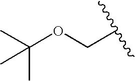 | 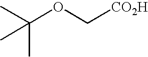 |
| 315 | 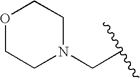 | 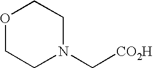 |
| 316 | 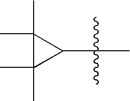 | 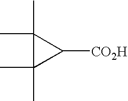 |
| 317 | 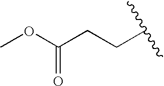 | 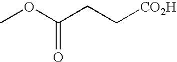 |
| 318 | 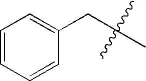 | 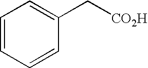 |
| 319 | 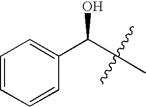 | 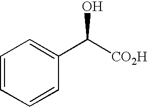 |
| 320 | 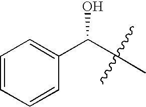 | 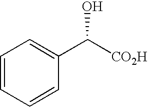 |
| 321 | 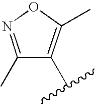 | 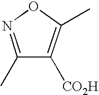 |
| 322 | 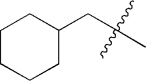 | 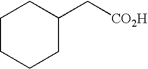 |
| 323 | 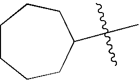 | 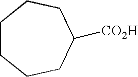 |
| 324 | 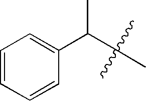 | 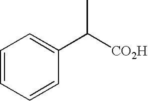 |
| 325 | 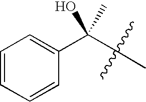 | 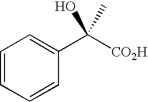 |
| 326 | 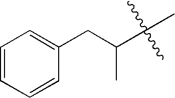 | 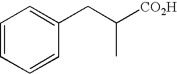 |
| 327 | 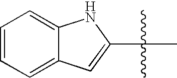 | 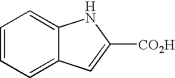 |
| 328 | 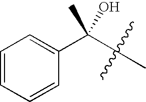 | 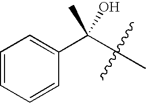 |
| 329 | 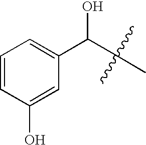 | 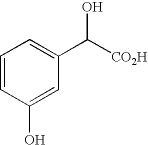 |
| 330 | 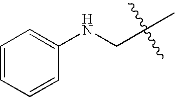 | 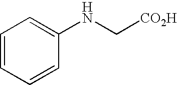 |
| 331 | 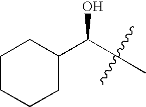 | 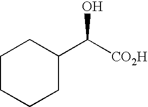 |
| 332 | 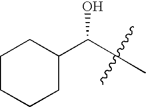 | 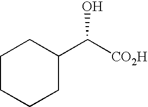 |
| 333 | 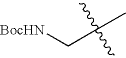 | 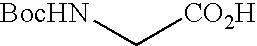 |
| 334 | 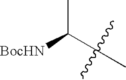 | 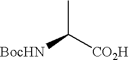 |
| 335 | 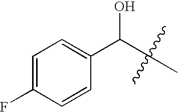 | 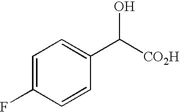 |
| 336 | 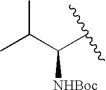 | 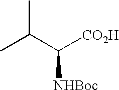 |
| 337 | 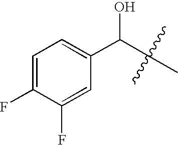 | 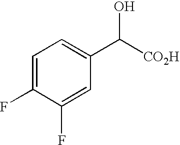 |
| 338 | 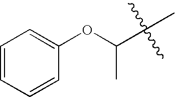 | 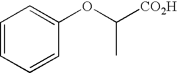 |
| 339 | 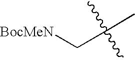 | 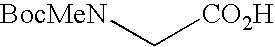 |
| 340 | 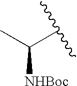 | 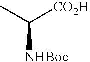 |
| 341 | 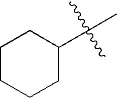 | 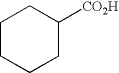 |
| 342 | 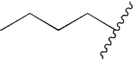 | 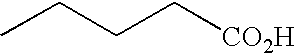 |
| 343 | 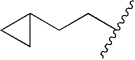 | 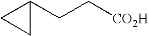 |
| 344 | 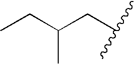 | 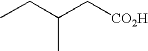 |
| 345 | 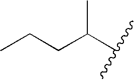 | 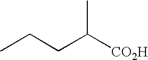 |
| 346 | 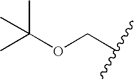 | 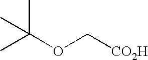 |
| 347 | 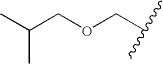 | 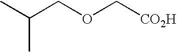 |
| 219 | 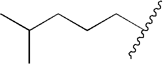 | 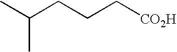 |
| 348 | 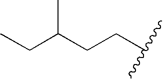 | 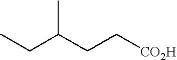 |
| 349 | 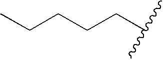 | 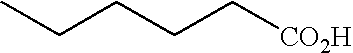 |
| 350 | 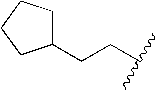 | 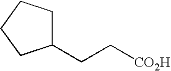 |
| 351 | 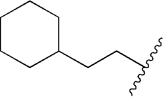 | 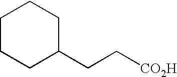 |
| 352 | 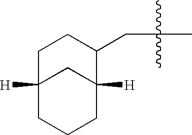 | 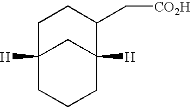 |

### Preparation of Example 353

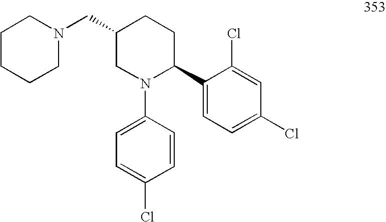

To a solution of BB (Step 1 Example 297) (35 mg, 0.078 mmol) in MeCN (15 mL) in a pressure tube was added piperidine (8 mg, 0.094 mmol) and iPr₂NEt (12 mg, 0.094 mmol). The tube was sealed and the solution was heated to 90° C. for 16 h. The solution was concentrated. The crude product was partitioned between CH₂Cl₂ and NaHCO₃ (aq.). The aqueous layer was extracted with CH₂Cl₂ (3×). The combined organic layers were dried over Na₂SO₄, filtered and concentrated. The crude product was purified by preparative TLC [SiO₂: 95:5:0.1 CH₂Cl₂:MeOH:7 N NH₃ (in MeOH)] to afford Example 353.

### Preparation of Example 354

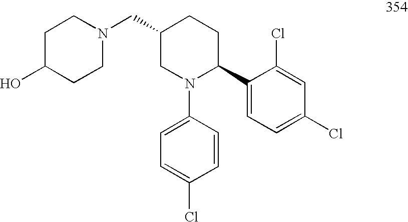

Example 354 was prepared using a procedure similar to that described above for Example 353, except 4-hydroxypiperidine was used instead of piperidine.

### Preparation of Example 355

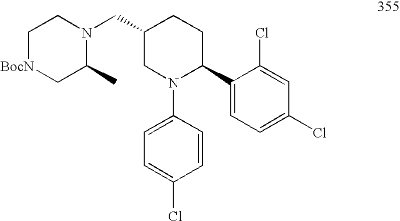

Example 355 was prepared using a procedure similar to that described above for Example 297, except 3-(S)-methyl-1 N-Boc-piperazine (WO2003084942) was used instead of N-Boc-piperazine.

### Preparation of Example 356

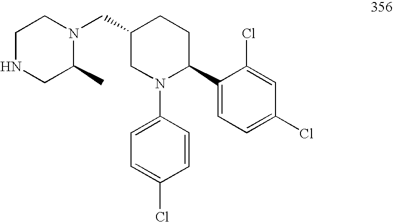

Example 356 was prepared using a procedure similar to that described above for Example 298, except Example 355 was used instead of Example 297.

### Preparation of Example 357

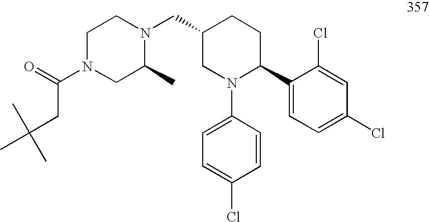

Example 357 was prepared using a procedure similar to that described above for Example 299, except Example 356 was used instead of Example 298.

### Preparation of Example 358

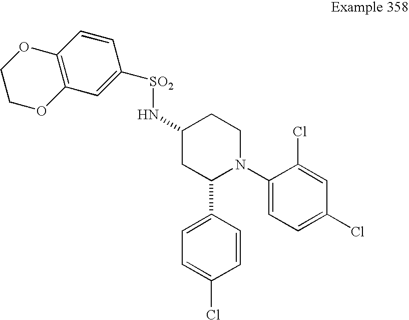

To a solution of Example 131 (10 mg, 0.028 mmol) in 1,2 dichloroethane (0.1 mL) was added iPr₂NEt (35 μL) followed by 2,3-dihydro-1,4-benzodioxane-8-sulfonyl chloride (Maybridge) (22 mg). The solution was stirred at RT overnight. The solution was concentrated and the crude product was purified by preparative TLC (SiO₂: 99:1 CH₂Cl₂:MeOH) to afford Example 358.

### Preparation of Example 359

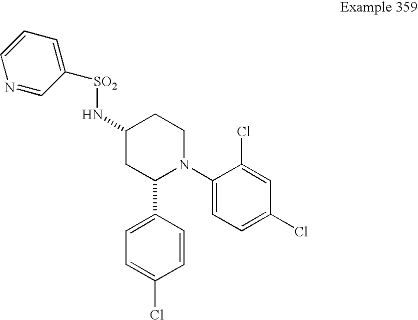

Example 359 was prepared using a procedure similar to that described above for Example 358, except 3-pyridyl sulfonyl chloride was used instead of 2,3-dihydro-1,4-benzodioxane-8-sulfonyl chloride.

### Preparation of Example 360

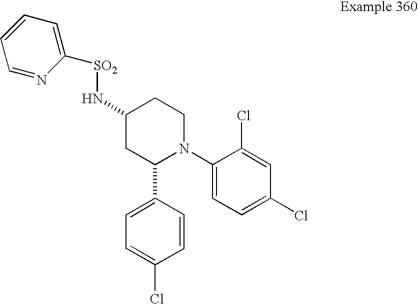

Example 360 was prepared using a procedure similar to that described above for Example 358, except 2-pyridyl sulfonyl chloride was used instead of 2,3-dihydro-1,4-benzodioxane-8-sulfonyl chloride.

### Preparation of Example 361

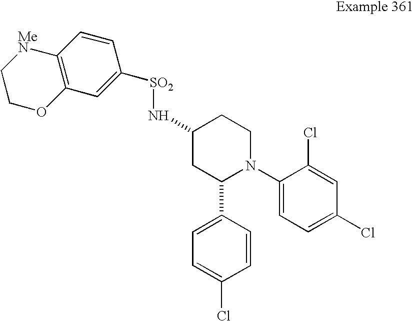

Example 361 was prepared using a procedure similar to that described above for Example 358, except 4-methyl-3,4-dihydro-2H-benzo[1,4]oxazine-7-sulfonyl chloride (Maybridge) was used instead of 2,3-dihydro-1,4-benzodioxane-8-sulfonyl chloride.

### Preparation of Example 362

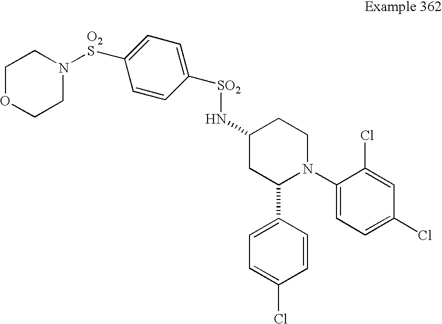

Example 362 was prepared using a procedure similar to that described above for Example 358, except 4-(morpholine-4-sulfonyl)-benzenesulfonyl chloride (Maybridge) was used instead of 2,3-dihydro-1,4-benzodioxane-8-sulfonyl chloride.

### Preparation of Example 363

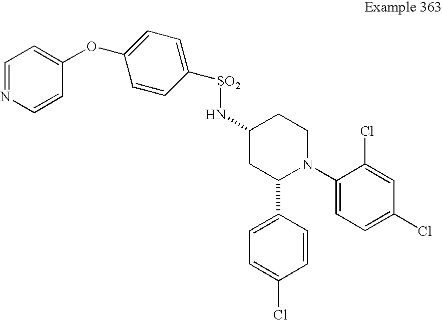

Example 363 was prepared using a procedure similar to that described above for Example 358, except 4-(pyridine-4-yloxy)-benzenesulfonyl chloride (Array Biopharma) was used instead of 2,3-dihydro-1,4-benzodioxane-8-sulfonyl chloride.

### Preparation of Example 364

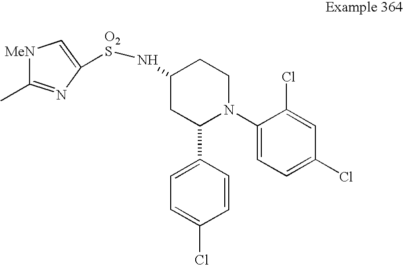

Example 364 was prepared using a procedure similar to that described above for Example 358, except 1,2-Dimethyl-1H-imidazole-4-sulfonyl chloride (Maybridge) was used instead of 2,3-dihydro-1,4-benzodioxane-8-sulfonyl chloride.

### Preparation of Example 365

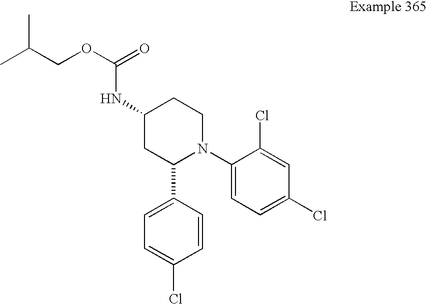

To a solution of Example 131 (5 mg, 0.014 mmol) in 1,2 dichloroethane (0.1 mL) at 4° C. was added Et₃N (5.7 mg, 0.056 mmol) followed by isobutyl chloroformate (3.8 mg, 0.028 mmol). The solution was stirred and allowed to slowly warm to RT overnight. The solution was diluted with CH₂Cl₂ and washed with NaHCO₃ (aq.). The organic layer was dried over Na₂SO₄, filtered and concentrated. The crude product was purified by preparative TLC (SiO₂: 1:1 Et₂O:hexanes) to afford 3.8 mg Example 365.

### Preparation of Example 366

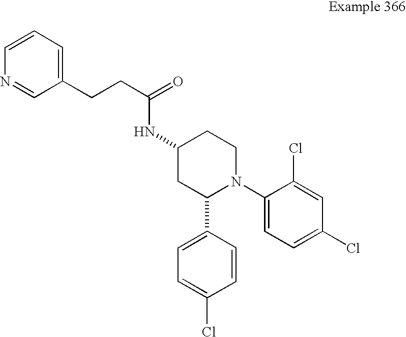

To a solution of Example 131 (5 mg, 0.014 mmol) in DMF (0.075 mL) was added N-methylmorpholine (3.6 mg, 0.035 mmol), HOBt (2.9 mg, 0.021 mmol), 3(3-pyridyl)propionic acid (4.3 mg, 0.028 mmol) followed by dicyclohexylcarbodiimide (8.0 mg, 0.042 mmol). The reaction mixture was stirred at RT overnight. The solution was concentrated and placed under vacuum for 3 days. The crude material was dissolved in CH₂Cl₂ and washed with NaHCO₃ (aq.) (2×). The organic layer was dried over Na₂SO₄, filtered and concentrated. The crude product was purified by preparative TLC (SiO₂: 80:1 CH₂Cl₂:MeOH) to afford 5.5 mg Example 366.

### Preparation of Example 367

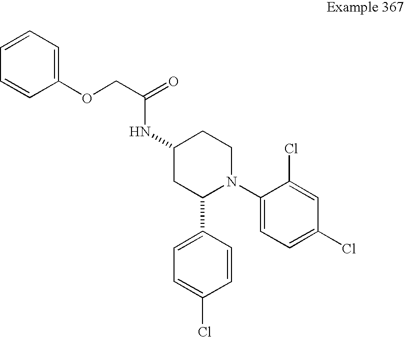

Example 367 was prepared using a procedure similar to that described above for Example 366, except phenoxyacetic acid was used instead of 3(3-pyridyl)propionic acid.

### Preparation of Example 368

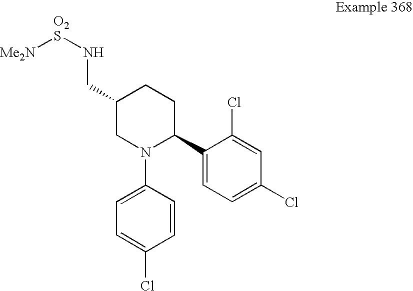

To a solution of Example 85 (26 mg, 0.070 mmol) in CH₂Cl₂ (1 mL) was added iPr₂NEt (11 mg, 0.084 mmol) and N,N-dimethylamino-sulfonyl chloride (12 mg, 0.084 mmol). The solution was stirred at RT for 3 days. The solution was diluted with NaHCO₃ (aq.). The aqueous layer was back extracted with CH₂Cl₂ (3×). The combined organic layers were dried over Na₂SO₄, filtered and concentrated. The crude product was purified by preparative TLC (SiO₂: 2:1 hexanes:EtOAc) to afford 20 mg Example 368.

### Preparation of Example 369

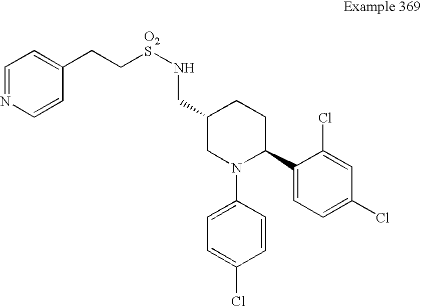

Example 369 was prepared using a procedure similar to that described above for Example 253, except 4-pyridylethanesulfonyl chloride hydrochloride (Chemical Synthesis Services: Graigavon, Northern Ireland) was used instead of 4-methyl-3,4-dihydro-2H-1,4-benzoxazine-7-sulfonyl chloride.

### Preparation of Example 370

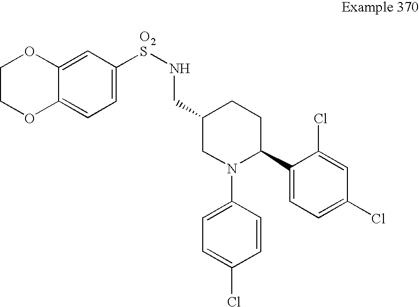

Example 370 was prepared using a procedure similar to that described above for Example 253, except 2,3-Dihydro-benzo[1,4]dioxine-6-sulfonyl chloride was used instead of 4-methyl-3,4-dihydro-2H-1,4-benzoxazine-7-sulfonyl chloride.

### Preparation of Example 371

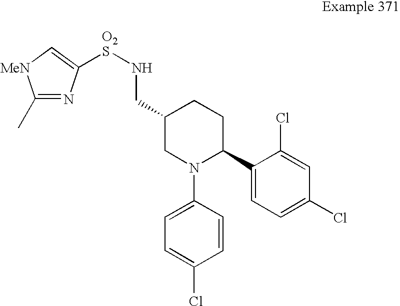

Example 371 was prepared using a procedure similar to that described above for Example 253, except 1,2-Dimethyl-1H-imidazole-4-sulfonyl chloride was used instead of 4-methyl-3,4-dihydro-2H-1,4-benzoxazine-7-sulfonyl chloride.

### Preparation of Example 372

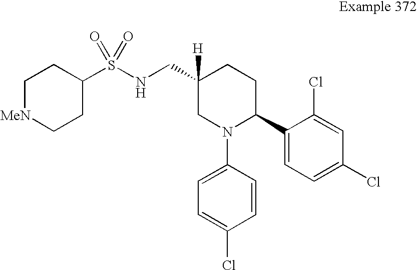

To a solution of Example 256 (50 mg, 0.10 mmol) in formic acid was added formalin (150 μL). The solution was heated to 98° C. for 2 h. The solution was basified with sat Na₂CO₃ (aq.). Water was added and the aqueous layer was extracted with EtOAc (3×). The combined organic layers were dried over Na₂SO₄, filtered and concentrated. The crude product was purified by flash chromatography (SiO₂: 95:7:0.5 CH₂Cl₂:MeOH:ammonium hydroxide) to afford Example 372.

Assay

Method for Evaluating Cannabinoid CB₁ and CB₂ Affinity

Competition binding assays for cannabinoid CB₁ and CB₂ affinity were performed by incubating commercially purchased membranes prepared from cells expressing each receptor subtype (8 μg pro) with 0.5 nM ³H-CP55,940, a non-selective cannabinoid agonist, along with concentrations of drug ranging from 0.0001-3 μM in Buffer A (5 mM MgCl₂, 2.5 mM EDTA and 013% BSA). Non-specific binding was defined in the presence of 10 μM CP55,940. For saturation studies, concentrations of ³H-CP55,940 ranging from 0.1-5 nM were incubated with membranes in the presence and absence of 10 μM CP55,940. Assays were terminated after incubation for 1½ hours by rapid filtration onto 0.3% polyethylenamine treated GF/C filterplates using a BRANDEL cell harvester. The plates were dried and MICROSCINT scintillation cocktail was added, after which the bound radioactivity was quantified using a TOPCOUNT scintillation counter.

The dissociation constant (K_{d}) of ³H-CP55,940 at the CB₁ and CB₂ receptor were determined by plotting specific binding at each concentration of radioligand, and analysis by non-linear regression. For competition studies, the concentration of each drug that inhibited 50 percent of ³H-CP55,940 binding (IC₅₀) was determined by non-linear regression analysis of the radioligand displacement curves. Affinity constants (Kᵢ) were calculated using the equation derived by Cheng and Prusoff (1973), defined as: IC₅₀/1+[conc. ligand/K_{d}].

GTPγS Binding Protocol

The functional efficacy of compounds to activate second messengers within the cell was determined utilizing the GTPγS binding assay. Guanine nucleotides are phosphorylated within the plasma membrane of the cell following binding and activation by agonists. A radiolabelled derivative of guanine triphosphate (GTP) is utilized in this assay as it cannot be dephosphorylated and therefore accumulates following agonist binding. The simultaneous presence of an antagonist into this system will shift the agonist concentration curve to the right, with increasing concentrations of antagonist producing a greater rightward shift in the dose-response curve of the agonist.

Commercially purchased membranes were incubated with 10 mM GDP to allow sufficient substrate for phosphorylation in the presence of agonist. The membranes were then pre-incubated with increasing concentrations of test compound for 30 minutes to determine if they were capable of stimulating phosphorylation alone. Increasing concentrations of the non-selective cannabinoid agonist WIN55,122 were then added in the presence or absence of each concentration of test compound. The assay was then incubated for 1 hour at room temperature. To complete the assay, ³⁵S-GTPγS was added and the assay incubated for another 30 minutes. Assays were terminated by rapid filtration onto 10 mM sodium phosphate-treated GF/C filterplates using a BRANDEL cell harvester. The plates were dried and Microscint scintillation cocktail was added, after which the bound radioactivity was quantified using a TOPCOUNT scintillation counter. The stimulation of ³⁵S-GTPγS binding as a function of the concentration of the agonist WIN55,122, in the absence and presence of test compound, was plotted and the EC₅₀ determined by nonlinear regression analysis using GraphPad Prism software. A Schild analysis of the rightward shift in the dose response curve of WIN55,122 in the presence of test compound was determined by plotting the concentration of test compound against the negative log of the dose ratio [1−(EC₅₀ agonist+test compound/EC50 of agonist alone)]. A linear regression analysis yields the Kb, defined as the X-intercept of the linear equation.

In one embodiment, the compounds of Formula (I) of the present invention, and salts, solvates, or esters thereof, have Kᵢ values of about 800 nM or less. In another embodiment, the compounds of Formula (I) of the present invention, and salts, solvates, or esters thereof, have Kᵢ values of about 100 nM or less. In another embodiment, the compounds of Formula (I) of the present invention, and salts, solvates, or esters thereof, have Kᵢ values of about 50 nM or less. In another embodiment, the compounds of Formula (I) of the present invention, and salts, solvates, or esters thereof, have Kᵢ values of about 20 nM or less. In another embodiment, the compounds of Formula (I) of the present invention, and salts, solvates, or esters thereof, have Kᵢ values of 10 nM or less. Examples 9, 14, 18, 29, 31, 33, 51, 52, 86, 90-92, 95, 97-99, 101, 107-109, 111, 112, 114, 116, 117, 119-121, 123, 131-137, 140, 147, 149, 162 have Kᵢ values of 10 nm or less. Examples 86, 91, 92, 112, and 120, respectively, have Kᵢ values of approximately 9, 4, 7, 2, and 2 nM.

We claim:

1. A compound of Formula (I): 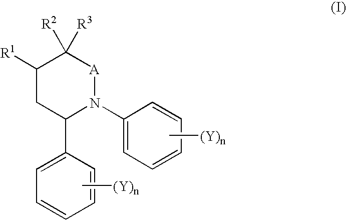
or a pharmaceutically acceptable salt, solvate, or ester thereof, wherein:
A is —CH₂— or —C(O)—;
R¹ is selected from the group consisting of H, —N(R⁴)(R⁵), unsubstituted heterocyclyl, heterocyclyl substituted with one or more X groups, —N₃, and —O—R⁷;
R² is selected from the group consisting of H, —(C(R⁶)₂)ₚ-aryl, cycloalkylalkyl, cycloalkylalkyl substituted with Z, —(C(R⁶)₂)_{q}-heterocyclyl, —(C(R⁶)₂)ₚ—S(O)₂-heterocyclyl, and —C(R⁶)₂—O—R⁷,
wherein the aryl portion of said —C(R⁶)₂-aryl of R² is unsubstituted or substituted with one or more Y groups,
wherein the heterocyclyl portion of said —(C(R⁶)₂)ₚ—S(O)₂-heterocyclyl of R² is unsubstituted or substituted with one or more X groups,
wherein the heterocyclyl portion of said —(C(R⁶)₂)_{q}-heterocyclyl of R² is unsubstituted or substituted with one or more X groups;
R³ is selected from the group consisting of H, —C(R⁶)₂-aryl, —C(R⁶)₂—O—R⁷, —(C(R⁶)₂)_{q}—C(O)—N(R²)₂, —(C(R⁶)₂)ₚ—N(R⁹)—C(O)—(C(R⁶)₂)_{q}—R¹⁶, —(C(R⁶)₂)_{q}—S(O)₂—N(R⁹)—(C(R⁶)₂)_{q}—R¹⁵, —(C(R⁶)₂)_{q}—N(R⁹)—S(O)₂—(C(R⁶)₂)_{q}—R¹⁵ and —(C(R⁶)₂)_{q}—N(R⁸)₂,
wherein the aryl portion of said —C(R⁶)₂-aryl of R³ is unsubstituted or substituted with one or more Y groups;
with the following independent provisos:
(i) at least one of R¹, R², and R³ is not H;
(ii) when R¹ is —OH, at least one of R² and R³ is not H; and
(iii) when A is —C(O)—, at least one of R² and R³ is not H;
or, R² and R³ together with the ring carbon atom to which they are shown attached form an unsubstituted heterocyclyl ring or a heterocyclyl ring substituted with one or more X groups;
R⁴ is selected from the group consisting of H, —C(O)alkyl, and alkyl;
R⁵ is selected from the group consisting of —C(R⁶)₂)ₘ-G, —S(O)₂-alkyl, —S(O)₂-cycloalkyl, alkyl, —S(O)-cycloalkyl, —C(O)cycloalkyl, —S(O)₂-aryl, —S(O)₂—(C(R⁶)₂)ₘ-aryl, —S(O)₂-heteroaryl, —C(O)-alkyl, —C(O)-aryl, —C(O)—O-alkyl, —C(O)O-aryl, —C(O)—(C(R⁶)₂)ₘ-aryl, —C(O)-cycloalkylene-aryl, —C(O)-heteroaryl, —C(O)heteroarylalkyl, —C(O)—(C(R⁶)₂)ₘ—O-aryl, —C(O)-(benzo-fused cycloalkyl), —S(O)₂-(benzo-fused heterocyclyl), —C(O)—N(R⁹)—(C(R⁶)₂)ₘ-aryl, —C(O)—N(R⁹)-aryl, cycloalkyl, benzo-fused cycloalkyl, unsubstituted aryl, aryl substituted with one or more Y groups, unsubstituted heterocyclyl, and heterocyclyl substituted with one or more X groups,
wherein the aryl or heteroaryl portion of said —S(O)₂-aryl, —S(O)₂—(C(R⁶)₂)ₘ-aryl, —S(O)₂— heteroaryl, —C(O)-aryl, —C(O)—(C(R⁶)₂)ₘ-aryl, —C(O)-cycloalkylene-aryl, —C(O)-heteroaryl, —C(O)—(C(R⁶)₂)ₘ—O-aryl, —C(O)—N(R⁹)—(C(R⁶)₂)ₘ-aryl, or —C(O)—N(R⁹)-aryl of R⁵ is unsubstituted or substituted with one or more Y groups;
wherein the heterocyclyl portion of —S(O)₂-(benzo-fused heterocyclyl)aryl of R⁵ is unsubstituted or substituted with one or more X groups;
each R⁶ is independently selected from the group consisting of H and alkyl;
R⁷ is selected from the group consisting of H, alkyl, unsubstituted heteroaryl and heteroaryl substituted with one or more Y groups, unsubstituted aryl, and aryl substituted with one or more Y groups;
each R⁸ is independently selected from the group consisting of H, alkyl, arylalkyl, heteroarylalkyl, unsubstituted aryl, unsubstituted heteroaryl, —C(O)-alkyl, —C(O)-aryl, —C(O)-cycloalkyl, —C(O)N(R⁹)₂, —S(O)₂-aryl, —S(O)₂-heteroaryl, —SO₂N(R⁹)₂, —S(O)₂-cycloalkyl, aryl and heteroaryl substituted with one or more Y groups, and —S(O)₂-alkyl,
wherein the aryl portion of said arylalkyl, —C(O)-aryl or —S(O)₂-aryl and the heteroaryl portion of said heteroarylalkyl, —S(O)₂-heteroaryl of R⁸ is unsubstituted or substituted with one or more Y groups,
wherein the alkyl portion of said arylalkyl and heteroarylalkyl is unsubstituted or substituted with one or more X groups with the proviso that X substituted on said alkyl portion is NOT Cbz or Boc;
each R⁹ is independently selected from the group consisting of H, alkyl, haloalkyl, hydroxyalkyl, cycloalkyl, unsubstituted aryl and unsubstituted heteroaryl;
each R¹² is independently selected from the group consisting of H, alkyl, cycloalkylalkyl, —(C(R⁶)₂)_{q}—C(O)R¹³, benzoheterocyclyl, benzocycloalkyl, —(C(R⁶)₂)_{q}—N(R⁹)—C(O)R¹³, —(C(R⁶)₂)_{q}—N(R¹⁴)₂, arylalkyl, heteroarylalkyl, HO-alkyl-, alkyl-O—, aryl-O—, Y-alkylenyl-O—, W—O-alkylenyl, heterocyclylalkyl, unsubstituted cycloalkyl, cycloalkyl substituted with one or more X groups, unsubstituted heterocyclyl, heterocyclyl substituted with one or more X groups, unsubstituted heteroaryl, heteroaryl substituted with one or more Y groups, unsubstituted aryl and aryl substituted with one or more Y groups, and
wherein the aryl and heteroaryl portion of said arylalkyl and heteroarylalkyl is unsubstituted or substituted with one or more Y groups,
wherein the alkyl portion of said cycloalkylalkyl, arylalkyl and heteroarylalkyl is unsubstituted or substituted with one or more X groups with the proviso that X substituted on said alkyl portion is NOT Cbz or Boc,
wherein the cycloalkyl of said cycloalkylalkyl is unsubstituted or substituted with one or more X groups,
wherein the benzo portion of said benzoheterocyclyl can be optionally substituted with one or more Y groups and the heterocyclyl portion of benzoheterocyclyl can be optionally substituted with one or more X groups,
wherein the benzo portion of said benzocycloalkyl can be optionally substituted with one or more Y groups and the cycloalkyl portion of benzocycloalkyl can be optionally substituted with one or more X groups;
with the following provisos that
for —N(R¹⁴)₂ of R¹², the two R¹⁴ groups, with the ring nitrogen atom to which they are shown attached, form an unsubstituted heterocyclyl ring or a heterocyclyl ring substituted with one or more X groups;
each R¹³ is independently selected from the group consisting of H, alkyl, cycloalkylalkyl, arylalkyl, heteroarylalkyl, HO-alkyl-, alkyl-O—, aryl-O—, unsubstituted cycloalkyl, cycloalkyl substituted with one or more X groups, unsubstituted heterocyclyl, heterocyclyl substituted with one or more X groups, unsubstituted heteroaryl, heteroaryl substituted with one or more Y groups, unsubstituted aryl and aryl substituted with one or more Y groups
wherein the aryl and heteroaryl portion of said arylalkyl and heteroarylalkyl is unsubstituted or substituted with one or more Y groups,
wherein the alkyl portion of said cycloalkylalkyl, arylalkyl and heteroarylalkyl is unsubstituted or substituted with one or more X groups with the proviso that X substituted on said alkyl portion is NOT Cbz or Boc,
wherein the cycloalkyl of said cycloalkylalkyl is unsubstituted or substituted with one or more X groups;
each R¹⁴ is independently selected from the group consisting of H, Boc, unsubstituted alkyl, alkyl substituted with one or more X groups, unsubstituted cycloalkyl, cycloalkyl substituted with one or more Y groups, unsubstituted aryl, aryl substituted with one or more Y groups, heterocyclyl, unsubstituted heteroaryl and heteroaryl substituted with one or more Y groups;
each R¹⁵ is independently selected from the group consisting of H, alkyl, —N(R⁴)(R⁵), (C(R⁶)₂)_{q}—N(R¹⁴)₂, alkylenyl-CF₃, —CF₃, cycloalkylalkyl, unsubstituted cycloalkyl, cycloalkyl substituted with one or more X groups, unsubstituted heterocyclyl, heterocyclyl substituted with one or more X groups, benzoheterocyclyl, benzocycloalkyl, unsubstituted heteroaryl, heteroaryl substituted with one or more Y groups, unsubstituted aryl and aryl substituted with one or more Y groups,
wherein the alkyl portion of said cycloalkylalkyl is unsubstituted or substituted with one or more X groups with the proviso that X substituted on said alkyl portion is NOT Cbz or Boc,
wherein the cycloalkyl of said cycloalkylalkyl is unsubstituted or substituted with one or more X groups,
wherein the benzo portion of said benzoheterocyclyl can be optionally substituted with one or more Y groups and the heterocyclyl portion of benzoheterocyclyl can be optionally substituted with one or more X groups,
wherein the benzo portion of said benzocycloalkyl can be optionally substituted with one or more Y groups and the cycloalkyl portion of benzocycloalkyl can be optionally substituted with one or more X groups;
each R¹⁶ is independently selected from the group consisting of H, alkyl, cycloalkylalkyl, —(C(R⁶)₂)ₚ—C(O)R³, —(C(R⁶)₂)ₚ—N(R⁹)—C(O)R¹³, —(C(R⁶)₂), —N(R¹⁴)₂, arylalkyl, heteroarylalkyl, HO-alkyl-, alkyl-O—, aryl-O—, unsubstituted cycloalkyl, cycloalkyl substituted with one or more X groups, unsubstituted heterocyclyl, heterocyclyl substituted with one or more X groups, unsubstituted heteroaryl, heteroaryl substituted with one or more Y groups, unsubstituted aryl and aryl substituted with one or more Y groups, and
wherein the aryl and heteroaryl portion of said arylalkyl and heteroarylalkyl is unsubstituted or substituted with one or more Y groups,
wherein the alkyl portion of said cycloalkylalkyl, arylalkyl and heteroarylalkyl is unsubstituted or substituted with one or more X groups with the proviso that X substituted on said alkyl portion is NOT Cbz or Boc,
wherein the cycloalkyl of said cycloalkylalkyl is unsubstituted or substituted with one or more X groups,
for —N(R¹⁴)₂, the two R¹⁴ groups, with the ring nitrogen atom to which they are shown attached, form an unsubstituted heterocyclyl ring or a heterocyclyl ring substituted with one or more X groups;
G is selected from the group consisting of H, alkyl, unsubstituted aryl, aryl substituted with one or more Y groups, —CN, cycloalkyl, —O—R⁷, —S—R⁷, unsubstituted heteroaryl, heteroaryl substituted with one or more Y groups, —N(R⁸)₂, unsubstituted heterocyclyl, and heterocyclyl substituted with one or more X groups;
each W is independently selected from the group consisting of hydrogen, alkyl, aryl, —C(O)-alkyl, —C(O)—O-alkyl, —C(R⁶)₂—N(R⁶)₂, and —C(R⁶)₂—N(R⁶)—S(O)₂—R⁶;
each X is independently selected from the group consisting of hydrogen, —OH, alkyl, arylalkyl, heteroarylalkyl, Cbz, Boc, alkylsulfonyl, acetyl, —C(O)—R¹², —C(O)—N(R⁹)₂, —C(O)-heteroaryl, heteroaryl, —S(O)₂-cycloalkyl, —C(O)-alkyl, —C(O)—O-alkyl, —C(R⁶)₂)ₘ-aryl and aryl
wherein the aryl and heteroaryl portion of said arylalkyl and heteroarylalkyl is unsubstituted or substituted with one or more Y groups,
wherein the alkyl portion of said arylalkyl and heteroarylalkyl is unsubstituted or substituted with one or more X groups with the proviso that X substituted on said alkyl portion is NOT Cbz or Boc,
wherein said heteroaryl or the heteroaryl portion of said —C(O)heteroaryl of X is unsubstituted or substituted with one or more substituents selected from the group consisting of halogen, —OH, —O-alkyl, haloalkyl, and —CN, and
wherein said aryl or the aryl portion of said —C(R⁶)₂)ₘ-aryl of X is unsubstituted or substituted with one or more substituents selected from the group consisting of halogen, —OH, —O-alkyl, haloalkyl, and —CN
wherein in a single X moiety, ═O, can replace two available hydrogens on the same carbon on a ring system;
each Y is independently selected from the group consisting of hydrogen, halogen, alkyl, aryl, —C(O)-alkyl, —O-alkyl, —O-heteroaryl, —O-aryl, —O—R⁹, haloalkyl, —O-haloalkyl, —CN, —C(O)—O-alkyl, —N(R⁶)₂, —C(R⁶)₂—N(R⁶)₂, —S(O)₂-heterocyclyl, —S(O)₂-heteroaryl and —C(R⁶)₂—N(R⁶)—S(O)₂—R⁶; or
two of said Y groups attached to adjacent carbon atoms form a O—CH₂—O— or —O—CH₂CH₂—O— group;
each Z is independently selected from the group consisting of hydrogen, alkyl, arylalkyl, heteroarylalkyl, —C(O)—N(R⁹)₂, —C(O)-heteroaryl, heteroaryl, —S(O)₂-cycloalkyl, —C(O)-alkyl, —C(R⁶)₂)ₘ-aryl, —N(R⁶)—S(O)₂—R⁹ and aryl
wherein the aryl and heteroaryl portion of said arylalkyl and heteroarylalkyl is unsubstituted or substituted with one or more Y groups,
wherein the alkyl portion of said arylalkyl and heteroarylalkyl is unsubstituted or substituted with one or more X groups with the proviso that X substituted on said alkyl portion is NOT Cbz or Boc,
wherein said heteroaryl or the heteroaryl portion of said —C(O)-heteroaryl of Z is unsubstituted or substituted with one or more substituents selected from the group consisting of halogen, —OH, —O-alkyl, haloalkyl, and —CN, and
wherein said aryl or the aryl portion of said —C(R⁶)₂)ₘ-aryl of Z is unsubstituted or substituted with one or more substituents selected from the group consisting of halogen, —OH, —O-alkyl, haloalkyl, and —CN;
wherein in a single Z moiety, ═O, can replace two available hydrogens on the same carbon on a ring system;
each n, p and q is independently an integer from 0-5; and
m is an integer from 1-5.

2. The compound of claim 1, or a pharmaceutically acceptable salt, solvate or ester thereof, wherein:
A is —CH₂— or —C(O)—;
R¹ is selected from the group consisting of H, —N(R⁴)(R⁵), unsubstituted (C₂-C₁₀)heterocyclyl, (C₂-C₁₀)heterocyclyl substituted with one or more X groups, —N₃, and —O—R⁷;
R² is selected from the group consisting of H, —(C(R⁶)₂)ₚ—(C₆-C₁₀)aryl, (C₃-C₆)cycloalkyl(C₁-C₆)alkyl, (C₃-C₆)cycloalkyl(C₁-C₆)alkyl substituted with Z, —(C(R⁶)₂)_{q}—(C₂-C₁₀)heterocyclyl, —(C(R⁶)₂)ₚ—S(O)₂—(C₂-C₁₀)heterocyclyl, and —C(R⁵)₂—O—R⁷,
wherein the (C₆-C₁₀)aryl portion of said —C(R⁶)₂—(C₆-C₁₀)aryl of R² is unsubstituted or substituted with one or more Y groups,
wherein the (C₂-C₁₀)heterocyclyl portion of said —(C(R⁶)₂)ₚ—S(O)₂—(C₂-C₁₀)heterocyclyl of R² is unsubstituted or substituted with one or more X groups,
wherein the (C₂-C₁₀)heterocyclyl portion of said —(C(R⁶)₂)_{q}—(C₂-C₁₀)heterocyclyl of R² is unsubstituted or substituted with one or more X groups;
R³ is selected from the group consisting of H, —C(R⁶)₂—(C₆-C₁₀)aryl, —C(R⁶)₂—O—R¹, —(C(R⁶)₂)_{q}—C(O)—N(R¹²)₂, —(C(R⁶)₂)ₚ—N(R⁹)—C(O)—(C(R⁶)₂)_{q}—R¹⁶, —(C(R⁶)₂)_{q}—S(O)₂—N(R⁹)—(C(R⁶)₂)_{q}—R¹⁵, —(C(R⁶)₂)_{q}—N(R⁹)—S(O)₂—(C(R⁶)₂)_{q}—R¹⁵ and —(C(R⁶)₂)_{q}—N(R⁸)₂,
wherein the (C₆-C₁₀)aryl portion of said —C(R⁶)₂—(C₆-C₁₀)aryl of R³ is unsubstituted or substituted with one or more Y groups;
with the following independent provisos:
(i) at least one of R¹, R², and R³ is not H;
(ii) when R¹ is —OH, at least one of R² and R³ is not H; and
(iii) when A is —C(O)—, at least one of R² and R³ is not H;
or, R² and R³ together with the ring carbon atom to which they are shown attached form an unsubstituted (C₂-C₁₀)heterocyclyl ring or a (C₂-C₁₀)heterocyclyl ring substituted with one or more X groups;
R⁴ is selected from the group consisting of H, —C(O)—(C₁-C₆)alkyl, and (C₁-C₆)alkyl;
R⁵ is selected from the group consisting of —C(R⁶)₂)ₘ-G, —S(O)₂—(C₁-C₆)alkyl, —S(O)₂—(C₃-C₆)cycloalkyl, (C₁-C₆)alkyl, —S(O)—(C₃-C₆)cycloalkyl, —C(O)—(C₃-C₆)cycloalkyl, —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₁-C₆)alkyl, —C(O)—(C₆-C₁₀)aryl, —C(O)—O—(C₁-C₆)alkyl, —C(O)—O—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C₂-C₁₀)heteroaryl(C₁-C₆)alkyl, —C(O)—(C(R⁶)₂)ₘ—O—(C₆-C₁₀)aryl, —C(O)-(benzo-fused (C₃-C₆)cycloalkyl), —S(O)₂-(benzo-fused (C₂-C₁₀)heterocyclyl), —C(O)—N(R⁹)—(C(R⁶)₂)ₘ—(C₆-C₁₀)aryl, —C(O)—N(R⁹)—(C₆-C₁₀)aryl, (C₃-C₆)cycloalkyl, benzo-fused (C₃-C₆)cycloalkyl, unsubstituted (C₆-C₁₀)aryl, (C₆-C₁₀)aryl substituted with one or more Y groups, unsubstituted (C₂-C₁₀)heterocyclyl, and (C₂-C₁₀)heterocyclyl substituted with one or more X groups,
wherein the (C₆-C₁₀)aryl or (C₂-C₁₀)heteroaryl portion of said —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₆-C₁₀)aryl, —C(O)—(C(R¹⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C(R⁶)₂)ₘ—O—(C₆-C₁₀)aryl, —C(O)—N(R⁹)—(C(R⁶)₂)ₘ—(C₆-C₁₀)aryl, or —C(O)—N(R⁹)—(C₆-C₁₀)aryl of R⁵ is unsubstituted or substituted with one or more Y groups;
wherein the heterocyclyl portion of —S(O)₂-(benzo-fused (C₂-C₁₀)heterocyclyl)aryl of R⁵ is unsubstituted or substituted with one or more X groups;
each R⁶ is independently selected from the group consisting of H and (C₁-C₆)alkyl;
R⁷ is selected from the group consisting of H, (C₁-C₆)alkyl, unsubstituted (C₂-C₁₀)heteroaryl and (C₂-C₁₀)heteroaryl substituted with one or more Y groups, unsubstituted (C₆-C₁₀)aryl, and (C₆-C₁₀)aryl substituted with one or more Y groups;
each R⁸ is independently selected from the group consisting of H, (C₁-C₆)alkyl, (C₆-C₁₀)aryl(C₁-C₆)alkyl, (C₂-C₁₀)heteroaryl(C₁-C₆)alkyl, unsubstituted (C₆-C₁₀)aryl, unsubstituted (C₂-C₁₀)heteroaryl, —C(O)—(C₁-C₆)alkyl, —C(O)—(C₆-C₁₀)aryl, —C(O)—(C₃-C₆)cycloalkyl, —C(O)N(R⁹)₂, —S(O)₂—(C₆-C₁₀)aryl, —S(O)₂—(C₂-C₁₀)heteroaryl, —SO₂N(R⁹)₂, —S(O)₂—(C₃-C₆)cycloalkyl, (C₆-C₁₀)aryl and (C₂-C₁₀)heteroaryl substituted with one or more Y groups, and —S(O)₂—(C₁-C₆)alkyl,
wherein the (C₆-C₁₀)aryl portion of said (C₆-C₁₀)aryl(C₁-C₆)alkyl, —C(O)—(C₆-C₁₀)aryl or —S(O)₂—C₆-C₁₀)aryl and the (C₂-C₁₀)heteroaryl portion of said (C₂-C₁₀)heteroaryl(C₁-C₆)alkyl, —S(O)₂—(C₂-C₁₀)heteroaryl of R⁸ is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each R¹² is independently selected from the group consisting of H, (C₁-C₆)alkyl, (C₃-C₆)cycloalkyl(C₁-C₆)alkyl, —(C(R⁶)₂)_{q}—C(O)R¹³, benzo(C₂-C₁₀)heterocyclyl, benzocyclo(C₁-C₆)alkyl, —(C(R⁶)₂)_{q}—N(R⁹)—C(O)R¹³, (C(R⁶)₂)_{q} N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, Y—(C₁-C₆)alkylenyl-O—, W—O—(C₁-C₆)alkylenyl, (C₂-C₁₀)heterocyclyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups,
wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
with the following provisos that
for —N(R¹⁴)₂ of R¹², the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each R¹³ is independently selected from the group consisting of H, (C₁-C₆)alkyl, (C₃-C₆)cycloalkyl(C₁-C₆)alkyl, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups;
each R¹⁴ is independently selected from the group consisting of H, Boc, unsubstituted (C₁-C₆)alkyl, (C₁-C₆)alkyl substituted with one or more X groups, unsubstituted (C₃-C₆)cycloalkyl, (C₃-C₆)cycloalkyl substituted with one or more Y groups, unsubstituted (C₆-C₁₀)aryl, (C₆-C₁₀)aryl substituted with one or more Y groups, (C₂-C₁₀)heterocyclyl, unsubstituted (C₂-C₁₀)heteroaryl and (C₂-C₁₀)heteroaryl substituted with one or more Y groups;
each R¹⁵ is independently selected from the group consisting of H, (C₁-C₆)alkyl, —N(R⁴)(R⁵), (C(R⁶)₂)_{q}—N(R¹⁴)₂, (C₁-C₆)alkylenyl-CF₃, —CF₃, (C₃-C₆)cycloalkyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, benzo(C₂-C₁₀)heterocyclyl, benzocyclo(C₁-C₆)alkyl, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups,
wherein the (C₁-C₆)alkyl portion of said (C₃-C₆)cycloalkyl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups,
wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
each R¹⁶ is independently selected from the group consisting of H, (C₁-C₆)alkyl, (C₃-C₆)cycloalkyl(C₁-C₆)alkyl, —(C(R⁶)₂)ₚ—C(O)R¹³, —(C(R⁶)₂)ₚ—N(R⁹)—C(O)R¹³, —(C(R⁶)₂)ₚ—N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups,
for —N(R¹⁴)₂, the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
G is selected from the group consisting of H, (C₁-C₆)alkyl, unsubstituted (C₆-C₁₀)aryl, (C₆-C₁₀)aryl substituted with one or more Y groups, —CN, (C₃-C₆)cycloalkyl, —O—R⁷, —S—R⁷, unsubstituted (C₂-C₁₀)heteroaryl, (C₂-C₁₀)heteroaryl substituted with one or more Y groups, —N(R⁸)₂, unsubstituted (C₂-C₁₀)heterocyclyl, and (C₂-C₁₀)heterocyclyl substituted with one or more X groups;
each W is independently selected from the group consisting of hydrogen, (C₁-C₆)alkyl, (C₆-C₁₀)aryl, —C(O)—(C₁-C₆)alkyl, —C(O)—O—(C₁-C₆)alkyl, —C(R⁶)₂—N(R⁶)₂, and —C(R⁶)₂—N(R⁶)—S(O)₂—R⁶;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)—O—(C₁-C₆)alkyl, —C(R⁶)₂)ₘ—(C₆-C₁₀)aryl and (C₆-C₁₀)aryl
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
wherein said (C₆-C₁₀)aryl or the (C₆-C₁₀)aryl portion of said —C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
wherein in a single X moiety, ═O, can replace two available hydrogens on the same carbon on a ring system;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each Z is independently selected from the group consisting of hydrogen, (C₁-C₆)alkyl, (C₆-C₁₀)aryl(C₁-C₆)alkyl, (C₂-C₁₀)heteroaryl(C₁-C₆)alkyl, —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(R⁶)₂)ₘ—(C₆-C₁₀)aryl, —N(R⁶)—S(O)₂—R⁹ and (C₆-C₁₀)aryl
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein said (C₂-C₁₀)heteroaryl or the (C₂-C₁₀)heteroaryl portion of said —C(O)—(C₂-C₁₀)heteroaryl of Z is unsubstituted or substituted with one or more substituents selected from the group consisting of halogen, —OH, —O—(C₁-C₆)alkyl, halo(C₁-C₆)alkyl, and —CN, and
wherein said (C₆-C₁₀)aryl or the (C₆-C₁₀)aryl portion of said —C(R⁶)₂)ₘ—(C₆-C₁₀)aryl of Z is unsubstituted or substituted with one or more substituents selected from the group consisting of halogen, —OH, —O—(C₁-C₆)alkyl, halo(C₁-C₆)alkyl, and —CN;
wherein in a single Z moiety, ═O, can replace two available hydrogens on the same carbon on a ring system;
each n, p and q is independently an integer from 0-5; and
m is an integer from 1-5.

3. The compound of claim 1 having the structural Formula (IA): 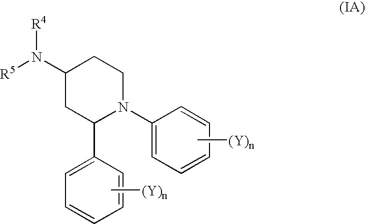
or a pharmaceutically acceptable salt, solvate or ester thereof, wherein:
R⁴ is selected from the group consisting of H, —C(O)—(C₁-C₆)alkyl, and (C₁-C₆)alkyl;
R⁵ is selected from the group consisting of —C(R⁶)₂)ₘ-G, —S(O)₂—(C₁-C₆)alkyl, —S(O)—(C₃-C₆)cycloalkyl, —C(O)—(C₃-C₆)cycloalkyl, —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₁-C₆)alkyl, —C(O)—(C₆-C₁₀)aryl, —C(O)—O—(C₁-C₆)alkyl, —C(O)—O—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C₂-C₁₀)heteroaryl(C₁-C₆)alkyl, —C(O)—(C(R⁶)₂)ₘ—O—(C₆-C₁₀)aryl, —C(O)-(benzo-fused (C₃-C₆)cycloalkyl), —S(O)₂-(benzo-fused (C₂-C₁₀)heterocyclyl), —C(O)—N(R⁹)—(C(R⁶)₂)ₘ—(C₆-C₁₀)aryl, —C(O)—N(R⁹)—(C₆-C₁₀)aryl, (C₃-C₆)cycloalkyl, benzo-fused (C₃-C₆)cycloalkyl, unsubstituted (C₆-C₁₀)aryl, (C₆-C₁₀)aryl substituted with one or more Y groups, unsubstituted (C₂-C₁₀)heterocyclyl, (C₂-C₁₀)heterocyclyl substituted with one or more X groups,
wherein the (C₆-C₁₀)aryl or heteroaryl portion of said —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C(R⁶)₂)ₘ—O—(C₆-C₁₀)aryl, —C(O)—N(R⁹)—(C(R⁶)₂)ₘ—(C₆-C₁₀)aryl, or —C(O)—N(R⁹)—(C₆-C₁₀)aryl of R⁵ is unsubstituted or substituted with one or more Y groups;
wherein the heterocyclyl portion of —S(O)₂-(benzo-fused (C₂-C₁₀)heterocyclyl)aryl of R⁵ is unsubstituted or substituted with one or more X groups;
each R⁶ is independently selected from the group consisting of H and (C₁-C₆)alkyl;
R⁷ is selected from the group consisting of H, (C₁-C₆)alkyl, unsubstituted (C₆-C₁₀)aryl, and (C₆-C₁₀)aryl substituted with one or more Y groups;
each R⁸ is independently selected from the group consisting of H, (C₁-C₆)alkyl, —C(O)—(C₆-C₁₀)aryl, —S(O)₂—(C₆-C₁₀)aryl, and —S(O)₂—(C₁-C₆)alkyl;
each R⁹ is independently selected from the group consisting of H, (C₁-C₆)alkyl, (C₃-C₆)cycloalkyl and unsubstituted (C₆-C₁₀)aryl;
G is selected from the group consisting of H, (C₁-C₆)alkyl, unsubstituted (C₆-C₁₀)aryl, (C₆-C₁₀)aryl substituted with one or more Y groups, —CN, (C₃-C₆)cycloalkyl, —O—R¹, —S—R⁷, unsubstituted (C₂-C₁₀)heteroaryl, (C₂-C₁₀)heteroaryl substituted with one or more Y groups, —N(R⁸)₂, unsubstituted (C₂-C₁₀)heterocyclyl, and (C₂-C₁₀)heterocyclyl substituted with one or more X groups;
each X is independently selected from the group consisting of (C₁-C₆)alkyl, —C(O)—N(R⁹)₂, —C(O)—(C₂-C₁₀)heteroaryl, (C₂-C₁₀)heteroaryl, —(C(R⁶)₂)ₘ—(C₆-C₁₀)aryl, and (C₆-C₁₀)aryl,
wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O-alkyl, haloalkyl, and —CN, and
wherein said (C₆-C₁₀)aryl or the (C₆-C₁₀)aryl portion of said —(C(R⁶)₂)ₘ—(C₆-C₁₀)aryl of X is unsubstituted or substituted with one or more substituents selected from the group consisting of halogen, —OH, —O-alkyl, haloalkyl, and —CN;
each Y is independently selected from the group consisting of halogen, (C₁-C₆)alkyl, (C₆-C₁₀)aryl, —C(O)—(C₁-C₆)alkyl, —O—R⁹, (C₁-C₆)haloalkyl, —O—(C₁-C₆)haloalkyl, —CN, —C(O)—O—(C₁-C₆)alkyl, —N(R⁶)₂, and —C(R⁶)₂—N(R⁶)₂; or
two of said Y groups attached to adjacent carbon atoms form a —O—CH₂—O— or —O—CH₂CH₂—O— group;
each n is independently an integer from 0-5; and
m is an integer from 1-5.

4. The compound of claim 3, or a pharmaceutically acceptable salt, solvate or ester thereof, wherein:
R⁴ is H.

5. The compound of claim 4, or a pharmaceutically acceptable salt, solvate or ester thereof, wherein:
R⁵ is selected from the group consisting of —(C(R⁶)₂)ₘ-G, —S(O)₂—(C₁-C₆)alkyl, —S(O)—(C₃-C₆)cycloalkyl, —C(O)—(C₃-C₆)cycloalkyl, —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₆-C₁₀)aryl, —C(O)—O—(C₁-C₆)alkyl, —C(O)—O—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C₂-C₁₀)heteroaryl(C₁-C₆)alkyl, —C(O)—(C(R⁶)₂)ₘ—O—(C₆-C₁₀)aryl, —C(O)-(benzo-fused (C₃-C₆)cycloalkyl), —S(O)₂-(benzo-fused (C₂-C₁₀)heterocyclyl), —C(O)—N(R⁹)—(C(R⁶)₂)ₘ—(C₆-C₁₀)aryl, —C(O)—N(R⁹)—(C₆-C₁₀)aryl, (C₃-C₆)cycloalkyl, and benzo-fused (C₃-C₆)cycloalkyl;
each R⁶ is independently selected from the group consisting of H and (C₁-C₆)alkyl;
R⁷ is selected from the group consisting of H, (C₁-C₆)alkyl and unsubstituted (C₆-C₁₀)aryl;
each R⁹ is independently selected from the group consisting of H, (C₁-C₆)alkyl, (C₃-C₆)cycloalkyl and unsubstituted (C₆-C₁₀)aryl;
G is selected from the group consisting of H, (C₁-C₆)alkyl, unsubstituted (C₆-C₁₀)aryl, (C₆-C₁₀)aryl substituted with one or more Y groups, —CN, (C₃-C₆)cycloalkyl, —O—R⁷, —S—R⁷, unsubstituted (C₂-C₁₀)heteroaryl, unsubstituted (C₂-C₁₀)heterocyclyl, and (C₂-C₁₀)heterocyclyl substituted with one or more X groups;
each X is independently selected from the group consisting of (C₁-C₆)alkyl and (C₆-C₁₀)aryl substituted with one or more halogen; and
each Y is independently selected from the group consisting halogen, (C₁-C₆)alkyl, (C₆-C₁₀)aryl, —O—R⁹, (C₁-C₆)haloalkyl, —O—(C₁-C₆)haloalkyl, —CN, —C(O)—O—(C₁-C₆)alkyl, —C(O)—(C₁-C₆)alkyl, —N(R⁶)₂, and —C(R⁶)₂—N(R⁶)₂; or
two of said Y groups attached to adjacent carbon atoms form a —O—CH₂—O— or —O—CH₂CH₂—O— group.

6. The compound of claim 5, or a pharmaceutically acceptable salt, solvate or ester thereof, wherein:
R⁵ is selected from the group consisting of —C(R⁶)₂)ₘ-G, —S(O)₂—CH₃, —S(O)₂-phenyl, —S(O)₂—C(R⁶)₂-phenyl, —S(O)₂-thiophenyl, —C(O)-phenyl, —C(O)—C(R⁶)₂-phenyl, —C(O)-cyclopropylene-phenyl, —C(O)-(benzo-fused cyclohexyl), —C(O)-furanyl, —C(O)—C(R⁶)₂—O-phenyl, —C(O)—(C(R⁶)₂)₂-phenyl, —C(O)—N(R⁹)-phenyl, —C(O)—N(R⁹)—C(R⁶)₂-phenyl, cyclobutyl, cyclopentyl, cyclohexyl, and indanyl,
wherein the phenyl, thiophenyl, and furanyl portions of said —S(O)₂-phenyl, —S(O)₂—C(R⁶)₂-phenyl, —S(O)₂-thiophenyl, —C(O)-phenyl, —C(O)C(R⁶)₂-phenyl, —C(O)-cyclopropylene-phenyl, —C(O)-furanyl, —C(O)—C(R⁶)₂—O-phenyl, —C(O)—(C(R⁶)₂)₂-phenyl, —C(O)—N(R⁹)-phenyl, or —C(O)—N(R⁹)—C(R⁶)₂-phenyl of R⁵ are unsubstituted or substituted with one or more Y groups;
each R⁶ is independently selected from the group consisting of H, —CH₃, —CH₂CH₃, and —CH₂(CH₃)₂;
R⁷ is selected from the group consisting of H, —CH₃, —CH(CH₃)₂, —CH₂CH₂CH₂CH₃, and unsubstituted phenyl;
each R⁹ is independently selected from the group consisting of H, —CH₃, —CH(CH₃)₂, —CH₂CH₂CH₂CH₃, and unsubstituted phenyl;
G is selected from the group consisting of H, —CH₃, —CH₂CH₃, —C(CH₃)₃, unsubstituted phenyl, phenyl substituted with one or more Y groups, —CN, cyclohexyl, —O—R⁷, —S—R⁷, furanyl, thiophenyl, pyridinyl, benzothiophenyl, and pyrrolidinyl substituted with one or more X groups;
each X is independently selected from the group consisting of —CH₃ and phenyl substituted with one or more Cl; and
each Y is independently selected from the group consisting of F, Cl, —OCF₃, —OCH₃, phenyl, —C(O)—CH₃, —CH₃, —CN, —NH₂, and —CF₃; or
two of said Y groups attached to adjacent carbon atoms form a —O—CH₂—O— group.

7. The compound of claim 1 having the structural Formula (IB): 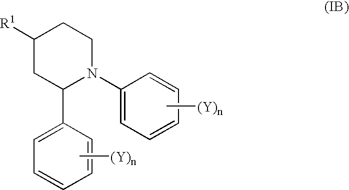
or a pharmaceutically acceptable salt, solvate or ester thereof, wherein:
R¹ is selected from the group consisting of unsubstituted (C₂-C₁₀)heterocyclyl, (C₂-C₁₀)heterocyclyl substituted with one or more X groups, —N₃, and —OR⁷;
each R⁶ is independently selected from the group consisting of H and (C₁-C₆)alkyl;
R⁷ is selected from the group consisting of (C₁-C₆)alkyl, unsubstituted (C₆-C₁₀)aryl, and (C₆-C₁₀)aryl substituted with one or more Y groups;
each R⁸ is independently selected from the group consisting of H, (C₁-C₆)alkyl, —C(O)—(C₆-C₁₀)aryl, —S(O)₂—(C₆-C₁₀)aryl, and —S(O)₂—(C₁-C₆)alkyl;
each X is independently selected from the group consisting of (C₁-C₆)alkyl, —C(O)—N(R⁸)₂, —C(O)—(C₂-C₁₀)heteroaryl, (C₂-C₁₀)heteroaryl, —(C(R⁶)₂)—(C₆-C₁₀)aryl, and (C₆-C₁₀)aryl
wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O-alkyl, haloalkyl, and —CN, and
wherein said (C₆-C₁₀)aryl or the (C₆-C₁₀)aryl portion of said —(C(R⁶)₂)—(C₆-C₁₀)aryl of X is unsubstituted or substituted with one or more substituents selected from the group consisting of halogen, —OH, —O-alkyl, haloalkyl, and —CN; and
n is an integer from 0-5.

8. The compound of claim 1 having the structural Formula (IC): 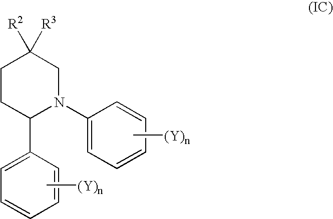
or a pharmaceutically acceptable salt, solvate or ester thereof, wherein:
R² is selected from the group consisting of H, —C(R⁶)₂—(C₆-C₁₀)aryl, and —C(R⁶)₂—O—R⁷,
wherein the (C₆-C₁₀)aryl portion of said —C(R⁶)₂—(C₆-C₁₀)aryl of R² is unsubstituted or substituted with one or more Y groups;
R³ is selected from the group consisting of H, —C(R⁶)₂—(C₆-C₁₀)aryl, —C(R⁶)₂—O—R⁷, and —C(R⁶)₂—N(R⁸)₂,
wherein the (C₆-C₁₀)aryl portion of said —C(R⁶)₂—(C₆-C₁₀)aryl of R³ is unsubstituted or substituted with one or more Y groups;
with the following proviso:
(i) at least one of R² and R³ is not H;
or, R² and R³ together with the ring carbon atom to which they are shown attached form an unsubstituted (C₂-C₁₀)heterocyclyl ring or a (C₂-C₁₀)heterocyclyl ring substituted with one or more X groups;
each R⁶ is independently selected from the group consisting of H and (C₁-C₆)alkyl;
R⁷ is selected from the group consisting of H, (C₁-C₆)alkyl, unsubstituted (C₆-C₁₀)aryl, and (C₆-C₁₀)aryl substituted with one or more Y groups;
each R⁸ is independently selected from the group consisting of H, (C₁-C₆)alkyl, —C(O)—(C₆-C₁₀)aryl, —S(O)₂—(C₆-C₁₀)aryl, —S(O)₂—(C₂-C₁₀)heteroaryl, and —S(O)₂—(C₁-C₆)alkyl,
wherein the (C₆-C₁₀)aryl portion of said —C(O)—(C₆-C₁₀)aryl or —S(O)₂—(C₆-C₁₀)aryl and the (C₂-C₁₀)heteroaryl portion of said —S(O)₂—(C₂-C₁₀)heteroaryl of R⁸ is unsubstituted or substituted with one or more Y groups;
each Y is independently selected from the group consisting of halogen, (C₁-C₆)alkyl, —C(O)—(C₁-C₆)alkyl, —O—R⁹, —O—C(R⁶)₂—O—, (C₁-C₆)haloalkyl, —O—(C₁-C₆)haloalkyl, —CN, —C(O)—O—(C₁-C₆)alkyl, —C(R⁶)₂—N(R⁶)₂, and —C(R⁶)₂—N(R⁶)—S(O)₂—R⁶; and
each n is independently an integer from 0-5.

9. The compound of claim 8, or a pharmaceutically acceptable salt, solvate or ester thereof, wherein:
R² is H
R³ is selected from the group consisting of —C(R⁶)₂—(C₆-C₁₀)aryl, —C(R⁶)₂—O—R⁷, and —C(R⁶)₂—N(R⁸)₂,
wherein the (C₆-C₁₀)aryl portion of said —C(R⁶)₂—(C₆-C₁₀)aryl of R³ is unsubstituted or substituted with one or more Y groups;
each R⁶ is H;
R⁷ is (C₆-C₁₀)aryl substituted with one or more Y groups;
each R⁸ is independently selected from the group consisting of H, —S(O)₂—(C₆-C₁₀)aryl, and —S(O)₂—(C₁-C₆)alkyl;
each R⁹ is independently selected from the group consisting of H, (C₁-C₆)alkyl, (C₃-C₆)cycloalkyl and unsubstituted (C₆-C₁₀)aryl;
each Y is independently selected from the group consisting of halogen, —C(R⁶)₂—N(R⁶)₂, and —C(R⁶)₂—N(R⁶)—S(O)₂—R⁶.

10. The compound of claim 1 having the structural Formula (ID): 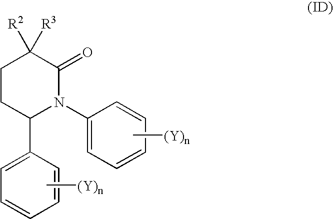
or a pharmaceutically acceptable salt, solvate or ester thereof, wherein:
R² is selected from the group consisting of H, —C(R⁶)₂—(C₆-C₁₀)aryl, and —C(R⁶)₂—O—R⁷,
wherein the (C₆-C₁₀)aryl portion of said —C(R⁶)₂—(C₆-C₁₀)aryl of R² is unsubstituted or substituted with one or more Y groups;
R³ is selected from the group consisting of —C(R⁶)₂—(C₆-C₁₀)aryl, —C(R⁶)₂—O—R⁷, and —C(R⁶)₂—N(R⁸)₂,
wherein the (C₆-C₁₀)aryl portion of said —C(R⁶)₂—(C₆-C₁₀)aryl of R³ is unsubstituted or substituted with one or more Y groups;
with the following proviso:
(i) at least one of R² and R³ is not H;
each R⁶ is independently selected from the group consisting of H and (C₁-C₆)alkyl;
R⁷ is selected from the group consisting of H, (C₁-C₆)alkyl, unsubstituted (C₆-C₁₀)aryl, and (C₆-C₁₀)aryl substituted with one or more Y groups;
each R⁸ is independently selected from the group consisting of H, (C₁-C₆)alkyl, —C(O)—(C₆-C₁₀)aryl, —S(O)₂—(C₆-C₁₀)aryl, and —S(O)₂—(C₁-C₆)alkyl;
each Y is independently selected from the group consisting of halogen, (C₁-C₆)alkyl, —O—R⁹, —O—C(R⁶)₂—O—, (C₁-C₆)haloalkyl, —CN, —C(R⁶)₂—N(R⁶)₂, and —C(R⁶)₂—N(R⁶)—S(O)₂—R⁶; and
each n is independently an integer from 0-5.

11. The compound of claim 1, or a pharmaceutically acceptable salt, solvate or ester thereof, with the following structural formula 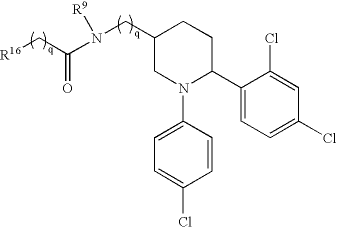
wherein:
each R⁶ is independently selected from the group consisting of H and (C₁-C₆)alkyl;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each R¹² is independently selected from the group consisting of H, (C₁-C₆)alkyl, (C₃-C₆)cycloalkyl(C₁-C₆)alkyl, —(C(R⁶)₂)_{q}—C(O)R¹³, benzo(C₂-C₁₀)heterocyclyl, benzocyclo(C₁-C₆)alkyl, —(C(R⁶)₂)_{q}—N(R⁹)—C(O)R¹³, —(C(R⁶)₂)_{q}—N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, Y—(C₁-C₆)alkylenyl-O—, W—O—(C₁-C₆)alkylenyl, (C₂-C₁₀)heterocyclyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups,
wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
with the following provisos that
for —N(R¹⁴)₂ of R¹², the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each R¹³ is independently selected from the group consisting of H, (C₁-C₆)alkyl, (C₃-C₆)cycloalkyl(C₁-C₆)alkyl, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups;
each R¹⁴ is independently selected from the group consisting of H, Boc, unsubstituted (C₁-C₆)alkyl, (C₁-C₆)alkyl substituted with one or more X groups, unsubstituted (C₃-C₆)cycloalkyl, (C₃-C₆)cycloalkyl substituted with one or more Y groups, unsubstituted (C₆-C₁₀)aryl, (C₆-C₁₀)aryl substituted with one or more Y groups, (C₂-C₁₀)heterocyclyl, unsubstituted (C₂-C₁₀)heteroaryl and (C₂-C₁₀)heteroaryl substituted with one or more Y groups;
each R¹⁶ is independently selected from the group consisting of H, (C₁-C₆)alkyl, (C₃-C₆)cycloalkyl(C₁-C₆)alkyl, —(C(R⁶)₂)ₚ—C(O)R³, —(C(R⁶)₂)ₚ—N(R⁹)—C(O)R¹³, —(C(R⁶)₂)ₚ—N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups,
for —N(R¹⁴)₂, the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each W is independently selected from the group consisting of hydrogen, (C₁-C₆)alkyl, (C₆-C₁₀)aryl, —C(O)—(C₁-C₆)alkyl, —C(O)—O—(C₁-C₆)alkyl, —C(R⁶)₂—N(R⁶)₂, and —C(R⁶)₂—N(R⁶)—S(O)₂—R⁶;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)—O—(C₁-C₆)alkyl, —(C(R⁶)₂)ₘ—(C₆-C₁₀)aryl and (C₆-C₁₀)aryl
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
wherein said (C₆-C₁₀)aryl or the (C₆-C₁₀)aryl portion of said —(C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
wherein in a single X moiety, ═O, can replace two available hydrogens on the same carbon on a ring system;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each n, p and q is independently an integer from 0-5; and
m is an integer from 1-5.

12. The compound of claim 1, or a pharmaceutically acceptable salt, solvate or ester thereof, with the following structural formula 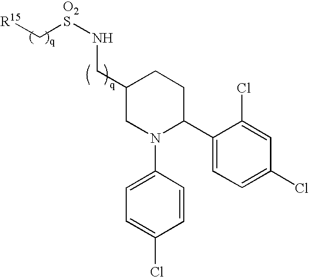
wherein:
R⁴ is selected from the group consisting of H, —C(O)—(C₁-C₆)alkyl, and (C₁-C₆)alkyl;
R⁵ is selected from the group consisting of —C(R⁶)₂)ₘ-G, —S(O)₂—(C₁-C₆)alkyl, —S(O)₂—(C₃-C₆)cycloalkyl, (C₁-C₆)alkyl, —S(O)—(C₃-C₆)cycloalkyl, —C(O)—(C₃-C₆)cycloalkyl, —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₁-C₆)alkyl, —C(O)—(C₆-C₁₀)aryl, —C(O)—O—(C₁-C₆)alkyl, —C(O)—O—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C₂-C₁₀)heteroaryl(C₁-C₆)alkyl, —C(O)—(C(R⁶)₂)ₘ—O—(C₆-C₁₀)aryl, —C(O)-(benzo-fused (C₃-C₆)cycloalkyl), —S(O)₂-(benzo-fused (C₂-C₁₀)heterocyclyl), —C(O)—N(R⁹)—(C(R⁶)₂)ₘ—(C₆-C₁₀)aryl, —C(O)—N(R⁹)—(C₆-C₁₀)aryl, (C₃-C₆)cycloalkyl, benzo-fused (C₃-C₆)cycloalkyl, unsubstituted (C₆-C₁₀)aryl, (C₆-C₁₀)aryl substituted with one or more Y groups, unsubstituted (C₂-C₁₀)heterocyclyl, and (C₂-C₁₀)heterocyclyl substituted with one or more X groups,
wherein the (C₆-C₁₀)aryl or (C₂-C₁₀)heteroaryl portion of said —S(O)₂—(C₆-C₁₀)aryl, —S(O)₂—(C(R⁶)₂)ₘ—(C₆-C₁₀)aryl, —S(O)₂—(C₂-C₁₀)heteroaryl, —C(O)—(C₆-C₁₀)aryl, —C(O)—(C(R⁶)₂)ₘ—(C₆-C₁₀)aryl, —C(O)—(C₃-C₆)cycloalkylene-(C₆-C₁₀)aryl, —C(O)—(C₂-C₁₀)heteroaryl, —C(O)—(C(R⁶)₂)ₘ—O—(C₆-C₁₀)aryl, —C(O)—N(R⁹)—(C(R⁶)₂)ₘ—(C₆-C₁₀)aryl, or —C(O)—N(R⁹)—(C₆-C₁₀)aryl of R⁵ is unsubstituted or substituted with one or more Y groups;
wherein the heterocyclyl portion of —S(O)₂-(benzo-fused (C₂-C₁₀)heterocyclyl)aryl of R⁵ is unsubstituted or substituted with one or more X groups;
each R⁶ is independently selected from the group consisting of H and (C₁-C₆)alkyl;
R⁷ is selected from the group consisting of H, (C₁-C₆)alkyl, unsubstituted (C₂-C₁₀)heteroaryl and (C₂-C₁₀)heteroaryl substituted with one or more Y groups, unsubstituted (C₆-C₁₀)aryl, and (C₆-C₁₀)aryl substituted with one or more Y groups;
each R⁸ is independently selected from the group consisting of H, (C₁-C₆)alkyl, (C₆-C₁₀)aryl(C₁-C₆)alkyl, (C₂-C₁₀)heteroaryl(C₁-C₆)alkyl, unsubstituted (C₆-C₁₀)aryl, unsubstituted (C₂-C₁₀)heteroaryl, —C(O)—(C₁-C₆)alkyl, —C(O(C₆-C₁₀)aryl, —C(O)—(C₃-C₆)cycloalkyl, —C(O)N(R⁹)₂, —S(O)₂—(C₆-C₁₀)aryl, —S(O)₂—(C₂-C₁₀)heteroaryl, —SO₂N(R⁹)₂, —S(O)₂—(C₃-C₆)cycloalkyl, (C₆-C₁₀)aryl and (C₂-C₁₀)heteroaryl substituted with one or more Y groups, and —S(O)₂—(C₁-C₆)alkyl,
wherein the (C₆-C₁₀)aryl portion of said (C₆-C₁₀)aryl(C₁-C₆)alkyl, —C(O)—(C₆-C₁₀)aryl or —S(O)₂—(C₆-C₁₀)aryl and the (C₂-C₁₀)heteroaryl portion of said (C₂-C₁₀)heteroaryl(C₁-C₆)alkyl, —S(O)₂—(C₂-C₁₀)heteroaryl of R⁸ is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each R¹² is independently selected from the group consisting of H, (C₁-C₆)alkyl, (C₃-C₆)cycloalkyl(C₁-C₆)alkyl, —(C(R⁶)₂)_{q}—C(O)R¹³, benzo(C₂-C₁₀)heterocyclyl, benzocyclo(C₁-C₆)alkyl, —(C(R⁶)₂)_{q}—N(R⁹)—C(O)R¹³, —(C(R⁶)₂)_{q}—N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, Y—(C₁-C₆)alkylenyl-O—, W—O—(C₁-C₆)alkylenyl, (C₂-C₁₀)heterocyclyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups,
wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
with the following provisos that
for —N(R¹⁴)₂ of R¹², the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each R¹³ is independently selected from the group consisting of H, (C₁-C₆)alkyl, (C₃-C₆)cycloalkyl(C₁-C₆)alkyl, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups;
each R¹⁴ is independently selected from the group consisting of H, Boc, unsubstituted (C₁-C₆)alkyl, (C₁-C₆)alkyl substituted with one or more X groups, unsubstituted (C₃-C₆)cycloalkyl, (C₃-C₆)cycloalkyl substituted with one or more Y groups, unsubstituted (C₆-C₁₀)aryl, (C₆-C₁₀)aryl substituted with one or more Y groups, (C₂-C₁₀)heterocyclyl, unsubstituted (C₂-C₁₀)heteroaryl and (C₂-C₁₀)heteroaryl substituted with one or more Y groups;
each R¹⁵ is independently selected from the group consisting of H, (C₁-C₆)alkyl, —N(R⁴)(R⁵), (C(R⁶)₂)_{q}—N(R¹⁴)₂, (C₁-C₆)alkylenyl-CF₃, —CF₃, (C₃-C₆)cycloalkyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, benzo(C₂-C₁₀)heterocyclyl, benzocyclo(C₁-C₆)alkyl, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups,
wherein the (C₁-C₆)alkyl portion of said (C₃-C₆)cycloalkyl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups,
wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
G is selected from the group consisting of H, (C₁-C₆)alkyl, unsubstituted (C₆-C₁₀)aryl, (C₆-C₁₀)aryl substituted with one or more Y groups, —CN, (C₃-C₆)cycloalkyl, —O—R⁷, —S—R⁷, unsubstituted (C₂-C₁₀)heteroaryl, (C₂-C₁₀)heteroaryl substituted with one or more Y groups, —N(R⁸)₂, unsubstituted (C₂-C₁₀)heterocyclyl, and (C₂-C₁₀)heterocyclyl substituted with one or more X groups;
each W is independently selected from the group consisting of hydrogen, (C₁-C₆)alkyl, (C₆-C₁₀)aryl, —C(O)(C₁-C₆)alkyl, —C(O)—O—(C₁-C₆)alkyl, —C(R⁶)₂—N(R⁶)₂, and —C(R⁶)₂—N(R⁶)—S(O)₂—R⁶;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)(C₁-C₆)alkyl, —C(O)—O—(C₁-C₆)alkyl, —C(R⁶)₂)ₘ—(C₆-C₁₀)aryl and (C₆-C₁₀)aryl
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
wherein said (C₆-C₁₀)aryl or the (C₆-C₁₀)aryl portion of said —C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
wherein in a single X moiety, ═O, can replace two available hydrogens on the same carbon on a ring system;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each n, p and q is independently an integer from 0-5; and
m is an integer from 1-5.

13. The compound of claim 1, or a pharmaceutically acceptable salt, solvate or ester thereof, with the following structural formula 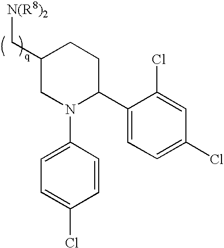
wherein:
each R⁶ is independently selected from the group consisting of H and (C₁-C₆)alkyl;
each R⁸ is independently selected from the group consisting of H, (C₁-C₆)alkyl, (C₆-C₁₀)aryl(C₁-C₆)alkyl, (C₂-C₁₀)heteroaryl(C₁-C₆)alkyl, unsubstituted (C₆-C₁₀)aryl, unsubstituted (C₂-C₁₀)heteroaryl, —C(O)—(C₁-C₆)alkyl, —C(O)—(C₆-C₁₀)aryl, —C(O)—(C₃-C₆)cycloalkyl, —C(O)N(R⁹)₂, —S(O)₂—(C₆-C₁₀)aryl, —S(O)₂—(C₂-C₁₀)heteroaryl, —SO₂N(R⁹)₂, —S(O)₂—(C₃-C₆)cycloalkyl, (C₆-C₁₀)aryl and (C₂-C₁₀)heteroaryl substituted with one or more Y groups, and —S(O)₂—(C₁-C₆)alkyl,
wherein the (C₆-C₁₀)aryl portion of said (C₆-C₁₀)aryl(C₁-C₆)alkyl, —C(O)—(C₆-C₁₀)aryl or —S(O)₂—(C₆-C₁₀)aryl and the (C₂-C₁₀)heteroaryl portion of said (C₂-C₁₀)heteroaryl(C₁-C₆)alkyl, —S(O)₂—(C₂-C₁₀)heteroaryl of R⁸ is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each q is independently an integer from 0 to 5.

14. The compound of claim 1, or a pharmaceutically acceptable salt, solvate or ester thereof, with the following structural formula 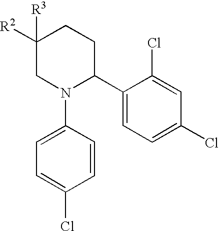
wherein
R² is selected from the group consisting of H, —(C(R⁶)₂)ₚ—(C₆-C₁₀)aryl, (C₃-C₆)cycloalkyl(C₁-C₆)alkyl, (C₃-C₆)cycloalkyl(C₁-C₆)alkyl substituted with Z, —(C(R⁶)₂)_{q}—(C₂-C₁₀)heterocyclyl, —(C(R⁶)₂)ₚ—S(O)₂—(C₂-C₁₀)heterocyclyl, and —C(R⁶)₂—O—R⁷,
wherein the (C₆-C₁₀)aryl portion of said —C(R⁶)₂—(C₆-C₁₀)aryl of R² is unsubstituted or substituted with one or more Y groups,
wherein the (C₂-C₁₀)heterocyclyl portion of said —(C(R⁶)₂)ₚ—S(O)₂—(C₂-C₁₀)heterocyclyl of R² is unsubstituted or substituted with one or more X groups,
wherein the (C₂-C₁₀)heterocyclyl portion of said —(C(R⁶)₂)_{q}—(C₂-C₁₀)heterocyclyl of R² is unsubstituted or substituted with one or more X groups;
R³ is selected from the group consisting of H, —(C(R⁶)₂)_{q}—C(O)—N(R¹²)₂ or —C(R⁶)₂)_{q}—N(R⁸)₂;
each R⁶ is independently selected from the group consisting of H and (C₁-C₆)alkyl;
R⁷ is selected from the group consisting of H, (C₁-C₆)alkyl, unsubstituted (C₂-C₁₀)heteroaryl and (C₂-C₁₀)heteroaryl substituted with one or more Y groups, unsubstituted (C₆-C₁₀)aryl, and (C₆-C₁₀)aryl substituted with one or more Y groups;
each R⁸ is independently selected from the group consisting of H, (C₁-C₆)alkyl, (C₆-C₁₀)aryl(C₁-C₆)alkyl, (C₂-C₁₀)heteroaryl(C₁-C₆)alkyl, unsubstituted (C₆-C₁₀)aryl, unsubstituted (C₂-C₁₀)heteroaryl, —C(O)—(C₁-C₆)alkyl, —C(O)—(C₆-C₁₀)aryl, —C(O)—(C₃-C₆)cycloalkyl, —C(O)N(R⁹)₂, —S(O)₂—(C₆-C₁₀)aryl, —S(O)₂—(C₂-C₁₀)heteroaryl, —SO₂N(R⁹)₂, —S(O)₂—(C₃-C₆)cycloalkyl, (C₆-C₁₀)aryl and (C₂-C₁₀)heteroaryl substituted with one or more Y groups, and —S(O)₂—(C₁-C₆)alkyl,
wherein the (C₆-C₁₀)aryl portion of said (C₆-C₁₀)aryl(C₁-C₆)alkyl, —C(O)—(C₆-C₁₀)aryl or —S(O)₂—(C₆-C₁₀)aryl and the (C₂-C₁₀)heteroaryl portion of said (C₂-C₁₀)heteroaryl(C₁-C₆)alkyl, —S(O)₂—(C₂-C₁₀)heteroaryl of R⁸ is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each R¹² is independently selected from the group consisting of H, (C₁-C₆)alkyl, (C₃-C₆)cycloalkyl(C₁-C₆)alkyl, —(C(R⁶)₂)_{q}—C(O)R¹³, benzo(C₂-C₁₀)heterocyclyl, benzocyclo(C₁-C₆)alkyl, —(C(R⁶)₂)_{q}—N(R⁹)—C(O)R¹³, —(C(R⁶)₂)_{q}—N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, Y—(C₁-C₆)alkylenyl-O—, W—O—(C₁-C₆)alkylenyl, (C₂-C₁₀)heterocyclyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups,
wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
with the following provisos that
for —N(R¹⁴)₂ of R¹², the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each R¹³ is independently selected from the group consisting of H, (C₁-C₆)alkyl, (C₃-C₆)cycloalkyl(C₁-C₆)alkyl, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups;
each R¹⁴ is independently selected from the group consisting of H, Boc, unsubstituted (C₁-C₆)alkyl, (C₁-C₆)alkyl substituted with one or more X groups, unsubstituted (C₃-C₆)cycloalkyl, (C₃-C₆)cycloalkyl substituted with one or more Y groups, unsubstituted (C₆-C₁₀)aryl, (C₆-C₁₀)aryl substituted with one or more Y groups, (C₂-C₁₀)heterocyclyl, unsubstituted (C₂-C₁₀)heteroaryl and (C₂-C₁₀)heteroaryl substituted with one or more Y groups;
each W is independently selected from the group consisting of hydrogen, (C₁-C₆)alkyl, (C₆-C₁₀)aryl, —C(O)—(C₁-C₆)alkyl, —C(O)—O—(C₁-C₆)alkyl, —C(R⁶)₂—N(R⁶)₂, and —C(R⁶)₂—N(R⁶)—S(O)₂—R⁶;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)—O—(C₁-C₆)alkyl, —C(R⁶)₂)ₘ—(C₆-C₁₀)aryl and (C₆-C₁₀)aryl
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
wherein said (C₆-C₁₀)aryl or the (C₆-C₁₀)aryl portion of said —C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
wherein in a single X moiety, ═O, can replace two available hydrogens on the same carbon on a ring system;
each Y is independently selected from the group consisting of hydrogen, halogen, (C₁-C₆)alkyl, (C₆-C₁₀)aryl, —C(O)(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each Z is independently selected from the group consisting of hydrogen, (C₁-C₆)alkyl, (C₆-C₁₀)aryl(C₁-C₆)alkyl, (C₂-C₁₀)heteroaryl(C₁-C₆)alkyl, —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(R⁶)₂)ₘ—(C₆-C₁₀)aryl, —N(R⁶)—S(O)₂—R⁹ and (C₆-C₁₀)aryl
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein said (C₂-C₁₀)heteroaryl or the (C₂-C₁₀)heteroaryl portion of said —C(O)—(C₂-C₁₀)heteroaryl of Z is unsubstituted or substituted with one or more substituents selected from the group consisting of halogen, —OH, —O—(C₁-C₆)alkyl, halo(C₁-C₆)alkyl, and —CN, and
wherein said (C₆-C₁₀)aryl or the (C₆-C₁₀)aryl portion of said —C(R⁶)₂)ₘ—(C₆-C₁₀)aryl of Z is unsubstituted or substituted with one or more substituents selected from the group consisting of halogen, —OH, —O—(C₁-C₆)alkyl, halo(C₁-C₆)alkyl, and —CN;
wherein in a single Z moiety, ═O, can replace two available hydrogens on the same carbon on a ring system;
each n, p and q is independently an integer from 0-5; and
m is an integer from 1-5.

15. The compound of claim 1, or a pharmaceutically acceptable salt, solvate or ester thereof, with the following structural formula 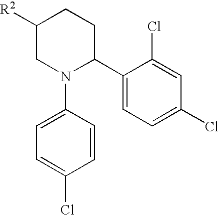
wherein;
R² is —(C(R⁶)₂)_{q}—(C₂-C₁₀)heterocyclyl;
wherein the (C₂-C₁₀)heterocyclyl portion of said —(C(R⁶)₂)_{q}—(C₂-C₁₀)heterocyclyl of R² is unsubstituted or substituted with one or more X groups;
each R⁶ is independently selected from the group consisting of H and (C₁-C₆)alkyl;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each R¹² is independently selected from the group consisting of H, (C₁-C₆)alkyl, (C₃-C₆)cycloalkyl(C₁-C₆)alkyl, —(C(R⁶)₂)_{q}—C(O)R¹³, benzo(C₂-C₁₀)heterocyclyl, benzocyclo(C₁-C₆)alkyl, —(C(R⁶)₂)_{q}—N(R⁹)—C(O)R¹³, (C(R⁶)₂)_{q} N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, Y—(C₁-C₆)alkylenyl-O—, W—O—(C₁-C₆)alkylenyl, (C₂-C₁₀)heterocyclyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups,
wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
with the following provisos that
for —N(R¹⁴)₂ of R¹², the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each R¹³ is independently selected from the group consisting of H, (C₁-C₆)alkyl, (C₃-C₆)cycloalkyl(C₁-C₆)alkyl, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups;
each R¹⁴ is independently selected from the group consisting of H, Boc, unsubstituted (C₁-C₆)alkyl, (C₁-C₆)alkyl substituted with one or more X groups, unsubstituted (C₃-C₆)cycloalkyl, (C₃-C₆)cycloalkyl substituted with one or more Y groups, unsubstituted (C₆-C₁₀)aryl, (C₆-C₁₀)aryl substituted with one or more Y groups, (C₂-C₁₀)heterocyclyl, unsubstituted (C₂-C₁₀)heteroaryl and (C₂-C₁₀)heteroaryl substituted with one or more Y groups;
each W is independently selected from the group consisting of hydrogen, (C₁-C₆)alkyl, (C₆-C₁₀)aryl, —C(O)—(C₁-C₆)alkyl, —C(O)—O—(C₁-C₆)alkyl, —C(R⁶)₂—N(R⁶)₂, and —C(R⁶)₂—N(R⁶)—S(O)₂—R⁶;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)—O—(C₁-C₆)alkyl, —C(R⁶)₂)ₘ—(C₆-C₁₀)aryl and (C₆-C₁₀)aryl
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
wherein said (C₆-C₁₀)aryl or the (C₆-C₁₀)aryl portion of said —C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
wherein in a single X moiety, ═O, can replace two available hydrogens on the same carbon on a ring system;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each n, p and q is independently an integer from 0-5; and
m is an integer from 1-5.

16. The compound of claim 15 wherein R³ is —C(R⁶)₂)_{q}—N(R⁸)₂ or —(C(R⁶)₂)_{q}—(C₂-C₁₀)heterocyclyl.

17. The compound of claim 1 having the structural formula: 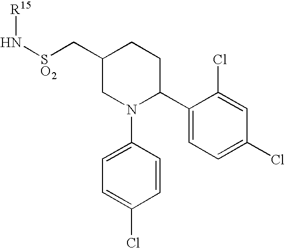
or a pharmaceutically acceptable salt, solvate or ester thereof, wherein:
R¹⁵ is alkyl.

18. The compound of claim 1 having the structural formula 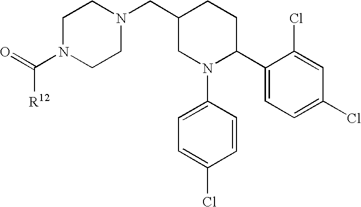
or a pharmaceutically acceptable salt, solvate or ester thereof, wherein:
each R⁶ is independently selected from the group consisting of H and (C₁-C₆)alkyl;
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
each R¹² is independently selected from the group consisting of H, (C₁-C₆)alkyl, (C₃-C₆)cycloalkyl(C₁-C₆)alkyl, —(C(R⁶)₂)_{q}—C(O)R¹³, benzo(C₂-C₁₀)heterocyclyl, benzocyclo(C₁-C₆)alkyl, —(C(R⁶)₂)_{q}—N(R⁹)—C(O)R¹³, —(C(R⁶)₂)_{q}—N(R¹⁴)₂, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, Y—(C₁-C₆)alkylenyl-O—, W—O—(C₁-C₆)alkylenyl, (C₂-C₁₀)heterocyclyl(C₁-C₆)alkyl, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups, and
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups,
wherein the benzo portion of said benzo(C₂-C₁₀)heterocyclyl can be optionally substituted with one or more Y groups and the (C₂-C₁₀)heterocyclyl portion of benzo(C₂-C₁₀)heterocyclyl can be optionally substituted with one or more X groups,
wherein the benzo portion of said benzocyclo(C₁-C₆)alkyl can be optionally substituted with one or more Y groups and the (C₃-C₆)cycloalkyl portion of benzocyclo(C₁-C₆)alkyl can be optionally substituted with one or more X groups;
with the following provisos that
for —N(R¹⁴)₂ of R¹², the two R¹⁴ groups, with the ring nitrogen atom to which they are shown attached, form an unsubstituted (C₂-C₁₀)heterocyclyl ring or a (C₂-C₁₀)heterocyclyl ring substituted with one or more X groups;
each R¹³ is independently selected from the group consisting of H, (C₁-C₆)alkyl, (C₃-C₆)cycloalkyl(C₁-C₆)alkyl, (C₆-C₁₀)aryl(C₁-C₆)alkyl, (C₂-C₁₀)heteroaryl(C₁-C₆)alkyl, HO—(C₁-C₆)alkyl-, (C₁-C₆)alkyl-O—, (C₆-C₁₀)aryl-O—, unsubstituted (C₃-C₆)cycloalkyl, (C₃-C₆)cycloalkyl substituted with one or more X groups, unsubstituted (C₂-C₁₀)heterocyclyl, (C₂-C₁₀)heterocyclyl substituted with one or more X groups, unsubstituted (C₂-C₁₀)heteroaryl, (C₂-C₁₀)heteroaryl substituted with one or more Y groups, unsubstituted (C₆-C₁₀)aryl and (C₆-C₁₀)aryl substituted with one or more Y groups
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₃-C₆)cycloalkyl(C₁-C₆)alkyl, (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein the (C₃-C₆)cycloalkyl of said (C₃-C₆)cycloalkyl(C₁-C₆)alkyl is unsubstituted or substituted with one or more X groups;
each R¹⁴ is independently selected from the group consisting of H, Boc, unsubstituted (C₁-C₆)alkyl, (C₁-C₆)alkyl substituted with one or more X groups, unsubstituted (C₃-C₆)cycloalkyl, (C₃-C₆)cycloalkyl substituted with one or more Y groups, unsubstituted (C₆-C₁₀)aryl, (C₆-C₁₀)aryl substituted with one or more Y groups, (C₂-C₁₀)heterocyclyl, unsubstituted (C₂-C₁₀)heteroaryl and (C₂-C₁₀)heteroaryl substituted with one or more Y groups;
each W is independently selected from the group consisting of hydrogen, (C₁-C₆)alkyl, (C₆-C₁₀)aryl, —C(O)—(C₁-C₆)alkyl, —C(O)—O—(C₁-C₆)alkyl, —C(R⁶)₂—N(R⁶)₂, and —C(R⁶)₂—N(R⁶)—S(O)₂—R⁶;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R¹², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)—O—(C₁-C₆)alkyl, —C(R⁶)₂)ₘ—(C₆-C₁₀)aryl and (C₆-C₁₀)aryl
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
wherein said (C₆-C₁₀)aryl or the (C₆-C₁₀)aryl portion of said —C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
wherein in a single X moiety, ═O, can replace two available hydrogens on the same carbon on a ring system;
each Y is independently selected from the group consisting of hydrogen, halogen, (C₁-C₆)alkyl, (C₆-C₁₀)aryl, —C(O)—(C₁-C₆)alkyl, —O—(C₁-C₆)alkyl, —O—(C₂-C₁₀)heteroaryl, —O—(C₆-C₁₀)aryl, —O—R⁹, halo(C₁-C₆)alkyl, —O-halo(C₁-C₆)alkyl, —CN, —C(O)—O—(C₁-C₆)alkyl, —N(R⁶)₂, —C(R⁶)₂—N(R⁶)₂, —S(O)₂—(C₂-C₁₀)heterocyclyl, —S(O)₂—(C₂-C₁₀)heteroaryl and —C(R⁶)₂—N(R⁶)—S(O)₂—R⁶; or
two of said Y groups attached to adjacent carbon atoms form a —O—CH₂—O— or —O—CH₂CH₂—O— group;
each n, p and q is independently an integer from 0-5; and
m is an integer from 1-5.

19. The compound of claim 1 having the structural formula 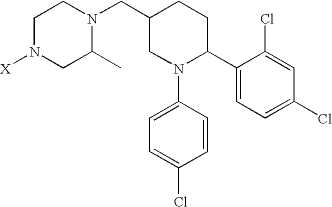
or a pharmaceutically acceptable salt, solvate or ester thereof, wherein:
each R⁹ is independently selected from the group consisting of H, (C₁-C₆)alkyl, halo(C₁-C₆)alkyl, hydroxy(C₁-C₆)alkyl, (C₃-C₆)cycloalkyl, unsubstituted (C₆-C₁₀)aryl and unsubstituted (C₂-C₁₀)heteroaryl;
R¹² is (C₁-C₆)alkyl;
each X is independently selected from the group consisting of hydrogen, —OH, (C₁-C₆)alkyl, (C₆-C₁₀)aryl(C₁-C₆)alkyl, (C₂-C₁₀)heteroaryl(C₁-C₆)alkyl, Cbz, Boc, (C₁-C₆)alkylsulfonyl, acetyl, —C(O)—R², —C(O)—N(R⁹)₂, —C(O)—(C₂-C₁₀)heteroaryl, (C₂-C₁₀)heteroaryl, —S(O)₂—(C₃-C₆)cycloalkyl, —C(O)—(C₁-C₆)alkyl, —C(O)—O—(C₁-C₆)alkyl, —(C(R⁶)₂)ₘ—(C₆-C₁₀)aryl and (C₆-C₁₀)aryl
wherein the (C₆-C₁₀)aryl and (C₂-C₁₀)heteroaryl portion of said (C₆-C₁₀)aryl(C₁-C₆)alkyl and (C₂-C₁₀)heteroaryl(C₁-C₆)alkyl is unsubstituted or substituted with one or more Y groups,
wherein the (C₁-C₆)alkyl portion of said (C₆-C₁₀)aryl(C₁-C₆)alkyl and (C₂-C₁₀)heteroaryl(C₁-C₆)alkyl is unsubstituted or substituted with one or more X groups with the proviso that X substituted on said (C₁-C₆)alkyl portion is NOT Cbz or Boc,
wherein said (C₂-C₁₀)heteroaryl or the (C₂-C₁₀)heteroaryl portion of said —C(O)—(C₂-C₁₀)heteroaryl of X is unsubstituted or substituted with one or more substituents selected from the group consisting of halogen, —OH, —O—(C₁-C₆)alkyl, halo(C₁-C₆)alkyl, and —CN, and
wherein said (C₆-C₁₀)aryl or the (C₆-C₁₀)aryl portion of said —C(R⁶)₂)ₘ—(C₆-C₁₀)aryl of X is unsubstituted or substituted with one or more substituents selected from the group consisting of halogen, —OH, —O—(C₁-C₆)alkyl, halo(C₁-C₆)alkyl, and —CN
wherein in a single X moiety, ═O, can replace two available hydrogens on the same carbon on a ring system.

20. The compound of claim 1 having the structural formula: 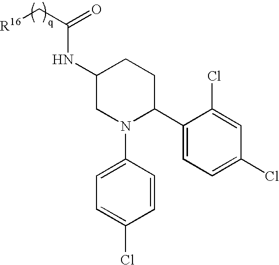
or a pharmaceutically acceptable salt, solvate or ester thereof, wherein:
R¹⁶ is —O—(C₁-C₆)alkyl or (C₂-C₁₀)heteroaryl(C₁-C₆)alkyl
q is 1 or 2.

21. The compound of claim 1, a pharmaceutically acceptable salt, solvate or ester thereof having a structure selected from the group consisting of:

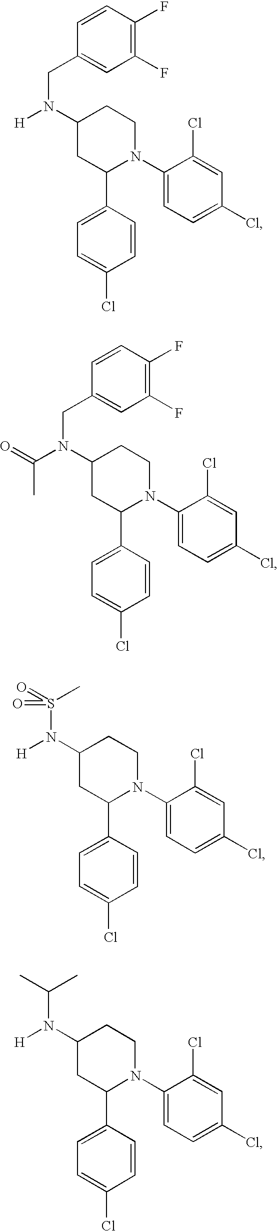

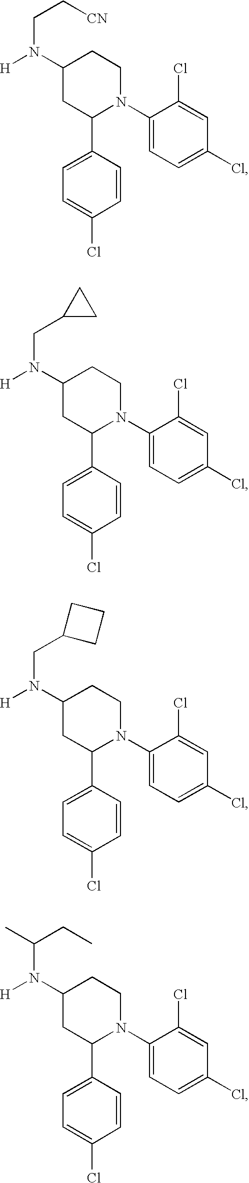

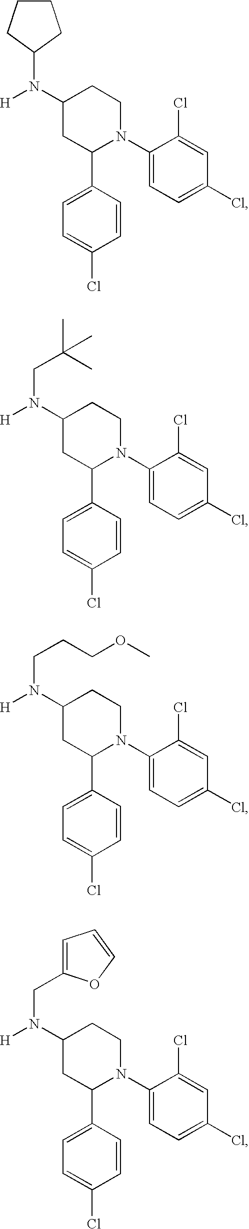

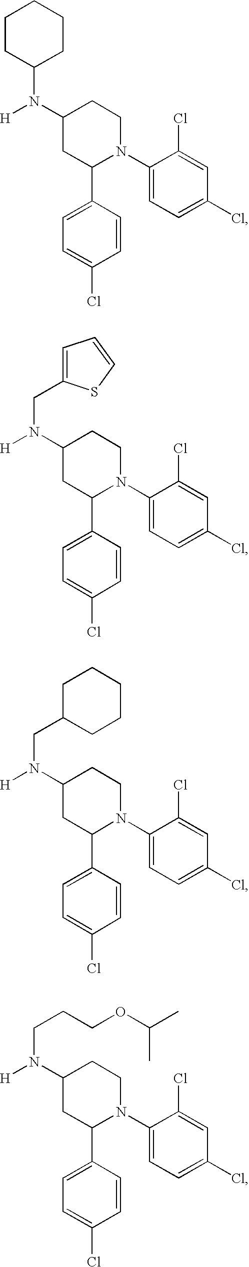

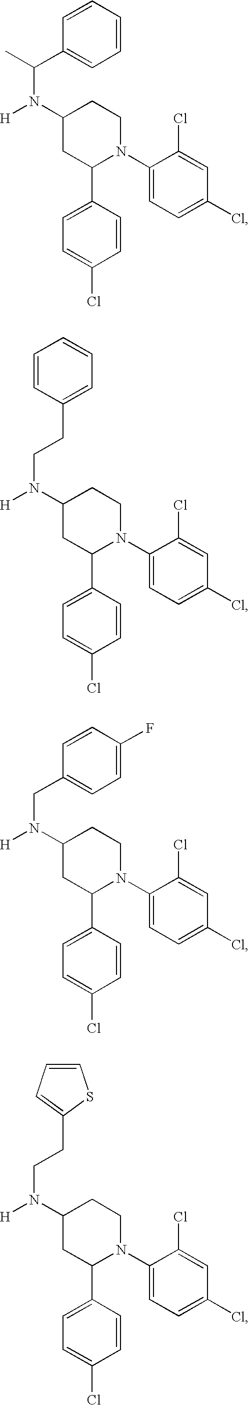

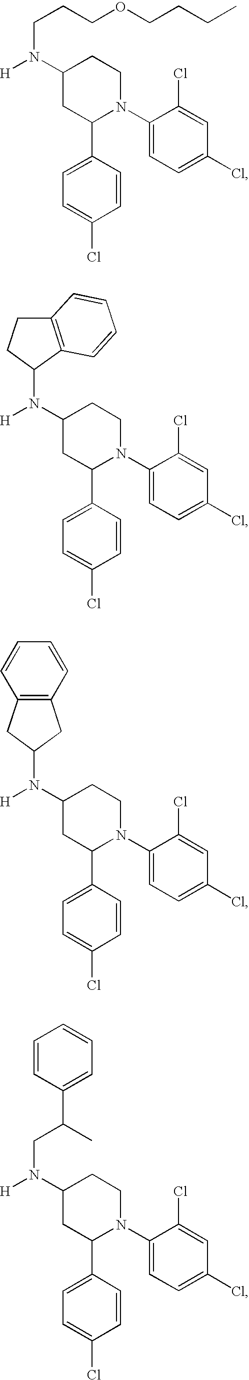

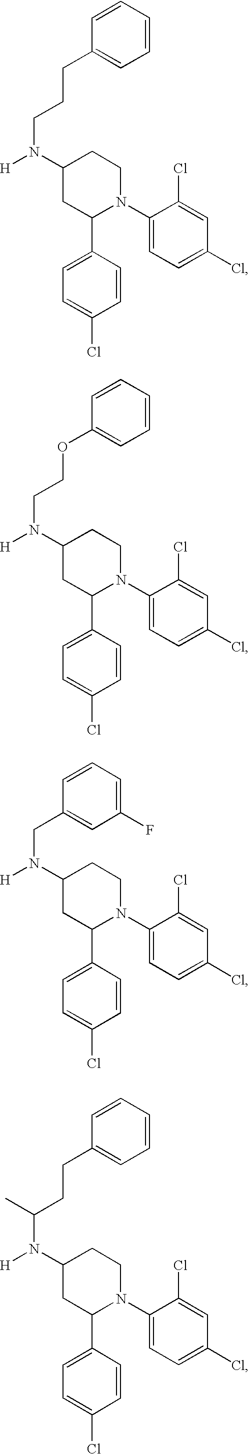

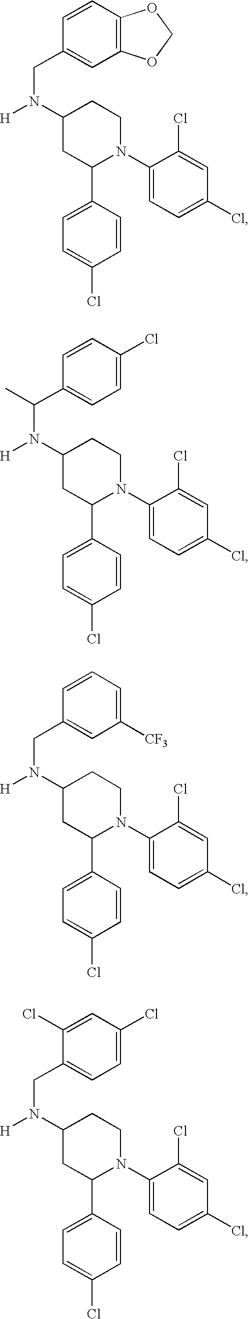

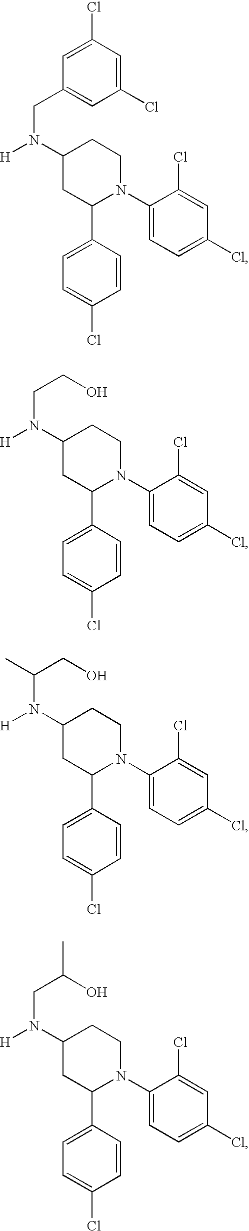

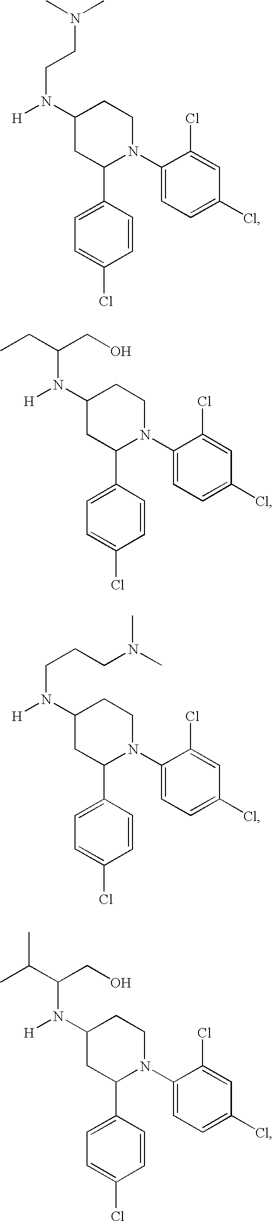

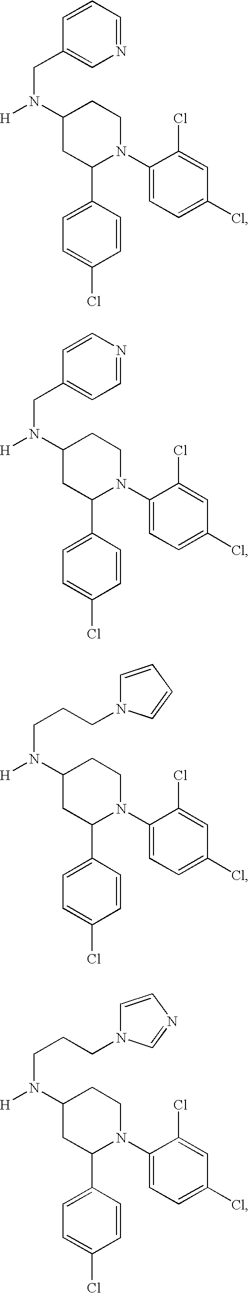

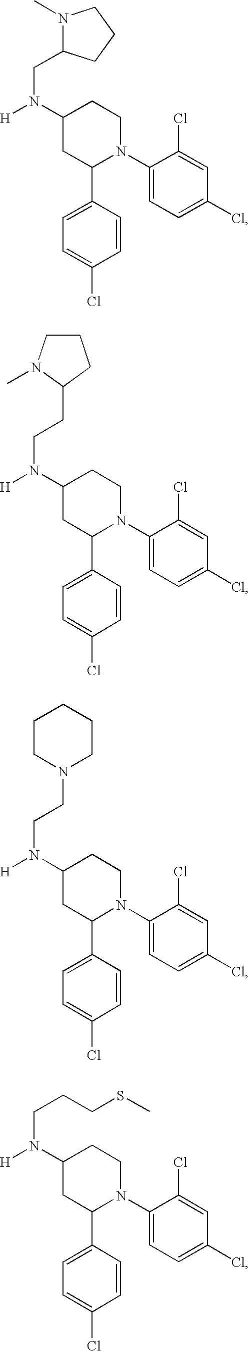

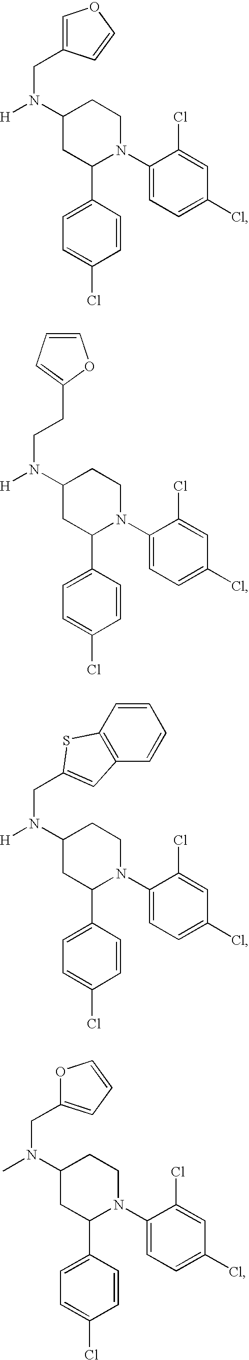

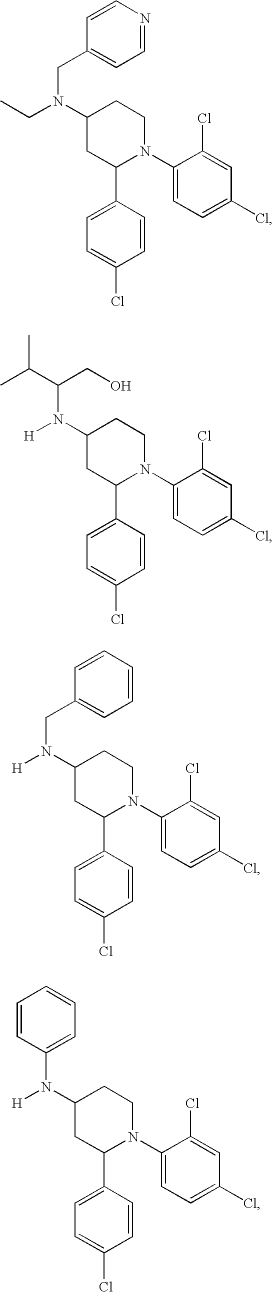

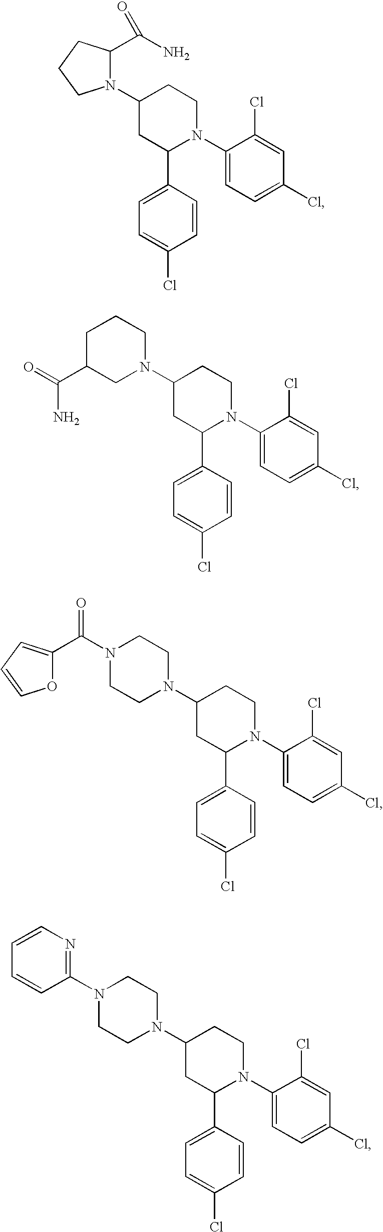

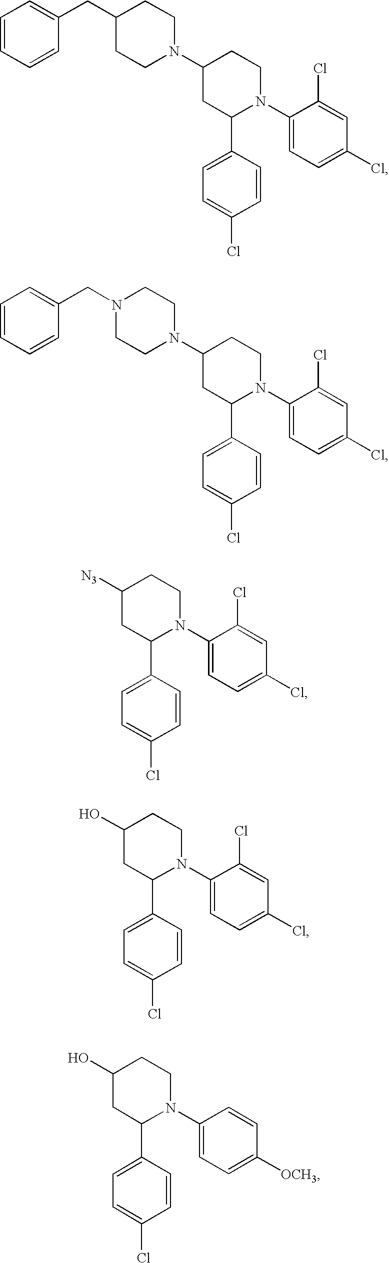

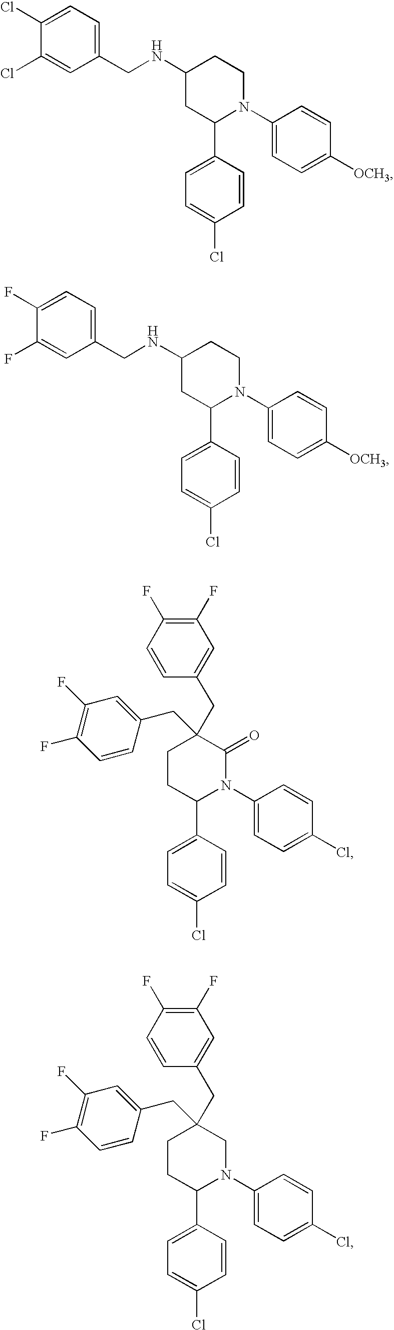

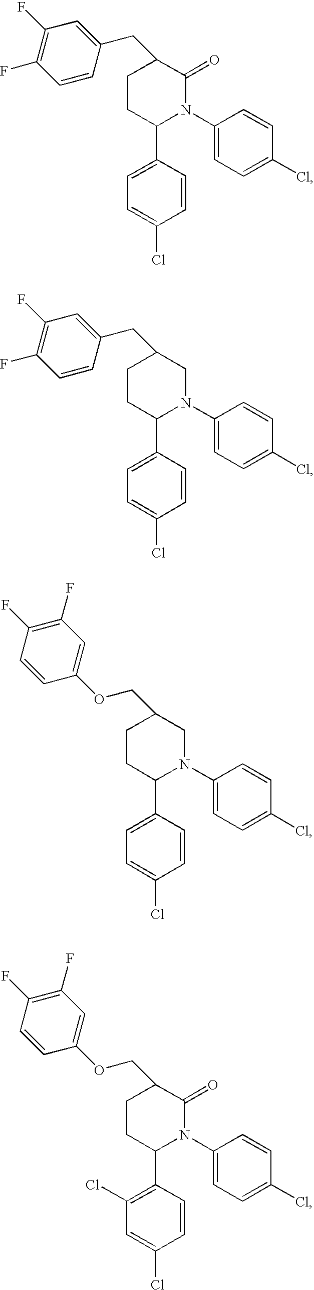

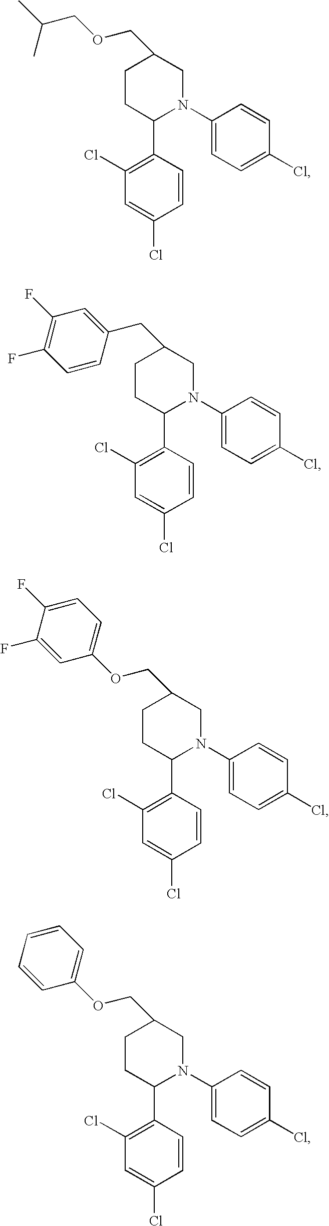

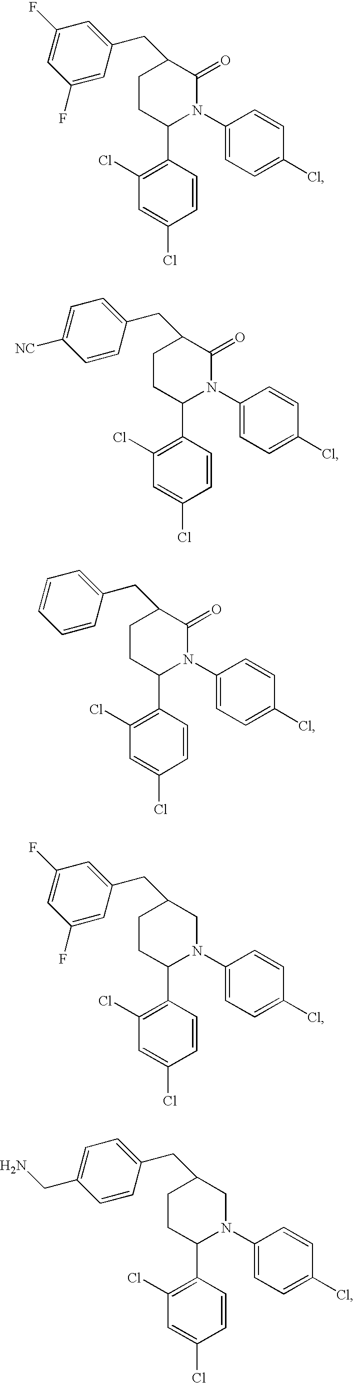

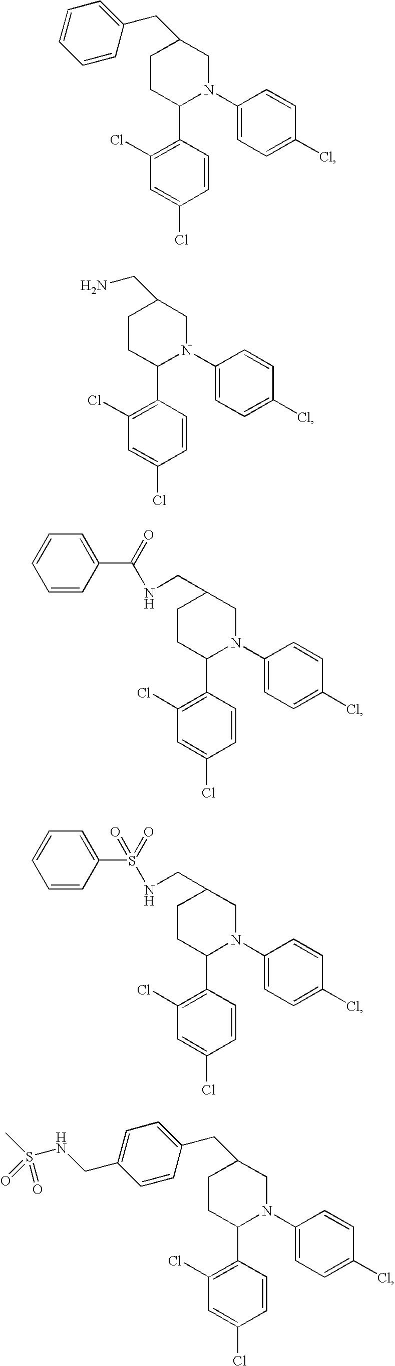

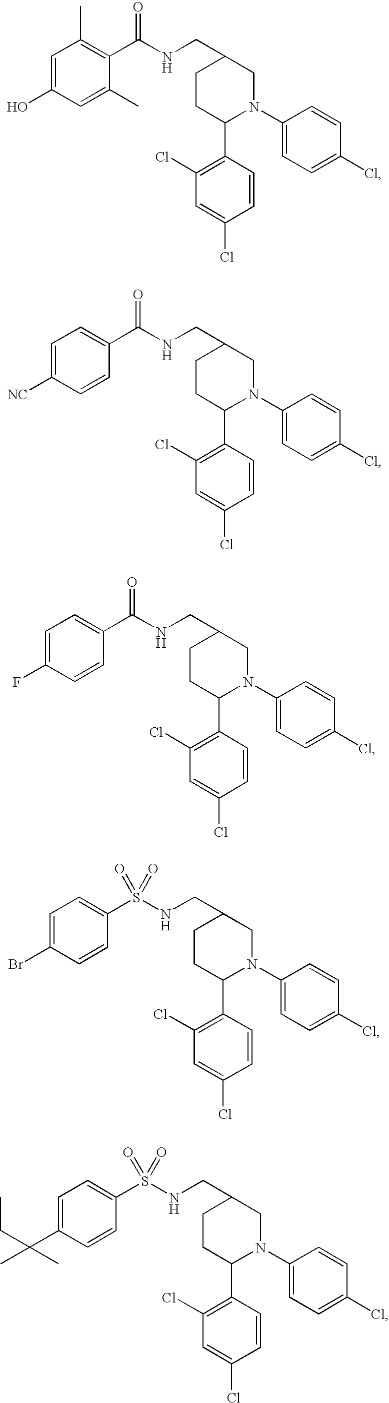

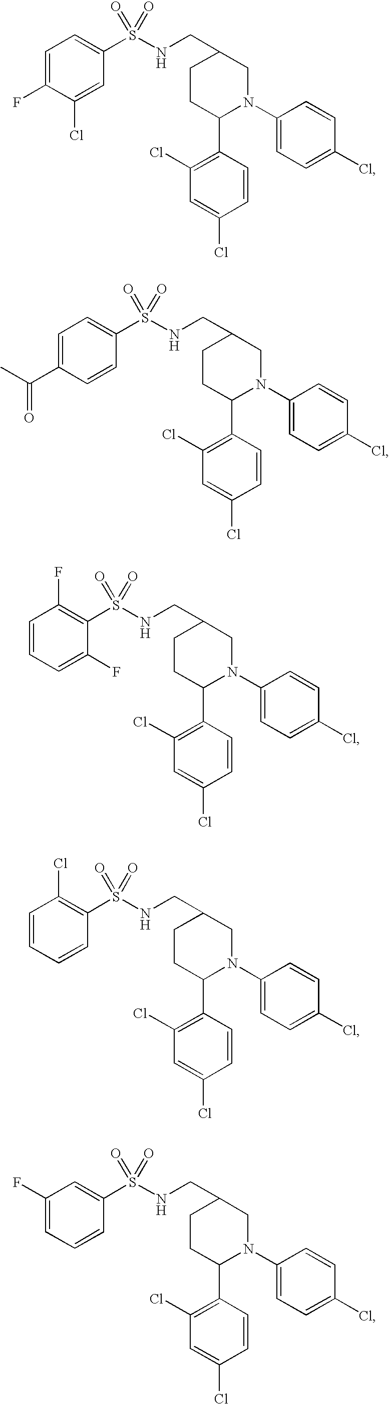

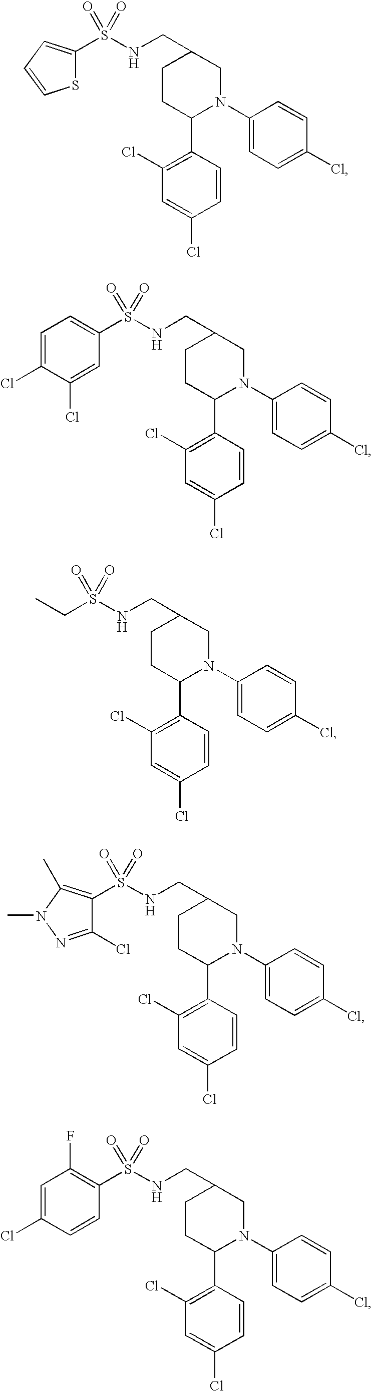

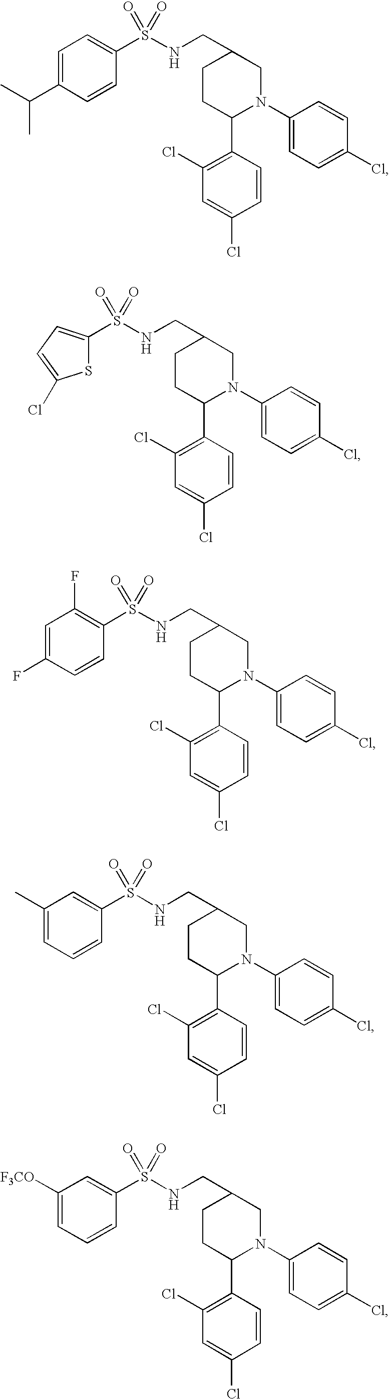

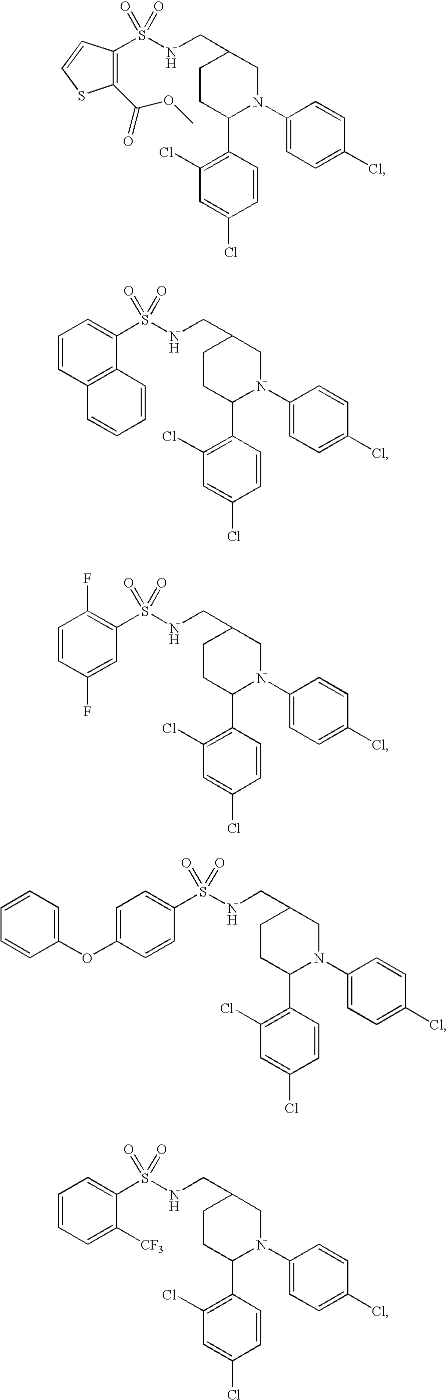

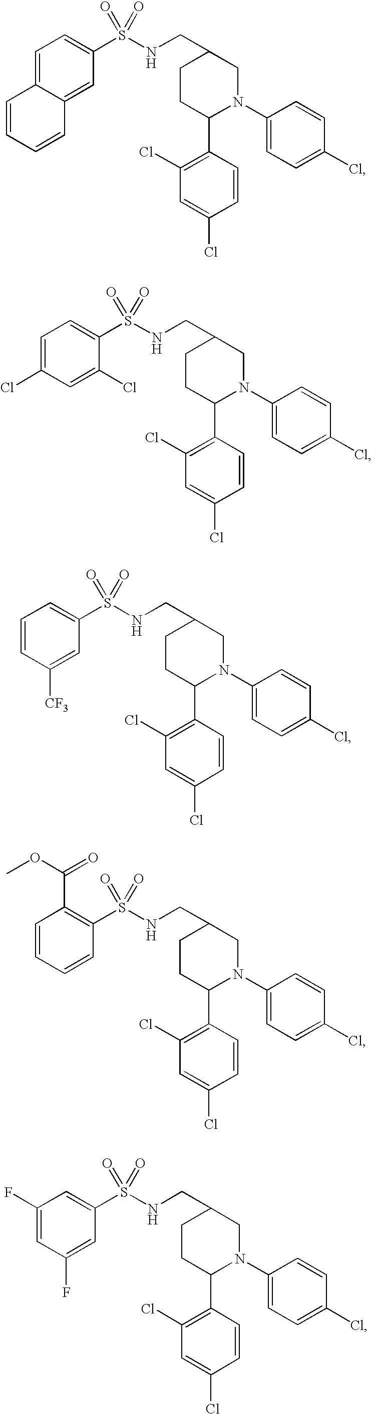

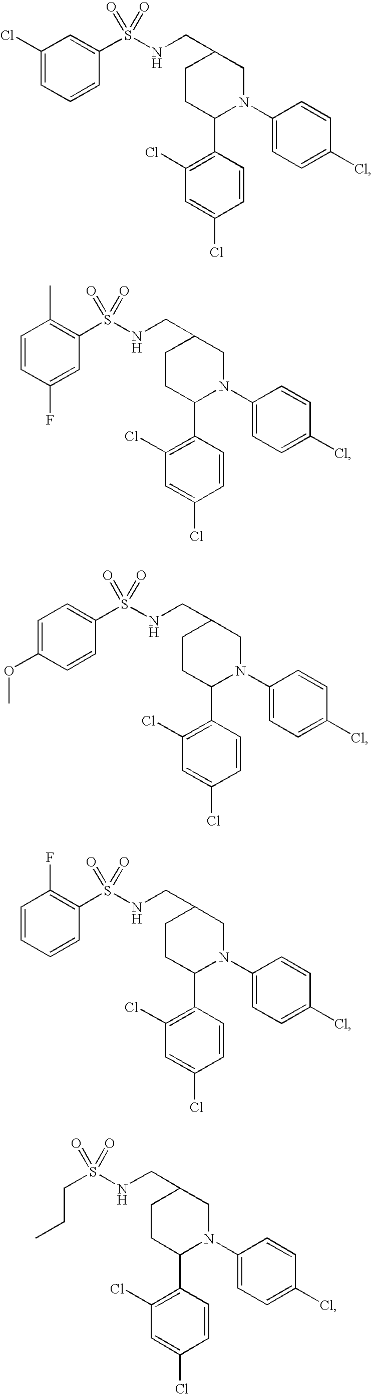

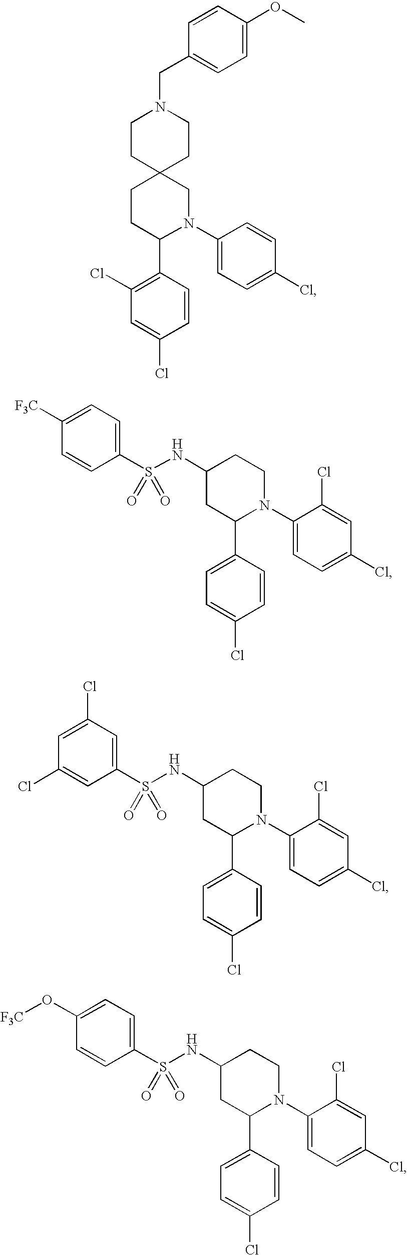

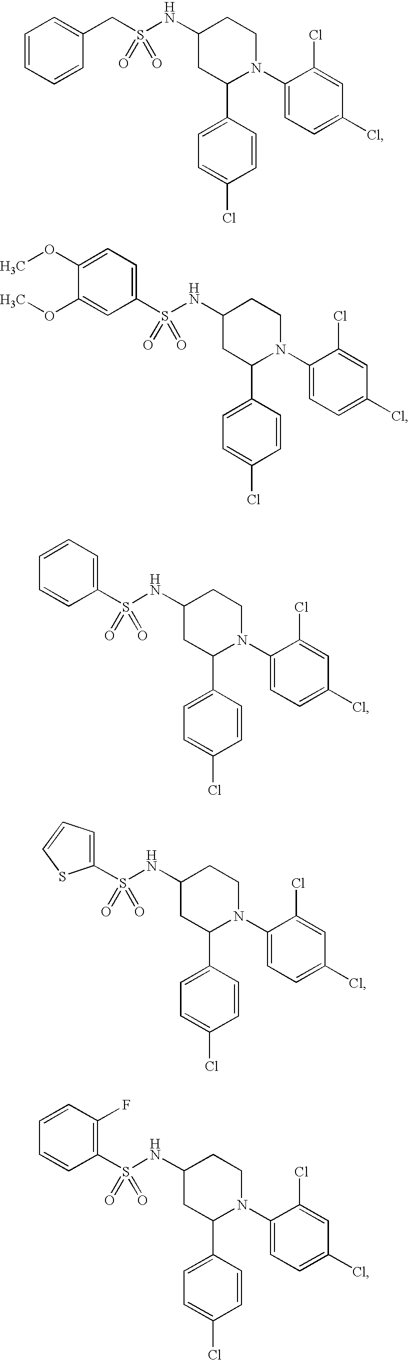

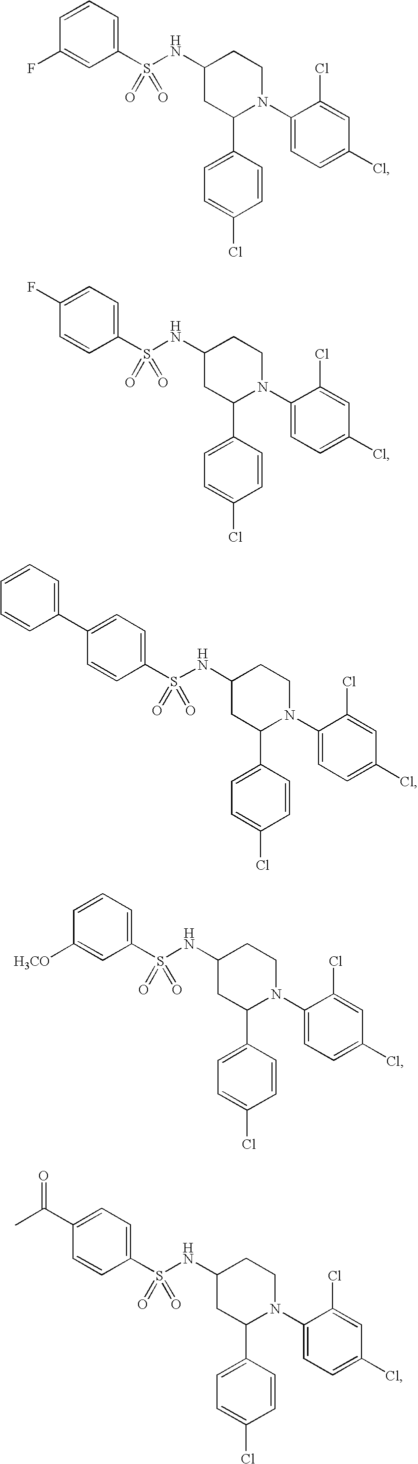

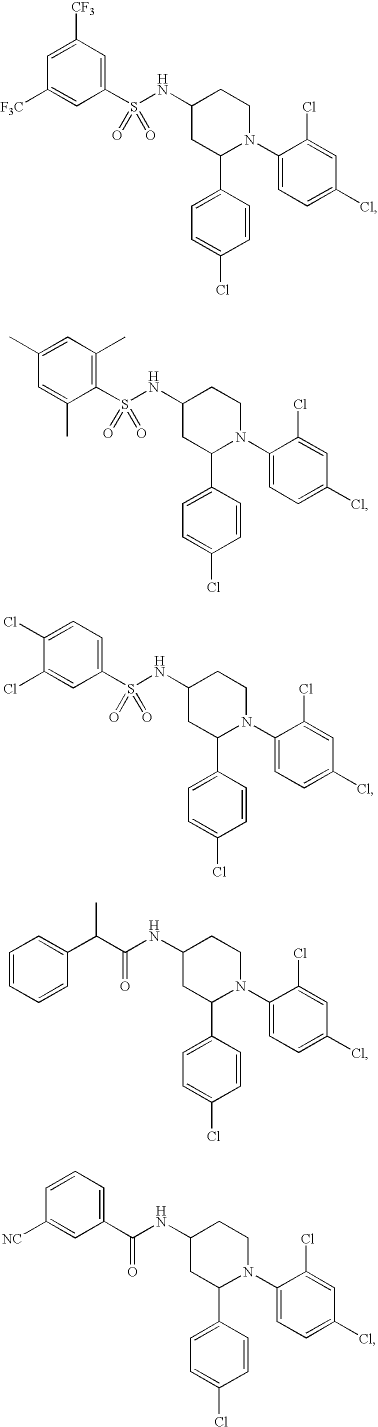

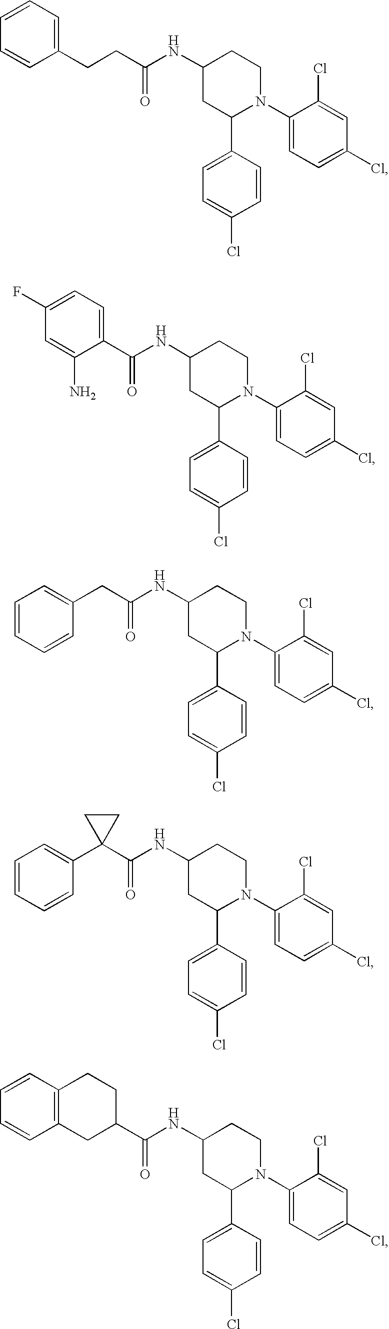

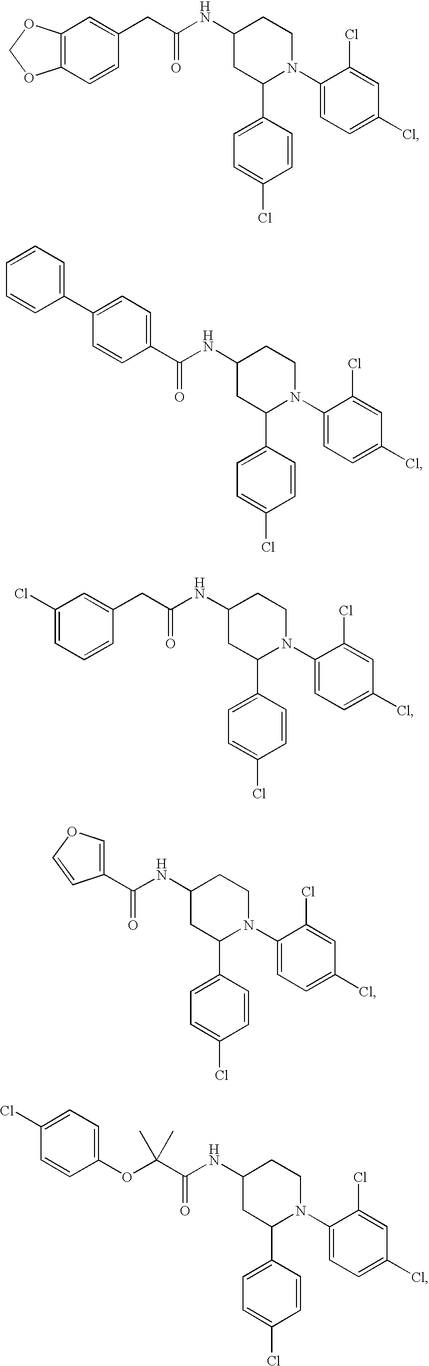

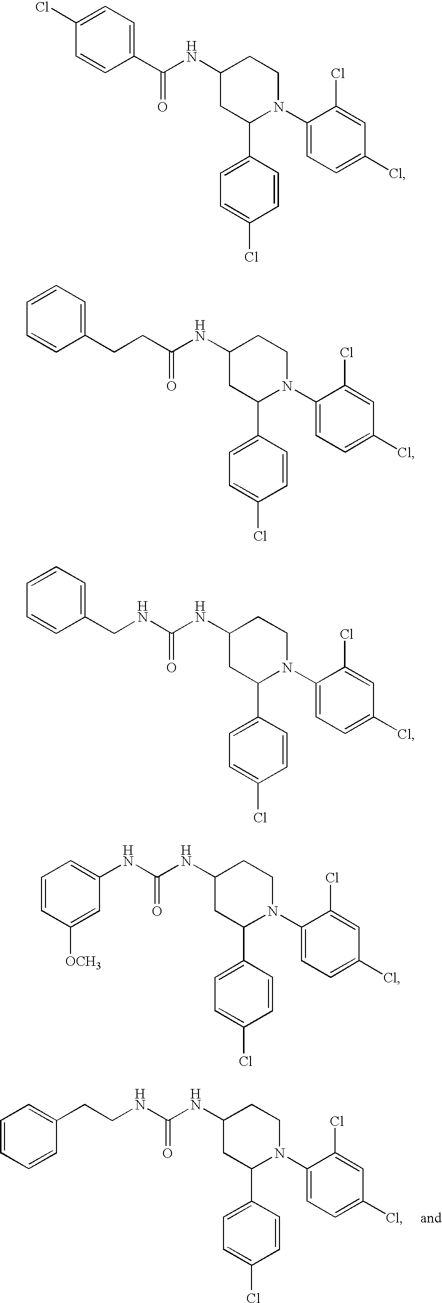

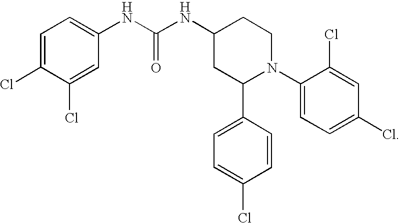

22. The compound of claim 1, a pharmaceutically acceptable salt, solvate or ester thereof having a structure selected from the group consisting of:

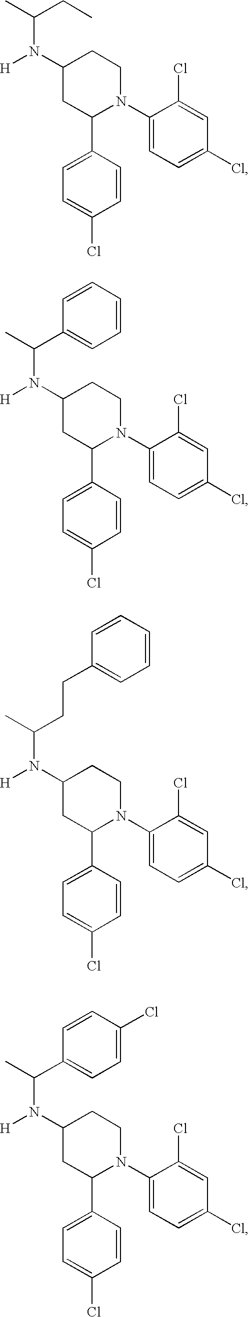

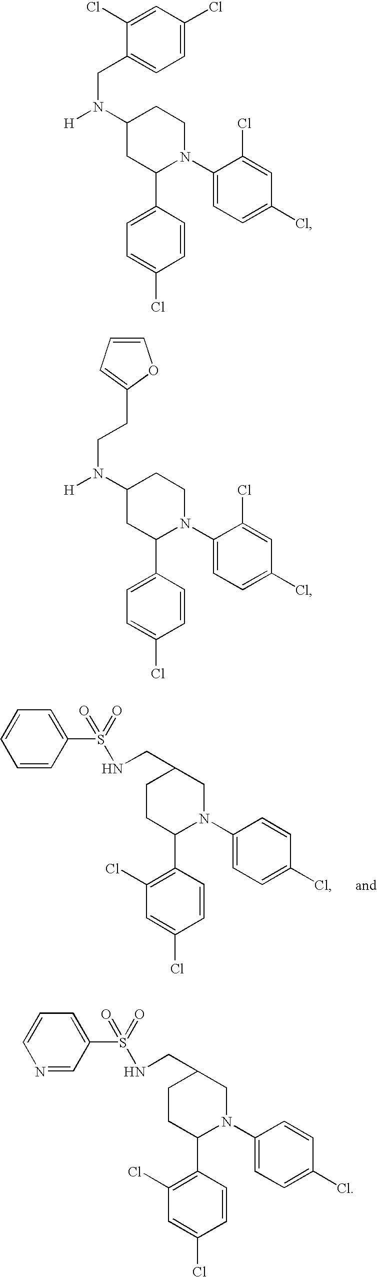

23. The compound of claim 1, a pharmaceutically acceptable salt, solvate or ester thereof having a structure selected from the group consisting of: 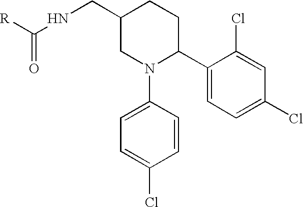
R

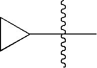

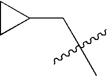

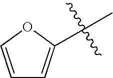

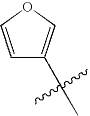

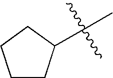

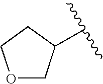

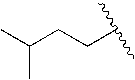

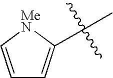

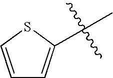

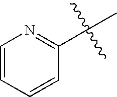

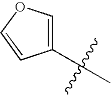

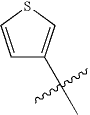

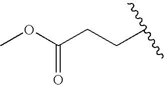

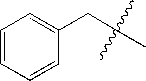

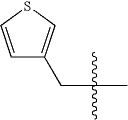

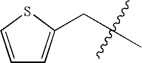

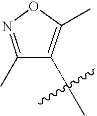

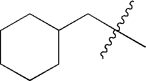

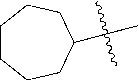

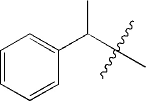

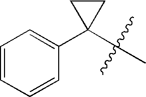

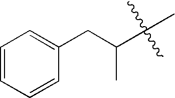

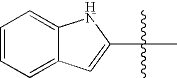

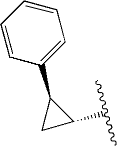

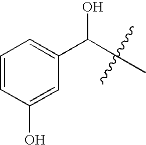

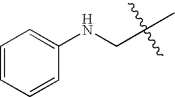

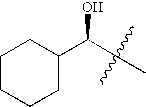

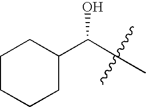

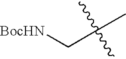

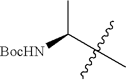

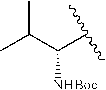

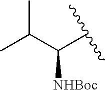

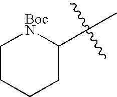

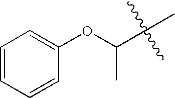

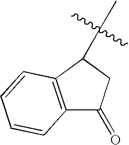

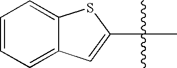

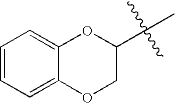

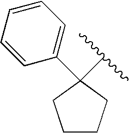

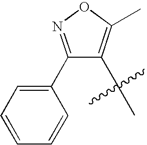

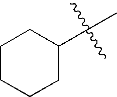

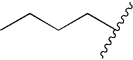

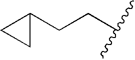

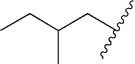

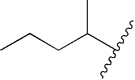

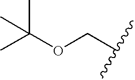

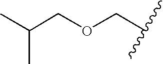

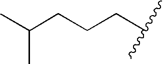

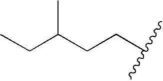

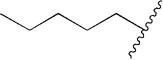

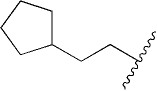

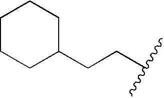

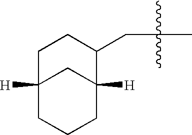

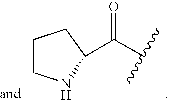

24. The compound of claim 1, a pharmaceutically acceptable salt, solvate or ester thereof having a structure selected from the group consisting of: 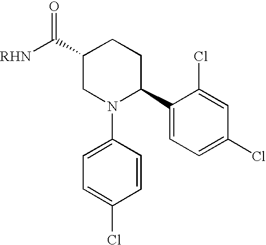
R

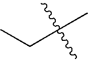

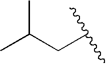

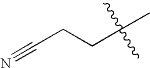

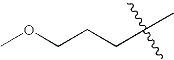

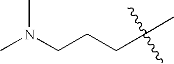

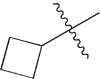

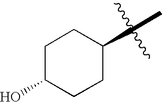

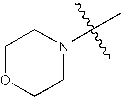

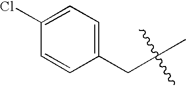

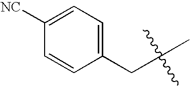

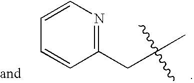

25. The compound of claim 1, a pharmaceutically acceptable salt, solvate or ester thereof having a structure selected from the group consisting of: 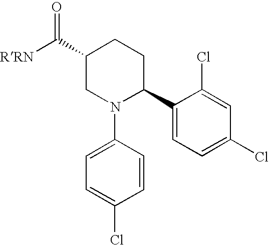
NRR′

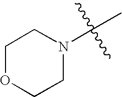

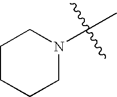

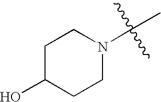

26. The compound of claim 1, a pharmaceutically acceptable salt, solvate or ester thereof having a structure selected from the group consisting of:

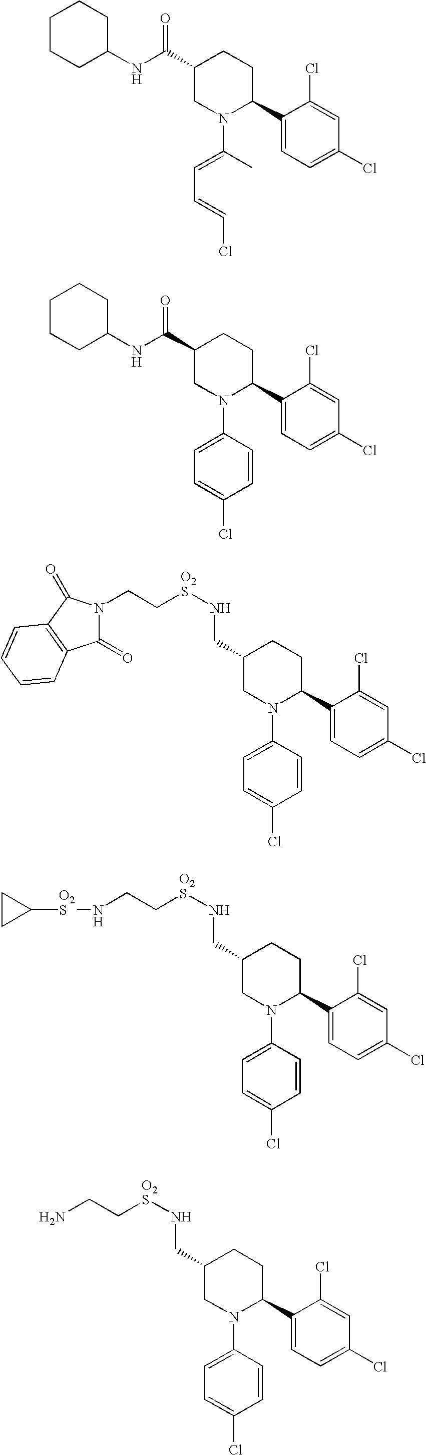

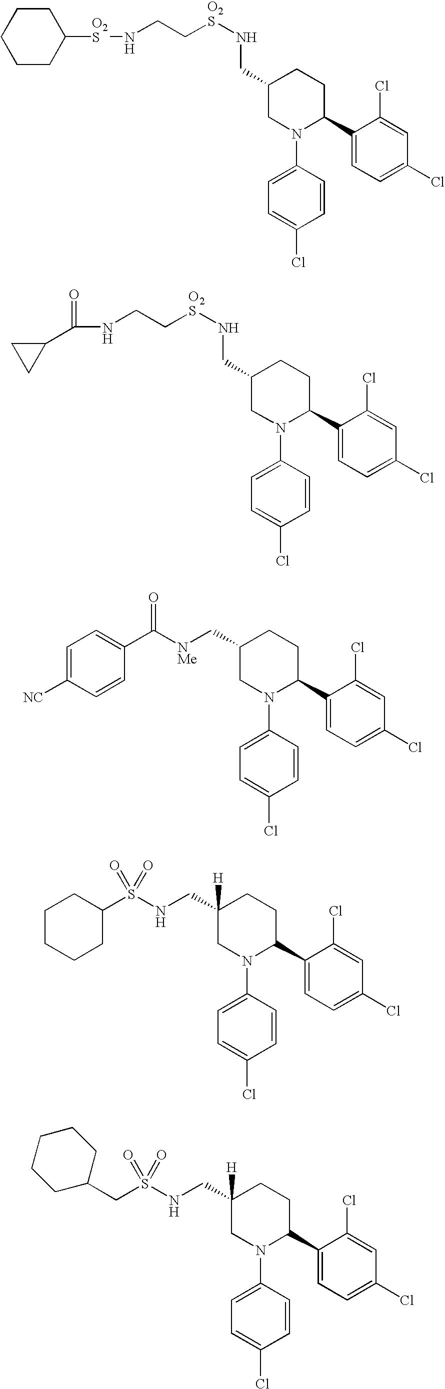

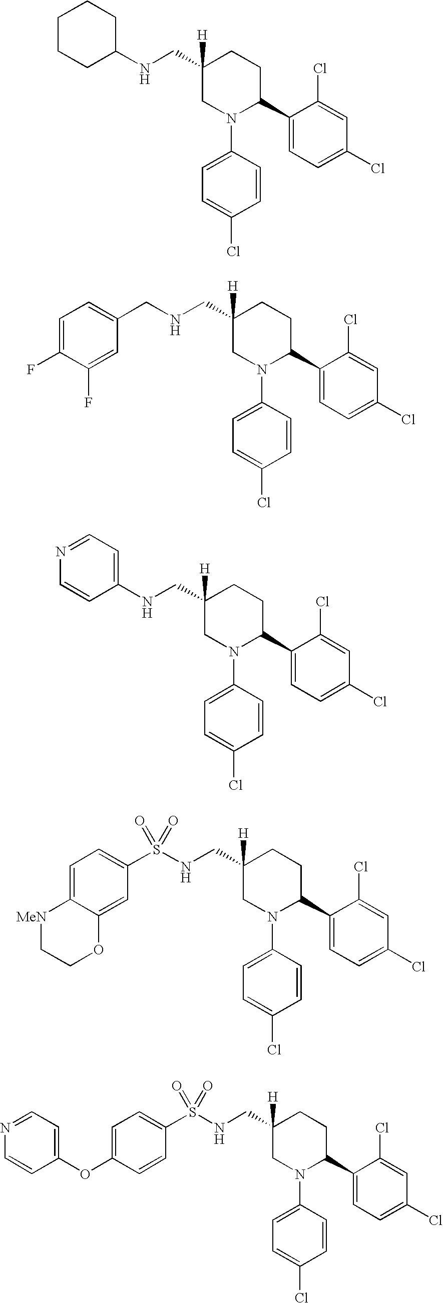

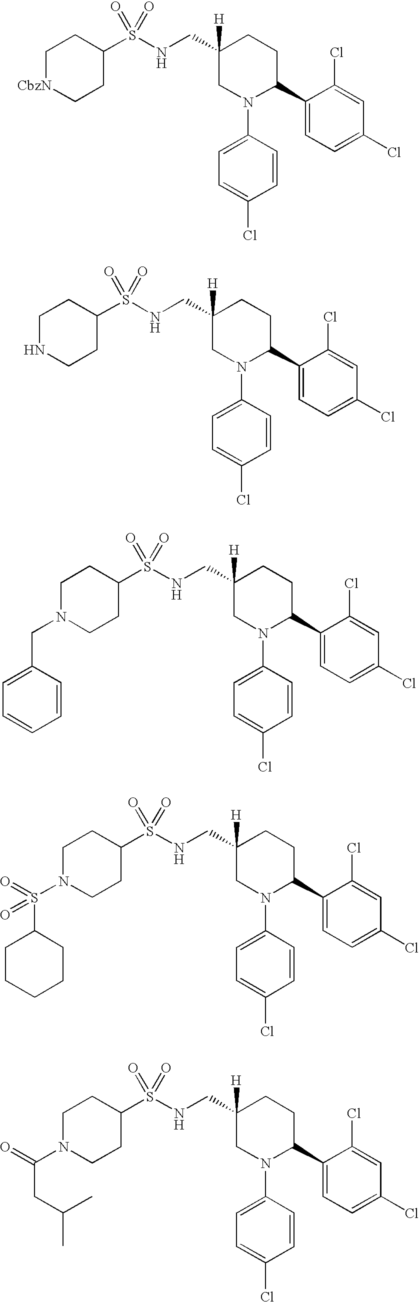

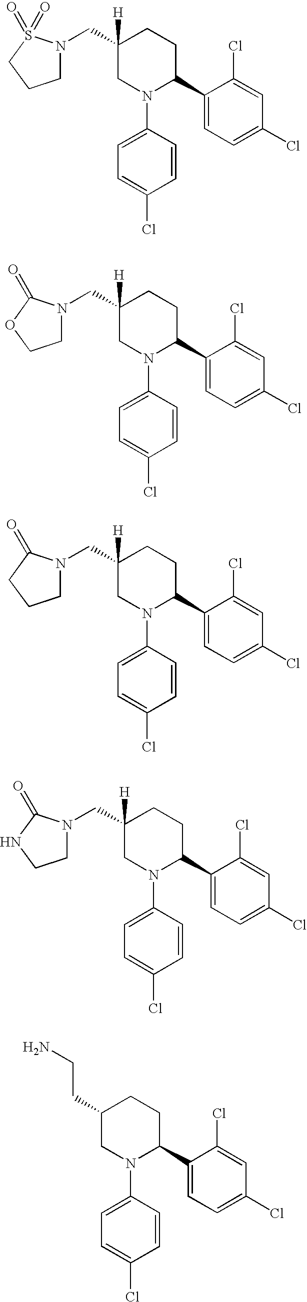

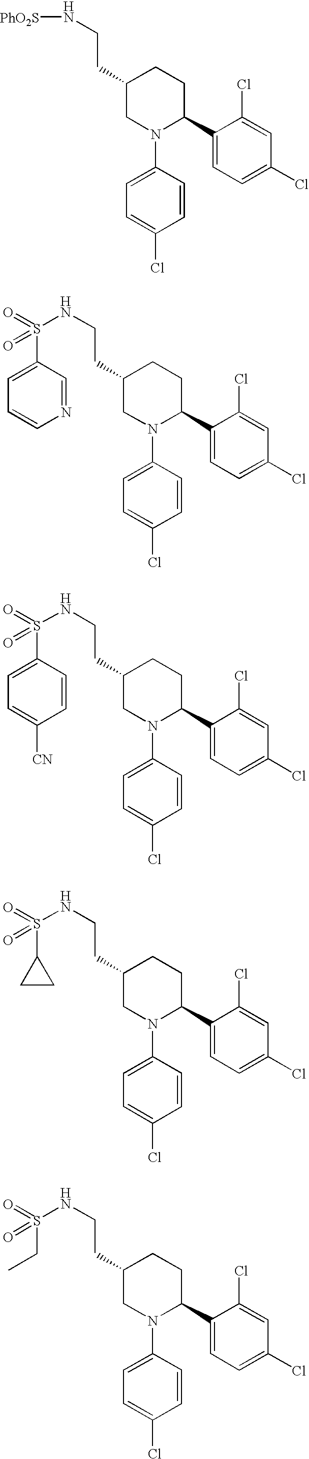

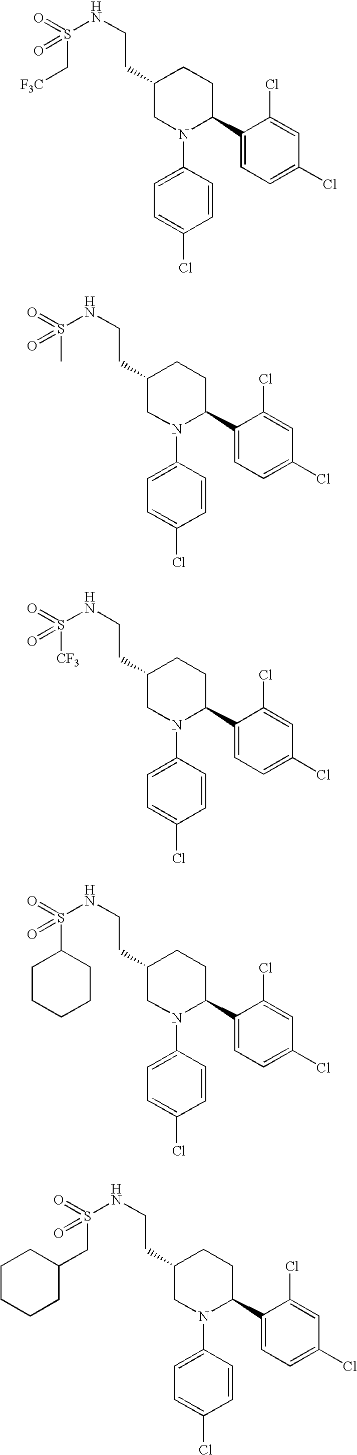

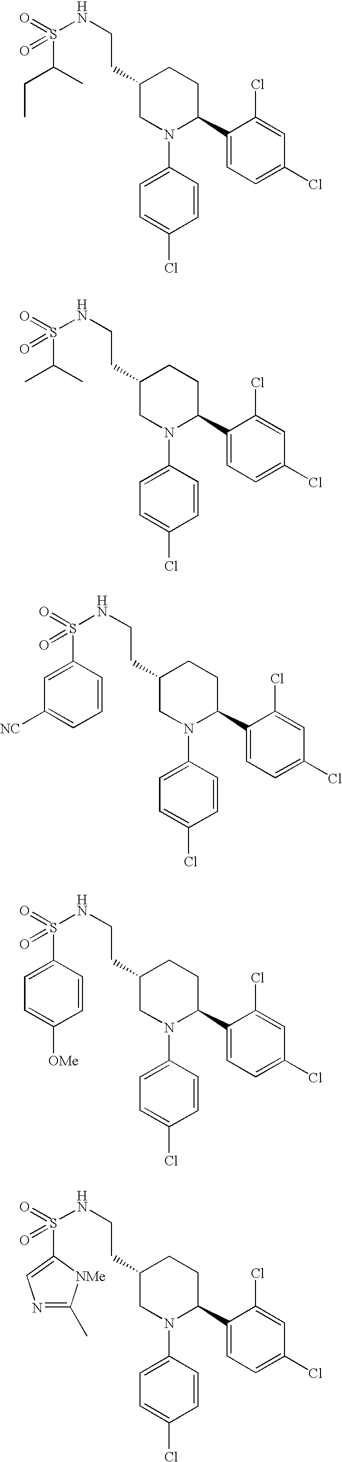

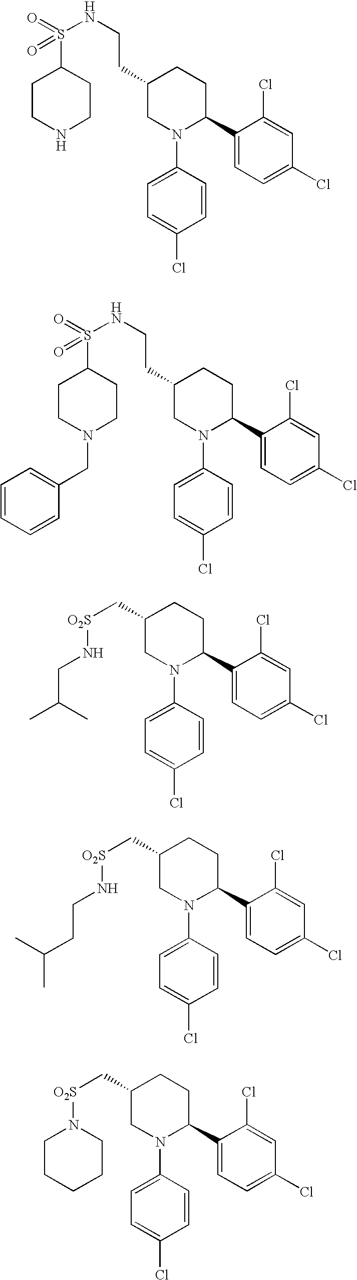

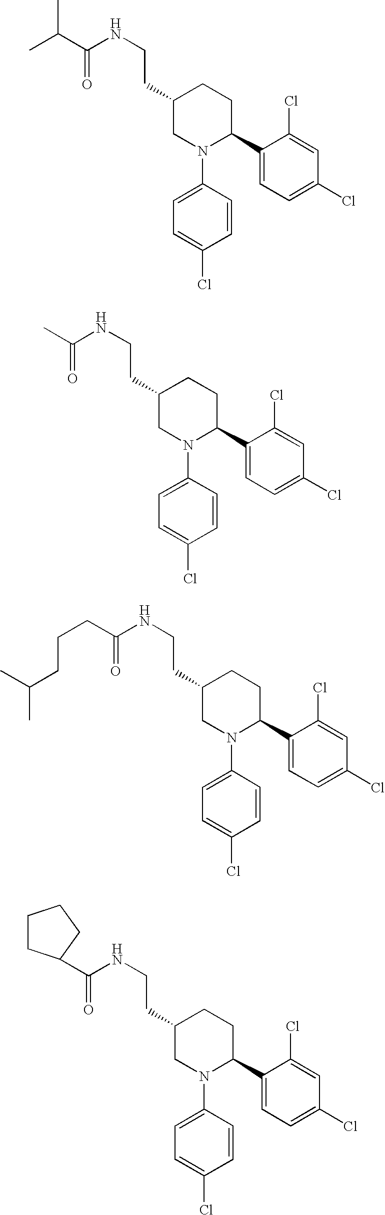

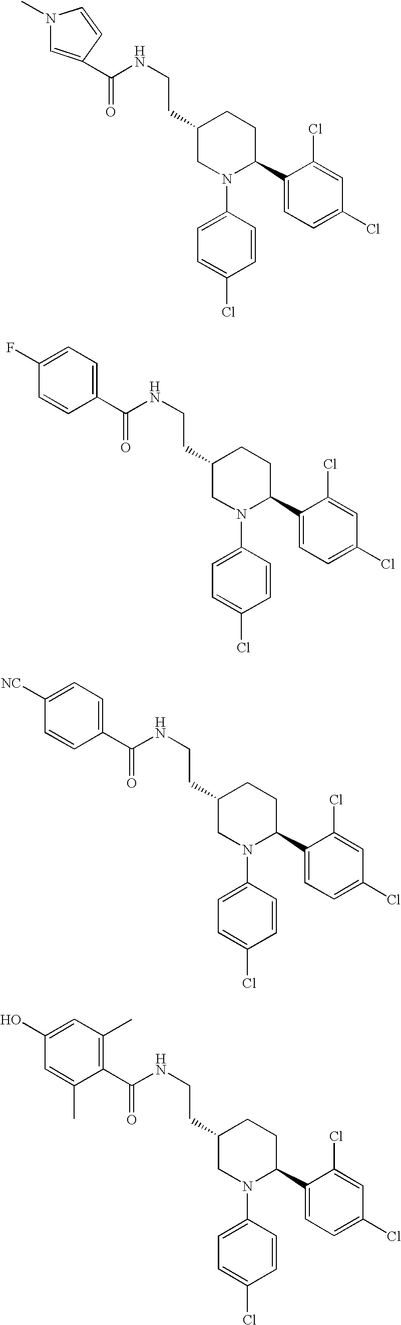

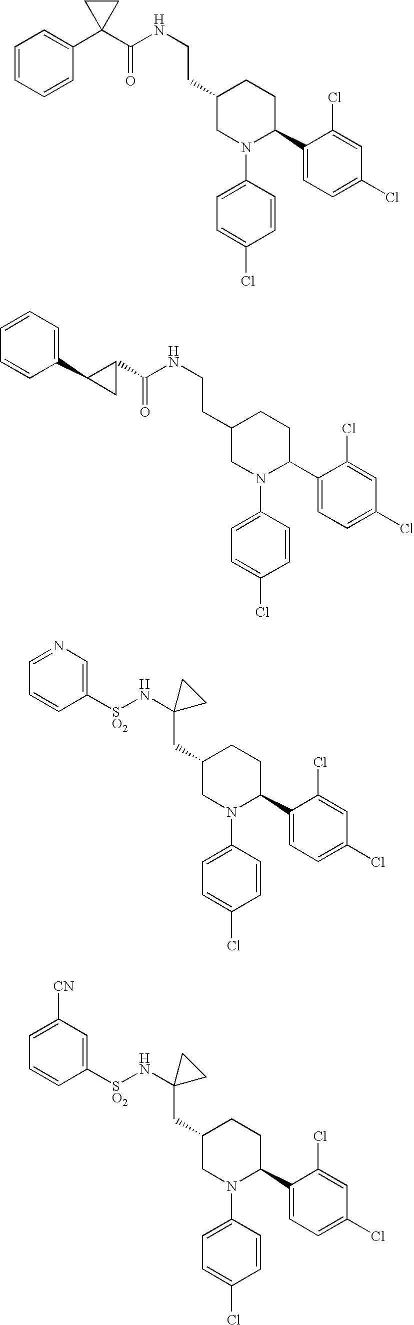

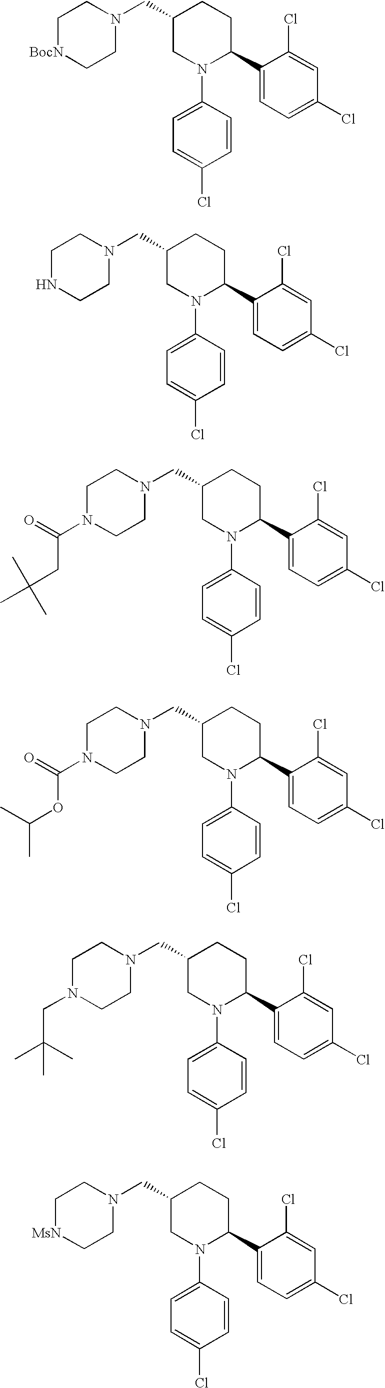

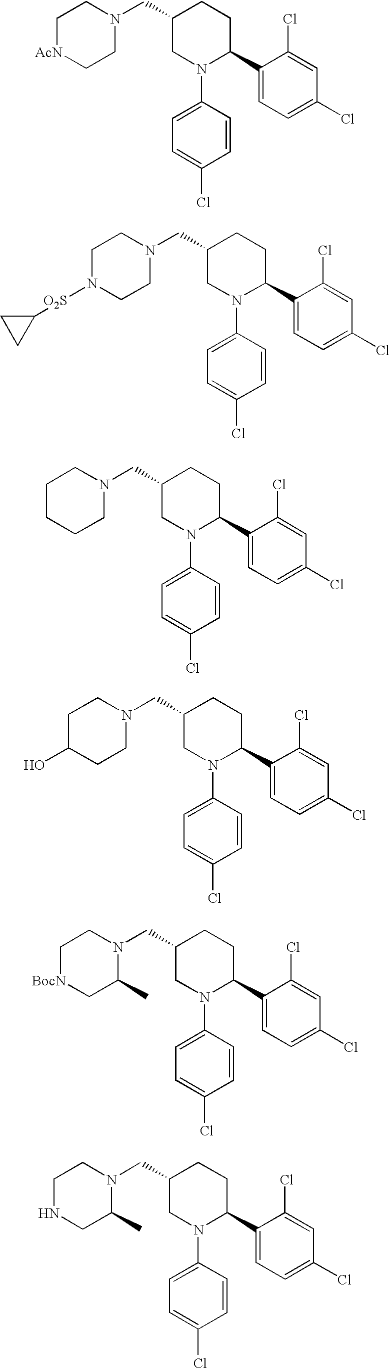

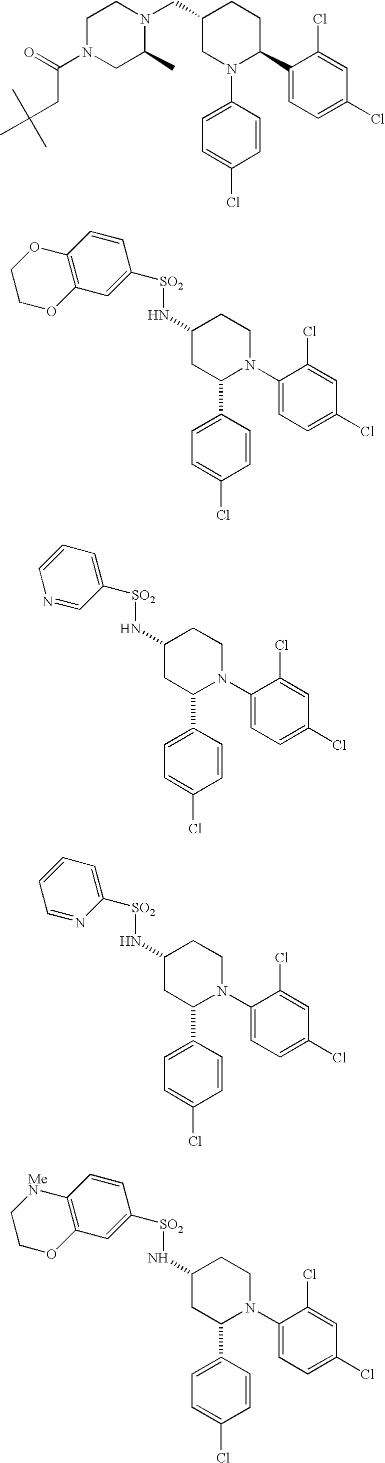

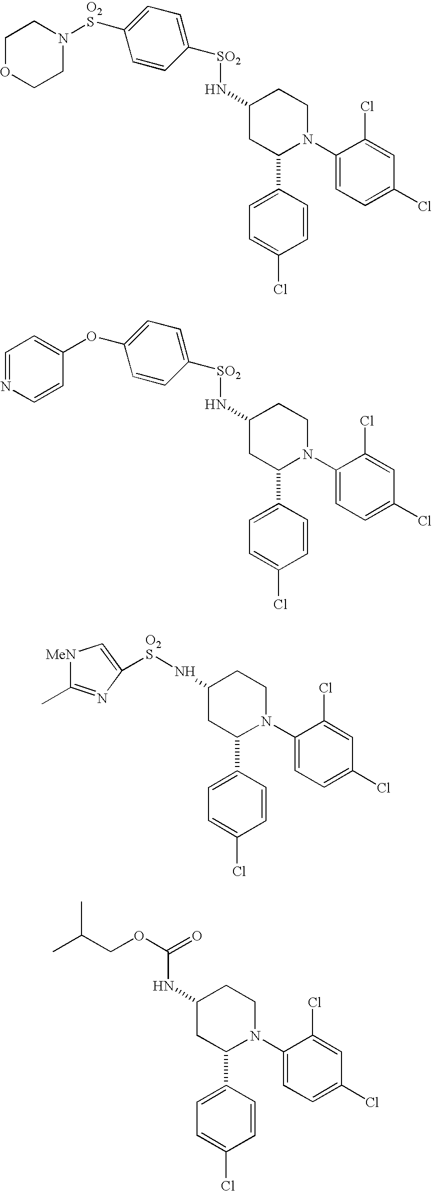

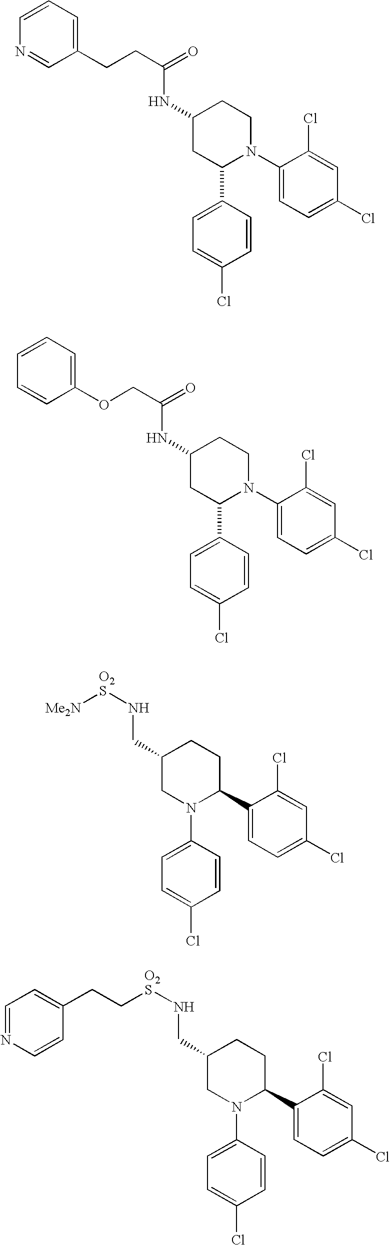

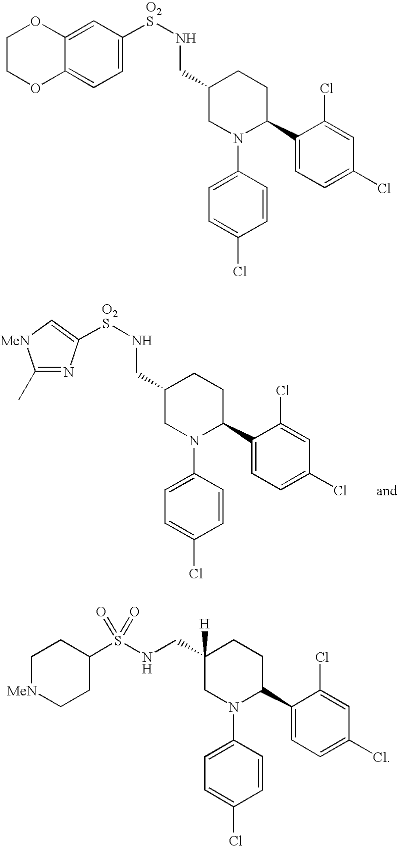

27. The compound of claim 1, a pharmaceutically acceptable salt, solvate or ester thereof having a structure selected from the group consisting of: 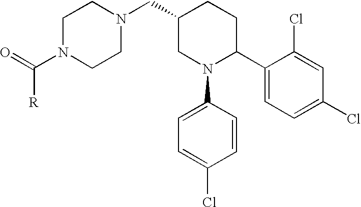
R

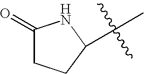

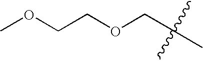

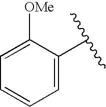

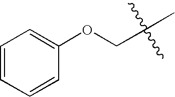

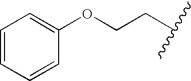

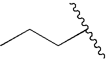

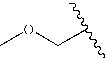

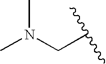

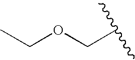

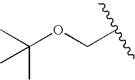

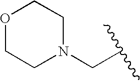

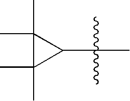

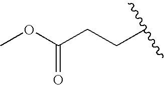

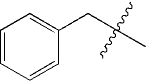

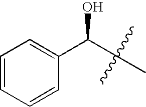

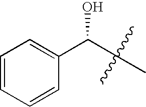

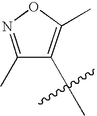

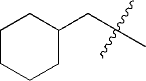

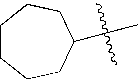

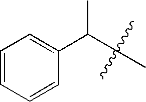

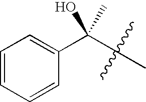

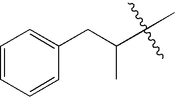

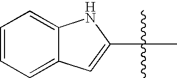

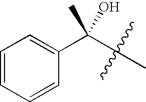

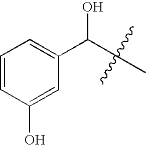

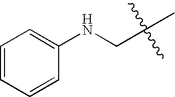

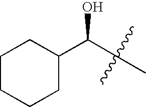

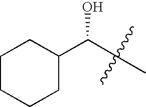

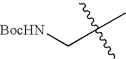

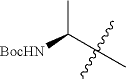

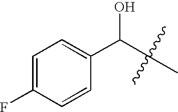

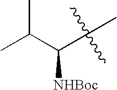

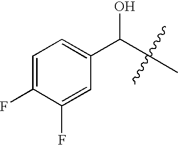

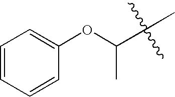

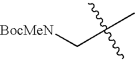

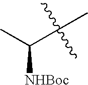

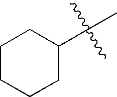

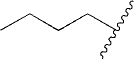

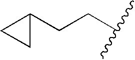

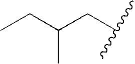

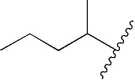

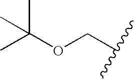

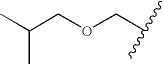

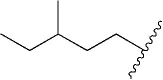

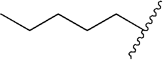

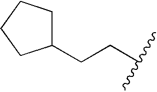

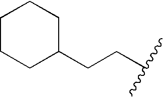

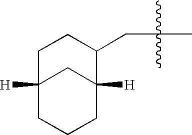

28. A purified compound of claim 1.

29. A compound having the following structural formula: 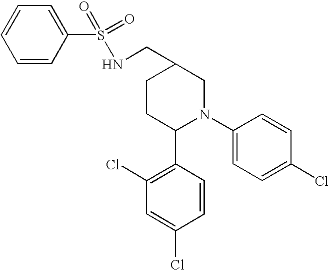
or a pharmaceutically acceptable salt, solvate, or ester thereof.

30. A compound having the following structural formula: 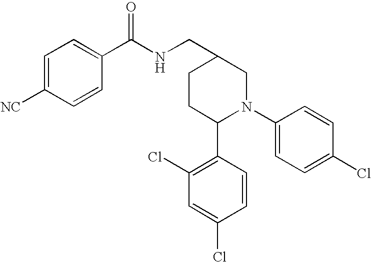
or a pharmaceutically acceptable salt, solvate, or ester thereof.

31. A compound having the following structural formula: 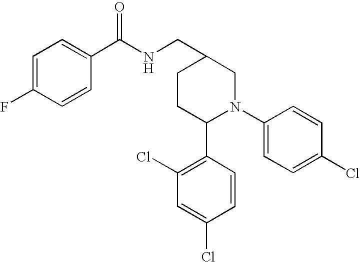
or a pharmaceutically acceptable salt, solvate, or ester thereof.

32. A compound having the following structural formula: 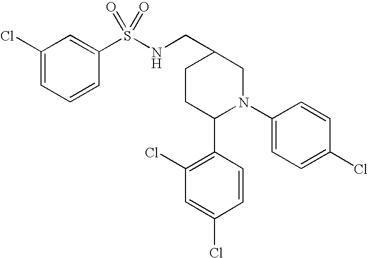
or a pharmaceutically acceptable salt, solvate, or ester thereof.

33. A compound having the following structural formula: 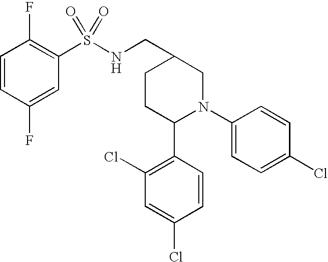
or a pharmaceutically acceptable salt, solvate, or ester thereof.

34. A compound having the following structural formula: 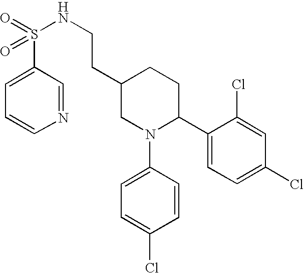
or a pharmaceutically acceptable salt, solvate, or ester thereof.

35. A compound having the following structural formula: 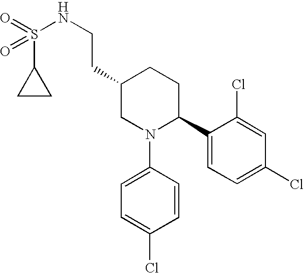
or a pharmaceutically acceptable salt, solvate, or ester thereof.

36. A composition comprising:
a compound of claim 1, or a pharmaceutically acceptable salt, solvate, or ester thereof; and
at least one pharmaceutically acceptable carrier.

37. A pharmaceutical composition comprising a compound of claim 1, or a pharmaceutically acceptable salt, solvate, or ester thereof, in combination with at least one additional therapeutic agent.

38. The pharmaceutical composition of claim 37, wherein said additional therapeutic agent comprises an antiobesity agent, an antidiabetic agent, or lipid lowering agent.

39. The pharmaceutical composition of claim 38, wherein:
said antiobesity agent is selected from the group consisting of rimonabant, orlistat, sibutramine, bromocriptine, ephedrine, leptin, pseudoephedrine, and PYY₃₋₃₆;
said antidiabetic agent is selected from the group consisting of PPARγ agonist, PPARα/γ dual agonist, biguanidine, sulfonylurea, meglitinide, insulin, insulin secretagogue, and a dipeptidyl peptidase IV inhibitor; and
said lipid lowering agent is selected from the group consisting of a bile acid sequesterant, an HMG-CoA reductase inhibitor, a cholesterol absorption inhibitor, an ACAT inhibitor, a CETP inhibitor, a PPARα agonist, niacin and a niacin receptor agonist.

40. A method of treating a disease, disorder, or condition comprising:
administering to a patient in need thereof a therapeutically effective amount of at least one compound of claim 1, or a pharmaceutically acceptable salt, solvate, or ester thereof;
wherein said disease, disorder, or condition is selected from the group consisting of metabolic syndrome, neuroinflammatory disorders, cognitive disorders, psychosis, addictive behavior, gastrointestinal disorders, and cardiovascular conditions.

41. The method of claim 40, wherein said disease, disorder, or condition is metabolic syndrome.

42. The method of claim 41, further comprising administering at least one additional therapeutic agent selected from the group consisting of an antiobesity agent, an antidiabetic agent, or lipid lowering agent.

43. The method of claim 42, wherein:
said antiobesity agent is selected from the group consisting of rimonabant, orlistat, sibutramine, bromocriptine, ephedrine, leptin, pseudoephedrine, and PYY₃₋₃₆;
said antidiabetic agent is selected from the group consisting of PPARγ agonist, dual agonist, biguanidine, sulfonylurea, meglitinide, insulin, insulin secretagogue, and a dipeptidyl peptidase IV inhibitor; and
said lipid lowering agent is selected from the group consisting of a bile acid sequesterant, an HMG-CoA reductase inhibitor, a cholesterol absorption inhibitor, an ACAT inhibitor, a CETP inhibitor, a PPARα agonist, niacin and a niacin receptor agonist.
